(19) United States
(12) Patent Application Publication
(10) Pub. No.: US 2003/0134302 A1
(43) Pub. Date: Jul. 17, 2003

(54) Libraries of expressible gene sequences

(75) Inventors: Joseph Manuel Fernandez, Carlsbad, CA (US); John Alastair Heyman, Cardiff-by-the-Sea, CA (US); James Paul Hoeffler, Carlsbad, CA (US)

Correspondence Address: Lisa A. Haile, J.D., Ph.D., GRAY CARY WARE & FREIDENRICH LLP, Suite 1100, 4365 Executive Drive, San Diego, CA, 92121-2133, US

(73) Assignee: INVITROGEN CORPORATION

(21) Appl. No.: 10/210,985

(22) Filed: Aug. 1, 2002

Related U.S. Application Data
(63) Continuation of application No. 10/003,021, filed on Nov. 14, 2001.
Application No. 10/003,021 is a continuation of application No. 09/285,386, filed on Apr. 2, 1999, now abandoned.
(60) Provisional application No. 60/096,981, filed on Aug. 18, 1998.
(60) Provisional application No. 60/080,626, filed on Apr. 3, 1998.

(51) Int. Cl. C12Q 1/68; C07H 21/04; C12P 21/02; C12N 5/06

(52) U.S. Cl. 435/6; 435/69.1; 435/320.1; 435/325; 536/23.2

(57) ABSTRACT

The invention described herein comprises libraries of expressible gene sequences. Such gene sequences are contained on plasmid vectors designed to endow the expressed proteins with a number of useful features such as affinity purification tags, epitope tags, and the like. The expression vectors containing such gene sequences can be used to transfect cells for the production of recombinant proteins. A further aspect of the invention comprises methods of identifying binding partners for the products of such expressible gene sequences.

### RELATED APPLICATIONS

This application relies for priority on U.S. Provisional Application No. 60/080,626, filed Apr. 3, 1998, and U.S. Provisional Application No. 60/096,981, filed Aug. 18, 1998, each of which is hereby incorporated herein in its entirety.

### FIELD OF THE INVENTION

The invention disclosed herein relates to the fields of genomics and molecular biology. More specifically the invention relates to libraries of expressible gene sequences and recombinant cells transfected therewith.

### BACKGROUND OF THE INVENTION

Recent breakthroughs in nucleic acid sequencing technology have made possible the sequencing of entire genomes from a variety of organisms, including humans. The potential benefits of a complete genome sequence are many, ranging from applications in medicine to a greater understanding of evolutionary processes. These benefits cannot be fully realized, however, without an understanding of how and where these newly sequenced genes function.

Traditionally, functional understanding started with recognizing an activity, isolating a protein associated with that activity, then identifying and isolating the gene, or genes, encoding that protein. Each gene of interest was identified, isolated and expressed separately, a relatively time consuming process.

Recently, breakthroughs in high through-put DNA sequencing technology have allowed massive amounts of gene sequence information to become available to the public. Yet methods of expressing these sequences to produce the proteins encoded by them for study have still required that each sequence be manipulated one at a time. Accordingly, a need exists for large numbers of expressible gene sequences. The invention described herein addresses this and related needs as will become apparent upon inspection of the specification and the appended claims.

### BRIEF DESCRIPTION OF THE INVENTION

The present invention comprises libraries of expressible gene sequences. Such gene sequences are contained on plasmid vectors designed to endow the expressed proteins with a number of useful features such as affinity purification tags, epitope tags, and the like. The expression vectors containing such gene sequences can be used to transfect cells for the production of recombinant proteins.

A further aspect of the invention comprises methods of identifying binding partners for the products of such expressible gene sequences.

### BRIEF DESCRIPTION OF THE FIGURES

FIG. 1 shows a schematic representation of the vaccinia topoisomerase type I cloning method used in the practice of the invention method.

### DETAILED DESCRIPTION OF THE INVENTION

The present invention comprises libraries of expressible gene sequences. Such gene sequences are contained on expression vectors which can be useful for transfecting cells and producing recombinant proteins. The expression vectors may additionally contain sequences that will endow the expressed proteins with a variety of useful features, such as peptides that aid in purification, epitope tags useful in identifying recombinant protein, and the like.

The libraries of the invention are created by employing a high through-put methodology comprised of several steps. In the first step, the gene sequences that are to be expressed are amplified. By “amplification” it is meant that the copy number of the gene sequence(s) is increased. One commonly used method of amplification is the polymerase chain reaction (PCR). In brief, starter DNA is heat-denatured into single strands. Two synthetic oligonucleotides, one complementary to sequence at the 3′ end of the sense strand of DNA segment of interest and the other complementary to the sequence at the 3′ end of the anti-sense strand of a DNA segment of interest, are added in excess to the DNA sequence to be amplified and the temperature is lowered to 50-60° C. The specific oligonucleotides hybridize with the complementary sequences in the DNA and then serve as primers of DNA chain synthesis, which requires the addition of a supply of deoxynucleotidesand a temperature-resistant DNA polymerase, such as Taq polymerase, which can extend the primers at temperatures up to 72° C. When synthesis is complete, the whole mixture is heated further (up to 95° C.) to melt the newly formed DNA duplexes. When the temperature is lowered again, another round of synthesis takes place, since an excess of primer should still be present. Repeated cycles of synthesis and melting quickly amplify the sequence of interest. A more detailed description of PCR can be found in Erlich, Ed, PCR Technology: Principles and Applications for DNA Amplification, W. H. Freeman and Co., 1992 and Erlich, et al., Eds, Polymerase Chain Reaction, Cold Spring Harbor Laboratory, 1989, both of which are incorporated by reference herein.

Starter DNA can come from a variety of sources. It can be total genomic DNA from an organism, for example, or can be cDNA that has been synthesized from cellular mRNA using reverse transcriptase. Genomic DNA and cDNA are distinguished in that genomic DNA contains introns, DNA which is spliced out during post-transcriptional RNA processing and cDNA does not. Sources of suitable RNA include normal and diseased tissues, cellular extracts, and the like.

The desired gene sequences can come from any source. The examples presented below show the amplification of all open reading frames (ORFs) from a single organism, Saccharomyces cerevisiae, for example. By “open reading frame” it is meant a segment of DNA that exists between a start codon and a stop codon and is likely to represent a gene. An open reading frame is also sometimes called a coding region to indicate that it contains only those nucleic acids that actually encode a protein. The examples presented below further show the amplification of a group of human genes thought to be important in the development of cancer.

Public databases exist that contain the entire or partial genome of a particular organism, for example yeast (Saccharomyces cerevisiae), prokaryotes (Bacillus subtilis, E. coli, Borrelia burgdorferi, Helicobacter pylori, Mycoplasma genitalium, and the like), fish (Fugu rubripes), mammals (human, mouse), plants (rice, cotton) and the like. Well known databases include GenBank, Unigene, EMBL, IMAGE and TIGR, for example. Public databases such as these can be used a source of gene sequences for use in the method of the invention. Such DNA sequence databases generally give each unique sequence an identifying number, such as a GenBank accession number. Generally, the organization creating and maintaining the database provides software tools for searching the database files for a particular record, such as by accession number, name, or sequence.

The primers employed in the amplification step are specific for each desired gene sequence and include a variety of unique features. For example, the 5′ “sense” primer starts with the sequence 5′-CACCATG . . . (the start codon is underlined). The CACC sequence is added as a Kozak consensus that aids in translational efficiency. When the gene sequence being amplified represents a full-length gene, the 3′ “antisense” codon is designed to make the amplification product end at the 3rd position of the last codon of the gene being amplified, plus a single adenine residue. This facilitates the fusion of the coding region in-frame with a heterologous peptide sequence such as an epitope tag, an affinity purification tag, and the like (see below). The sequence specific primers used in the practice of the invention are designed to prime sequence between the start and stop codon of an open reading frame. The use of such primers will produce a specific coding region that can be further processed according to the methods disclosed herein. Methods of designing sequence specific primers are well known in the art.

The gene sequence need not encode a full-length sequence, however, as the invention methods are equally suitable for any gene sequence, including Expressed Sequence Tags (ESTs). The primers can be synthesized and dried in multiwell formats, such as 96-well microtiter plates to facilitate identification and further processing.

The amplified gene products are next isolated from the other components of the amplification reaction mixture. This purification can be accomplished using a variety of methodologies such as column chromatography, gel electrophoresis, and the like. A preferred method of purification utilizes low-melt agarose gel electrophoresis. The reaction mixture is separated and visualized by suitable means, such as ethidium bromide staining. DNA bands that represent correctly sized amplification products are cut away from the rest of the gel and placed into appropriate corresponding wells of a 96-well microtiter plate. These plugs are subsequently melted and the DNA contained therein utilized as cloning inserts. The use of gel electrophoresis has the advantage that the practitioner can purify the desired amplified gene sequence while additionally verifying that the sequence is of the correct size, i.e., represents the entire desired gene sequence.

The purified, amplified gene sequences are next inserted into an expression vector. A variety of expression vectors are suitable for use in the practice of the present invention, both for prokaryotic expression and eukaryotic expression. In general, the expression vector will have one or more of the following features: a promoter-enhancer sequence, a selection marker sequence, an origin of replication, an affinity purification tag sequence, an inducible element sequence, an epitope-tag sequence, and the like.

Promoter-enhancer sequences are DNA sequences to which RNA polymerase binds and initiates transcription. The promoter determines the polarity of the transcript by specifying which strand will be transcribed. Bacterial promoters consist of consensus sequences, −35 and −10 nucleotides relative to the transcriptional start, which are bound by a specific sigma factor and RNA polymerase. Eukaryotic promoters are more complex. Most promoters utilized in expression vectors are transcribed by RNA polymerase II. General transcription factors (GTFs) first bind specific sequences near the start and then recruit the binding of RNA polymerase II In addition to these minimal promoter elements, small sequence elements are recognized specifically by modular DNA-binding/trans-activating proteins (eg. AP-1, SP-1) which regulate the activity of a given promoter. Viral promoters serve the same function as bacterial or eukaryotic promoters and either provide a specific RNA polymerase in trans (bacteriophage T7) or recruit cellular factors and RNA polymerase (SV40, RSV, CMV) Viral promoters are preferred as they are generally particularly strong promoters.

Promoters may be, furthermore, either constitutive or, more preferably, regulatable (i.e., inducible or derepressible). Inducible elements are DNA sequence elements which act in conjunction with promoters and bind either repressors (eg. lacO/LAC Iq repressor system in E. coli) or inducers (eg. gal1/GAL4 inducer system in yeast). In either case, transcription is virtually “shut off” until the promoter is derepressed or induced, at which point transcription is “turned-on”.

Examples of constitutive promoters include the int promoter of bacteriophage λ, the bla promoter of the β-lactamase gene sequence of pBR322, the CAT promoter of the chlorampheicol acetyl transferase gene sequence of pPR325, and the like. Examples of inducible prokaryotic promoters include the major right and left promoters of bacteriophage (P_{L} and P_{R}), the trp, reca, lacZ, LacI, AraC and gal promoters of E. coli, the α-amylase (Ulmanen Ett at., J. Bacteriol. 162:176-182, 1985) and the sigma-28-specific promoters of B. subtilis (Gilman et al., Gene sequence 32:11-20(1984)), the promoters of the bacteriophages of Bacillus (Gryczan, In: The Molecular Biology of the Bacilli, Academic Press, Inc., NY (1982)), Streptomyces promoters (Ward et at., Mol. Gen. Genet. 203:468-478, 1986), and the like. Exemplary prokaryotic promoters are reviewed by Glick (J. Ind. Microtiot. 1:277-282, 1987); Cenatiempo (Biochimie 68:505-516, 1986); and Gottesman (Ann. Rev Genet. 18:415442, 1984).

Preferred eukaryotic promoters include, for example, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gal1 gene sequence promoter (Johnston et al., Proc. Natl. Acad. Sci. (USA) 79:6971-6975, 1982); Silver et al., Proc. Natl. Acad. Sci. (USA) 81:5951-5955, 1984), the CMV promoter, the EF-1 promoter, Ecdysone-responsive promoter(s), and the like.

Selection marker sequences are valuable elements in expression vectors as they provide a means to select, for growth, only those cells which contain a vector. Such markers are of two types: drug resistance and auxotrophic. A drug resistance marker enables cells to detoxify an exogenously added drug that would otherwise kill the cell. Auxotrophic markers allow cells to synthesize an essential component (usually an amino acid) while grown in media which lacks that essential component.

Common selectable marker gene sequences include those for resistance to antibiotics such as ampicillin, tetracycline, kannamycin, bleomycin, streptomycin, hygromycin, neomycin, Zeocin™, and the like. Selectable auxotrophic gene sequences include, for example, hisD, which allows growth in histidine free media in the presence of histidinol.

A preferred selectable marker sequence for use in yeast expression systems is URA3. Laboratory yeast strains carrying mutations in the gene which encodes orotidine-5′-phosphate decarboxylase, an enzyme essential for uracil biosynthesis, are unable to grow in the absence of exogenous uracil. A copy of the wild-type gene (ura4+ in S. pombe and URA3 in S. cerevisiae) will complement this defect in trans.

A further element useful in an expression vector is an origin of replication sequence. Replication origins are unique DNA segments that contain multiple short repeated sequences that are recognized by multimeric origin-binding proteins and which play a key role in assembling DNA replication enzymes at the origin site. Suitable origins of replication for use in expression vectors employed herein include E. coli oriC, 2 μ and ARS (both useful in yeast systems), sf1, SV40 (useful in mammalian systems), and the like.

Additional elements that can be included in an expression vector employed in accordance with the present invention are sequences encoding affinity purification tags or epitope tags. Affinity purification tags can are generally peptide sequences that can interact with a binding partner immobilized on a solid support. Synthetic DNA sequences encoding multiple consecutive single amino acids, such as histidine, when fused to the expressed protein, may be used for one-step purification of the recombinant protein by high affinity binding to a resin column, such as nickel sepharose. An endopeptidase recognition sequence can be engineered between the polyamino acid tag and the protein of interest to allow subsequent removal of the leader peptide by digestion with Enterokinase, and other proteases. Sequences encoding peptides such as the chitin binding domain (which binds to chitin), glutathione-S-transferase (which binds to glutathione), biotin (which binds to avidin and strepavidin), and the like can also be used for facilitating purification of the protein of interest. The affinity purification tag can be separated from the protein of interest by methods well known in the art, including the use of inteins (protein self-splicing elements, Chong, et al, Gene 192:271-281, 1997).

Epitope tags are short peptide sequences that are recognized by epitope specific antibodies. A fusion protein comprising a recombinant protein and an epitope tag can be simply and easily purified using an antibody bound to a chromatography resin. The presence of the epitope tag furthermore allows the recombinant protein to be detected in subsequent assays, such as Western blots, without having to produce an antibody specific for the recombinant protein itself. Examples of commonly used epitope tags include V5, glutathione-S-transferase (GST), hemaglutinin (HA), the peptide Phe-His-His-Thr-Thr, chitin binding domain, and the like.

A further useful element in an expression vector is a multiple cloning site or polylinker. Synthetic DNA encoding a series of restriction endonuclease recognition sites is inserted into a plasmid vector downstream of the promoter element. These sites are engineered for convenient cloning of DNA into the vector at a specific position.

The foregoing elements can be combined to produce expression vectors useful in creating the libraries of the invention. Suitable prokaryotic vectors include plasmids such as those capable of replication in E. coil (for example, pBR322, ColE1, pSC101, PACYC 184, itVX, pRSET, pBAD (Invitrogen, Carlsbad, Calif.) and the like). Such plasmids are disclosed by Sambrook (cf. “Molecular Cloning: A Laboratory Manual”, second edition, edited by Sambrook, Fritsch, & Maniatis, Cold Spring Harbor Laboratory, (1989)). Bacillus plasmids include pC194, pC221, pT127, and the like, and are disclosed by Gryczan (In: The Molecular Biology of the Bacilli, Academic Press, NY (1982), pp. 307-329). Suitable Streptomyces plasmids include plJlOl (Kendall et al., J. Bacteriol. 169:4177-4183,1987), and streptomyces bacteriophages such as φC31 (Chater et al., In: Sixth International Symposium on Actinomycetales Biology, Akaderiiai Kaido, Budapest, Hungary (1986), pp. 45-54). Pseudomonas plasmids are reviewed by John et al. (Rev. Infect. Dis. 8:693-704, 1986), and Izaki (Jpn. J. Bacteriol. 33:729-742, 1978).

Suitable eukaryotic plasmids include, for example, BPV, vaccinia, SV40, 2-micron circle, pcDNA3.1, pcDNA3.I/GS, pYES2/GS, pMT, p IND, pIND(Sp1), pVgRXR (Invitrogen), and the like, or their derivatives. Such plasmids are well known in the art (Botstein et al., Miami Wntr. Symp. 19:265-274, 1982; Broach, In: “The Molecular Biology of the Yeast Saccharomyces: Life Cycle and Inheritance”, Cold Spring Harbor Laboratory, Cold Spring Harbor, N.Y., p. 445-470, 1981; Broach, Cell 28:203-204, 1982; Dilon et at., J. Clin. Hematol. Oncol. 10:39-48, 1980; Maniatis, In: Cell Biology: A Comprehensive Treatise, Vol. 3, Gene Sequence Expression, Academic Press, NY, pp. 563-608,1980.

Construction of chimaeric DNA molecules in vitro relies traditionally on two enzymatic steps catalyzed by separate protein components. PCR amplification or site-specific restriction endonucleases are used to generate linear DNAs with defined termini that can then be joined covalently at their ends via the action of DNA ligase. DNA ligase has limitations, however, in that it is relatively slow acting and temperature sensitive.

Thus, when inserting the purified, amplified gene sequence into the expression vector the use of an enzyme that can both cleave and religate DNA in a site specific manner is preferred. Any site-specific enzyme of this type is suitable, for example, a type I topoisomerase or a site-specific recombinase. Examples of suitable site-specific recombinases include lambda integrase, FLP recombinase, P1-Cre protein, Kw recombinase, and the like (Pan, et al, J. Biol. Chem. 268:3683-3689, 1993; Nunes-Duby, et al, EMBO J. 13:4421-4430, 1994; Hallet and Sherratt, FEMS Microbio. Revs 21:157-178, 1997; Ringrose, et al, Eur J. Biochem 248:903-912, 1997).

A particularly suitable enzyme for use in creating the libraries of the invention is a type I topoisomerase, particularly vaccinia DNA topoisomerase. Vaccinia DNA topoisomerase binds to duplex DNA and cleaves the phosphodiester backbone of one strand. The enzyme exhibits a high level of sequence specificity, akin to that of a restriction endonuclease. Cleavage occurs at a consensus pentapyrimidine element 5′-(C/T)CCTT in the scissile strand. In the cleavage reaction, bond energy is conserved via the formation of a covalent adduct between the 3′ phosphate of the incised strand and a tyrosyl residue of the protein. Vaccinia topoisomerase can religate the covalently held strand across the same bond originally cleaved (as occurs during DNA relaxation) or it can religate to a heterologous acceptor DNA and thereby create a recombinant molecule.

When the substrate is configured such that the scissile bond is situated near (within 10 basepairs of) the 3′ end of a DNA duplex, cleavage is accompanied by the spontaneous dissociation of the downstream portion of the cleaved strand. The resulting topoisomerase-DNA complex, containing a 5′ single-stranded tail, can religate to an acceptor DNA if the acceptor molecule has a 5′ OH tail complementary to that of the activated donor complex.

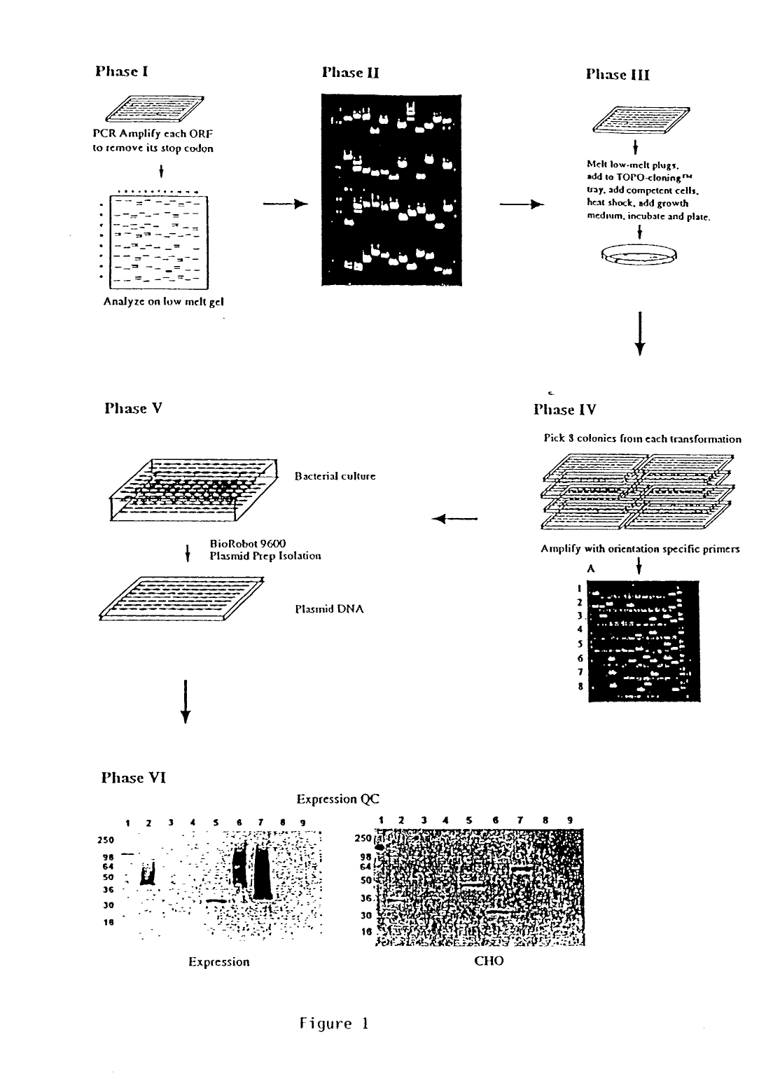

In accordance with the present invention, this reaction has been optimized for joining PCR-amplified DNA fragments into plasmid vectors (See FIG. 1). PCR fragments are naturally good surrogate substrates for the topoisomerase I religation step because they generally have 5′ hydroxyl residues from the primers used for the amplification reaction. The 5′ hydroxyl is the substrate for the religation reactions. The use of vaccinia topoisomerase type I for cloning is described in detail in copending U.S. patent application Ser. No. 08/358,344, filed Dec. 19, 1994, incorporated by reference herein in its entirety.

The gene sequence being inserted into the expression vector can insert in either the sense or antisense direction. Therefore, the creation of a useful library should include verification of both the size and orientation of the insert to insure that the gene sequence will express the desired protein. Preferably, the insert plus vector is utilized in a standard bacterial transformation reaction and the contents of the transformation plated onto a selective growth media. Bacterial transformation and growth selection procedures are well known in the art and described in detail in, for example, Ausubel, et al, Short Protocols in Molecular Biology, 3rd ed. 1995.

Individual bacterial colonies are picked and grown in individual wells of a 96 well microtiter plate containing selective growth media. An aliquot of these cells is used directly in a diagnostic PCR reaction. Primers for this reaction are designed such that only plasmids with correctly oriented inserts give amplification product. The amplified DNA is separated and visualized by SDS-PAGE gel electrophoresis using standard protocols (see Ausubel, et al, Short Protocols in Molecular Biology, 3rd ed. 1995).

Performing the PCR reaction directly from the cultured cell lysates, rather than first preparing DNA from the bacteria, is a particular advantage as it significantly reduces both the time needed to generate the required data and the cost of doing so.

Once plasmids containing the gene sequence insert in the correct orientation have been identified, plasmid DNA is prepared for use in the transformation of host cells for expression Methods of preparing plasmid DNA and transformation of cells are well known to those skilled in the art. Such methods are described, for example, in Ausubel, et al, supra.

Prokaryotic hosts are, generally, very efficient and convenient for the production of recombinant proteins and are, therefore, one type of preferred expression system. Prokaryotes most frequently are represented by various strains of E. coli. However, other organisms may also be used, including other bacterial strains.

Recognized prokaryotic hosts include bacteria such as E. coli and those from genera such as Bacillus, Streptomyces, Pseudomonas, Salmonella, Serratia, and the like. However, under such conditions, the polypeptide will not be glycosylated. The prokaryotic host selected for use herein must be compatible with the replicon and control sequences in the expression plasmid.

Suitable hosts may often include eukaryotic cells. Preferred eukaryotic hosts include, for example, yeast, fungi, insect cells, and mammalian cells either in vivo, or in tissue culture. Mammalian cells which may be useful as hosts include HeLa cells, cells of fibroblast origin such as VERO, 3T3 or CHOK1, HEK 293 cells or cells of lymphoid origin (such as 32D cells) and their derivatives. Preferred mammalian host cells include nonadherent cells such as CHO, 32D, and the like. Preferred yeast host cells include S. pombe, Pichia pastoris, S. cerevisiae (such as INVSc1), and the like.

In addition, plant cells are also available as hosts, and control sequences compatible with plant cells are available, such as the cauliflower mosaic virus 35S and 19S, nopaline synthase promoter and polyadenylation signal sequences, and the like. Another preferred host is an insect cell, for example the Drosophila larvae. Using insect cells as hosts, the Drosophila alcohol dehydrogenase promoter can be used. Rubin, Science 240:1453-1459, 1988). Alternatively, baculovirus vectors can be engineered to express large amounts of peptide encoded by a desire gene sequence in insects cells (Jasny, Science 238:1653, 1987); Miller et al., In: Genetic Engineering (1986), Setlow, J. K., et al., eds., Plenum, Vol. 8, pp. 277-297). The present invention also features the purified, isolated or enriched versions of the expressed gene products produced by the methods described above.

Kits comprising one or more containers or vials containing components for using the libraries of the present invention are also within the scope of the invention. Kits can comprise any one or more of the following elements: one or more expressible gene sequences, cells which are, or can be, transfected with such gene sequences, and antibodies recognizing the expressed gene product or an epitope tag associated therewith. Cells suitable for inclusion in such a kit include bacterial cells, yeast cells (such as INVSc1), insect cells or mammalian cells (such as CHO).

In one embodiment, such a kit comprises a detergent solution, preferably the Trax® lysing reagent (6% NP-40 and 9% Triton X-100 in 1×PBS). Also included in the kit can be one or more binding partners, e.g., an antibody or antibodies, preferably a pair of antibodies to the same expressed gene product, which preferably do not compete for the same binding site on the expressed gene product.

In another embodiment, a kit comprises more than one pair of such antibodies or other binding partners, each pair directed against a different target molecule, thus allowing the detection or measurement of a plurality of such target molecules in a sample. In a specific embodiment, one binding partner of the kit may be pre-adsorbed to a solid phase matrix, or alternatively, the binding partner and matrix are supplied separately and the attachment is performed as part of the assay procedure. The kit preferably contains the other necessary washing reagents well-known in the art. For EIA, the kit contains the chromogenic substrate as well as a reagent for stopping the enzymatic reaction when color development has occurred. The substrate included in the kit is one appropriate for the enzyme conjugated to one of the antibody preparations. These are well-known in the art The kit can optionally also comprise a target molecule standard; i.e., an amount of purified target molecule that is the target molecule being detected or measured.

In a specific embodiment, a kit of the invention comprises in one or more containers: (1) a solid phase carrier, such as a microtiter plate coated with a first binding partner; (2) a detectably labeled second binding partner which binds to the same expressed gene product as the first binding partner; (3) a standard sample of the expressed gene product recognized by the first and second binding partners; (4) concentrated detergent solution; and (5) optionally, diluent.

In another embodiment, the invention features methods of screening cells for binding partners of an expressed gene product of the invention. By “natural binding partner” it is meant a molecule that interacts specifically with the expressed gene product. Binding partners include ligands, agonists, antagonists and downstream signaling molecules such as adaptor proteins and may be identified by techniques well known in the art such as co-immunoprecipitation or by using, for example, a two-hybrid screen. (Fields and Song, U.S. Pat. No. 5,283,173, issued Feb. 1, 1994 and, incorporated be reference herein.).

Binding partners contemplated by the invention may additionally be antibodies. The term “antibody” is used herein in the broadest sense and specifically includes intact monoclonal antibodies, polyclonal antibodies, multispecific antibodies (e g bispecific antibodies) formed from at least two intact antibodies, and antibody fragments, including single chain antibodies, so long as they exhibit the desired binding properties as described herein Various procedures well-known in the art may be used for the production of polyclonal antibodies to an epitope or antigen of interest. A host animal of any of a number of species, such as rabbit, goat, sheep, horse, cow, mice, rat, etc. is immunized by injection with an antigenic preparation which may be derived from cells or microorganisms, or may be recombinantly or synthetically produced. Various adjuvants well known in the art may be used to enhance the production of antibodies by the immunized host, for example, Freund's adjuvant (complete and incomplete), mineral gels such as aluminum hydroxide, surface active substances such as lysolecithin, pluronic polyols, polyanions, peptides, oil emulsions, keyhole limpet hemocyanins, dinitrophenol, liposomes, potentially useful human adjuvants such as BCG (Bacille Calmette-Guerin) and Propionibacterium acanes, and the like.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. Preferred antibodies are mAbs, which may be of any immunoglobulin class including IgG, IgM, IgE, IgA, and any subclass or isotype thereof.

In addition to their specificity, monoclonal antibodies are advantageous in that they are synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al, Nature, 256:495 (1975), or may be made by recombinant DNA methods (see, eg., U.S. Pat. No. 4,816,567, incorporated by reference herein). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies contemplated for use herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl Acad Sci USA, 81:6851-6855 (1984)).

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are not found in either the recipient antibody or in the imported CDR or framework sequences. These modifications are made to further refine and maximize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525 (1986); Reichmann et al., Nature, 332:323-329 (1988); and Presta, Curr Op. Struct. Biol., 2:593-596 (1992). The humanized antibody includes a PRIMATIZED™ antibody wherein the antigen-binding region of the antibody is derived from an antibody produced by immunizing macaque monkeys with the antigen of interest.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al. Protein Eng. 8(10):1057-1062 (1995)); single-chain antibody molecules, multispecific antibodies formed from antibody fragments, and the like.

Particularly preferred in the practice of the invention are single-chain antibodies. “Single-chain” or “sFv” antibodies are antibody fragments comprising the V_{H} and V_{L} domains of an antibody, wherein these domains are present in a single polypeptide chain. Preferably, the Fv polypeptide further comprises a polypeptide linker between the V_{H} and V_{L} domains which enables the sFv to form the desired structure for antigen binding. For a review of sfvs see Pluckthun in The Pharmacology of Monoclonal Antibodies, vol. 113, Rosenburg and Moore Eds., Springer-Verlag, New York, pp. 269-315 (1994).

Large quantities of single chain antibodies with uncharacterized randomized binding specificity can be produced using a number of methodologies known in the art. Random peptide libraries can be created in filamentous phage particles (Daniels and Lane, Methods 9(3):494-507, 1996; Reichmann and Weill, Biochemistry 32(34):8848-8855; Rader and Barbas, Curr Opin Biotechnol 9(4):503-508, 1997; Iba and Kurosawa, Immunol Cell Biol 75(2):217-221, 1997), for example, or similarly in yeast, bacteria, and the like. Other methods for creating random libraries of sFvs include various solid state synthesis methods.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Nail. Acad. Sci. USA, 90:6444-6448 (1993).

Methods of identifying specific antibodies are well known in the art and include methods such as ELISAs, Western blots, immunoprecipitation, and the like (see, for example, Ausubel, et al, Short Protocols in Molecular Biology, 3rd ed. 1995, incorporated herein in its entirety). One method of large scale, high through-put screening for specific antibodies is described in co-pending US application entitled Microarrays and Uses Therefor, filed Feb. 4, 1999, U.S. Ser. No. 09/245,615.

The invention will now be described in greater detail by reference to the following non-limiting examples.

### EXAMPLES

### Example 1

### High-Throughput Expression of Yeast ORFs

The following example illustrates the creation of a library of expressible yeast gene sequences.

Amplification

6,032 yeast ORFs and a corresponding gene-specific primer of the 3′ end of each were obtained from Research Genetics (Huntsville, Ala.) in a 96-well microtiter plate format at a concentration of 0.3 ng/μl. Each gene specific primer was designed to exclude the gene's stop codon. Since the templates each contain a common sequence immediately 5′ of the start ATG (5′-GCAGTCCTGGAATTCCAGCTGACCACC) (SEQ. ID. NO.: 1), each template could be amplified with a common 5′ primer.

5 μl of ORF template was added to a fresh 96-well microtiter plate (polycarbonate Thermowell Thinwall, Model M. Cat #6511) using a 12 channel pipetter. 6 μl of specific 3′ primer solution (2 μM) was added and the total volume per well brought to 30 μl with PCR cocktail, immediately after which the plate was placed on ice. (PCR cocktail for 120 reactions—720 μl 5×Buffer J, 48 μl dNTPs (50 mM stock), 12 μl common 5′ primer (1 μg/μl stock), 48 μl Taq DNA polymerase (Boeringer-Mannheim or Promega, 5 units/μl), 1.92 μl Pfu DNA polymerase (Stratagene, cat. #600153-81, 2.5 units/μl) and 1464 μl distilled water. 5×Buffer J: 300 mM Tris (pH 9.5), 75 mM ammonium sulfate, 10 mM MgCl₂). The rubber Hybaid Micromat lid was washed by soaking in 0.1 M HCl, the rinsed for 2 minutes with distilled water and dried completely before applying to the 96-well plate.

The PCR reaction was performed using a Hybaid, Ltd. (Middlesex, UK) thermo-cycler according to the manufacturer's instructions. The conditions used were as follows: pre-melt step: 94° C.×4 min; melt step: 94° C.×30 sec, anneal step: 58° C.×45 sec, extend step: 72° C.×3 min—repeated for 25 cycles; final extension: 72° C.×4 min; final block temperature set to room temp (approx. 22° C.). The plates were stored at 4° C.

Purification

The plates were spun briefly at 1000 rpm, then 10 μl of 6×gel loading dye was added to each well (6×gel loading dye: 6 mM Tris (pH 8), 6 mM EDTA, 0.03% Bromphenol Blue, 30% glycerol). The entire contents of each well were loaded onto a 1% low melt agarose (Invitrogen #46-0150) gel (plus ethidium bromide at 20 μl of a 10 mg/ml solution added to 400 mls of agarose) in IX TAE (50×TAB=242 g Tris base, 57.1 ml glacial acetic acid, 100 ml 0.5 M EDTA, pH 8.0 per liter (water)) and run at 110-120 volts for 1.25 to 1.5 hours. A UV light box was used to visualize the amplification products and ensure that only correct-sized PCR products are used in the insertion step.

Insertion into Expression Vector(s)

The portion of each lane containing the amplified gene sequence was cut from the gel and transferred to a well in a 96-well microtiter plate, melted on a heat block (75° C.), and a portion of the melt multi-channel pipetted into a 96-well microtiter plate (7 μl/well) containing one of two expression vectors: TOPO-adapted pcDNA3.1/GS or pYES2/GS (see Example 3, below). The plate was covered with parafilm and incubated at 37° C. for 7 minutes. Top 10 Chemically Competent Cells (Invitrogen) were added to each well (45 μl/well, O.D.=4.7), whereupon the plate was re-covered and incubated on ice for 5 minutes. The cells were then heat shocked on a 42° C. block for 1 minute and returned to ice for 1 minute. An aliquot of SOC medium was added to each well (150 μl, 20 g tryptone, 5 g yeast extract, 0.5 g NaCl, 250 mM KCl, 20 ml 1M glucose/liter), and the plate was incubated at 37° C. for 90 to 120 minutes.

The contents of each well were plated onto a LB(10 g tryptone, 5 g yeast extract, 10 g NaCl per liter)/1.5% agar petrie plate containing the appropriate selection marker (ampicillin (50 μg/ml) for pYES2/GS and Zeocin™ (25 μg/ml) for pcDNA3.1/GS). The petrie plates were grown overnight at 37° C.

Verification of Size and Orientation

Contamination is a potentially serious problem in this step. Care should be taken to guard against contaminating the process through airborne contamination, unsterile reagents or equipment, or well-to-well contamination.

Eight colonies were picked from each petrie plate and placed in eight individual wells of a 96-well microtiter plate. Each well contained 100 μl of 2×LB plus 100 μg/ml ampicillin or 50 μg/ml Zeocin™ as appropriate for the expression vector used. The plates were incubated overnight at 37° C.

The plates were spun briefly at 1000 rpm. The cells were stirred by pipetting up and down in a pipetter, then 2 μl from each well was transferred to a corresponding well in a PCR reaction plate containing 28 pi/well PCR cocktail (PCR cocktail for 840 reactions—5040 μl 5×Buffer J, 336 ill dNTPs (50 mM stock), 84 μl common 5′ primer (1 μg/μl stock, Dalton Chemical Lab. Inc, Ont. CAN), 84 μl 3′ H6stopprevu primer (1 μg/μl, Dalton Chemical Lab. Inc, Ont. CAN), 336 μl Taq DNA polymerase (Boeringer Mannheim or Promega 5 units/μl), and 17.64 mls distilled water. H6stopprevu primer has the sequence 5′ AAA CTC AAT GGT GAT GGT GAT GAT GACC-3′) (SEQ. ID. NO.: 2).

The PCR reaction was run essentially as described above with the following cycle: pre-melt step: 94° C.×10 min; melt step: 94° C.×1 min, anneal step: 67° C.×1 min, extend step: 72° C.×3 min−35 cycles; final extension: 72° C.×4 min; final block temp set to room temp (approximately 22° C.). The plates were spun briefly at 100 rpm and 6 μl of 6×gel loading dye added to each well. Samples were run on a 1% agarose gel which was subsequently stained with ethidium bromide Only plasmids with correctly oriented inserts give an amplification product in this step.

The location of the positive clones was entered into a database and a spreadsheet of positive clones generated. The spreadsheet was downloaded onto a Qiagen BioRobot 9600™ to direct the re-racking of the positive cultures into deep-well culture blocks. Essentially, a single positive culture for each clone was grown and used to prepare plasmid DNA according to the Quia-Prep Turbo protocol.

CHO cells or were transfected with the prepared plasmid DNA using the Pfx-6 PerFect Lipid system (Invitrogen, Cat #T930-16). Yeast cells (INVSc1) were transfected using the S.C. EasyComp Transformation kit (Invitrogen, Cat #K5050-01). Expression was verified by Western blot using anti-V5 antibody to detect the epitope tag. All of the yeast ORFS were expressed in either pYES or pDNA3.1. Table 1 below lists the yeast proteins successfully produced using the yeast ORFs.

TABLE 1
Yeast ORFs

| Plate Name | ORF Identifer | Protein description |
|---|---|---|
| M12 E2 | YAL003W | 447-987 Translation |
| | | elongation factor EF- |
| | | 1beta GDP\/GTP |
| | | exchange factor for |
| | | Teflp\/Tef2p |
| | | (22.77/40) |
| M12 D4 | YAL005C | Heat shock protein of |
| | | HSP70 family |
| | | cytoplasmic |
| | | (70.65/45) |
| M12 E5 | YAL007C | (23.68/32) |
| M11 H1 | YAL009W | Protein required for |
| | | meiosis (28.60/30) |
| M135 F2 | YAL012W | cystathionine gammalyase |
| | | (43.45/43) |
| M11 D2 | YAL013W | (39.93/40) |
| M11 E2 | YAL014C | (22.58/31) |
| M12 F5 | YAL015C | DNA glycosylase |
| | | (43.92/48) |
| M12 D6 | YAL016W | protein phosphatase |
| | | 2A regulatory subunit |
| | | A (69.96/56) |
| M136 H1 | YAL020C | (36.66/47) |
| M12 F4 | YAL022C | (56.90/56) |
| M11 E4 | YAL030W | 216-467 vesicle- |
| | | associated membrane |
| | | protein |
| | | (synaptobrevin) |
| | | homolog (12.98/20) |
| M136 D1 | YAL034W-A | (31.9/28) |
| M12 H5 | YAL037W | (29.48/33) |
| M136 H4 | YAL038W | Pyruvate kinase |
| | | (55.11/60) |
| M136 G5 | YAL039C | cytochrome c heme |
| | | lyase (CCHL) |
| | | (29.62/45) |
| M136 C4 | YAL044C | H-protein subunit of |
| | | the glycine cleavage |
| | | system (19.50/36) |
| M12 G6 | YAL045C | (11.25/11) |
| M11 C7 | YAL049C | (27.09/37) |
| M135 H4 | YAL053W | (86.24/64) |
| M136 B5 | YAL055W | (19.91/28) |
| M12 G7 | YAL056W | (93.28/98) |
| M12 B3 | YAL059W | (23.43/34) |
| M11 D8 | YAL060W | (42.13/45) |
| M12 A5 | YAL061W | (45.98/40) |
| M11 F8 | YAL062W | (50.48/50) |
| M11 H9 | YAR002W | (59.4/60) |
| M12 F1 | YAR003W | (46.97/53) |
| M12 C3 | YAR008W | 34 kDa subunit of the |
| | | tetrameric tRNA |
| | | splicing endonuclease |
| | | (30.46/38) |
| M12 B5 | YAR010C | (48.43/45) |
| M11 C11 | YAR023C | (19.72/30) |
| M12 B4 | YAR027W | (25.96/40) |
| M12 C5 | YAR028w | (25.85/30) |
| M12 B7 | YAR030C | (12.46/12) |
| M135 E2 | YAR035W | Outer carnitine |
| | | acetyltransferase |
| | | mitochondrial |
| | | (75.68/76) |
| M12 E3 | YAR037W | (21.34/25) |
| M12 C4 | YAR040C | (13.12/20) |
| M138 E1 | YAR052C | (13.89/36) |
| M14 F1 | YAR062W | (21.89/36) |
| M138 H5 | YBL001C | ExtraCellular Mutant |
| | | (11.47/10) |
| M137 B1 | YBL002W | Histone H2B (HTB1 |
| | | and HTB2 code for |
| | | nearly identical |
| | | proteins) (14.52/25) |
| M14 B3 | YBL003C | Histone H2A (HTA1 |
| | | and HTA2 code for |
| | | nearly identical |
| | | proteins) (14.55/15) |
| M13 F3 | YBL005W-A | (48.51/48) |
| M13 C2 | YBL010C | (30.83/35) |
| M333 D1 | YBL011W | (83.6/93) |
| M138 A6 | YBL015W | acetyl CoA hydrolase |
| | | (57.97/60) |
| M137 C1 | YBL016W | cdc2+\/CDC28 |
| | | related kinase with |
| | | positive role in |
| | | conjugation |
| | | (38.94/45) |
| M310 B1 | YBL019W | (57.31/64) |
| M13 G3 | YBL020W | 67 kDa integral |
| | | membrane protein |
| | | (63.25/70) |
| M13 D4 | YBL021C | transcriptional |
| | | activator protein of |
| | | CYC1 (15.87/20) |
| M13 G2 | YBL027W | 387-954 Ribosomal |
| | | protein YL14 (rat |
| | | L19) (rp33) (RPL19A |
| | | and RPL19B code for |
| | | identical genes) |
| | | (20.9/32) |
| M137 C4 | YBL028C | (11.69/20) |
| M137 G6 | YBL031W | (37.29/38) |
| M14 B7 | YBL033C | GTP cyclohydrolase |
| | | II (37.98/38) |
| M137 D3 | YBL035C | B subunit of DNA |
| | | polymerase alpha- |
| | | primase complex |
| | | (77.58/80) |
| M13 H3 | YBL036C | (28.30/32) |
| M138 C5 | YBL038W | Mitochondrial |
| | | ribosomal protein |
| | | MRPL16 (25.63/30) |
| M137 B2 | YBL041W | proteasome subunit |
| | | (26.62/36) |
| M13 H2 | YBL043W | ExtraCellular Mutant |
| | | (28.48/45) |
| M13 A4 | YBL044W | (13.53/17) |
| M13 C5 | YBL046W | (48.62/55) |
| M138 F2 | YBL050W | 147-995 peripheral |
| | | membrane protein |
| | | required for vesicular |
| | | transport between ER |
| | | and Golgi (32.23/35) |
| M137 C2 | YBL057C | (23.57/36) |
| M14 C10 | YBL058W | isolated as a |
| | | suppressor of the |
| | | lethality caused by |
| | | overexpression of the |
| | | phosphoprotein |
| | | phosphatase 1 |
| | | catalytic subunits |
| | | encoded by GLC7 |
| | | (46.64/50) |
| M13 B4 | YBL060W | (75.68/75) |
| M137 D1 | YBL064C | (28.74/36) |
| M15 A1 | YBL080C | 62-kDa protein |
| | | (59.54/60) |
| M141 C1 | YBL081W | (40.59/60) |
| M16 F1 | YBL082C | Resistance to |
| | | Hansenula Killer 1 |
| | | hypothetical F-458 |
| | | protein (50.41/50) |
| M15 H5 | YBL086C | (51.29/?) |
| M15 H4 | YBL093C | nuclear protein |
| | | (24.23/36) |
| M15 D7 | YBL095W | (29.81/50) |
| M140 E3 | YBL099W | mitochondrial F1F0- |
| | | ATPase alpha subunit |
| | | (60.06/60) |
| M15 B6 | YBL101W-A | (48.29/48) |
| M310 C1 | YBL105C | putative protein |
| | | kinase (126.64/150) |
| M16 A3 | YBL107C | (21.59/31) |
| M15 C4 | YBR002C | (31.49/32) |
| M15 B5 | YBR003W | hexaprenyl |
| | | pyrophosphate |
| | | synthetase (52.14/55) |
| M16 H3 | YBR004C | (47.66/48) |
| M15 F7 | YBR005W | (23.54/40) |
| M140 D4 | YBR010W | Histone H3 (HHT1 |
| | | and HHT2 code for |
| | | identical proteins) |
| | | (15.07/20) |
| M15 C5 | YBR011C | Inorganic |
| | | pyrophosphatase |
| | | (31.60/50) |
| M15 E6 | YBR012C | (15.32/18) |
| M15 G7 | YBR012W-A | (48.51/64) |
| M15 A3 | YBR014C | (22.46/22) |
| M15 E4 | YBR016W | (14.19/14) |
| M15 F6 | YBR018C | galactose-1-phosphate |
| | | uridyl transferase |
| | | (40.29/45) |
| M15 H7 | YBR019C | UDP-glucose 4- |
| | | epimerase (76.92/76) |
| M141 A3 | YBR024W | (33.22/37) |
| M16 D3 | YBR025C | (43.47/43) |
| M15 G6 | YBR026C | Nuclear protein that |
| | | binds to T-rich strand |
| | | of core consensus |
| | | sequence of |
| | | autonomously |
| | | replicating sequence |
| | | (41.83/41) |
| M140 G3 | YBR031W | large ribosomal |
| | | subunit protein 2A |
| | | (39.93/42) |
| M140 F6 | YBR034C | protein arginine |
| | | methyltransferase |
| | | (mono- and |
| | | asymmetrically |
| | | dimethylating |
| | | enzyme) (38.31/50) |
| M15 A8 | YBR035C | pyridoxine |
| | | (pyridoxiamine) |
| | | phosphate oxidase |
| | | (25.11/32) |
| M255 C1 | YBR036C | contains 9 or 10 |
| | | putative membrane |
| | | spanning regions\ |
| | | putative Ca2+ |
| | | binding protein |
| | | (homology to EF- |
| | | hand Ca2+binding |
| | | site) (45.13/55) |
| M16 H1 | YBR046C | (36.77/35) |
| M15 A7 | YBR050C | (37.21/50) |
| M140 E1 | YBR052C | (23.13/34) |
| M15 G5 | YBR057C | Muddled Meiosis |
| | | (40.29/70) |
| M19 B1 | YBR061C | (34.13/40) |
| M143 A4 | YBR063C | (44.47/48) |
| M143 C7 | YBR066C | (24.23/25) |
| M19 A2 | YBR068C | probable amino acid |
| | | permease for leucine |
| | | valine and isoleucine |
| | | (67.02/67) |
| M19 C2 | YBR070C | (26.10/40) |
| M143 B4 | YBR071W | (23.32/33) |
| M142 E6 | YBR072W | heat shock protein 26 |
| | | (23.65/32) |
| M143 B2 | YBR077C | (17.85/32) |
| M142 B5 | YBR079C | (106.07/106) |
| M19 E3 | YBR080C | cytoplasmic protein |
| | | involved in protein |
| | | transport between ER |
| | | and Golgi\ |
| | | ATPase (83.41/080) |
| M19 F3 | YBR081C | transcription factor |
| | | (146.55/050) |
| M19 G3 | YBR082C | 143-542 ubiquitin- |
| | | conjugating enzyme |
| | | (16.49/16) |
| M202 D1 | YBR083W | transcriptional |
| | | regulator of Ty1 |
| | | expression (53.57/64) |
| M19 A4 | YBR084C-A | 509-1076 Ribosomal |
| | | protein YL14 (rat |
| | | L19) (rp33) (RPL19A |
| | | and RPL19B code for |
| | | identical genes) |
| | | (20.9/?) |
| M19 C4 | YBR085W | mitochondrial |
| | | ADP\/ATP |
| | | translocator |
| | | (33.88/50) |
| M143 D6 | YBR088C | profilerating cell |
| | | nuclear antigen |
| | | (28.41/0) |
| M19 A5 | YBR090C-A | 11-kDa nonhistone |
| | | chromosomal protein |
| | | (10.92/10) |
| M143 C2 | YBR091C | (12.02/16) |
| M143 D3 | YBR092C | Acid phosphatase |
| | | constitutive |
| | | (51.40/50) |
| M19 E5 | YBR094W | (82.94/80) |
| M19 F5 | YBR095C | (49.86/50) |
| M20 E1 | YBR098W | (51.92/48) |
| M143 D4 | YBR101C | (31.93/32) |
| M142 E10 | YBR105C | (39.85/45) |
| M143 F1 | YBR106W | May be a membrane |
| | | protein involved in |
| | | inorganic phosphate |
| | | transport and |
| | | regulation of Pho81p |
| | | function (20.79/0) |
| M143 E4 | YBR109C | Calmodulin (16.20/12) |
| M20 G7 | YBR111C | (25.44/30) |
| M143 A8 | YBR112C | General repressor of |
| | | transcription (with |
| | | Tup1p)\mediates |
| | | glucose repression |
| | | (106.29/100) |
| M19 H7 | YBR113W | (17.71/70) |
| M20 F6 | YBR118W | translational |
| | | elongation factor EF- |
| | | 1 alpha (50.49/55) |
| M19 F8 | YBR119W | 98-986 U1 snRNP A |
| | | protein (32.89/36) |
| M143 B8 | YBR120C | (17.85/36) |
| M19 H8 | YBR121C | Glycyl-tRNA |
| | | synthase (73.40/90) |
| M19 A9 | YBR122C | Mitochondrial |
| | | ribosomal protein |
| | | MRPL36 (YmL36) |
| | | (21.59/30) |
| M142 A3 | YBR123C | transcription factor |
| | | tau (TFIIIC) subunit |
| | | 95 (71.42/75) |
| M19 E9 | YBR126C | 56 kD synthase |
| | | subunit of trehalose- |
| | | 6-phosphate |
| | | synthase\/phosphatase |
| | | complex (54.48/55) |
| M19 F9 | YBR127C | (56.90/36) |
| M20 D9 | YBR128C | (37.87/37) |
| M143 B9 | YBR129C | (36.11/40) |
| M19 A10 | YBR130C | (46.78/50) |
| M143 E2 | YBR131W | (77.55/72) |
| M19 F10 | YBR135W | subunit of the Cdc28 |
| | | protein kinase |
| | | (16.61/16) |
| M143 C9 | YBR137W | (19.8/30) |
| M143 F2 | YBR139W | (55.99/0) |
| M20 E9 | YBR144C | (11.47/32) |
| M19 A12 | YBR146W | Probable |
| | | mitochondrial |
| | | ribosomal protein S9 |
| | | (30.69/31) |
| M265 B1 | YBR149W | (37.95/40) |
| M20 F9 | YBR152W | (32.12/50) |
| M19 H12 | YBR153W | Protein involved in |
| | | the biosynthesis of |
| | | riboflavin second step |
| | | in the riboflavin |
| | | biosynthesis pathway |
| | | (26.95/40) |
| M145 C4 | YBR158W | (60.5/98) |
| M144 B9 | YBR161W | (41.47/52) |
| M144 B1 | YBR162C | (50.08/60) |
| M145 B2 | YBR162W-A | Protein that |
| | | participates in |
| | | secretory pathway |
| | | (7.36/10) |
| M145 D4 | YBR165W | (30.58/36) |
| M144 H6 | YBR166C | Prephenate |
| | | dehydrogenase |
| | | (NADP+) (49.75/50) |
| M145 C1 | YBR169C | Member of the 70- |
| | | kDa heat-shock |
| | | protein family |
| | | (76.36/98) |
| M144 C3 | YBR171W | integral membrane |
| | | glycoprotein |
| | | (22.77/32) |
| M145 E4 | YBR173C | (16.31/35) |
| M144 D9 | YBR176W | ExtraCellular Mutant |
| | | (34.43/45) |
| M145 D1 | YBR177C | (49.64/60) |
| M144 D3 | YBR179C | Yeast fzo homolog |
| | | (drosophila |
| | | melanogaster fuzzy |
| | | onions |
| | | gene) Reference:Hales |
| | | K.G. and Fuller M.T. |
| | | -1997 |
| | | Developmentally |
| | | regulated |
| | | mitochondrial fusion |
| | | mitochondrial fusion |
| | | mediated by a |
| | | conserved novel |
| | | predicted GTPase. |
| | | Cell. 90 121-129. |
| | | (94.08/98) |
| M144 H5 | YBR181C | 359-1063 40S |
| | | ribosomal gene |
| | | product S10 |
| | | (26.07/32) |
| M145 F5 | YBR182C | (49.75/0) |
| M144 E3 | YBR187W | (30.91/36) |
| M144 E4 | YBR188C | (15.43/20) |
| M144 A6 | YBR189W | 421-1001 Ribosomal |
| | | protein |
| | | SUP46\/RPS13 |
| | | (YS11A) (YP28) (E. |
| | | coli S4) (rat |
| | | S9) (21.56/33) |
| M144 F1 | YBR193C | (24.56/34) |
| M144 F1 | YBR193C | (24.56/34) |
| M145 F2 | YBR194W | (13.64/20) |
| M144 F4 | YBR196C | phoshogluco- |
| | | isomerase |
| | | (60.97/60) |
| M144 B6 | YBR197C | (23.90/32) |
| M145 E6 | YBR199W | Putative alpha-1 2- |
| | | mannosyltransferase |
| | | (51.15/64) |
| M144 G9 | YBR200W | contains two SH3 |
| | | domains (60.72/64) |
| M144 G4 | YBR204C | (41.38/38) |
| M144 C6 | YBR205W | Putative alpha-1 2- |
| | | mannosyltransferase |
| | | (44.55/48) |
| M145 F1 | YBR209W | (11.66/16) |
| M144 F2 | YBR210W | (15.73/16) |
| M144 H4 | YBR212W | negative growth |
| | | regulatory protein |
| | | (74.03/74) |
| M144 D6 | YBR213W | (30.35/38) |
| M145 H5 | YBR214W | (58.08/90) |
| M144 E6 | YBR221C | beta subunit of |
| | | pyruvate |
| | | dehydrogenase (E1 |
| | | beta) (40.39/42) |
| M202 E1 | YBR222C | (59.76/70) |
| M145 A6 | YBR230C | 109-502 (14.85/18) |
| M144 H8 | YBR231C | (33.46/42) |
| M145 H1 | YBR233W | (45.54/60) |
| M144 C5 | YBR236C | RNA (guanine-7-) |
| | | methyltransferase |
| | | (cap |
| | | methyltransferase) |
| | | (47.99/52) |
| M144 F6 | YBR237W | RNA helicase |
| | | homolog (93.5/95) |
| M144 H2 | YBR242W | (26.39/32) |
| M255 D1 | YBR243C | UDP-N-acetyl- |
| | | glucosamine-1-P |
| | | transferase (GPT) |
| | | (49.31/38) |
| M145 B4 | YBR244W | (17.93/0) |
| M144 A9 | YBR247C | Putative 57 kDa |
| | | protein with an |
| | | apparent MW of 70 |
| | | kDa by SDS-PAGE |
| | | (53.26/55) |
| M144 | YBR248C | glutamine |
| D10 | | amidotransferase:cycl |
| | | ase (60.75/61) |
| M147 A1 | YBR249C | 3-deoxy-D-arabino- |
| | | heptulosonate 7- |
| | | phosphate (DAHP) |
| | | synthase isoenzyme |
| | | (40.73/50) |
| M24 C1 | YBR251W | Probable |
| | | mitochondrial |
| | | ribosomal protein S5 |
| | | (33.88/40) |
| M146 B4 | YBR253W | transcription factor |
| | | (13.42/18) |
| M146 D7 | YBR256C | Riboflavin synthase |
| | | alpha-chain |
| | | (26.21/26) |
| M25 D1 | YBR258C | (15.65/20) |
| M146 C4 | YBR261C | (25.55/38) |
| M146 D5 | YBR262C | (11.69/50) |
| M147 F6 | YBR263W | Serine |
| | | hydroxymethyltransfe |
| | | rase mitochondrial |
| | | (62.36/62) |
| M24 A3 | YBR265W | (35.31/35) |
| M25 F1 | YBR267W | (32.56/45) |
| M146 E5 | YBR270C | (59.98/64) |
| M146 B1 | YBR273C | (47.99/70 m) |
| M146 F1 | YBR274W | (58.08/50) |
| M303 C1 | YBR283C | (53.93/48) |
| M146 F4 | YBR285W | (15.95/30) |
| M24 B6 | YBR290W | (35.42/55) |
| M25 G1 | YBR291C | citrate tranporter in |
| | | mitochondrial inner |
| | | membrane (32.92/35) |
| M274 D1 | YBR295W | Putative P-type |
| | | Cu(2+)-transporting |
| | | ATPase (133.87/125) |
| M25 H1 | YCL005W | (28.05/40) |
| M147 B7 | YCL009C | Acetolactate synthase |
| | | regulatory subunit |
| | | (34.02/34) |
| M146 B5 | YCL016C | (34.02/34) |
| M25 G3 | YCL018W | beta-IPM |
| | | (isopropylmalate) |
| | | dehydrogenase |
| | | (40.25/55) |
| M147 E8 | YCL027W | MAP kinase involved |
| | | in pheromone signal |
| | | transduction G(sub)1 |
| | | arrest (56.43/50) |
| M146 C2 | YCL029C | Microtubule- |
| | | associated protein |
| | | required for |
| | | microtubule function |
| | | during mating and |
| | | mitosis (48.43/70) |
| M147 E5 | YCL032W | Protein that interacts |
| | | with Gpalp Ste4 and |
| | | Ste18 to regulate |
| | | adaptation to |
| | | pheromone |
| | | (38.27/48) |
| M146 D8 | YCL035C | (12.13/16) |
| M147 F2 | YCL037C | Suppressor of rho3 |
| | | (51.39/55) |
| M146 C7 | YCL042W | (13.09/30) |
| M25 F4 | YCL043C | protein disulfide |
| | | isomerase (57.45/62) |
| M22 A1 | YCL044C | (45.90/46) |
| M265 F1 | YCL045C | (83.63/85) |
| M148 D5 | YCL049C | (34.45/64) |
| M148 D6 | YCL050C | diadenosine 5′ 5′″-P1 |
| | | P4-tetraphosphate |
| | | phosphorylase I |
| | | (35.34/50) |
| M148 E3 | YCL055W | May assist Ste12p in |
| | | pheromone- |
| | | dependent expression |
| | | of KAR3 and CIK1 |
| | | (36.96/45) |
| M149 H7 | YCL059C | Protein essential for |
| | | cell division and |
| | | spore germination |
| | | (34.79/36) |
| M149 C1 | YCL060C | (34.90/34) |
| M148 A3 | YCL062W | (21.56/36) |
| M148 F3 | YCL063W | (14.29/20) |
| M22 E3 | YCL064C | catabolic serine |
| | | (threonine) |
| | | dehydratase |
| | | (39.63/40) |
| M148 G7 | YCL067C | (23.13/36) |
| M148 B1 | YCL068C | (20.93/33) |
| M22 C4 | YCL073C | (67.68/56) |
| M149 H4 | YCL074W | (33.99/40) |
| M21 H2 | YCL075W | (16.27/20) |
| M148 G6 | YCLX01W | (14.63/30) |
| M22 A5 | YCLX03C | (14.00/18) |
| M148 H6 | YCLX09W | (14.41/36) |
| M148 A4 | YCR002C | conserved potential |
| | | GTP-ginding protein |
| | | (35.45/48) |
| M22 E6 | YCR003W | Mitochondrial |
| | | ribosomal protein |
| | | MRPL32 (YmL32) |
| | | (20.24/55) |
| M148 G5 | YCR004C | (27.20/27) |
| M148 A7 | YCR005C | non-mitochondrial |
| | | citrate synthase |
| | | (50.63/50) |
| M148 E1 | YCR007C | (26.32/40) |
| M22 E7 | YCR011C | ATP-dependent |
| | | permease (115.42/?) |
| M148 H5 | YCR012W | 3-phosphoglycerate |
| | | kinase (45.87/45) |
| M148 A6 | YCR020C | (23.68/35) |
| M22 C9 | YCR024C | (54.25/36) |
| M148 B5 | YCR025C | (14.99/25) |
| M148 H1 | YCR036W | ribokinase (36.74/50) |
| M148 D4 | YCR039C | (23.13/33) |
| M148 C5 | YCR040W | transcripton factor |
| | | involved in the |
| | | regulation of the |
| | | alpha-specific genes |
| | | (19.46/33) |
| M21 F1 | YCR045C | (54.04/57) |
| M149 E5 | YCR047C | (30.38/40) |
| M151 C3 | YCR054C | (61.96/61) |
| M151 B1 | YCR060W | (12.32/16) |
| M151 D3 | YCR062W | (13.31/30) |
| M151 G4 | YCR063W | (17.48/17) |
| M26 H4 | YCR065W | Dosage-dependent |
| | | suppressor of cmd1-1 |
| | | mutation\shows |
| | | homology to fork |
| | | head family of DNA- |
| | | binding proteins |
| | | (58.63/60) |
| M151 G8 | YCR066W | Zn finger protein |
| | | putative ATPase |
| | | (53.68/53) |
| M150 C1 | YCR068W | (47.3/51) |
| M151 D6 | YCR072C | (56.68/64) |
| M151 H8 | YCR074C | (11.69/16) |
| M151 E2 | YCR077C | Necessary for |
| | | accurate chromosome |
| | | transmission during |
| | | cell division |
| | | (87.70/98) |
| M26 A5 | YCR082W | (14.29/16) |
| M151 A9 | YCR083W | (14.08/17) |
| M150 F2 | YCR086W | (21.01/36) |
| M150 C5 | YCR088W | Actin binding protein |
| | | (65.23/65) |
| M151 G7 | YCR090C | (20.05/36) |
| M23 G5 | YCR091W | Putative |
| | | serine\/threonine |
| | | protein kinase most |
| | | similar to cyclic |
| | | nucleotide-dependent |
| | | protein kinase |
| | | subfamily and the |
| | | protein kinase C |
| | | subfamily (79.97/80?) |
| M151 C5 | YCR096C | (13.12/20) |
| M151 B9 | YCR098C | (57.01/20) |
| M151 G2 | YCR101C | (20.05/36) |
| M151 H3 | YCR102C | (40.51/50) |
| M151 C9 | YCR106W | (91.63/33) |
| M151 A4 | YCRX03C | (11.25/16) |
| M150 C9 | YCRX07W | (20.45/30) |
| M150 H6 | YCRX13W | multicopy suppressor |
| | | of los1-1 (34.76/50) |
| M26 C5 | YCRX14W | (11.66/14) |
| M150 E10 | YCRX16C | (16.86/28) |
| M26 F1 | YCRX17W | (13.64/20) |
| M151 G5 | YCRX20C | (11.46/64) |
| M150 E9 | YDL002C | HMG1-box |
| | | containing protein |
| | | (22.46/36) |
| M151 D4 | YDL006W | serine-threonine |
| | | protein phosphatase |
| | | (31.02/36) |
| M324 D1 | YDL008W | (18.36/34) |
| M150 F9 | YDL010W | (25.52/34) |
| M151 B2 | YDL012C | 132-410 (11.88/18) |
| M150 F4 | YDL014W | nucleolar protein |
| | | homologous to |
| | | mammalian fibrillarin |
| | | (36.08/40) |
| M151 E8 | YDL017W | protein kinase |
| | | required for initiation |
| | | of mitotic DNA |
| | | synthesis (55.88/64) |
| M152 A1 | YDL020C | Suppressor of sec63 |
| | | (58.44/68) |
| M153 B2 | YDL021W | (34.32/40) |
| M152 D3 | YDL022W | glycerol-3-phosphate |
| | | dehydrogenase |
| | | (43.12/45) |
| M152 F2 | YDL029W | 146-1299 actin- |
| | | related protein |
| | | (43.12/45) |
| M152 E3 | YDL030W | RNA splicing factor |
| | | (58.41/64) |
| M153 E4 | YDL031W | (109.56/109) |
| M153 H6 | YDL033C | (45.90/55) |
| M153 H5 | YDL040C | N-terminal |
| | | acetyltransferase |
| | | (93.97/36) |
| M152 E8 | YDL042C | regulator of silent |
| | | mating loci |
| | | (61.85/68) |
| M152 D1 | YDL044C | Necessary for the |
| | | stability and\/or |
| | | processing of some |
| | | large mitochondrial |
| | | transcripts (48.43/55) |
| M153 D2 | YDL045C | FAD synthetase |
| | | (33.69/50) |
| M152 C6 | YDL048C | (55.58/60) |
| M153 B7 | YDL049C | KRE9 homolog |
| | | (29.51/50) |
| M152 | YDL051W | Protein homologous |
| A10 | | to human La (SS-B) |
| | | autoantigen |
| | | (30.46/40) |
| M152 E1 | YDL052C | putative 1-acyl-sn- |
| | | gylcerol-3-phosphate |
| | | acyl transferase |
| | | (33.46/40) |
| M152 B5 | YDL055C | NDP-hexose |
| | | pyrophosphorylase |
| | | (39.74/45) |
| M27 E5 | YDL056W | transcription factor |
| | | (91.74/90) |
| M274 B2 | YDL059C | (26.21/36) |
| M153 H4 | YDL063C | (68.23/60) |
| M153 B6 | YDL064W | 149-584 (17.48/20) |
| M153 D7 | YDL065C | (38.53/38) |
| M152 G8 | YDL066W | Mitochondrial form |
| | | of NADP-specific |
| | | isocitrate |
| | | dehydrogenase |
| | | (47.29/50) |
| M153 F2 | YDL069C | translational activator |
| | | of cytochrome b |
| | | (25.66/36) |
| M153 G3 | YDL070W | (70.39/70) |
| M28 E2 | YDL072C | (22.46/30) |
| M153 | YDL075W | 479-763 (12.54/20) |
| C10 | | |
| M152 D5 | YDL079C | 705-1798 MDS1 |
| | | related protein kinase |
| | | (55.22/55) |
| M152 F7 | YDL081C | ribosomal protein |
| | | large subunit L12\ |
| | | also known as |
| | | L12eIIA (11.69/18) |
| M152 A2 | YDL084W | (49.27/50) |
| M28 E1 | YDL085W | (60.06/60) |
| M153 A4 | YDL086W | (30.14/33) |
| M152 E5 | YDL087C | EXit from Mitosis |
| | | (28.74/38) |
| M27 F9 | YDL089W | (53.45/45) |
| M333 C2 | YDL090C | component of protein |
| | | prenyltransferase |
| | | (47.44/55) |
| M153 E10 | YDL091C | (50.08/50) |
| M27 A10 | YDL092W | Signal recognition |
| | | particle subunit |
| | | (16.27/16) |
| M152 B3 | YDL093W | dolichyl phosphate- |
| | | D-mannose:protein |
| | | O-D- |
| | | mannosyltransferase |
| | | (81.84/82) |
| M153 B4 | YDL094C | (18.62/40) |
| M255 H1 | YDL097C | (47.77/54) |
| M152 C9 | YDL098C | (21.47/32) |
| M333 D2 | YDL099W | (37.62/42) |
| M152 C2 | YDL100C | (38.97/45) |
| M152 C3 | YDL101C | protein kinase |
| | | (56.46/60) |
| M153 E5 | YDL103C | (52.50/60) |
| M153 F6 | YDL104C | (44.80/50) |
| M153 A8 | YDL105W | (44.43/44) |
| M202 A2 | YDL106C | Homeobox-domain |
| | | containing protein |
| | | which is a positive |
| | | regulator of PHO5 |
| | | and other genes |
| | | (61.52/100) |
| M153 | YDL107W | cox1 pre-mRNA |
| G10 | | splicing factor |
| | | (38.72/40) |
| M152 D2 | YDL108W | 110-1002 serine- |
| | | threonine kinase |
| | | (33.77/40) |
| M152 E4 | YDL110C | (16.53/32) |
| M29 E1 | YDL120W | Mitochondrial protein |
| | | that regulates |
| | | mitochondrial iron |
| | | accumulation iron |
| | | accumulation |
| | | (19.35/19) |
| M155 C6 | YDL121C | (16.42/33) |
| M155 F8 | YDL123W | (15.51/19) |
| M155 B1 | YDL124W | (34.43/40) |
| M30 D2 | YDL125C | 209-588 (17.49/28) |
| M29 C2 | YDL126C | Microsomal protein |
| | | of |
| | | CDC48\/PAS1\/SEC1 |
| | | 8 family of ATPases\ |
| | | full length homology |
| | | to mammalian protein |
| | | VCP\involved in |
| | | secretion peroxisome |
| | | formation and gene |
| | | expression (91.88/92) |
| M155 D6 | YDL129W | (32.12/32) |
| M29 H2 | YDL131W | (48.51/48) |
| M29 A3 | YDL132W | Cell division cycle |
| | | blocked at 36 degree |
| | | C.YDR009 |
| (89.76/90) | | |
| M29 C3 | YDL134C | serine-threonine |
| | | protein phosphatase |
| | | 2A (40.62/40) |
| M29 G3 | YDL137W | ADP-ribosylation |
| | | factor 2 (20.02/20) |
| M29 H3 | YDL138W | (84.04/84) |
| M155 D1 | YDL139C | (27.20/37) |
| M29 C4 | YDL141W | Biotin:apoprotein |
| | | ligase (76.01/76) |
| M274 C2 | YDL142C | Phosphatidylglycerop |
| | | hosphate Synthase |
| | | (31.26/48) |
| M29 F4 | YDL144C | (39.52/40) |
| M155 F7 | YDL145C | (132.14/98) |
| M29 H4 | YDL146W | (54.12/54) |
| M29 D5 | YDL150W | RNA polymerase III |
| | | (C) subunit |
| | | (46.53/46) |
| M155 G7 | YDL153C | Something About |
| | | Silencing 10 |
| | | (67.13/70) |
| M29 H5 | YDL154W | (99.22/99) |
| M30 D1 | YDL155W | G(sub)2-specific B- |
| | | type cyclin (47.08/40) |
| M29 C6 | YDL157C | (13.01/13) |
| M29 E6 | YDL159W | MEK homolog |
| | | (56.76/56) |
| M29 F6 | YDL160C | (55.69/55) |
| M30 C4 | YDL165W | (21.12/32) |
| M155 F4 | YDL166C | (21.70/36) |
| M155 G6 | YDL168W | Long-chain alcohol |
| | | dehydrogenase |
| | | (glytathione- |
| | | dependent |
| | | formaldehyde |
| | | dehydrogenase) |
| | | (42.57/32) |
| M29 H7 | YDL170W | zinc-finger |
| | | transcription factor of |
| | | the Zn(2)-Cys(6) |
| | | binuclear cluster |
| | | domain type |
| | | (58.29/58) |
| M155 G4 | YDL174C | mitochondrial |
| | | enzyme D-lactate |
| | | ferricytochrome c |
| | | oxidoreductase |
| | | (64.60/98) |
| M29 E8 | YDL175C | (37.87/38) |
| M155 A8 | YDL177C | (18.73/29) |
| M155 C9 | YDL178W | D-Lactate |
| | | Dehydrogenase |
| | | (Cytochrome) |
| | | (58.41/64) |
| M29 D9 | YDL182W | (47.29/47) |
| M29 E9 | YDL183C | (35.23/35) |
| M29 F9 | YDL184C | Ribosomal protein |
| | | RPL47 (YL41) |
| | | (RPL47A and |
| | | RPL47B code for |
| | | identical |
| | | proteins)(2.786/5) |
| M154 G1 | YDL187C | (12.02/27) |
| M29 E10 | YDL191W | 495-854 Ribosomal |
| | | protein (rat L35) |
| | | (13.41/31) |
| M255 D2 | YDL194W | glucose transporter |
| | | (97.45/150) |
| M30 G4 | YDL197C | Anti-silencing protein |
| | | that causes depression |
| | | of silent loci when |
| | | overexpressed |
| | | (57.78/60) |
| M29 D11 | YDL198C | (33.03/33) |
| M29 E11 | YDL199C | (75.60/75) |
| M310 G1 | YDL200C | 6-O-methylguanine- |
| | | DNA methylase |
| | | (22.69/34) |
| M155 D8 | YDL201W | (31.57/42) |
| M30 B11 | YDL202W | (27.5/27) |
| M155 B3 | YDL204W | (43.34/54) |
| M29 C12 | YDL205C | phorphobilinogen |
| | | deaminase |
| | | (uroporphyrinogen |
| | | synthase) the third |
| | | step in heme |
| | | biosynthesis |
| | | (36.00/36) |
| M154 C5 | YDL206W | (83.93/115) |
| M155 C7 | YDL208W | HMG-like nuclear |
| | | protein (19.14/30) |
| M31 A1 | YDL211C | (40.95/41) |
| M31 C1 | YDL213C | (24.78/25) |
| M156 B3 | YDL214C | (76.92/98) |
| M156 G4 | YDL216C | (50.08/64) |
| M156 G6 | YDL218W | (34.98/34) |
| M32 E2 | YDL221W | (20.24/28) |
| M31 D2 | YDL222C | (34.02/34) |
| M31 F2 | YDL224C | (71.42/70) |
| M156 H5 | YDL225W | (60.72/90) |
| M31 H2 | YDL226C | ADP-ribosylation |
| | | factor GTPase- |
| | | activating protein |
| | | (ARF GAP) |
| | | (38.75/38) |
| M31 F3 | YDL232W | 3.6-kDa protein |
| | | probably membrane- |
| | | located (4.07/4) |
| M157 B8 | YDL234C | (82.09/98) |
| M157 D1 | YDL235C | Two-component |
| | | phosphorelay |
| | | intermediate |
| | | (18.40/28) |
| M156 F1 | YDL236W | p-nitrophenyl |
| | | phosphatase |
| | | (34.43/40) |
| M157 E3 | YDL237W | (43.01/50) |
| M31 G4 | YDL241W | (13.64/13) |
| M156 B7 | YDL242W | (12.98/18) |
| M156 G1 | YDL244W | (37.51/52) |
| M156 D3 | YDL246C | (39.30/53) |
| M156 B5 | YDL248W | Protein with strong |
| | | similarity to other |
| | | subtelomerically- |
| | | encoded proteins such |
| | | as Cos5p Ybr302p |
| | | Cos3p Cos1p Cos4p |
| | | Cos8p Cos6p Cos9p |
| | | (42.24/48) |
| M156 B6 | YDR001C | (82.64/60) |
| M31 H5 | YDR002W | (22.22/22) |
| M32 C1 | YDR003W | (23.21/38) |
| M31 C6 | YDR005C | 87-1268 (43.56/46) |
| M31 D6 | YDR006C | (99.14/99) |
| M31 F6 | YDR008C | (12.79/13) |
| M31 G6 | YDR009W | galactokinase |
| | | (57.31/57) |
| M32 A2 | YDR012W | large ribosomal |
| | | subunit protein L2B\ |
| | | highly similar to |
| | | ribosomal protein |
| | | L2A (Rpl2bp) |
| | | (39.93/45) |
| M31 F7 | YDR016C | (10.47/10) |
| M31 A8 | YDR019C | glycine cleavage T |
| | | protein (T subunit of |
| | | glycine decarboxylase |
| | | complex (44.03/44) |
| M31 B8 | YDR020C | (25.55/25) |
| M156 G2 | YDR021W | (44/48) |
| M31 D8 | YDR022C | cik1 suppressor |
| | | (21./5922) |
| M156 C4 | YDR023W | seryl-tRNA |
| | | synthetase (50.9/350) |
| M31 F8 | YDR024W | (17.82/18) |
| M31 G8 | YDR025W | 385-810 ribosomal |
| | | protein S18 |
| | | (17.27/20) |
| M156 G3 | YDR030C | Protein involved in |
| | | the same pathway as |
| | | Rad26p has beta- |
| | | transducin (WD-40) |
| | | repeats (55.69/55) |
| M156 D4 | YDR031W | (12.98/18) |
| M31 F9 | YDR032C | (21.81/30) |
| M157 H1 | YDR035W | DAHP synthase\ |
| | | a.k.a. phospho-2- |
| | | dehydro-3- |
| | | deoxyheptonate |
| | | aldolase |
| | | phenylalanine- |
| | | inhibited\phospho-2- |
| | | keto-3- |
| | | deoxyheptonate |
| | | aldolase\2-dehydro- |
| | | 3- |
| | | deoxyphosphohepton |
| | | ate aldolase\3-deoxy- |
| | | D-arabine- |
| | | heptulosonate-7- |
| | | phosphate synthase |
| | | (40.81/48) |
| M156 B2 | YDR036C | (55.03/55) |
| M32 B3 | YDR037W | lysyl-tRNA |
| | | synthetase (65.12/55) |
| M156 E7 | YDR042C | (22.03/28) |
| M31 A11 | YDR043C | (25.44/25) |
| M156 C2 | YDR044W | Coproporphyrinogen |
| | | III oxidase (36.29/44) |
| M32 C5 | YDR047W | (39.93/45) |
| M31 F11 | YDR048C | (11.47/12) |
| M31 G11 | YDR049W | (69.63/?69) |
| M156 F7 | YDR050C | triosephosphate |
| | | isomerase (27.31/33) |
| M32 G1 | YDR051C | (36.77/40) |
| M32 D3 | YDR053W | (14.52/16) |
| M32 F4 | YDR054C | ubiquitin-conjugating |
| | | enzyme E2 (32.48/50) |
| M31 F12 | YDR056C | (22.58/21) |
| M34 D2 | YDR060W | (112.86/112) |
| M33 C1 | YDR061W | (59.4/60) |
| M159 A5 | YDR063W | (16.5/30) |
| M158 G6 | YDR066C | (21.59/33) |
| M158 B1 | YDR067C | (24.67/35) |
| M34 C4 | YDR070C | (10.36/12) |
| M159 B5 | YDR071C | (21.04/30) |
| M34 B1 | YDR075W | protein phosphatase |
| | | type 2A (33.99/40) |
| M34 C1 | YDR083W | (44.33/41) |
| M255 G2 | YDR087C | (30.61/48) |
| M158 A5 | YDR088C | involved in 3′ splice |
| | | site choices |
| | | (42.05/52) |
| M158 C2 | YDR092W | 299-730 ubiquitin- |
| | | conjugating enzyme |
| | | (16.94/30) |
| M301 F1 | YDR097C | (136.65/140) |
| M158 C6 | YDR113C | 42-kDa nuclear |
| | | protein (41.06/55) |
| M34 E7 | YDR114C | (11.03/16) |
| M159 F3 | YDR117C | (62.28/64) |
| M33 G8 | YDR121W | (21.67/40) |
| M33 H10 | YDR138W | (82.83/80) |
| M34 C2 | YDR147W | (58.85/45) |
| M159 B6 | YDR151C | (35.78/48) |
| M158 F6 | YDR153C | (45.24/60) |
| M35 A1 | YDR155C | cyclophilin peptidyl- |
| | | prolyl cis-trans |
| | | isomerase (17.85/18) |
| M160 B2 | YDR156W | RNA polymerase I |
| | | subunit A14 |
| | | (15.28/25) |
| M36 A4 | YDR158W | aspartic beta semi- |
| | | aldehyde |
| | | dehydrogenase |
| | | (40.36/45) |
| M161 C6 | YDR161W | PTC1-Interacting |
| | | Protein (42.68/50) |
| M36 A8 | YDR162C | (25.99/36) |
| M160 B1 | YDR163W | (19.46/32) |
| M160 D4 | YDR167W | TFIID subunit |
| | | (22.77/34) |
| M160 D5 | YDR168W | (55.77/60) |
| M160 E6 | YDR169C | sin3 binding protein |
| | | (56.46/66) |
| M160 C1 | YDR171W | Similar to HSP26\ |
| | | expression is |
| | | regulated by stress |
| | | conditions (41.46/50) |
| M35 C3 | YDR173C | (39.08/?) |
| M160 F3 | YDR174W | (27.27/28) |
| M161 C4 | YDR175C | (35.12/45) |
| M36 E7 | YDR177W | ubiquitin-conjugating |
| | | enzyme (23.76/34) |
| M161 D1 | YDR179C | (17.85/31) |
| M36 E6 | YDR183W | (25.41/36) |
| M160 G6 | YDR184C | (32.47/45) |
| M35 A5 | YDR186C | (96.50/?) |
| M160 H2 | YDR188W | Cytoplasmic |
| | | chaperonin of the Cct |
| | | ring complex |
| | | (previously called |
| | | TCP1 or TRiC) |
| | | distantly related to |
| | | Tcp1p and to Hsp60 |
| | | (60.27/64) |
| M35 D5 | YDR189W | (73.47/75) |
| M161 D4 | YDR190C | (50.96/55) |
| M161 E5 | YDR191W | (40.81/50) |
| M36 F7 | YDR192C | nucleoporin (47.33/53) |
| M35 A6 | YDR194C | Mitochondrial RNA |
| | | helicase of the DEAD |
| | | box family (73.07/?) |
| M35 B6 | YDR195W | (58.74/60) |
| M161 B3 | YDR196C | (26.54/33) |
| M35 D6 | YDR197W | cytochrome b |
| | | translational activator |
| | | (42.9/?) |
| M161 E4 | YDR198C | (52.72/34) |
| M160 G7 | YDR201W | (18.36/34) |
| M160 F1 | YDR202C | (38.64/45) |
| M161 C3 | YDR204W | responsible for |
| | | restoring ubiquinone |
| | | biosynthesis in coq4 |
| | | mutant (36.96/45) |
| M161 F1 | YDR210W | (8.46/16) |
| M35 D8 | YDR213W | (100.5/4?) |
| M160 A5 | YDR214W | (38.61/50) |
| M36 G3 | YDR220C | (10.70/14) |
| M160 B6 | YDR223W | (51.48/66) |
| M160 B7 | YDR224C | Histone H2B (HTB1 |
| | | and HTB2 code for |
| | | nearly identical |
| | | proteins) (14.44/18) |
| M160 A8 | YDR225W | Histone H2A (HTA1 |
| | | and HTA2 code for |
| | | nearly identical |
| | | proteins) (14.63/18) |
| M35 A10 | YDR226W | cytosolic adenylate |
| | | kinase (24.53/25) |
| M35 B10 | YDR227W | regulator of silent |
| | | mating loci (149.49/?) |
| M160 C4 | YDR229W | (49.94/55) |
| M35 H12 | YDR249C | (41.06/?) |
| M38 H1 | YDR251W | (91.41/98) |
| M162 E2 | YDR252W | Negative effect on |
| | | expression of several |
| | | genes transcribed by |
| | | RNA polymerase II\ |
| | | BTF3 homolog |
| | | (16.5/20) |
| M37 D1 | YDR253C | zinc finger DNA |
| | | binding factor |
| | | transcriptional |
| | | regulator of sulfur |
| | | amino acid |
| | | metabolism (21.04/?) |
| M163 G6 | YDR255C | (46.34/60) |
| M37 G1 | YDR256C | catalase A (56.68/60) |
| M163 C9 | YDR257C | (54.47/64) |
| M163 D2 | YDR259C | (42.26/60) |
| M162 D4 | YDR262W | (30.03/36) |
| M37 F2 | YDR263C | DNA-damage |
| | | inducible gene |
| | | (47.33/?) |
| M162 A6 | YDR264C | (84.07/55) |
| M163 B1 | YDR266C | (70.32/80) |
| M163 E3 | YDR268W | mitochondrial |
| | | tryptophanyl-tRNA |
| | | synthetase (41.8/49) |
| M37 F3 | YDR271C | (13.56/14) |
| M163 C8 | YDR272W | Cytoplasmic |
| | | glyoxylase-II |
| | | (30.35/40) |
| M38 E8 | YDR273W | (40.36/50) |
| M162 B2 | YDR275W | (25.96/38) |
| M163 F3 | YDR276C | (6.086/6) |
| M37 F4 | YDR279W | (38.61/38) |
| M163 D1 | YDR282C | (45.57/55) |
| M162 H2 | YDR284C | Diacylglycerol |
| | | Pyrophosphate |
| | | Phosphatase |
| | | (31.82/38) |
| M38 E6 | YDR287W | (32.12/38) |
| M163 G9 | YDR289C | (45.02/64) |
| M38 D1 | YDR290W | (12.1/15) |
| M38 C4 | YDR293C | putative protein |
| | | phosphatase |
| | | (137.5/3130) |
| M37 E6 | YDR294C | (64.82/?) |
| M163 F1 | YDR298C | ATP synthase subunit |
| | | 5\oligomycin |
| | | sensitivity-conferring |
| | | protein (23.45/30) |
| M163 A4 | YDR300C | gamma-glutamyl |
| | | kinase (47.11/48) |
| M37 F7 | YDR303C | (97.48/?) |
| M37 G7 | YDR304C | Cyclophilin D |
| | | Peptidyl-prolyl cis- |
| | | trans isomerase D |
| | | (24.86/40) |
| M163 | YDR305C | 177-743 Yeast |
| A10 | | member of the |
| | | Histidine Triad |
| | | protein family (HIT) |
| | | (27.27/35) |
| M163 G2 | YDR307W | (72.93/75) |
| M163 B4 | YDR308C | RNA polymerase II |
| | | holoenzyme |
| | | component (15.43/31) |
| M38 E4 | YDR309C | (42.26/48) |
| M163 H1 | YDR314C | (76.25/85) |
| M163 H2 | YDR315C | (30.94/40) |
| M163 G8 | YDR320C | (73.51/110) |
| M163 | YDR321W | Asparaginase I |
| C10 | | intracellular isozyme |
| | | (42.02/50) |
| M163 D4 | YDR324C | (85.49/95) |
| M163 A2 | YDR330W | (55.11/70) |
| M163 A3 | YDR331W | Protein involved in |
| | | the attachment of |
| | | glycosylphosphatidyli |
| | | nositol (GPI) anchors |
| | | to proteins (45.32/62) |
| M163 A9 | YDR336W | (34.65/45) |
| M163 E10 | YDR337W | Mitochondrial |
| | | ribosomal protein |
| | | MRPS28 (E. coli |
| | | S15) (31.57/40) |
| M163 B2 | YDR338C | (76.48/76) |
| M163 B3 | YDR339C | (20.82/33) |
| M163 E5 | YDR341C | (66.80/70) |
| M170 A1 | YDR346C | (52.94/60) |
| M170 C9 | YDR353W | Thioredoxin |
| | | reductase (35.2/48) |
| M170 B1 | YDR354W | anthranilate |
| | | phosphoribosyl |
| | | transferase (41.91/50) |
| M274 C3 | YDR354W | anthranilate |
| | | phosphoribosyl |
| | | transferase (41.91/45) |
| M171 F2 | YDR356W | putative |
| | | nucleoskeleton |
| | | component |
| | | (103.95/120) |
| M171 F3 | YDR357C | (13.45/20) |
| M170 D2 | YDR363W | (50.37/55) |
| M171 B2 | YDR363W | (50.37/54) |
| M171 G3 | YDR365C | (69.11/72) |
| M171 H5 | YDR367W | 132-767 (24.42/38) |
| M274 E3 | YDR369C | DNA repair protein |
| | | (93.97/98) |
| M39 A4 | YDR370C | (48.65/45) |
| M171 H3 | YDR373W | (21.01/32) |
| M171 C7 | YDR376W | adrenodoxin |
| | | oxidoreductase |
| | | homolog (54.34/64) |
| M171 E8 | YDR377W | ATP synthase subunit |
| | | f (11.22/20) |
| M171 D1 | YDR378C | (13.56/20) |
| M171 A4 | YDR381W | Nuclear RNA-binding |
| | | RNA annealing |
| | | protein (11.77/18) |
| M40 B6 | YDR383C | (27.75/34) |
| M40 H7 | YDR385W | translation elongation |
| | | factor 2 (EF-2) |
| | | (92.73/85) |
| M39 A6 | YDR386W | (69.63/70) |
| M39 D6 | YDR389W | GTPase activating |
| | | protein (GAP) for |
| | | RHO1 (72.05/72) |
| M171 E1 | YDR394W | (47.29/50) |
| M171 C4 | YDR397C | 130-533 (16.27/30) |
| M171 C5 | YDR398W | (70.84/70) |
| M171 C6 | YDR399W | (24.42/34) |
| M171 F7 | YDR400W | (41.69/50) |
| M171 G7 | YDR408C | glycinamide ribotide |
| | | transformylase |
| | | (23.57/33) |
| M171 C3 | YDR412W | (25.96/50) |
| M171 D6 | YDR415C | (41.27/50) |
| M171 E6 | YDR423C | (45.02/51) |
| M171 D3 | YDR428C | (28.74/38) |
| M171 E4 | YDR429C | (30.27/40) |
| M171 E5 | YDR430C | (108.82/120) |
| M170 A9 | YDR432W | RNA binding protein |
| | | involved in |
| | | mitochondrial protein |
| | | targeting (45.65/48) |
| M171 H1 | YDR434W | (58.85/98) |
| M171 E2 | YDR435C | (36.11/46) |
| M171 E3 | YDR436W | serine-threonine |
| | | phosphatase Z |
| | | (78.21/88) |
| M171 G6 | YDR439W | (38.38/52) |
| M41 C1 | YDR444W | (75.68/100) |
| M173 B6 | YDR446W | ExtraCellular Mutant |
| | | (33.33/42) |
| M41 F1 | YDR447C | 318-725 Ribosomal |
| | | protein RP51B (rat |
| | | S17) (15.07/26) |
| M42 B8 | YDR448W | (47.85/55) |
| M41 A2 | YDR450W | 483-876 (16.27/20) |
| M173 E3 | YDR452W | (74.35/98) |
| M41 D2 | YDR453C | (21.59/21) |
| M173 C6 | YDR454C | guanylate kinase |
| | | (20.60/31) |
| M173 G3 | YDR468C | (24.67/38) |
| M42 C7 | YDR471W | 416-795 60S |
| | | ribosomal protein |
| | | L27 identical to |
| | | Yhr010p (15.07/28) |
| M42 H9 | YDR473C | (51.62/55) |
| M42 E1 | YDR474C | (61.08/60) |
| M173 H3 | YDR476C | (24.67/33) |
| M172 D6 | YDR478W | interstrand crosslink |
| | | repair protein |
| | | (21.89/36) |
| M173 H7 | YDR479C | (60.97/64) |
| M172 A9 | YDR480W | MAP kinase- |
| | | associated protein |
| | | (35.64/52) |
| M173 F10 | YDR481C | repressible alkaline |
| | | phosphatase |
| | | (62.39/70) |
| M172 B1 | YDR482C | (14.88/28) |
| M173 C5 | YDR485C | (89.13/89) |
| M173 G6 | YDR486C | (28.85/38) |
| M172 G7 | YDR487C | 3 4-dihydroxy-2- |
| | | butanone 4-phosphate |
| | | synthase (22.91/34) |
| M42 G8 | YDR488C | (58.66/48) |
| M173 | YDR489W | (32.45/36) |
| G10 | | |
| M173 D5 | YDR493W | (13.64/23) |
| M173 E1 | YDR498C | membrane |
| | | glycoprotein sorted |
| | | by HDEL retrieval |
| | | system (42.26/60) |
| M173 H2 | YDR499W | (82.38/92) |
| M173 A7 | YDR502C | S-adenosylmethionine |
| | | synthetase (42.37/33) |
| M41 E9 | YDR510W | Suppressor of Mif |
| | | Two (11.22/20) |
| M173 G9 | YDR512C | (20.60/28) |
| M172 F10 | YDR513W | Glutaredoxin |
| | | (thioltransferase) |
| | | (glutathione |
| | | reductase) (15.84/28) |
| M173 A3 | YDR515W | regulates the copper- |
| | | dependent |
| | | mineralization of |
| | | copper sulfide |
| | | complexes on the cell |
| | | surface in cells |
| | | cultured in medium |
| | | containing copper |
| | | salts (49.38/50) |
| M173 C4 | YDR516C | (55.03/64) |
| M172 E5 | YDR517W | (41.03/55) |
| M173 C7 | YDR518W | Protein disulfide |
| | | isomerase homolog |
| | | (56.98/56) |
| M173 E8 | YDR519W | FKBP (FK506 |
| | | binding protein) 13\ |
| | | peptidylprolyl cis- |
| | | trans isomerase |
| | | activity (14.96/16) |
| M173 D4 | YDR524C | (53.05/60) |
| M173 F8 | YDR527W | (48.4/48) |
| M172 H1 | YDR530C | 5′ 5″′-P-1 P-4- |
| | | tetraphosphate |
| | | phosphorylase II |
| | | (35.78/50) |
| M173 C3 | YDR531W | (40.48/50) |
| M173 A6 | YDR533C | (26.10/26) |
| M42 G6 | YDR534C | (58.11/58) |
| M174 E3 | YDR540C | (19.72/34) |
| M175 G3 | YEL003W | Polypeptide 2 of a |
| | | Yeast Non-native |
| | | Actin Binding |
| | | Complex homolog of |
| | | a component of the |
| | | bovine NABC |
| | | complex (13.64/30) |
| M175 B5 | YEL004W | (37.73/42) |
| M175 A9 | YEL007W | (73.47/97) |
| M174 C1 | YEL009C | transcriptional |
| | | activator of amino |
| | | acid biosynthetic |
| | | genes (30.94/55) |
| M175 C5 | YEL012W | ubiquitin-conjugating |
| | | enzyme\ubiquitin- |
| | | protein ligase |
| | | (22.77/36) |
| M3 G7 | YEL015W | (60.72/64) |
| M175 | YEL016C | (54.36/90) |
| C10 | | |
| M175 F2 | YEL017W | (37.28/47) |
| M175 A4 | YEL018W | (30.8/38) |
| M175 D5 | YEL019C | Protein involved in |
| | | DNA repair |
| | | (29.40/36) |
| M3 G6 | YEL021W | orotidine-5′- |
| | | phosphate |
| | | decarboxylase |
| | | (29.48/35) |
| M3 D1 | YEL024W | Rieske iron-sulfur |
| | | protein of the |
| | | mitochondrial |
| | | cytochrome bcl |
| | | complex (23.76/30) |
| M175 B4 | YEL026W | (13.97/16) |
| M175 A8 | YEL029C | (34.45/47) |
| M174 H8 | YEL030W | Extra Cellular Mutant |
| | | (70.95/98) |
| M310 C2 | YEL034W | Translation initiation |
| | | factor eIF-5A |
| | | (17.48/30) |
| M174 E5 | YEL035C | (18.39/28) |
| M174 G6 | YEL036C | Mannan 8\Protein of |
| | | the endoplasmic |
| | | reticulum with a role |
| | | in retention of |
| | | glycosyltransferases |
| | | in the Golgi also |
| | | involved in osmotic |
| | | sensitivity and |
| | | resistance to |
| | | aminonitrophenyl |
| | | propanediol |
| | | (55.03/55) |
| M174 H7 | YEL037C | ubiquitin-like protein |
| | | (43.81/60) |
| M175 F10 | YEL039C | iso-2-cytochrome c |
| | | (12.46/22) |
| M3 A3 | YEL041W | (54.56/60) |
| M3 C6 | YEL044W | (18.47/20) |
| M3 C8 | YEL046C | Threonine Aldolase |
| | | (42.60/50) |
| M175 G1 | YEL048C | (16.75/26) |
| M3 B3 | YEL049W | member of the |
| | | seripauperin |
| | | protein\/gene family |
| | | (see Gene₋class |
| | | PAU) (13.31/15) |
| M174 G5 | YEL051W | Vacuolar H-ATPase |
| | | D subunit of the V1 |
| | | catalytic sector |
| | | (28.37/40) |
| M3 D6 | YEL052W | ATPase family gene |
| | | (56.1/60) |
| M175 H1 | YEL056W | subunit of a |
| | | cytoplasmic histone |
| | | acetyltransferase |
| | | (44.22/54) |
| M3 C4 | YEL058W | Phosphoacetylglucosa |
| | | mine Mutase |
| | | (61.38/64) |
| M3 E8 | YEL061C | (114.21/114) |
| M3 D9 | YEL062W | Non-membrane- |
| | | embedded PEST |
| | | sequence-containing |
| | | protein (68.75/68) |
| M174 A2 | YEL063C | arginine permease |
| | | (64.93/55) |
| M3 C3 | YEL064C | (52.83/36) |
| M174 H5 | YEL066W | (19.8/32) |
| M3 E9 | YEL070W | (55.33/60) |
| M3 B2 | YEL071W | (54.67/55) |
| M3 D3 | YEL072W | (25.52/32) |
| M175 H4 | YEL073C | (11.80/26) |
| M3 E3 | YER001W | Alpha-1 3- |
| | | mannosyltransferase |
| | | (83.93/83) |
| M174 H4 | YER002W | (25.52/38) |
| M175 F7 | YER004W | (25.52/34) |
| M310 D2 | YER005W | (69.41/79) |
| M174 D9 | YER006W | (57.31/64) |
| M177 F2 | YER010C | (25.77/40) |
| M177 C6 | YER014W | protoporphyrinogen |
| | | oxidase (59.4/64) |
| M265 B3 | YER015W | Acyl-CoA synthetase |
| | | (fatty acid activator 2) |
| | | (81.95/91) |
| M176 E1 | YER016W | (37.95/50) |
| M5 E8 | YER020W | nucleotide binding |
| | | regulatory protein |
| | | (49.5/42) |
| M176 F1 | YER023W | delta 1-pyrroline-5- |
| | | carboxylate reductase |
| | | (31.57/38) |
| M176 F6 | YER029C | (21.59/34) |
| M177 A1 | YER030W | (17.71/42) |
| M176 H3 | YER034W | (20.46/36) |
| M176 G6 | YER037W | (35.42/48) |
| M177 F3 | YER042W | (20.45/32) |
| M176 C3 | YER048C | DnaJ homolog with a |
| | | leucine zipper |
| | | (43.04/52) |
| M176 D2 | YER055C | ATP |
| | | phosphoribosyltransfe |
| | | rase (32.70/36) |
| M176 A6 | YER058W | Required for |
| | | assembly of active |
| | | cytochrome c oxidase |
| | | (11.88/16) |
| M176 D3 | YER062C | DL-glycerol-3- |
| | | phosphatase (27.53/36) |
| M177 H3 | YER063W | (24.09/50) |
| M5 A6 | YER069W | N-acetyl-gamma- |
| | | glutamyl-phosphate |
| | | reductase and |
| | | acetylglutamate |
| | | kinase (95.04/100) |
| M176 F2 | YER076C | (33.35/50) |
| M5 B6 | YER077C | (75.71/80) |
| M177 B5 | YER079W | (23.21/36) |
| M5 C2 | YER089C | Protein phosphatase |
| | | type 2C (51.07/55) |
| M178 C4 | YER101C | (47.33/60) |
| M179 B6 | YER102W | (22.11/36) |
| M179 B7 | YER103W | member of 70 kDa |
| | | heat shock protein |
| | | family (70.73/98) |
| M179 A1 | YER104W | (22.99/38) |
| M178 D4 | YER109C | putative |
| | | transcriptional |
| | | activator of FLO1 |
| | | (15.54/28) |
| M7 A3 | YER112W | U6 snRNA-associated |
| | | protein (20.68/55) |
| M179 C6 | YER118C | Transmembrane |
| | | osmosensor |
| | | (40.40/50) |
| M178 B6 | YER119C | (49.31/49) |
| M178 D5 | YER125W | Suppressor of |
| | | mutations in SPT3 |
| | | (89.1/89) |
| M178 D1 | YER127W | (39.48/50) |
| M255 C3 | YER128W | (22.44/37) |
| M8 D3 | YER130C | (48.76/55) |
| M8 D4 | YER131W | (13.2/20) |
| M178 H1 | YER136W | GDP dissociation |
| | | inhibitor (49.72/98) |
| M178 C2 | YER137C | (16.31/29) |
| M202 A3 | YER150W | (16.49/60) |
| M179 C2 | YER152C | (48.76/60) |
| M178 D2 | YER153C | translational activator |
| | | of cytochrome c |
| | | oxidase subunit III |
| | | (27.97/35) |
| M178 E3 | YER154W | (44.33/50) |
| M178 G4 | YER156C | (37.21/45) |
| M178 E1 | YER159C | transcription factor |
| | | (15.65/30) |
| M178 E2 | YER161C | non-specific DNA |
| | | binding protein (sin1) |
| | | (36.66/50) |
| M178 G5 | YER165W | Poly(A) binding |
| | | protein cytoplasmic |
| | | and nuclear |
| | | (63.58/98) |
| M178 F3 | YER170W | Adenylate kinase |
| | | (mitochondrial |
| | | GTP:AMP |
| | | phosphotransferase) |
| | | (24.86/33) |
| M9 F3 | YFL-TYA | (/75) |
| M267 A1 | YFL001W | Similar to rRNA |
| | | methyltransferase |
| | | (Caenorhabditis |
| | | elegans) and |
| | | hypothetical 28K |
| | | protein (alkaline |
| | | endoglucanase gene |
| | | 5′ region) from |
| | | Bacillus sp. |
| | | (4/8.7353) |
| M267 E2 | YFL002C | ATP-dependent RNA |
| | | helicase (66./6966) |
| M275 B5 | YFL005W | Ras-like small GTP- |
| | | binding protein |
| | | (23.7/633) |
| M275 E6 | YFL006W | (28.05/35) |
| M267 F2 | YFL010C | (23.24/34) |
| M267 C6 | YFL012W | (16.49/23) |
| M275 F6 | YFL013C | (76.25/89) |
| M267 G2 | YFL016C | DnaJ homolog |
| | | involved in |
| | | mitochondrial |
| | | biogenesis and |
| | | protein folding |
| | | (56.24/56) |
| M10 C3 | YFL017C | (17.52/?) |
| M275 C4 | YFL018C | dihydrolipoamide |
| | | dehydrogenase |
| | | precursor (mature |
| | | protein is the E3 |
| | | component of alpha- |
| | | ketoacid |
| | | dehydrogenase |
| | | complexes) |
| | | (54.92/54) |
| M9 A10 | YFL021W | transcriptional |
| | | activator with GATA- |
| | | 1-type Zn finger |
| | | DNA-binding motif |
| | | (56.21/40) |
| M267 D1 | YFL022C | Phenylalanyl-tRNA |
| | | synthetase beta |
| | | subunit cytoplasmic |
| | | (55.46/59) |
| M267 H2 | YFL023W | (87.67/105) |
| M267 A4 | YFL024C | (91.55/101) |
| M267 F7 | YFL027C | (54.70/60) |
| M267 G8 | YFL028C | ABC ATPase |
| | | (31.82/34) |
| M267 H9 | YFL029C | Cyclin-dependent |
| | | kinase-activating |
| | | kinase (40.51/50) |
| M267 G7 | YFL035C | Mob1p-like protein |
| | | (28.52/37) |
| M10 B6 | YFL037W | beta-tubulin (50.48/?) |
| M9 E4 | YFL038C | Ras-like GTP-binding |
| | | protein\most similar |
| | | to mammalian Rab1A |
| | | protein (22.69/30) |
| M274 G3 | YFL039C | 319-1436 Actin |
| | | (41.46/49) |
| M275 F5 | YFL040W | (59.51/50) |
| M275 A7 | YFL041W | (68.53/98) |
| M275 A9 | YFL043C | (15.98/27) |
| M9 G1 | YFL044C | (33.14/40) |
| M9 C3 | YFL045C | phospho- |
| | | mannomutase |
| | | (27.97/35) |
| M9 F4 | YFL046W | (22.88/32) |
| M10 D7 | YFL047W | (78.65/98) |
| M267 F6 | YFL048C | 47 kDa type I |
| | | transmembrane |
| | | protein localized to |
| | | the Golgi (48.98/55) |
| M267 A9 | YFL050C | ALuminium |
| | | Resistance 2 |
| | | (94.41/104) |
| M275 B9 | YFL051C | (17.63/34) |
| M275 G1 | YFL052W | (51.36/49) |
| M255 D3 | YFL053W | (65.12/98) |
| M275 H3 | YFL054C | (71.09/65) |
| M275 H5 | YFL056C | (23.45/32) |
| M275 B7 | YFL057C | (16.75/25) |
| M267 B9 | YFL058W | a thiamine regulated |
| | | pyrimidine precursor |
| | | biosynthesis enzyme |
| | | (37.51/42) |
| M275 C9 | YFL059W | (32.89/47) |
| M275 H1 | YFL060C | SNZ3 proximal ORF |
| | | stationary phase |
| | | induced gene family |
| | | (24.45/33) |
| M275 B3 | YFL061W | (24.86/32) |
| M275 A4 | YFL062W | Protein with strong |
| | | similarity to |
| | | subtelomerically- |
| | | encoded proteins such |
| | | as Cos5p Ybr302p |
| | | Cos3p Cos1p Cos4p |
| | | Cos8p Cos6p Cos9p |
| | | (41.8/48) |
| M9 B2 | YFL068W | (17.71/25) |
| M267 G5 | YFR001W | (22.55/32) |
| M255 E3 | YFR003C | (17.08/41) |
| M275 C8 | YFR004W | Similar to S. pombe |
| | | PAD1 gene product |
| | | (33.77/46) |
| M275 D9 | YFR005C | (49.31/56) |
| d A11 | YFR006W | (58.96/?) |
| M9 G3 | YFR007W | (38.94/45) |
| M267 G4 | YFR008W | (24.42/36) |
| M275 H4 | YFR009W | Member of ATP- |
| | | binding cassette |
| | | (ABC) family of |
| | | proteins (82.83/80) |
| M10 E11 | YFR010W | (55/55) |
| M267 E8 | YFR011C | (18./7320) |
| M275 D8 | YFR012W | (22.33/27) |
| M275 E9 | YFR013W | (86.68/170) |
| M9 D2 | YFR014C | calmodulin dependent |
| | | protein kinase |
| | | (49.09/40) |
| M202 C3 | YFR015C | Glycogen synthase |
| | | (UDP-gluocse-starch |
| | | glucosyltransferase) |
| | | (77.91/110) |
| M275 A5 | YFR017C | (21.48/35) |
| M267 C7 | YFR018C | (39.96/52) |
| M275 F9 | YFR021W | (55.11/96) |
| M272 A1 | YFR022W | (80.74/101) |
| M272 E2 | YFR023W | poly(A) binding |
| | | protein\related to |
| | | PES4 protein |
| | | homolog YHR015w |
| | | (67.32/70) |
| M273 A4 | YFR024C | (41.06/50) |
| M43 A6 | YFR025C | Histidinolphosphatase |
| | | (36.88/45) |
| M273 B8 | YFR026C | (18.62/34) |
| M272 | YFR028C | soluble tyrosine- |
| D10 | | specific protein |
| | | phosphatase |
| | | (60.64/62) |
| M43 B6 | YFR032C | (31.82/34) |
| M272 H7 | YFR033C | ubiquinol-cytochrome |
| | | c oxidoreductase |
| | | subunit 6 (17 kDa) |
| | | (16.20/26) |
| M43 C8 | YFR034C | myc-type helix-loop- |
| | | helix transcription |
| | | factor (34.45/48) |
| M272 G2 | YFR037C | (61.30/70) |
| M272 B5 | YFR039C | (56.13/64) |
| M43 C6 | YFR040W | 155 kDa SIT4 protein |
| | | phosphatase- |
| | | associated protein |
| | | (99.66/100) |
| M273 H6 | YFR040W | 155 kDa SIT4 protein |
| | | phosphatase- |
| | | associated protein |
| | | (99.66/99) |
| M272 A8 | YFR041C | (32.48/40) |
| M272 F10 | YFR043C | (26.10/33) |
| M272 D1 | YFR044C | (52.94/54) |
| M255 G3 | YFR045W | (19.69/50) |
| M272 C4 | YFR046C | (39.74/52) |
| M43 B5 | YFR047C | (32.48/40) |
| M255 B4 | YFR048W | (72.93/81) |
| M333 C3 | YFR049W | mitochondrial |
| | | ribosomal protein |
| | | (precursor) (13.64/14) |
| M255 C4 | YFR050C | proteasome subunit |
| | | necessary for peptidyl |
| | | glutamyl peptide |
| | | hydrolyzing activity |
| | | (29.39/40) |
| M273 E1 | YFR052W | cytoplasmic 32-34 |
| | | kDa protein |
| | | (30.35/55) |
| M272 A3 | YFR053C | Hexokinase I (PI) |
| | | (also called |
| | | Hexokinase A) |
| | | (53.48/55) |
| M273 G5 | YFR055W | (37.51/45) |
| M274 A4 | YGL001C | (38.42/49) |
| M272 | YGL002W | (23.87/29) |
| H10 | | |
| M273 F1 | YGL003C | (62.39/63) |
| M273 B3 | YGL004C | (45.90/52) |
| M272 E4 | YGL005C | (30.72/40) |
| M272 D8 | YGL008C | plasma membrane |
| | | H+-ATPase |
| | | (101.01/105) |
| M272 F9 | YGL009C | isopropylmalate |
| | | isomerase (85.72/95) |
| M273 G1 | YGL011C | Proteasome subunit |
| | | YC7alpha\/Y8 |
| | | (protease yscE |
| | | subunit 7) (27.75/31) |
| M273 C3 | YGL012W | Sterol C-24 reductase |
| | | (52.14/50) |
| M324 E2 | YGL014W | (97.79/105) |
| M273 | YGL017W | arginyl-tRNA-protein |
| A10 | | transferase (55.44/70) |
| M272 | YGL018C | (20.37/30) |
| A11 | | |
| M272 H1 | YGL019W | casein kinase II beta |
| | | chain (30.69/40) |
| M273 D3 | YGL020C | (25.88/34) |
| M272 B7 | YGL023C | (69.88/79) |
| M272 H9 | YGL025C | Probable transcription |
| | | factor polyglutamine |
| | | domain protein |
| | | (47.44/50) |
| M272 | YGL026C | tryptophan synthetase |
| B11 | | (77.80/82) |
| M273 A2 | YGL027C | (91.66/104) |
| M273 E3 | YGL028C | (59.65/64) |
| M272 F4 | YGL029W | (13.31/22) |
| M273 B6 | YGL030W | 234-548 large |
| | | ribosomal subunit |
| | | protein 32 (11.66/32) |
| M272 C7 | YGL031C | Ribosomal protein |
| | | RPL30A (rp29) |
| | | (YL21) (17.08/25) |
| M272 E8 | YGL032C | adhesion subunit of a- |
| | | agglutinin (9.606/20) |
| M43 B2 | YGL035C | Zinc-finger protein |
| | | (55.47/60) |
| M272 H5 | YGL038C | membrane-bound |
| | | mannosyltransferase |
| | | (52.83/64) |
| M272 D7 | YGL039W | (38.49/45) |
| M272 C2 | YGL043W | RNA polymerase II |
| | | elongation factor |
| | | (34.1/43) |
| M44 F3 | YGL044C | (32.59/40) |
| M272 A6 | YGL046W | (28.93/35) |
| M273 H7 | YGL047W | (22.33/31) |
| M273 | YGL049C | mRNA cap-binding |
| D10 | | protein (eIF-4F) |
| | | 130K subunit |
| | | (100.57/101) |
| M43 C10 | YGL050W | (30.14/35) |
| M301 A2 | YGL052W | (11.22/18) |
| M272 A5 | YGL053W | (26.28/36) |
| M272 B6 | YGL054C | (15.21/20) |
| M273 A8 | YGL055W | delta-9-fatty acid |
| | | desaturase (56.21/60) |
| M43 A8 | YGL056C | (58.00/60) |
| M272 | YGL057C | (31.60/34) |
| C10 | | |
| M202 E3 | YGL058W | Ubiquitin conjugating |
| | | enzyme involved in |
| | | an error-prone DNA- |
| | | damage recovery |
| | | pathway (19.03/33) |
| M310 A3 | YGL059W | (49.06/60) |
| M45 E1 | YGL061C | (27.20/?) |
| M180 B1 | YGL067W | (42.45/50) |
| M180 A2 | YGL068W | (21.45/32) |
| M180 C1 | YGL075C | (42.60/50) |
| M181 E5 | YGL080W | (14.41/33) |
| M181 G7 | YGL082W | (42.02/62) |
| M181 B5 | YGL087C | 97-499 homolog of |
| | | human CROC-1 gene |
| | | protects yeast cells |
| | | from DNA damage |
| | | (15.28/25) |
| M180 H8 | YGL090W | (46.42/64) |
| M181 G5 | YGL096W | (30.47/20) |
| M180 D3 | YGL101W | (23.76/34) |
| M180 C4 | YGL102C | (15.65/16) |
| M180 H7 | YGL105W | associated with tRNA |
| | | and amino acyl-tRNA |
| | | synthetases. |
| | | (41.47/52) |
| M180 B9 | YGL106W | myosin light chain |
| | | (16.5/20) |
| M180 E1 | YGL107C | (71.09/68) |
| M180 E2 | YGL108C | (15.43/20) |
| M180 D5 | YGL111W | (51.04/51) |
| M180 A8 | YGL113W | (73.59/54) |
| M181 D1 | YGL115W | Protein involved in |
| | | derepression of |
| | | glucose-repressed |
| | | genes (35.53/48) |
| M310 B3 | YGL116W | beta-transducin |
| | | homolog (67.21/70) |
| M180 C9 | YGL122C | nuclear |
| | | polyadenylated RNA |
| | | binding protein |
| | | (57.78/64) |
| M180 G1 | YGL123W | ribosomal protein S4 |
| | | (28.05/34) |
| M180 G3 | YGL125W | methlyene |
| | | tetrahydrofolate |
| | | reductase (mthfr) |
| | | (66/66) |
| M181 D4 | YGL126W | (41./9141) |
| M45 A2 | YGL134W | PHO85 cyclin |
| | | (47.7/450) |
| M44 G7 | YGL138C | (37.98/52) |
| M44 H7 | YGL146C | (34.24/36) |
| M181 F1 | YGL147C | Ribosomal protein |
| | | YL11 (rp25) (rp24) |
| | | (E. coli L6) (rat L9) |
| | | (21.04/40) |
| M181 G2 | YGL148W | Chorismate synthase |
| | | (41.47/50) |
| M181 E7 | YGL153W | Peroxisomal |
| | | peripheral membrane |
| | | protein (peroxin) |
| | | (37.62/57) |
| M182 A1 | YGL155W | polypeptide subunit |
| | | of a yeast type 1 |
| | | protein |
| | | geranylgeranyltransfe |
| | | rase (41.47/41) |
| M183 C3 | YGL157W | (38.38/38) |
| M182 A2 | YGL164C | (48.43/64) |
| M182 D4 | YGL166W | regulator of |
| | | metallothionein |
| | | (CUP1) expression |
| | | (24.86/36) |
| M183 A8 | YGL169W | Protein involved in |
| | | translation initiation |
| | | (46.97/46) |
| M182 B1 | YGL171W | Contains domains |
| | | found in the DEAD |
| | | protein family of |
| | | ATP-dependent RNA |
| | | helicases\high-copy |
| | | suppressor of kem1 |
| | | null mutant |
| | | (62.25/64) |
| M183 B2 | YGL172W | nuclear pore complex |
| | | protein with GLFG |
| | | repetitive sequence |
| | | motif (52.03/60) |
| M182 E4 | YGL174W | (29.47/36) |
| M182 G5 | YGL175C | (37.98/55) |
| M183 B1 | YGL179C | (61.63/77) |
| M183 E3 | YGL181W | Glycine-threonine- |
| | | serine repeat protein |
| | | (43.67/54) |
| M182 A7 | YGL184C | (51.28/51) |
| M183 C8 | YGL185C | (41.72/48) |
| M183 H5 | YGL191W | subunit VIa of |
| | | cytochrome c oxidase |
| | | (14.3/16) |
| M183 D8 | YGL193C | (11.46/16) |
| M182 E9 | YGL194C | Protein with |
| | | similarity to Hda1p |
| | | Rpd3p Hos1p and |
| | | Hos3p (49.75/64) |
| M182 C7 | YGL200C | type I |
| | | transmembrane |
| | | protein component of |
| | | COPII-coated ER- |
| | | derived transport |
| | | vesicles (22.46/32) |
| M183 H3 | YGL205W | fatty-acyl coenzyme |
| | | A oxidase (82.49/82) |
| M183 B6 | YGL207W | transcription factor |
| | | (113.96/113) |
| M182 G9 | YGL210W | ras-like GTPase |
| | | (24.53/32) |
| M274 B4 | YGL213C | antiviral protein |
| | | (43.70/52) |
| M183 G7 | YGL216W | (88.66/90) |
| M182 G2 | YGL220W | (13.31/16) |
| M183 B4 | YGL221C | (31.71/36) |
| M183 A5 | YGL222C | (19.38/19) |
| M183 D6 | YGL223C | (45.90/54) |
| M182 F7 | YGL224C | (30.83/38) |
| M183 G9 | YGL226W | (13.64/13) |
| M182 H2 | YGL228W | (63.58/63) |
| M183 B5 | YGL230C | (16.20/27) |
| M182 G7 | YGL232W | 116-928(31.9/38) |
| M182 | YGL234W | glycinamide ribotide |
| B10 | | synthetase and |
| | | aminoimidazole |
| | | ribotide synthetase |
| | | (88.33/88) |
| M182 B4 | YGL237C | transcriptional |
| | | activator protein of |
| | | CYC1 (29.28/36) |
| M182 H7 | YGL240W | (31.24/38) |
| M182 | YGL242C | (19.94/32) |
| C10 | | |
| M182 H1 | YGL243W | (44.11/50) |
| M183 B3 | YGL244W | nuclear protein |
| | | unknown function |
| | | (61.49/68) |
| M183 D4 | YGL245W | (79.75/79) |
| M183 G6 | YGL247W | (21.78/30) |
| M182 | YGL250W | (27.06/38) |
| D10 | | |
| M256 D3 | YGL253W | Hexokinase II (PII) |
| | | (also called |
| | | Hexokinase B) |
| | | (53.57/55) |
| M256 E4 | YGL254W | Sulfur permease II |
| | | (33/36) |
| M184 G6 | YGL256W | alcohol |
| | | dehydrogenase |
| | | isoenzyme IV |
| | | (51./2651) |
| M46 A6 | YGL258W | (22.7/723) |
| M184 A1 | YGL259W | (18.36/28) |
| M256 G6 | YGR001C | 443-675(21.34/31) |
| M256 A8 | YGR002C | (52.49/58) |
| M256 B9 | YGR003W | (81.95/90) |
| M256 B1 | YGR004W | (50.93/55) |
| M256 D2 | YGR005C | transcription initiation |
| | | factor TFIIF middle |
| | | subunit (44.03/60) |
| M184 F4 | YGR007W | choline phosphate |
| | | cytidylyltransferase |
| | | (also called |
| | | phosphoethanolamine |
| | | cytidylyltransferase |
| | | or phosphocholine |
| | | cytidylyltransferase) |
| | | (35.64/36) |
| M184 H5 | YGR008C | (9.376/9) |
| M256 B8 | YGR010W | (43.56/51) |
| M256 E2 | YGR013W | (68.31/75) |
| M46 H2 | YGR015C | (36.11/40) |
| M184 A6 | YGR016W | (21.01/30) |
| M184 B7 | YGR017W | (32.78/38) |
| M256 C8 | YGR018C | (12.02/17) |
| M256 D9 | YGR019W | gamma- |
| | | aminobutyrate |
| | | (GABA) transaminase |
| | | (4-aminobutyrate |
| | | aminotransferase) |
| | | (51.92/54) |
| M184 E2 | YGR021W | (32.01/38) |
| M184 B6 | YGR024C | (26.10/34) |
| M256 E9 | YGR027C | (11.91/16) |
| M256 E1 | YGR028W | 40 kDa putative |
| | | membrane-spanning |
| | | ATPase (39.93/50) |
| M256 G2 | YGR029W | (12.98/16) |
| M184 A5 | YGR031W | (37.73/38) |
| M256 C7 | YGR033C | (26.32/31) |
| M256 F9 | YGR035C | (12.79/17) |
| M256 H2 | YGR037C | Acyl-CoA-binding |
| | | protein |
| | | (ACBP)\/Diazepam |
| | | binding inhibitor |
| | | (DBI)\/endozepine |
| | | (EP) (9.606/14) |
| M184 A4 | YGR038W | (24.53/34) |
| M184 B5 | YGR039W | (11.44/11) |
| M184 C6 | YGR040W | MAP protein kinase |
| | | homolog involved in |
| | | pheromone signal |
| | | transduction |
| | | (40.59/50) |
| M256 D7 | YGR041W | (60.38/63) |
| M184 F8 | YGR042W | (29.92/32) |
| M256 G9 | YGR043C | (36.66/44) |
| M184 F1 | YGR044C | negative regulator of |
| | | meiosis (33.03/36) |
| M256 C5 | YGR047C | transcription factor |
| | | tau (TFIIIC) subunit |
| | | 131 (112.78/115) |
| M184 D6 | YGR048W | (39.82/50) |
| M256 E7 | YGR049W | Protein that |
| | | suppresses ts allele of |
| | | CDC4 when |
| | | overexpressed |
| | | (20.68/30) |
| M184 | YGR051C | (11.80/12) |
| A10 | | |
| M184 G1 | YGR068C | (64.49/48) |
| M46 A4 | YGR072W | up-frameshift |
| | | suppressor (42.68/45) |
| M256 G8 | YGR074W | Homolog of human |
| | | core snRNP protein |
| | | D1 involved in |
| | | snRNA |
| | | maturation (16.27/24) |
| M256 | YGR075C | RNA splicing factor |
| A10 | | (26.65/37) |
| M46 B1 | YGR076C | Mitochondrial |
| | | ribosomal protein |
| | | MRPL25 (YmL25) |
| | | (17.30/30) |
| M184 D4 | YGR078C | Polypeptide 3 of a |
| | | Yeast Non-native |
| | | Actin Binding |
| | | Complex homolog of |
| | | a component of the |
| | | bovine NABC |
| | | complex (21.92/30) |
| M256 E6 | YGR080W | (36.63/44) |
| M184 H7 | YGR081C | (23.13/33) |
| M184 A9 | YGR082W | 20 kDa mitochondrial |
| | | outer membrane |
| | | protein import |
| | | receptor (20.24/20) |
| M46 B7 | YGR083C | translational repressor |
| | | of GCN4 protein |
| | | (71.64/90) |
| M185 E3 | YGR103W | (66.66/66) |
| M185 B7 | YGR106C | (29.28/42) |
| M185 A2 | YGR109C | B-type cyclin |
| | | (41.83/47) |
| M185 G2 | YGR110W | (49.06/50) |
| M186 B5 | YGR112W | Mitochondrial protein |
| | | necessary for |
| | | respiration (42.9/50) |
| M185 G3 | YGR119C | Contains GLFG |
| | | repeats in N-terminal |
| | | half and heptad |
| | | repeats in C-terminal |
| | | half (59.54/62) |
| M185 H4 | YGR120C | (30.38/40) |
| M185 D7 | YGR122W | (44.33/54) |
| M186 C1 | YGR124W | asparagine synthetase |
| | | (63.03/63) |
| M186 C4 | YGR127W | (34.43/40) |
| M185 C6 | YGR129W | (23.76/34) |
| M185 E7 | YGR130C | (89.79/110) |
| M185 C1 | YGR132C | Prohibitin(31.60/40) |
| M186 D4 | YGR135W | proteasome |
| | | component Y13 |
| | | (28.49/36) |
| M185 B5 | YGR136W | (26.62/58) |
| M55 A4 | YGR137W | (13.75/15) |
| M185 C5 | YGR144W | component of the |
| | | biosynthetic pathway |
| | | producing the |
| | | thiazole precursor of |
| | | thiamine(35.97/60) |
| M185 E6 | YGR145W | (77.88/70) |
| M185 H8 | YGR147C | N alpha- |
| | | acetyltransferase that |
| | | acts on methionine |
| | | termini (31.71/35) |
| M54 D1 | YGR148C | Ribosomal protein |
| | | RPL30B (rp29) |
| | | (YL21) (17.08/20) |
| M54 A5 | YGR152C | GTP-binding protein |
| | | of the ras superfamily |
| | | involved in bud site |
| | | selection(29.95/38) |
| M185 F6 | YGR153W | (23.98/34) |
| M186 G7 | YGR154C | (39.29/50) |
| M55 D5 | YGR155W | Cystathionine beta- |
| | | synthase (55.88/55) |
| M55 E1 | YGR156W | (46.86/45) |
| M185 C3 | YGR158C | (27.53/34) |
| M186 F4 | YGR159C | nuclear localization |
| | | sequence binding |
| | | protein (45.57/64) |
| M186 F5 | YGR160W | (22.44/48) |
| M185 G6 | YGR161C | (28.96/38) |
| M186 H8 | YGR163W | (37.62/45) |
| M185 D4 | YGR167W | Clathrin light chain |
| | | (25.74/28) |
| M185 H6 | YGR169C | (44.47/60) |
| M54 G6 | YGR171C | mitochondrial |
| | | methionyl-tRNA |
| | | synthetase (63.38/63) |
| M55 G1 | YGR172C | (27.31/30) |
| M185 D3 | YGR174C | Ubiquinol- |
| | | cytochrome c |
| | | reductase assembly |
| | | factor (18.73/27) |
| M185 A7 | YGR177C | Alcohol |
| | | acetyltransferase |
| | | (58.88/66) |
| M185 C8 | YGR178C | (79.45/79) |
| M277 A1 | YGR180C | Ribonucleotide |
| | | Reductase (37.98/48) |
| M276 C2 | YGR181W | (11.66/14) |
| M276 B3 | YGR182C | (12.90/17) |
| M277 F6 | YGR185C | tyrosyl-tRNA |
| | | synthetase |
| | | cytoplasmic |
| | | (43.47/50) |
| M276 C8 | YGR187C | (43.47/51) |
| M276 D2 | YGR189C | (55.80/55) |
| M255 C5 | YGR192C | Glyceraldehyde-3- |
| | | phosphate |
| | | dehydrogenase 3 |
| | | (36.55/47) |
| M277 E5 | YGR192C | Glyceraldehyde-3- |
| | | phosphate |
| | | dehydrogenase 3 |
| | | (36.55/48) |
| M276 E6 | YGR193C | Protein X component |
| | | of mitochondrial |
| | | pyruvate |
| | | dehydrogenase |
| | | complex (45.13/63) |
| M276 D7 | YGR194C | (66.03/64) |
| M277 B1 | YGR196C | (89.90/115) |
| M276 E2 | YGR197C | involved in |
| | | nitrosoguanidine |
| | | resistance (60.20/63) |
| M277 F3 | YGR198W | (89.98/98) |
| M276 E7 | YGR202C | phosphorylcholine |
| | | transferase\or |
| | | cholinephosphate |
| | | cytidylyltransferase |
| | | (46.67/58) |
| M276 E8 | YGR203W | (16.49/20) |
| M277 C1 | YGR204W | C1-5 6 7 8- |
| | | tetrahydrofolate |
| | | synthase (104.27/105) |
| M276 F2 | YGR205W | (32.01/37) |
| M56 H5 | YGR210C | (45.24/50) |
| M276 F8 | YGR211W | (53.57/70) |
| M276 D1 | YGR212W | (51.59/52) |
| M276 D4 | YGR215W | (12.54/16) |
| M277 G4 | YGR219W | (12.21/16) |
| M276 E1 | YGR220C | Mitochondrial |
| | | ribosomal protein |
| | | MRPL9 (YmL9) |
| | | (E. coli L3) |
| | | (human |
| | | MRL3) (29.62/35) |
| M276 E4 | YGR223C | (49.31/53) |
| M276 F1 | YGR228W | (12.65/13) |
| M277 A3 | YGR229C | 57 kDa nuclear |
| | | protein (55.58/55) |
| M276 F3 | YGR230W | (15.28/20) |
| M276 F5 | YGR232W | (25.29/26) |
| M276 H7 | YGR234W | Flavohemoglobin(44/ |
| | | 48) |
| M276 H8 | YGR235C | (25./6636) |
| M276 G4 | YGR239C | (31.71/38) |
| M277 B6 | YGR240C | phosphofructokinase |
| | | alpha subunit |
| | | (108.60/108) |
| M276 A8 | YGR242W | (11.33/13) |
| M276 A9 | YGR243W | (16.27/18) |
| M276 H1 | YGR244C | (47.00/50) |
| M276 H3 | YGR246C | RNA polymerase III |
| | | transcription factor |
| | | with homology to |
| | | TFIIB (65.59/90) |
| M276 H4 | YGR247W | (26.4/35) |
| M276 H5 | YGR248W | Similar to SOL3 |
| | | (28.26/36) |
| M277 C7 | YGR249W | (50.27/60) |
| M276 B8 | YGR250C | (85.94/100) |
| M276 B9 | YGR251W | (21.67/35) |
| M265 D4 | YGR252W | positive regulator of |
| | | GCN4 expression and |
| | | activity of the HAP2- |
| | | -HAP3--HAP4 |
| | | transcriptional |
| | | activation complex |
| | | (48.4/56) |
| M276 H2 | YGR253C | Proteasome subunit |
| | | (28.63/36) |
| M202 B4 | YGR254W | enolase I(48.28/55) |
| M276 A5 | YGR255C | COQ6 |
| | | monooxygenase |
| | | (52.72/53) |
| M276 A6 | YGR256W | 6-phosphogluconate |
| | | dehydrogenase |
| | | (54.23/55) |
| M276 A7 | YGR257C | (40.39/47) |
| M276 C9 | YGR259C | (16.09/19) |
| M276 A4 | YGR262C | (28.74/35) |
| M277 E6 | YGR264C | methionyl tRNA |
| | | synthetase (82.64/84) |
| M276 D9 | YGR267C | GTP-cyclohydrolase I |
| | | (26.76/35) |
| M56 G1 | YGR268C | (21.81/25) |
| M276 A3 | YGR269W | (11.99/17) |
| M301 F2 | YGR274C | Component of the |
| | | TAFII complex |
| | | required for activated |
| | | transcription |
| | | (117.39/120) |
| M277 F9 | YGR275W | (20.57/31) |
| M260 D2 | YGR277C | (33.58/42) |
| M260 G3 | YGR278W | (63.58/64) |
| M190 A5 | YGR279C | (42.49/51) |
| M49 H3 | YGR280C | (29.84/45) |
| M260 B1 | YGR284C | (34.13/35) |
| M260 E2 | YGR285C | Zuotin putative Z- |
| | | DNA binding protein |
| | | (47.66/55) |
| M190 G3 | YGR286C | Biotin synthase |
| | | (41.38/49) |
| M190 B5 | YGR287C | (64.82/64) |
| M260 F6 | YGR288W | (52.14/60) |
| M190 H3 | YGR294W | (13.31/18) |
| M190 H8 | YHL002W | (49.83/60) |
| M260 E10 | YHL003C | (45.24/45) |
| M260 D1 | YHL004W | mitochondrial |
| | | ribosomal protein |
| | | (43.45/48) |
| M260 E5 | YHL007C | putative |
| | | serine\/threonine |
| | | protein kinase |
| | | (103.32/125) |
| M190 F7 | YHL009C | (36.33/48) |
| M260 E1 | YHL012W | (54.34/57) |
| M190 H2 | YHL013C | (33.80/48) |
| M260 H2 | YHL013C | (33.80/45) |
| M190 E5 | YHL015W | (13.42/19) |
| M260 E9 | YHL018W | (13.31/18) |
| M190 E10 | YHL019C | homologous to the |
| | | medium chain of |
| | | mammalian clathrin- |
| | | associated protein |
| | | complex (66.58/64) |
| M190 F1 | YHL020C | negative regulator of |
| | | phospholipid |
| | | biosynthesis |
| | | (44.47/50) |
| M260 A3 | YHL021C | (51.28/51) |
| M260 D4 | YHL022C | meiotic |
| | | recombination protein |
| | | (43.81/52) |
| M260 B7 | YHL024W | (78.54/85) |
| M190 H7 | YHL025W | transcriptional |
| | | regulator (36.63/50) |
| M260 | YHL027W | Rim 101 protein is |
| H10 | | similar to the |
| | | Aspergillus pH- |
| | | response regulator |
| | | PacC (68.86/80) |
| M260 B3 | YHL029C | (74.72/75) |
| M190 G5 | YHL031C | (24.56/30) |
| M190 A8 | YHL033C | Ribosomal protein |
| | | RPL4A (rp6) (YL5) |
| | | (human L7a) (mouse |
| | | L7a) (rat L7a) |
| | | (RPL4A and RPL4B |
| | | code for nearly |
| | | identical proteins) |
| | | (28.29/32) |
| M260 G9 | YHL034C | (32.47/44) |
| M260 H5 | YHL039W | (64.46/75) |
| M260 H9 | YHL042W | (16.61/20) |
| M49 E1 | YHL044W | (25.96/30) |
| M260 F4 | YHL046C | (13.33/22) |
| M260 E7 | YHL048W | Protein with |
| | | similarity to |
| | | subtelomerically- |
| | | encoded proteins such |
| | | as Cos5p Ybr302p |
| | | Cos3p Cos1p Cos4p |
| | | Cos8p Cos6p Cos9p |
| | | (42.02/45) |
| M190 C8 | YHL049C | (29.84/38) |
| M190 F9 | YHL050C | 1414-2866(76.78/98) |
| M260 | YHR001W | (48.28/52) |
| B11 | | |
| M260 E3 | YHR002W | (39.48/59) |
| M260 | YHR007C | cytochrome P450 |
| B10 | | lanosterol 14a- |
| | | demethylase |
| | | (58.33/60) |
| M190 | YHR008C | Manganese- |
| A11 | | containing superoxide |
| | | dismutase (25.66/33) |
| M190 B2 | YHR009C | (57.56/58) |
| M260 H4 | YHR011W | (49.27/50) |
| M260 C6 | YHR012W | 168-968(31.13/47) |
| M49 F4 | YHR013C | subunit of the major |
| | | N alpha- |
| | | acetyltransferase |
| | | (26.21/30) |
| M260 H8 | YHR014W | (32.12/42) |
| M260 | YHR016C | 216-1575(51.59/55) |
| D11 | | |
| M260 C2 | YHR017W | (42.46/55) |
| M190 F3 | YHR018C | argininosuccinate |
| | | lyase (50.96/53) |
| M190 H4 | YHR019C | Asparaginyl-tRNA |
| | | synthetase (60.97/60) |
| M260 D6 | YHR020W | (75.79/85) |
| M190 F8 | YHR022C | (28.29/35) |
| M188 A1 | YHR025W | homoserine synthase |
| | | (39.48/39) |
| M261 G3 | YHR027C | (109.2/125) |
| M188 B5 | YHR029C | (32.47/36) |
| M261 A8 | YHR030C | putative protein |
| | | kinase (53.37/58) |
| M188 B1 | YHR033W | (46.64/50) |
| M261 D5 | YHR036W | (51.92/60) |
| M261 G6 | YHR037W | delta-1-pyrroline-5- |
| | | carboxylate |
| | | dehydrogenase |
| | | (63.46/58) |
| M188 C7 | YHR039BC | (12.65/18) |
| M61 C1 | YHR040W | (40.47/50) |
| M188 C1 | YHR040W | (40.47/40) |
| M261 E5 | YHR043C | (27.09/34) |
| M61 B7 | YHR045W | (61.71/45) |
| M61 D1 | YHR048W | (56.65/60) |
| M188 A3 | YHR049W | (26.84/35) |
| M188 E6 | YHR052W | (41.47/41) |
| M261 F9 | YHR053C | copper-binding |
| | | metallothionein |
| | | (6.746/15) |
| M261 | YHR054C | (38.97/36) |
| B11 | | |
| M261 E1 | YHR055C | copper-binding |
| | | metallothionein |
| | | (6.746/18) |
| M261 C4 | YHR057C | Peptidylprolyl |
| | | isomerase |
| | | (cyclophilin) ER or |
| | | secreted (22.58/32) |
| M188 B4 | YHR058C | (32.48/38) |
| M188 F6 | YHR060W | required for V- |
| | | ATPase activity |
| | | (20.02/30) |
| M188 F7 | YHR061C | (34.57/38) |
| M261 | YHR062C | Protein subunit of |
| C11 | | nuclear ribonuclease |
| | | P (RNase P) |
| | | (32.36/42) |
| M61 E2 | YHR064C | Hsp70 Protein |
| | | (62.95/64) |
| M261 D4 | YHR065C | (59.76/60) |
| M261 C7 | YHR067W | (30.91/30) |
| M188 A9 | YHR070W | (55/55) |
| M261 G1 | YHR071W | PHO85 cyclin |
| | | (25./333) |
| M188 D4 | YHR074W | (78.6/598) |
| M261 D7 | YHR075C | (44.03/45) |
| M261 G8 | YHR076W | (41.25/43) |
| M61 F8 | YHR077C | Protein involved in |
| | | decay of mRNA |
| | | containing nonsense |
| | | codons (120.04/120) |
| M61 F2 | YHR079C | putative protein |
| | | kinase (122.68/40) |
| M261 A6 | YHR081W | (20.45/31) |
| M261 A2 | YHR086W | putative RNA binding |
| | | protein (57.64/63) |
| M188 F4 | YHR089C | small nucleolar RNP |
| | | proteins (22.58/30) |
| M188 A6 | YHR090C | (31.05/38) |
| M261 C6 | YHR097C | 250-1225(40.47/48) |
| M261 | YHR100C | (20.48/25) |
| D10 | | |
| M261 | YHR101C | 198-1095(36.96/48) |
| H11 | | |
| M61 H2 | YHR103W | (93.83/90) |
| M261 A5 | YHR104W | (36.08/40) |
| M261 D6 | YHR105W | (23.65/34) |
| M188 B6 | YHR106W | Thioredoxin |
| | | reductase (37.73/38) |
| M61 H7 | YHR107C | Component of 10 nm |
| | | filaments of mother- |
| | | bud neck (septin) |
| | | (44.80/50) |
| M261 E10 | YHR108W | (64.46/64) |
| M188 F9 | YHR109W | (64.46/64) |
| M261 F3 | YHR111W | (48.51/59) |
| M261 B5 | YHR112C | (41.61/47) |
| M188 A5 | YHR113W | (54.01/54) |
| M261 H7 | YHR114W | (69.74/75) |
| M261 B9 | YHR115C | (45.79/56) |
| M189 C4 | YHR121W | (20.68/32) |
| M189 B2 | YHR127W | (26.84/36) |
| M189 D3 | YHR128W | UPRTase(27.72/36) |
| M189 A7 | YHR132C | ExtraCellular Mutant |
| | | (47.33/50) |
| M63 F1 | YHR135C | membrane-bound |
| | | casein kinase I |
| | | homolog (59.21/60) |
| M189 E3 | YHR136C | 17 kDa protein |
| | | (16.31/28) |
| M189 D4 | YHR137W | aromatic amino acid |
| | | aminotransferase II |
| | | (56.54/64) |
| M189 D5 | YHR138C | (12.57/13) |
| M189 E5 | YHR144C | dCMP deaminase |
| | | (34.45/38) |
| M191 | YHR147C | Mitochondrial |
| D10 | | ribosomal protein |
| | | MRPL6 (YmL6) |
| | | (23.57/32) |
| M189 E4 | YHR151C | (57.89/66) |
| M191 F1 | YHR156C | (37.43/57) |
| M191 A8 | YHR161C | (70.10/80) |
| M64 G4 | YHR163W | weak multicopy |
| | | suppressor of los1-1 |
| | | (30.91/32) |
| M191 D5 | YHR167W | (28.82/35) |
| M189 E6 | YHR169W | (47.52/50) |
| M310 F3 | YHR170W | putative Upflp- |
| | | interacting protein |
| | | (57.09/64) |
| M64 H4 | YHR171W | (69.41/64) |
| M255 F5 | YHR172W | Spindle Pole Body |
| | | component with an |
| | | molecular weight of |
| | | 97 kDa(90.64/100) |
| M189 G3 | YHR174W | enolase(48.28/48) |
| M189 F6 | YHR177W | (49.94/52) |
| M189 C8 | YHR179W | NAPDH |
| | | dehydrogenase (old |
| | | yellow enzyme) |
| | | isoform 2 (44.11/50) |
| M191 D4 | YHR182W | (86.46/86) |
| M63 G3 | YHR183W | Phosphogluconate |
| | | Dehydrogenase |
| | | (Decarboxylating) |
| | | (53.9/50) |
| M189 G1 | YHR188C | (67.13/67) |
| M189 H5 | YHR192W | (30.69/36) |
| M191 E8 | YHR193C | GAL4 enhancer |
| | | protein homolog of |
| | | human alpha NAC |
| | | subunit of the |
| | | nascent-polypeptide- |
| | | associated complex |
| | | (19.17/34) |
| M191 | YHR195W | (35.42/40) |
| B11 | | |
| M191 H1 | YHR196W | (63.46/66) |
| M189 B5 | YHR199C | (34.13/38) |
| M191 F8 | YHR201C | Cytosolic |
| | | exopolyphosphatase |
| | | (43.70/43) |
| M191 G9 | YHR202W | (66.33/40) |
| M189 B3 | YHR205W | cAMP-dependent |
| | | protein kinase |
| | | homolog suppressor |
| | | of cdc25ts (90.75/98) |
| M189 B4 | YHR206W | (68.53/68) |
| M191 A6 | YHR207C | (57.89/67) |
| M189 A6 | YHR208W | Branched-Chain |
| | | Amino Acid |
| | | Transaminase |
| | | (43.34/48) |
| M191 G8 | YHR209W | (32.12/32) |
| M191 H9 | YHR210C | (37.54/48) |
| M194 G1 | YHR213W | (21.89/32) |
| M194 G3 | YHR214W | (22.44/34) |
| M65 C4 | YHR216W | (57.64/60) |
| M65 C3 | YIL003W | (32.34/32) |
| M194 H3 | YIL010W | (23.76/36) |
| M192 F7 | YIL020C | (28.74/36) |
| M194 C1 | YIL022W | 48.8 kDa protein |
| | | involved in |
| | | mitochondrial protein |
| | | import (47.52/50) |
| M65 F3 | YIL026C | Irregular(126.53/50) |
| M192 D6 | YIL027C | (15.54/20) |
| M192 E4 | YIL033C | regulatory subunit of |
| | | cAMP-dependent |
| | | protein kinase |
| | | (45.79/55) |
| M65 E1 | YIL038C | General negative |
| | | regulator of |
| | | transcription\may |
| | | inhibit RNA |
| | | polymerase II |
| | | transcription |
| | | machinery (91.99/60) |
| M192 F3 | YIL040W | (15.39/22) |
| M65 G2 | YIL041W | (35.97/20) |
| M192 E5 | YIL042C | (43.47/50) |
| M310 G3 | YIL043C | cytochrome b |
| | | reductase (35.45/42) |
| M192 D9 | YIL053W | DL-glycerol-3- |
| | | phosphatase(29.92/38) |
| M194 E4 | YIL057C | (18.07/20) |
| M194 D2 | YIL063C | Yeast Ran-Binding |
| | | protein 2 (36.00/40) |
| M192 G3 | YIL064W | (28.48/36) |
| M192 F9 | YIL069C | 413-817 40S |
| | | ribosomal protein |
| | | S24E (RP50) |
| | | (14.96/18) |
| M65 G1 | YIL070C | (29.39/36) |
| M65 A2 | YIL071W | (/35) |
| M192 D8 | YIL076W | (3/9.649) |
| M192 G9 | YIL077C | (35/.2339) |
| M192 B5 | YIL082W | (32./0140) |
| M192 B7 | YIL083C | (40.2/848) |
| M67 A1 | YIL086C | (11.35/16) |
| M67 D2 | YIL087C | (17.30/20) |
| M195 A4 | YIL089W | (22.66/33) |
| M67 A9 | YIL093C | (29.07/30) |
| M67 B1 | YIL094C | (40.84/50) |
| M67 E2 | YIL095W | probable |
| | | serine\/threonine- |
| | | protein kinase |
| | | (89.21/90) |
| M195 A3 | YIL096C | (36.99/50) |
| M195 B4 | YIL097W | (56.87/60) |
| M67 E5 | YIL098C | (17.08/17) |
| M67 F6 | YIL099W | intracellular |
| | | glucoamylase |
| | | (60.5/60) |
| M195 A2 | YIL103W | (46.86/56) |
| M195 B3 | YIL104C | (55.80/55) |
| M195 C4 | YIL105C | (75.49/75) |
| M195 A5 | YIL106W | Mps One Binder |
| | | (26.07/28) |
| M195 A6 | YIL107C | 6-Phosphofructose-2- |
| | | kinase (91.00/100) |
| M195 B2 | YIL111W | 90-544 Cytochrome-c |
| | | oxidase chain Vb |
| | | (16.72/20) |
| M255 A6 | YIL113W | (23.1/33) |
| M195 B5 | YIL114C | voltage dependent |
| | | anion channel |
| | | (YVDAC2) |
| | | (30.94/40) |
| M195 B7 | YIL116W | histidinol-phosphate |
| | | aminotransferase |
| | | (42.46/54) |
| M67 D9 | YIL117C | (35.01/35) |
| M67 E1 | YIL118W | ras homolog-GTP |
| | | binding protein |
| | | (25.52/30) |
| M195 C2 | YIL119C | inhibitor of ras |
| | | (44.80/54) |
| M66 D4 | YIL122W | (38.72/38) |
| M195 C7 | YIL124W | (32.78/45) |
| M67 E9 | YIL125W | alpha-ketoglutarate |
| | | dehydrogenase |
| | | (111.65/116) |
| M195 D2 | YIL127C | (22.69/38) |
| M195 B6 | YIL131C | (53.37/38) |
| M195 D7 | YIL132C | (23.46/33) |
| M66 F1 | YIL134W | mitochondrial inner |
| | | membrane carrier |
| | | protein for FAD |
| | | (34.32/36) |
| M195 D3 | YIL136W | 45-kDa mitochondrial |
| | | outer membrane |
| | | protein (43.34/43) |
| M66 E4 | YIL138C | Tropomyosin isoform |
| | | 2 (17.74/17) |
| M67 G9 | YIL141W | (14.3/14) |
| M195 E1 | YIL142W | Cytoplasmic |
| | | chaperonin of the Cct |
| | | ring complex related |
| | | to Tcp1p\subunit |
| | | beta (58.08/65) |
| M67 H3 | YIL144W | (76.12/80) |
| M67 C7 | YIL147C | histidine kinase |
| | | osmosensor that |
| | | regulates an |
| | | osmosensing MAP |
| | | kinase cascade and is |
| | | similar to bacterial |
| | | two-component |
| | | regulators |
| | | (134.23/135) |
| M67 H9 | YIL149C | (184.72/185) |
| M195 E3 | YIL152W | (25.96/34) |
| M66 B4 | YIL153W | (43.34/43) |
| M196 G5 | YIL154C | Protein involved in |
| | | nucleo-mitochondrial |
| | | control of maltose |
| | | galactose and |
| | | raffinose |
| | | utilization (38.09/38) |
| M195 D6 | YIL155C | glycerol-3-phosphate |
| | | dehydrogenase |
| | | mitochondrial |
| | | (71.42/40) |
| M67 F8 | YIL156W | Ubiquitin-specific |
| | | protease (117.92/117) |
| M195 F3 | YIL160C | peroxisomal 3- |
| | | oxoacyl CoA thiolase |
| | | (45.90/55) |
| M196 H5 | YIL162W | invertase (sucrose |
| | | hydrolyzing enzyme) |
| | | (58.63/64) |
| M196 B8 | YIL164C | (21.92/32) |
| M67 E7 | YIL171W | (12.1/12) |
| M67 C2 | YIL174W | (8.46/8) |
| M67 D4 | YIL176C | (13.23/20) |
| M195 G5 | YIR001C | (27.53/38) |
| M67 H8 | YIR003W | (74.8/75) |
| M195 A9 | YIR004W | (47.63/89) |
| M197 B2 | YIR006C | PAB-dependent |
| | | poly (A) ribonuclease |
| | | (162.83/180) |
| M310 H3 | YIR007W | (84.15/94) |
| M198 B4 | YIR008C | p48 polypeptide of |
| | | DNA primase |
| | | (45.02/55) |
| M197 B7 | YIR010W | (63.47/70) |
| M68 C7 | YIR011C | restores protein |
| | | transport when |
| | | overexpressed and |
| | | rRNA stability to a |
| | | sec23 mutation |
| | | (35.12/40) |
| M69 C5 | YIR012W | (47.52/50) |
| M69 B1 | YIR014W | (30.47/36) |
| M198 G5 | YIR017C | Transcriptional |
| | | activator of sulfur |
| | | amino acid |
| | | metabolism |
| | | (20.60/32) |
| M197 C7 | YIR018W | (27.06/40) |
| M69 A4 | YIR026 | nitrogen starvation |
| | | induced protein |
| | | phosphatase |
| | | (40.07/48) |
| M198 H7 | YIR027C | allantoinase (50.63/52) |
| M197 D1 | YIR029W | allantoicase (37.84/48) |
| M197 E2 | YIR030C | (26.87/33) |
| M198 E4 | YIR032C | ureidoglycolate |
| | | hydrolase (21.48/34) |
| M197 E7 | YIR034C | saccharopine |
| | | dehydrogenase |
| | | (41.06/48) |
| M68 E7 | YIR035C | (27.97/36) |
| M197 F9 | YIR036C | (28.96/34) |
| M197 E1 | YIR037W | putative glutathione- |
| | | peroxidase (18.04/33) |
| M197 F2 | YIR038C | (25.77/35) |
| M197 F7 | YIR042C | (25.99/35) |
| M197 A4 | YJL003W | (13.09/18) |
| M68 E4 | YJL004C | Multicopy suppressor |
| | | of ypt6 null mutation |
| | | (22.46/30) |
| M197 H9 | YJL008C | Component of |
| | | Chaperonin |
| | | Containing T- |
| | | complex subunit eight |
| | | (62.51/62) |
| M197 B4 | YJL011C | (17.74/27) |
| M310 A4 | YJL013C | Checkpoint protein |
| | | required for cell cycle |
| | | arrest in response to |
| | | loss of microtubule |
| | | function (56.68/65) |
| M197 H7 | YJL014W | Cytoplasmic |
| | | chaperonin subunit |
| | | gamma (58.85/60) |
| M69 F5 | YJL016W | (18.92/28) |
| M197 C4 | YJL019W | (68.31/70) |
| M68 G5 | YJL021C | (40.28/50) |
| M197 A2 | YJL025W | (56.65/60) |
| M68 H5 | YJL029C | (90.45/90) |
| M69 D4 | YJL030W | putative calcium |
| | | binding protein |
| | | (21.67/32) |
| M197 A9 | YJL031C | Geranylgeranyltransfe |
| | | rase Type II alpha |
| | | subunit (PGGTase-II |
| | | alpha subunit) |
| | | (31.93/40) |
| M197 E4 | YJL035C | (27.53/37) |
| M197 H5 | YJL036W | (46.64/50) |
| M68 G2 | YJL043W | (28.48/40) |
| M197 | YJL049W | (49.61/60) |
| D10 | | |
| M197 F2 | YJL052W | Glyceraldehyde-3- |
| | | phosphate |
| | | dehydrogenase 1 |
| | | (36.63/45) |
| M197 A7 | YJL054W | (52.69/34) |
| M197 B8 | YJL055W | (27.06/36) |
| M268 B2 | YJL059W | Homolog of human |
| | | CLN3 (44.99/45) |
| M199 F4 | YJL061W | 82-kDa protein with |
| | | putative coiled-coil |
| | | domain has carboxy- |
| | | terminal domain |
| | | containing heptad |
| | | repeats that binds |
| | | Nsp1p\nucleoporin |
| | | (78.54/80) |
| M268 C5 | YJL062W | (91.41/98) |
| M199 E9 | YJL065C | (18.40/35) |
| M199 B1 | YJL066C | (27.75/40) |
| M199 F9 | YJL073W | DnaJ-like protein of |
| | | the endoplasmic |
| | | reticulum membrane |
| | | (76.23/70) |
| M199 B7 | YJL079C | Similar to plant PR-1 |
| | | class of pathogen |
| | | related proteins |
| | | (32.92/60) |
| M199 D1 | YJL082W | (80.52/80) |
| M268 E2 | YJL083W | (66.55/74) |
| M71 B3 | YJL084C | (115.09/80) |
| M199 E8 | YJL088W | Ornithine |
| | | carbamoyltransferase |
| | | (48.51/40) |
| M268 D1 | YJL090C | (84.07/98) |
| M199 F8 | YJL096W | (24.75/30) |
| M268 D9 | YJL097W | (23.98/29) |
| M199 G3 | YJL100W | (66.88/70) |
| M265 E5 | YJL103C | (68.01/70) |
| M199 G8 | YJL104W | (16.5/20) |
| M268 E9 | YJL105W | (61.71/64) |
| M268 F1 | YJL106W | Homolog of the |
| | | human core snRNP |
| | | protein E\ |
| | | Serine\/Threonine |
| | | protein |
| | | kinase (71.06/88) |
| M268 G5 | YJL110C | GATA zinc finger |
| | | protein 3 (60.64/70) |
| M71 A5 | YJL111W | (60.61/60) |
| M71 F5 | YJL112W | (78.65/80) |
| M268 G1 | YJL114W | (45.65/52) |
| M71 D2 | YJL115W | Anti-silencing protein |
| | | that causes depression |
| | | of silent loci when |
| | | overexpressed |
| | | (30.8/50) |
| M268 A3 | YJL115W | Anti-silencing protein |
| | | that causes depression |
| | | of silent loci when |
| | | overexpressed |
| | | (30.8/46) |
| M71 H3 | YJL117W | Putative inorganic |
| | | phosphate transporter |
| | | (34.32/40) |
| M199 H1 | YJL122W | (19.46/28) |
| M71 E2 | YJL123C | (52.61/50) |
| M199 B4 | YJL124C | (18.95/32) |
| M70 G3 | YJL126W | (33.88/40) |
| M268 G8 | YJL128C | protein kinase |
| | | homolg (73.51/80) |
| M199 D3 | YJL131C | (39.19/48) |
| M199 E5 | YJL133W | mitochondrial carrier |
| | | protein (34.65/40) |
| M71 F1 | YJL138C | translation initiation |
| | | factor (43.48/44) |
| M268 D3 | YJL139C | (47.11/53) |
| M199 D4 | YJL140W | fourth-largest subunit |
| | | of RNA polymerase |
| | | II (24.42/34) |
| M199 A8 | YJL143W | 16.5 kDa inner |
| | | membrane protein |
| | | required for import of |
| | | mitochondrial |
| | | precursor proteins |
| | | (17.49/20) |
| M70 E6 | YJL145W | (32.45/36) |
| M199 B2 | YJL146W | IME2-Dependent |
| | | Signalling (51.7/30) |
| M199 F3 | YJL147C | (42.05/47) |
| M199 E4 | YJL148W | RNA polymerase I |
| | | subunit not shared |
| | | (A34.5) (25.74/40) |
| M199 B8 | YJL151C | (14.66/28) |
| M201 D2 | YJL155C | Frutose-2 6- |
| | | bisphosphatase |
| | | (49.75/55) |
| M73 H3 | YJL156C | (75.60/80) |
| M201 B5 | YJL157C | Factor arrest protein |
| | | (91.33/98) |
| M73 G5 | YJL158C | Protein with |
| | | homology to |
| | | Hsp150p and Pir1p |
| | | Pir2p and Pir3p |
| | | (25.00/60) |
| M73 B1 | YJL162C | (53.05/64) |
| M200 D6 | YJL166W | Ubiquinol |
| | | cytochrome-c |
| | | reductase subunit 8 |
| | | (11 kDa protein) |
| | | (10.45/12) |
| M201 B7 | YJL167W | Farnesyl diphosphate |
| | | synthetase (FPP |
| | | synthetase) |
| | | (38.83/48) |
| M201 B1 | YJL170C | An a-specific gene |
| | | that is induced to a |
| | | higher expression |
| | | level by alpha factor |
| | | (20.26/28) |
| M201 B4 | YJL172W | carboxypeptidase |
| | | yscS (63.47/75) |
| M72 D5 | YJL174W | (30.47/34) |
| M201 C7 | YJL175W | (18.81/28) |
| M73 F8 | YJL176C | transcription factor |
| | | (90.78/90) |
| M72 C1 | YJL178C | (21.59/28) |
| M73 B4 | YJL180C | (35.78/36) |
| M201 D5 | YJL181W | (67.32/67) |
| M73 G8 | YJL184W | (13.64/20) |
| M200 E1 | YJL186W | (64.57/77) |
| M73 C6 | YJL190C | Ribosomal protein |
| | | RPS24 (14.33/16) |
| M73 H8 | YJL192C | (25.77/50) |
| M201 A3 | YJL195C | (25.66/25) |
| M73 A9 | YJL200C | (86.82/45) |
| M200 C3 | YJL203W | RNA splicing factor |
| | | (30.91/43) |
| M72 F8 | YJL208C | mitochondrial |
| | | nuclease (36.22/42) |
| M201 G1 | YJL210W | Required for |
| | | peroxisome |
| | | biogenesis (29.92/32) |
| M201 C3 | YJL211C | (16.20/22) |
| M200 A2 | YJL218W | (21.67/31) |
| M201 G7 | YJL223C | (13.23/20) |
| M73 B2 | YJR002W | (65.34/70) |
| M201 A2 | YJR002W | (65.34/80) |
| M72 C6 | YJR006W | (53.68/55) |
| M72 E7 | YJR007W | Translation initiation |
| | | factor eIF-2 alpha |
| | | subunit (33.55/40) |
| M72 H8 | YJR008W | (37.39/42) |
| M255 E6 | YJR009C | glyceraldehyde 3- |
| | | phosphate |
| | | dehydrogenase |
| | | (36.55/50) |
| M201 F3 | YJR010W | ATP sulfurylase |
| | | (56.32/68) |
| M73 A5 | YJR011C | (28.74/35) |
| M201 G5 | YJR012C | (22.80/34) |
| M200 D8 | YJR014W | (21.89/34) |
| M72 C10 | YJR016C | dihydroxyacid |
| | | dehydratase |
| | | (64.48/64) |
| M201 C2 | YJR017C | Peptidyl-prolyl |
| | | cis\/trans isomerase |
| | | (PPIase) (20.93/33) |
| M201 A5 | YJR019C | peroxisomal acyl- |
| | | CoA thioesterase |
| | | (38.42/45) |
| M200 E8 | YJR022W | (14.29/22) |
| M206 A1 | YJR025C | 3-hydroxyanthranilic |
| | | acid dioxygenase |
| | | (19.50/34) |
| M205 B2 | YJR026W | (48.51/50) |
| M205 E4 | YJR028W | (48.51/55) |
| M205 C2 | YJR034W | Required for |
| | | assembly of active |
| | | cytochrome c oxidase |
| | | (11.99/16) |
| M205 D3 | YJR043C | (38.53/47) |
| M205 G5 | YJR045C | Mitochondrial matrix |
| | | protein involved in |
| | | protein import\ |
| | | subunit of SceI |
| | | endonuclease |
| | | (71.97/72) |
| M205 B7 | YJR046W | (66.55/75) |
| M205 G9 | YJR048C | iso-1-cytochrome c |
| | | (12.1/16) |
| M205 C1 | YJR049C | (58.33/64) |
| M205 E3 | YJR051W | osmotic growth |
| | | protein (55.22/60) |
| M205 F4 | YJR052W | (62.36/64) |
| M205 C7 | YJR054W | (54.78/55) |
| M205 E8 | YJR055W | Protein required for |
| | | growth at high |
| | | temperature |
| | | (18.25/32) |
| M205 H9 | YJR056C | (25.99/36) |
| M205 D1 | YJR057W | thymidylate kinase |
| | | (23.87/33) |
| M202 B5 | YJR060W | basic helix-loop-helix |
| | | protein (38.72/38) |
| M205 A6 | YJR061W | (102.96/100) |
| M205 D7 | YJR062C | 52-kDa amidase |
| | | specific for N- |
| | | terminal asparagine |
| | | and glutamine |
| | | (50.30/50) |
| M205 F3 | YJR067C | (15.54/25) |
| M205 B6 | YJR069C | (21.70/33) |
| M205 E7 | YJR070C | (35.78/45) |
| M52 D2 | YJR074W | (24.09/32.0) |
| M205 G3 | YJR075W | putative |
| | | mannosyltransferase |
| | | (43.67/44) |
| M52 E4 | YJR076C | Component of 10 nm |
| | | filaments of mother- |
| | | bud neck (45.68/50) |
| M52 E5 | YJR077C | mitochondrial protein |
| | | importer receptor |
| | | (34.24/38) |
| M205 F7 | YJR078W | (49.94/50) |
| M205 | YJR080C | (43.47/40) |
| C10 | | |
| M205 F1 | YJR080C | (12.46/20) |
| M205 G2 | YJR083C | (34.02/63) |
| M205 D6 | YJR086w | gamma subunit of G |
| | | protein coupled to |
| | | mating factor |
| | | receptors (12.21/16) |
| M205 H8 | YJR088C | (32.25/48) |
| M205 | YJR089W | (105.05/100) |
| D10 | | |
| M205 B5 | YJR093C | (36.00/36) |
| M303 F3 | YJR094C | meiotic gene |
| | | expression\meiosis |
| | | inducing protein |
| | | (39.63/50) |
| M205 A9 | YJR095W | protein related to |
| | | mitochondrial carriers |
| | | (35.53/40) |
| M205 E10 | YJR096W | (31.13/35) |
| M205 B4 | YJR099W | ubiquitin hydrolase |
| | | (26.07/35) |
| M205 C5 | YJR100C | (36.00/37) |
| M205 F6 | YJR101W | (29.47/37) |
| M205 H7 | YJR102C | (22.35/28) |
| M206 F7 | YJR103W | CTP synthase |
| | | (62.25/48) |
| M205 H1 | YJR105W | (37.51/47) |
| M205 C4 | YJR107W | (36.29/50) |
| M205 C9 | YJR111C | (31.26/36) |
| M205 | YJR112W | (22.22/30) |
| G10 | | |
| M205 A2 | YJR113C | (27.20/35) |
| M206 E4 | YJR116W | (30.8/34) |
| M205 A8 | YJR118C | (22.46/36) |
| M205 D9 | YJR119C | (80.11/90) |
| M209 F2 | YJR123W | ribosomal protein |
| | | RPS5 (mammalian |
| | | S5) (previously called |
| | | rp14 S2 or |
| | | YS8)(24.86/30) |
| M62 D2 | YJR125C | (44.91/64) |
| M209 B1 | YJR129C | (37.32/40) |
| M209 G2 | YJR131W | specific alpha- |
| | | mannosidase |
| | | (60.5/60) |
| M53 A4 | YJR132W | (115.4/9115) |
| M208 F4 | YJR133W | (23.1/33) |
| M310 E4 | YJR134C | (77.80/98) |
| M208 E6 | YJR135C | Required for |
| | | maintenance of |
| | | chromosomes and |
| | | minichromosomes |
| | | (26.32/36) |
| M208 E7 | YJR144W | (29.7/36) |
| M208 C1 | YJR145C | 271-1042 Ribosomal |
| | | protein RPS7B (YS6) |
| | | (rp5) (Rat S4) (human |
| | | S4) (RPS7A and |
| | | RPS7B code for |
| | | identical proteins) |
| | | (28.82/36) |
| M209 A3 | YJR147W | (39.49/49) |
| M53 B4 | YJR148W | Branched-Chain |
| | | Amino Acid |
| | | Transaminase |
| | | (41.47/45) |
| M209 D1 | YJR153W | (39.82/40) |
| M208 B2 | YJR154W | (38.27/50) |
| M208 B3 | YJR155W | (31.79/40) |
| M208 A4 | YJR156C | Thiamine |
| | | biosynthetic enzyme |
| | | (37.43/50) |
| M209 E1 | YJR161C | Protein with |
| | | similarity to members |
| | | of the |
| | | Ybr302p\/Ycr007p\/C |
| | | os8p\/Cos9p family |
| | | coded from |
| | | subtelomeric region |
| | | (42.26/42) |
| M208 C3 | YKL001C | adenylylsulfate kinase |
| | | (22.35/33) |
| M208 B4 | YKL002W | (16.72/36) |
| M208 E1 | YKL006W | 528-815 probable 60S |
| | | ribosomal protein |
| | | L14EA (15.39/18) |
| M209 D2 | YKL007W | alpha subunit of |
| | | capping protein |
| | | (29.59/38) |
| M324 C3 | YKL009W | (26.07/40) |
| M208 H5 | YKL011C | cruciform cutting |
| | | endonuclease |
| | | (38.86/36) |
| M208 H7 | YKL013C | Arp Complex Subunit |
| | | (18.84/30) |
| M208 B5 | YKL018W | (36.3/48) |
| M208 A6 | YKL019W | CAAX |
| | | farnesyltransferase |
| | | alpha subunit |
| | | (34.87/45) |
| M208 E2 | YKL023W | (30.58/33) |
| M209 D3 | YKL024C | uridine- |
| | | monophosphate |
| | | kinase (uridylate |
| | | kinase) (22.47/34) |
| M208 C5 | YKL026C | (18.40/31) |
| M208 B6 | YKL027w | (49.38/60) |
| M202 C5 | YKL035W | (55/60) |
| M208 G3 | YKL040C | (28./2936) |
| M208 C4 | YKL041W | (24.7/540) |
| M208 D5 | YKL042W | Component of the |
| | | spindle pole body |
| | | (40.04/40) |
| M202 D5 | YKL043W | putative transcription |
| | | factor (40.47/55) |
| M208 D6 | YKL051W | (38.94/47) |
| M209 H6 | YKL052C | (32.25/48) |
| M58 A1 | YKL055C | (30.61/33) |
| M58 B2 | YKL056C | (18.40/20) |
| M58 B3 | YKL059C | (48.54/58) |
| M59 E6 | YKL060C | aldolase(39.52/39) |
| M210 E6 | YKL061W | (12.54/16) |
| M210 F1 | YKL063C | (18.40/30) |
| M210 D3 | YKL065C | Yeast endoplasmic |
| | | reticulum 25 kDa |
| | | transmembrane |
| | | protein (22.69/30) |
| M210 H4 | YKL067W | Nucleoside |
| | | diphosphate kinase |
| | | (16.94/28) |
| M210 B1 | YKL070W | (18.7/29) |
| M58 C1 | YKL071W | (28.37/45) |
| M59 A5 | YKL074C | involved in early pre- |
| | | mRNA splicing |
| | | (58.00/60) |
| M59 F5 | YKL075C | (49.53/64) |
| M59 G7 | YKL077W | (43.23/55) |
| M211 C4 | YKL081W | 532-1565 Translation |
| | | elongation factor EF- |
| | | 1gamma (45.43/48) |
| M210 H1 | YKL087C | cytochrome c1 heme |
| | | lyase (24.67/37) |
| M58 C2 | YKL088W | (62.92/70) |
| M210 F4 | YKL090W | (48.84/64) |
| M210 F6 | YKL093W | (37.4/55) |
| M59 B1 | YKL094W | (34.54/45) |
| M210 F2 | YKL096W | cell wall |
| | | mannoprotein |
| | | (26.4/50) |
| M211 E3 | YKL103C | vacuolar |
| | | aminopeptidase ysc1 |
| | | (56.57/64) |
| M59 A6 | YKL106W | aspartate |
| | | aminotransferase |
| | | mitochondrial |
| | | (49.72/50) |
| M210 G5 | YKL107W | (34.1/40) |
| M202 F5 | YKL109W | transcriptional |
| | | activator protein of |
| | | CYC1 (component of |
| | | HAP2\/HAP3 |
| | | heteromer) (61.05/70) |
| M210 G6 | YKL116C | (57.01/70) |
| M210 A3 | YKL119C | 25.2 kDa protein |
| | | involved in assembly |
| | | of vacuolar H(+) |
| | | ATPase (23.68/34) |
| M59 C6 | YKL122C | (18.40/20) |
| M210 H6 | YKL124W | suppressor of SHR3 |
| | | (63.8/100) |
| M210 A7 | YKL132C | (47.33/55) |
| M210 B2 | YKL134C | (84.73/98) |
| M210 B7 | YKL140W | succinate |
| | | dehydrogenase |
| | | cytochrome b subunit |
| | | (60.49/64) |
| M210 C2 | YKL142W | mitochondrial |
| | | ribosomal protein |
| | | (24.2/36) |
| M269 A1 | YKL149C | debranching enzyme |
| | | (44.58/50) |
| M213 G1 | YKL150W | NADH-cytochrome |
| | | b5 reductase |
| | | (33.33/38) |
| M269 A3 | YKL151C | (37.10/49) |
| M269 D4 | YKL152C | Phosphoglycerate |
| | | mutase (27.20/35) |
| M213 G4 | YKL153W | (18.7/30) |
| M77 E6 | YKL154W | (26.95/30) |
| M269 F7 | YKL156W | 354-599 40S |
| | | ribosomal protein |
| | | S27-1 (9.13/10) |
| M213 G2 | YKL159C | (23.24/32) |
| M213 H3 | YKL160W | (16.06/36) |
| M269 F5 | YKL161C | (47.66/52) |
| M78 A8 | YKL163W | Protein containing |
| | | tandem internal |
| | | repeats (35.86/40) |
| M77 H3 | YKL167C | 16 kDa mitochondrial |
| | | ribosomal large |
| | | subunit protein |
| | | (15.10/18) |
| M213 A4 | YKL168C | (80.77/50) |
| M77 G6 | YKL170W | mitochondrial |
| | | ribosomal protein |
| | | L14 (15.39/16) |
| M77 B8 | YKL171W | (102.29/?) |
| M213 B6 | YKL172W | (47.08/64) |
| M77 A4 | YKL175W | (55.44/20) |
| M269 A8 | YKL180W | 616-861 |
| | | (20.45/32) |
| M77 D1 | YKL181W | ribose-phosphate |
| | | pyrophosphokinase |
| | | (47.08/50) |
| M213 A3 | YKL183W | (33.77/34) |
| M77 A5 | YKL184W | Ornithine |
| | | decarboxylase |
| | | (51.47/51) |
| M78 A7 | YKL186C | mRNA transport |
| | | regulator (20.37/32) |
| M269 D1 | YKL189W | (44/55) |
| M77 G2 | YKL190W | 129-604 Type 2B |
| | | protein phosphatase\ |
| | | regulatory B subunit |
| | | of calcineurin |
| | | (19./4620) |
| M78 B4 | YKL191W | (58.8/560) |
| M269 H4 | YKL192C | (13.78/14) |
| M78 H5 | YKL193C | Interacts with and |
| | | may be a positive |
| | | regulator of GLC7 |
| | | which encodes type 1 |
| | | protein phosphatase |
| | | (37.21/40) |
| M77 B7 | YKL194C | mitochondrial |
| | | threonine-tRNA |
| | | synthetase (50.85/51) |
| M269 D7 | YKL195W | (47.08/58) |
| M213 C2 | YKL206C | (29.40/36) |
| M213 C4 | YKL208W | (29.92/38) |
| M77 H1 | YKL213C | (78.68/70) |
| M78 B3 | YKL214C | (22.46/32) |
| M213 D4 | YKL216W | dihydroorotate |
| | | dehydrogenase |
| | | (34.65/40) |
| M77 B6 | YKL217W | carboxylic acid |
| | | transporter protein |
| | | homolog (67.87/70) |
| M77 E7 | YKL218C | (35.89/36) |
| M77 F8 | YKL219W | Protein with |
| | | similarity to |
| | | subtelomerically- |
| | | encoded proteins such |
| | | as Cos5p Ybr302p |
| | | Cos3p Cos1p Cos4p |
| | | Cos8p Cos6p Cos9p |
| | | (44.88/50) |
| M213 E4 | YKL224C | (13.56/16) |
| M77 F7 | YKR001C | putative GTP-binding |
| | | protein\similar to |
| | | mammalian Mx |
| | | proteins (77.47/80) |
| M77 F4 | YKR006C | mitochondrial |
| | | ribosomal protein |
| | | YmL13 (30.38/35) |
| M213 F4 | YKR007W | (20.45/36) |
| M77 E3 | YKR013W | Similar to plant PR-1 |
| | | class of pathogen |
| | | related proteins |
| | | (36.3/60) |
| M78 G4 | YKR014C | (25.77/32) |
| M77 A9 | YKR018C | (79.78/80) |
| M214 A1 | YKR020W | (18.25/34) |
| M218 G1 | YKR021W | (100.76/110) |
| M76 H3 | YKR022C | (35.45/40) |
| M214 B5 | YKR023W | (58.41/68) |
| M214 C7 | YKR025W | (31.13/40) |
| M75 A4 | YKR030W | (30.14/32) |
| M75 A10 | YKR035C | (23.46/23) |
| M218 B1 | YKR036C | CCR4 associated |
| | | factor (72.52/85) |
| M76 F2 | YKR037C | (32.48/40) |
| M214 E7 | YKR041W | (27.61/36) |
| M75 B10 | YKR043C | (29.84/36) |
| M76 G2 | YKR045C | (21.04/30) |
| M75 C4 | YKR046C | (31.26/36) |
| M76 A6 | YKR048C | nucleosome assembly |
| | | protein I (45.90/50) |
| M75 C10 | YKR051W | (46.09/48) |
| M214 D1 | YKR052C | mitochondrial carrier |
| | | protein (33.47/48) |
| M214 E5 | YKR055W | ras homolog-GTP |
| | | binding protein |
| | | (32.12/48) |
| M218 F5 | YKR056W | (67.98/67) |
| M218 E7 | YKR058W | (52.91/80) |
| M75 D10 | YKR059W | translation initiation |
| | | factor (43.56/50) |
| M218 D1 | YKR060W | (30.35/45) |
| M218 C2 | YKR061W | putative |
| | | mannosyltransferase\ |
| | | type 2 membrane |
| | | protein (46.86/58) |
| M76 D4 | YKR062W | Small subunit of |
| | | TFIIE transcription |
| | | factor (36.29/45) |
| M218 F7 | YKR066C | Cytochrome-c |
| | | peroxidase (39.74/39) |
| M76 D10 | YKR067W | (81.84/43) |
| M218 D2 | YKR069W | siroheme synthase |
| | | (65.34/65) |
| M214 D4 | YKR070W | (38.83/40) |
| M214 H6 | YKR072C | sit4 suppressor |
| | | (61.85/61) |
| M75 C9 | YKR074W | (17.26/20) |
| M214 | YKR075C | (33.80/48) |
| B10 | | |
| M218 E2 | YKR077W | (40.04/50) |
| M76 F4 | YKR078W | (64.46/64) |
| M310 C5 | YKR079C | (92.21/100) |
| M76 E6 | YKR080W | NAD-dependent 5 |
| | | 10- |
| | | methylenetetrahydraf |
| | | olate dehydrogenase |
| | | (35.31/40) |
| M218 A7 | YKR081C | (37.87/52) |
| M218 E8 | YKR083C | (14.66/35) |
| M75 D3 | YKR085C | 22.3 kDa |
| | | mitochondrial |
| | | ribsomal large subunit |
| | | protien YmL20\ |
| | | homologous to L17 of |
| | | E. coli (21.48/32) |
| M218 H4 | YKR087C | (34.57/44) |
| M218 F8 | YKR091W | (16.83/35) |
| M214 C8 | YKR097W | phosphoenolpyruvate |
| | | carboxylkinase |
| | | (60.5/65) |
| M202 H5 | YKR099W | (89.32/110) |
| M76 F3 | YKR101W | repressor of silent |
| | | mating loci |
| | | (74.69/35) |
| M75 C2 | YLL002W | (48.07/48) |
| M218 C6 | YLL006W | mitochondrial outer |
| | | membrane protein |
| | | (46.97/60) |
| M218 C7 | YLL007C | (73.28/75) |
| M218 G8 | YLL009C | (7.626/10) |
| M79 D2 | YLL011W | nucleolar snRNP |
| | | protein (53.9/55) |
| M80 E3 | YLL012W | (63.14/63) |
| M219 E1 | YLL019C | protein kinase |
| | | homolog (81.10/90) |
| M220 F5 | YLL022C | (42.48/53) |
| M220 B1 | YLL026W | heat shock protein |
| | | 104 (99.99/99) |
| M219 F1 | YLL027W | (27.61/40) |
| M79 G3 | YLL028W | (64.57/63) |
| M219 H7 | YLL033W | (25.41/40) |
| M219 G1 | YLL035W | (69.63/80) |
| M79 H3 | YLL036C | RNA splicing factor |
| | | (55.46/64) |
| M219 H5 | YLL039C | ubiquitin |
| | | (41.94/50) |
| M219 A8 | YLL041C | Succinate |
| | | dehydrogenase |
| | | (ubiquinone) iron- |
| | | sulfur protein subunit |
| | | (29.39/34) |
| M219 H1 | YLL043W | Suppressor of |
| | | tps1\/fdp1 and |
| | | member of the MIP |
| | | family of |
| | | transmembrane |
| | | channels\may be |
| | | involved in glycerol |
| | | efflux (73.7/81) |
| M80 D5 | YLL045C | Ribosomal protein |
| | | RPL4B (rp6) (YL5) |
| | | (human L7a) (mouse |
| | | L7a) (rat L7a) |
| | | (RPL4A and RPL4B |
| | | code for nearly |
| | | identical proteins) |
| | | (28.29/36) |
| M80 F1 | YLL050C | 194-611 Cofilin actin |
| | | binding and severing |
| | | protein (15.84/30) |
| M80 D9 | YLL056C | (32.81/32) |
| M79 G1 | YLL058W | (63.46/64) |
| M79 C4 | YLL060C | (25.66/32) |
| M219 A7 | YLR005W | (50.82/60) |
| M79 E9 | YLR006C | Two-component |
| | | signal transducer that |
| | | with Sln 1p regulates |
| | | osmosensing MAP |
| | | kinase |
| | | cascade(suppressor of |
| | | sensor kinase) |
| | | (78.45/78) |
| M79 E4 | YLR009W | (22/32) |
| M219 D4 | YLR010C | (17./6330) |
| M219 D5 | YLR011W | (21.1/230) |
| M219 D1 | YLR015W | (55.66/64) |
| M219 D2 | YLR016C | (22.47/40) |
| M219 D3 | YLR017W | Protein that regulates |
| | | ADH2 gene |
| | | expression (37.18/48) |
| M219 E5 | YLR019W | (43.78/50) |
| M219 E8 | YLR022C | (27.53/38) |
| M80 A6 | YLR026C | Sed5p is a t-SNARE |
| | | (soluble NSF |
| | | attachment protein |
| | | receptor) required in |
| | | ER to Golgi transport. |
| | | (37.43/25) |
| M219 F5 | YLR027C | aspartate |
| | | aminotransferase |
| | | cytosolic (47.55/50) |
| M79 F8 | YLR029C | Ribosomal protein |
| | | RPL13A (YL10A) |
| | | (rat L15) (22.47/30) |
| M219 F8 | YLR030W | (29.04/40) |
| M80 C2 | YLR031W | (20.57/32) |
| M219 F3 | YLR033W | (55.33/55) |
| M219 F6 | YLR036C | (22.46/33) |
| M80 B10 | YLR037C | (13.67/13) |
| M223 E1 | YLR040C | (24.67/38) |
| M82 C6 | YLR043C | thioredoxin |
| | | (11.46/12) |
| M81 F7 | YLR044C | pyruvate |
| | | decarboxylase |
| | | (61.96/62) |
| M82 D6 | YLR051C | (23.90/30) |
| M222 G7 | YLR053C | (11.91/22) |
| M82 C10 | YLR054C | (56.45/56) |
| M223 B1 | YLR055C | transcription factor |
| | | (66.35/70) |
| M81 D2 | YLR056W | C-5 sterol desaturase |
| | | (40.36/55) |
| M81 H3 | YLR057W | (93.5/98) |
| M81 D5 | YLR058C | serine |
| | | hydroxymethyl- |
| | | transferase |
| | | (51.62/55) |
| M82 E6 | YLR059C | (29.62/30) |
| M81 H7 | YLR060W | Phenylalanyl-tRNA |
| | | synthetase alpha |
| | | subunit cytoplasmic |
| | | (65.56/65) |
| M82 H8 | YLR061W | 402-755 |
| | | (13.42/28) |
| M222 A5 | YLR066W | signal peptidase |
| | | subunit (20.45/34) |
| M222 H3 | YLR073C | (22.03/34) |
| M81 E5 | YLR074C | (18.39/28) |
| M222 A6 | YLR075W | Ubiquinol- |
| | | cytochrome C |
| | | reductase complex |
| | | subunit VI requiring |
| | | protein (24.42/33) |
| M82 A8 | YLR076C | (15.43/16) |
| M222 H7 | YLR077W | (64.24/67) |
| M81 D1 | YLR079W | P40 inhibitor of |
| | | Cdc28p-Clb5 protein |
| | | kinase complex |
| | | (31.45/50) |
| M223 G3 | YLR082C | Smc4 protein member |
| | | of SMC family |
| | | (43.25/55) |
| M222 B6 | YLR083C | integral membrane |
| | | protein\p24a protein |
| | | (73.40/90) |
| M222 B4 | YLR089C | (65.25/65) |
| M81 G5 | YLR090W | Homolog of E. coli |
| | | DnaJ closely related |
| | | to Ydj1p (50.6/60) |
| M81 H6 | YLR091W | (32.34/40) |
| M81 H6 | YLR091W | (32.34/40) |
| M222 B8 | YLR093C | (27.64/39) |
| M223 H2 | YLR097C | (37.87/52) |
| M81 H5 | YLR098C | DNA binding |
| | | activator (71.31/75) |
| M222 D6 | YLR099C | (43.47/48) |
| M82 C8 | YLR100W | (38.38/?) |
| M81 A11 | YLR102C | (29.28/45) |
| M82 F1 | YLR103C | omosomal DNA |
| | | replication initiation |
| | | protein (71.53/?) |
| M222 E6 | YLR107W | (44.55/48) |
| M222 D8 | YLR109W | (19.47/38) |
| M81 G4 | YLR113W | mitogen-activated |
| | | protein kinase (MAP |
| | | kinase) (47.96/60) |
| M81 A6 | YLR114C | (84.07/100) |
| M223 D1 | YLR119W | suppressor of rna1-1 |
| | | mutation (23.54/33) |
| M222 D7 | YLR124W | (12.65/16) |
| M222 F8 | YLR125W | (15.07/40) |
| M82 A2 | YLR127C | APC (anaphase |
| | | promoting complex) |
| | | component (93.86/94) |
| M82 D7 | YLR131C | activator of CUP1 |
| | | expression (84.73/40) |
| M222 E7 | YLR132C | (31.93/40) |
| M221 A3 | YLR137W | (40.48/52) |
| M84 C6 | YLR139C | (70.76/70) |
| M86 G9 | YLR141W | Upstream activation |
| | | factor subunit |
| | | (40.04/55) |
| M221 E6 | YLR142W | proline oxidase |
| | | (52.47/60) |
| M84 C2 | YLR144C | Identified as an |
| | | activity necessary for |
| | | actin polymerization |
| | | in permeabilized cells |
| | | (85.72/90) |
| M79 E4 | YLR009W | (22/32) |
| M219 D4 | YLR010C | (17./6330) |
| M219 D5 | YLR011W | (21.1/230) |
| M219 D1 | YLR015W | (55.66/64) |
| M219 D2 | YLR016C | (22.47/40) |
| M219 D3 | YLR017W | Protein that regulates |
| | | ADH2 gene |
| | | expression (37.18/48) |
| M219 E5 | YLR019W | (43.78/50) |
| M219 E8 | YLR022C | (27.53/38) |
| M80 A6 | YLR026C | Sed5p is a t-SNARE |
| | | (soluble NSF |
| | | attachment protein |
| | | receptor) required in |
| | | ER to Golgi transport. |
| | | (37.43/25) |
| M219 F5 | YLR027C | aspartate |
| | | aminotransferase |
| | | cytosolic (47.55/50) |
| M79 F8 | YLR029C | Ribosomal protein |
| | | RPL13A (YL10A) |
| | | (rat L15) (22.47/30) |
| M219 F8 | YLR030W | (29.04/40) |
| M80 C2 | YLR031W | (20.57/32) |
| M219 F3 | YLR033W | (55.33/55) |
| M219 F6 | YLR036C | (22.46/33) |
| M80 B10 | YLR037C | (13.67/13) |
| M223 E1 | YLR040C | (24.67/38) |
| M82 C6 | YLR043C | thioredoxin |
| | | (11.46/12) |
| M81 F7 | YLR044C | pyruvate |
| | | decarboxylase |
| | | (61.96/62) |
| M82 D6 | YLR051C | (23.90/30) |
| M222 G7 | YLR053C | (11.91/22) |
| M82 C10 | YLR054C | (56.45/56) |
| M223 B1 | YLR055C | transcription factor |
| | | (66.35/70) |
| M81 D2 | YLR056W | C-5 sterol desaturase |
| | | (40.36/55) |
| M81 H3 | YLR057W | (93.5/98) |
| M81 D5 | YLR058C | serine |
| | | hydroxymethyltransfe |
| | | rase (51.62/55) |
| M82 E6 | YLR059C | (29.62/30) |
| M81 H7 | YLR060W | Phenylalanyl-tRNA |
| | | synthetase alpha |
| | | subunit cytoplasmic |
| | | (65.56/65) |
| M82 H8 | YLR061W | 402-755 |
| | | (13.42/28) |
| M222 A5 | YLR066W | signal peptidase |
| | | subunit (20.45/34) |
| M222 H3 | YLR073C | (22.03/34) |
| M81 E5 | YLR074C | (18.39/28) |
| M81 E5 | YLR074C | (18.39/28) |
| M222 A6 | YLR075W | Ubiquinol- |
| | | cytochrome C |
| | | reductase complex |
| | | subunit VI requiring |
| | | protein (24.42/33) |
| M222 A6 | YLR075W | Ubiquinol- |
| | | cytochrome C |
| | | reductase complex |
| | | subunit VI requiring |
| | | protein (24.42/33) |
| M82 A8 | YLR076C | (15.43/16) |
| M222 H7 | YLR077W | (64.24/67) |
| M223 G5 | YLR077W | (64.24/60) |
| M81 D1 | YLR079W | P40 inhibitor of |
| | | Cdc28p-Clb5 protein |
| | | kinase complex |
| | | (31.45/50) |
| M223 G3 | YLR082C | Smc4 protein member |
| | | of SMC family |
| | | (43.25/55) |
| M222 B6 | YLR083C | integral membrane |
| | | protein\p24a protein |
| | | (73.40/90) |
| M222 B4 | YLR089C | (65.25/65) |
| M222 B4 | YLR089C | (65.25/65) |
| M81 G5 | YLR090W | Homolog of E. coli |
| | | DnaJ closely related |
| | | to Ydj1p (50.6/60) |
| M81 H6 | YLR091W | (32.34/40) |
| M81 H6 | YLR091W | (32.34/40) |
| M222 B8 | YLR093C | (27.64/39) |
| M223 H2 | YLR097C | (37.87/52) |
| M81 H5 | YLR098C | DNA binding |
| | | activator (71.31/75) |
| M222 D6 | YLR099C | (43.47/48) |
| M82 C8 | YLR100W | (38.38/?) |
| M82 C8 | YLR100W | (38.38/?) |
| M81 A11 | YLR102C | (29.28/45) |
| M82 F1 | YLR103C | omosomal DNA |
| | | replication initiation |
| | | protein (71.53/?) |
| M222 E6 | YLR107W | (44.55/48) |
| M222 D8 | YLR109W | (19.47/38) |
| M81 G4 | YLR113W | mitogen-activated |
| | | protein kinase (MAP |
| | | kinase) (47.96/60) |
| M81 A6 | YLR114C | (84.07/100) |
| M81 A6 | YLR114C | (84.07/100) |
| M223 D1 | YLR119W | suppressor of rnal-1 |
| | | mutation (23.54/33) |
| M222 D7 | YLR124W | (12.65/16) |
| M222 F8 | YLR125W | (15.07/40) |
| M82 A2 | YLR127C | APC (anaphase |
| | | promoting complex) |
| | | component (93.86/94) |
| M82 D7 | YLR131C | activator of CUP1 |
| | | expression (84.73/40) |
| M222 E7 | YLR132C | (31.93/40) |
| M221 A3 | YLR137W | (40.48/52) |
| M84 C6 | YLR139C | (70.76/70) |
| M86 G9 | YLR141W | Upstream activation |
| | | factor subunit |
| | | (40.04/55) |
| M221 E6 | YLR142W | proline oxidase |
| | | (52.47/60) |
| M84 C2 | YLR144C | Identified as an |
| | | activity necessary for |
| | | actin polymerization |
| | | in permeabilized cells |
| | | (85.72/90) |
| M221 F6 | YLR150W | (30.14/42) |
| M255 H6 | YLR151C | (37.43/52) |
| M84 G3 | YLR153C | acetyl-coenzyme A |
| | | synthetase (75.16/75) |
| M221 G4 | YLR155C | nitrogen catabolite- |
| | | regulated cell-wall L- |
| | | asparaginase II |
| | | (39.85/50) |
| M221 A2 | YLR160C | nitrogen catabolite- |
| | | regulated cell-wall L- |
| | | asparaginase II |
| | | (39.85/50) |
| M84 A8 | YLR164W | (18.59/19) |
| M221 B1 | YLR167W | ubiquitin |
| | | (16.83/16) |
| M221 B2 | YLR168C | (25.33/35) |
| M86 G8 | YLR172C | S-adenosylmethionine |
| | | (AdoMet)-dependent |
| | | methyltransferase of |
| | | diphthamide |
| | | biosynthesis |
| | | (33.03/40) |
| M224 F1 | YLR175W | major low affinity 55 |
| | | kDa |
| | | Centromere\/microtub |
| | | ule binding protein |
| | | (53.24/60) |
| M221 C2 | YLR176C | (89.24/96) |
| M86 H5 | YLR178C | suppressor of cdc25 |
| | | (24.12/38) |
| M221 H4 | YLR179C | (22.14/33) |
| M221 F5 | YLR180W | S-adenosylmethionine |
| | | synthetase (42.13/48) |
| M221 E4 | YLR186W | (27.83/36) |
| M84 A7 | YLR187W | (112.97/114) |
| M84 D8 | YLR188W | ATP-binding cassette |
| | | (ABC) transporter |
| | | family member |
| | | (76.56/76) |
| M84 H9 | YLR189C | (131.81/?) |
| M84 D11 | YLR190W | (54.12/70) |
| M84 G1 | YLR191W | Peroxisomal |
| | | membrane protein |
| | | that contains Src |
| | | homology 3 (SH3) |
| | | domain (42.57/45) |
| M221 F3 | YLR193C | (19.38/30) |
| M84 B7 | YLR195C | N-myristoyl |
| | | transferase (50.08/32) |
| M84 A10 | YLR197W | homology to |
| | | microtubule binding |
| | | proteins and to |
| | | X90565_5.cds |
| | | (55.55/55) |
| M221 D1 | YLR199C | (24.23/36) |
| M221 E2 | YLR200W | Polypeptide 6 of a |
| | | Yeast Non-native |
| | | Actin Binding |
| | | Complex homolog of |
| | | a component of the |
| | | bovine NABC |
| | | complex (12.65/18) |
| M84 D4 | YLR201C | (28.63/40) |
| M84 C7 | YLR203C | Protein involved in |
| | | maturation of COX1 |
| | | and COB mRNA |
| | | (47.99/48) |
| M224 H9 | YLR206W | (67.54/76) |
| M86 F3 | YLR208W | cytoplasmic protein |
| | | involved in relase of |
| | | transport vesicles |
| | | from the ER |
| | | (32.78/34) |
| M84 E4 | YLR209C | (34.24/35) |
| M84 D7 | YLR211C | (15.43/25) |
| M224 H8 | YLR213C | (46.45/54) |
| M221 E1 | YLR215C | (39.63/40) |
| M86 G3 | YLR216C | a cyclophilin related |
| | | to the mammalian |
| | | CyP-40\physically |
| | | interacts with RPD3 |
| | | gene product |
| | | (40.84/48) |
| M84 A6 | YLR218C | (16.53/17) |
| M84 D10 | YLR221C | (24.23/45) |
| M84 D3 | YLR224W | (40.7/40) |
| M84 F7 | YLR227C | (54.26/12) |
| M85 D4 | YLR243W | (30.03/40) |
| M225 B2 | YLR248W | Serine\/threonine |
| | | protein kinase |
| | | (67.21/100) |
| M226 D8 | YLR254C | (20.82/34) |
| M85 C1 | YLR255C | (12.90/20) |
| M83 H2 | YLR257W | (35.42/?) |
| M83 B4 | YLR258W | Glycogen synthase |
| | | (UDP-gluocse-starch |
| | | glucosyltransferase) |
| | | (77.66/78) |
| M85 F4 | YLR259C | heat shock protein 60\ |
| | | chaperonin protein |
| | | (62.95/65) |
| M274 E6 | YLR260W | (75.68/78) |
| M85 C7 | YLR261C | Ras-like GTP binding |
| | | protein (11.91/20) |
| M85 G2 | YLR265C | (37.65/37) |
| M85 G4 | YLR267W | (62.81/64) |
| M85 B6 | YLR268W | Synaptobrevin (v- |
| | | SNARE) homolog |
| | | present on ER to |
| | | Golgi vesicles |
| | | (23.65/33) |
| M226 E8 | YLR270W | (38.61/48) |
| M85 E1 | YLR271W | (30.35/40) |
| M83 B3 | YLR273C | Protein similar to |
| | | Gac1p a putative type |
| | | 1 protein phosphatase |
| | | targeting subunit |
| | | (71.31/45) |
| M85 C6 | YLR276C | (65.47/70) |
| M85 F1 | YLR279W | (14.3/20) |
| M85 C2 | YLR280C | (12.79/20) |
| M225 E5 | YLR283W | (34.65/35) |
| M85 D6 | YLR284C | (30.83/37) |
| M85 A9 | YLR286C | Endochitinase |
| | | (61.85/66) |
| M225 F1 | YLR287C | (39.08/50) |
| M265 F6 | YLR288C | involved in |
| | | checkpoint control |
| | | and DNA repair |
| | | (52.27/60) |
| M225 F5 | YLR290C | (30.50/36) |
| M225 F6 | YLR291C | negative regulator of |
| | | GCN4 expression |
| | | (41.94/56) |
| M226 F7 | YLR300W | Exo-1 3-beta- |
| | | glucanase (49.49/49) |
| M83 D2 | YLR303W | (48.95/?) |
| M225 G6 | YLR307W | (33.22/51) |
| M225 A6 | YLR314C | Component of 10 nm |
| | | filaments of mother- |
| | | bud neck (57.23/80) |
| M85 B8 | YLR316C | (17.96/28) |
| M85 B4 | YLR321C | (46.89/60) |
| M226 A7 | YLR323C | (28.52/38) |
| M225 D8 | YLR324W | (57.64/98) |
| M88 F8 | YLR332W | Protein required for |
| | | mating (41.47/55) |
| M88 F3 | YLR336C | (98.92/98) |
| M88 E7 | YLR339C | (20.26/25) |
| M88 G8 | YLR340W | 60S ribosomal protein |
| | | P0 (L10E). (34.43/36) |
| M88 A5 | YLR345W | (56.1/64) |
| M88 D10 | YLR349W | (18.59/50) |
| M228 C1 | YLR350W | (23.87/33) |
| M88 F2 | YLR351C | (32.04/38) |
| M88 B5 | YLR353W | (66.44/80) |
| M88 E6 | YLR354C | Transaldolase enzyme |
| | | in the pentose |
| | | phosphate pathway |
| | | (36.88/45) |
| M228 E2 | YLR359W | Adenylosuccinate |
| | | Lyase (53.13/68) |
| M88 C5 | YLR361C | (63.61/75) |
| M88 H7 | YLR363C | (24.01/34) |
| M230 H3 | YLR369W | (72.48/64) |
| M88 E5 | YLR377C | fructose-1 6- |
| | | bisphosphatase |
| | | (38.31/45) |
| M333 E4 | YLR396C | (76.04/78) |
| M230 D7 | YLR412W | (30.35/40) |
| M89 A2 | YLR423C | (45.90/50) |
| M89 D5 | YLR426W | 152-1052 |
| | | (35.97/36) |
| M89 B2 | YLR431C | (49.86/55) |
| M89 A3 | YLR432W | (57.64/57) |
| M231 E2 | YLR433C | Calcineurin subunit |
| | | A\type 2B protein |
| | | serine\/threonine |
| | | phosphatase catalytic |
| | | subunit A1\ |
| | | cytoplasmic |
| | | (60.86/61) |
| M89 G6 | YLR435W | (27.5/36) |
| M89 E8 | YLR437C | (14.66/14) |
| M89 A1 | YLR438W | ornithine |
| | | aminotransferase |
| | | (46.75/46) |
| M229 H1 | YLR439W | Mitochondrial 60S |
| | | ribosomal protein L4 |
| | | (35.2/45) |
| M89 C4 | YLR441C | Ribosomal protein |
| | | analogous to rat S3A |
| | | (28.08/38) |
| M231 B1 | YLR446W | (47.74/50) |
| M89 D4 | YLR449W | (43.23/45) |
| M229 B2 | YLR455W | (33.55/45) |
| M89 D3 | YLR456W | (22.55/22) |
| M229 H2 | YLR457C | (35.12/55) |
| M229 A6 | YLR460C | (41.49/50) |
| M89 H8 | YLR461W | member of the |
| | | seripauperin |
| | | protein\/gene family |
| | | (see Gene_class PAU) |
| | | (13.31/13) |
| M231 C1 | YLR462W | (22.33/34) |
| M89 E3 | YLR464W | 793-930 |
| | | (23.87/23) |
| M231 C2 | YML004C | lactoylglutathione |
| | | lyase (glyoxalase I) |
| | | (35.89/48) |
| M89 F3 | YML005W | (50.93/50) |
| M89 F4 | YML006C | (85.27/100) |
| M231 B4 | YML007W | jun-like transcription |
| | | factor (71.61/80) |
| M89 C7 | YML008C | S- |
| | | adenoslymethionine: |
| | | delta 24- |
| | | methyltransferase |
| | | (42.26/50) |
| M89 G3 | YML011C | (19.50/20) |
| M231 C3 | YML012W | Component of the |
| | | COPII coat of certain |
| | | ER-derived vesicles |
| | | (23.32/32) |
| M231 H5 | YML014W | (30.8/38) |
| M89 E1 | YML016C | serine-threonine |
| | | phosphatase Z |
| | | (76.25/76) |
| M89 D6 | YML028W | Thiol-specific |
| | | antioxidant (21.67/32) |
| M231 D1 | YML032C | (55.47/63) |
| M89 B5 | YML034W | (72.37/72) |
| M231 E1 | YML038C | (48.65/49) |
| M89 B4 | YML040W | (48.51/55) |
| M89 C5 | YML041C | (30.83/40) |
| M229 F4 | YML042W | Carnitine O- |
| | | acetyltransferase |
| | | peroxisomal and |
| | | mitochondrial |
| | | (73.81/90) |
| M231 A7 | YML046W | RNA splicing factor |
| | | associated with U1 |
| | | snRNP (69.3/83) |
| M92 B2 | YML048W | ExtraCellular Mutant |
| | | (44.44/50) |
| M90 D5 | YML050W | (34.32/40) |
| M227 F7 | YML053C | (23.45/36) |
| M90 A1 | YML054C | Cytochrome b2 [L- |
| | | lactate cytochrome-c |
| | | oxidoreductase] |
| | | (65.04/65) |
| M90 C2 | YML055W | (19.69/20) |
| M92 C7 | YML060W | 43-kDa 8-oxo- |
| | | guanine DNA |
| | | glycosylase |
| | | (41.47/50) |
| M90 B1 | YML061C | 5′ to 3′ DNA helicase |
| | | (94.52/95) |
| M227 D2 | YML062C | (43.25/64) |
| M90 H4 | YML064C | (26.98/27) |
| M90 F5 | YML065W | 120-kDa (largest) |
| | | subunit of origin |
| | | recognition complex |
| | | (ORC)\shows |
| | | homology to Cdc6p |
| | | Cdc18p and Sir3p and |
| | | to proteins from K. |
| | | lactis S. pombe and |
| | | humans (100.65/100) |
| M90 C1 | YML069W | Binds to catalytic |
| | | subunit of DNA |
| | | polymerase alpha |
| | | (Pol1p) (60.83/65) |
| M90 E2 | YML070W | (64.45/64) |
| M90 F2 | YML078W | cyclophilin-3 |
| | | (cyclosporin-sensitive |
| | | proline rotamase-3) |
| | | (20.13/20) |
| M227 A4 | YML079W | (22.22/33) |
| M227 B5 | YML080W | (46.64/55) |
| M92 E1 | YML085C | 142-1460 alpha- |
| | | tubulin (49.38/65) |
| M333 H4 | YML095C | (23.13/34) |
| M227 A7 | YML098W | TFIID subunit |
| | | (18.48/33) |
| M90 B4 | YML101C | (12.90/20) |
| M92 G5 | YML102W | p60 subunit of the |
| | | yeast omatin |
| | | Assembly Factor-I |
| | | (CAF-I) (51.59/55) |
| M90 B6 | YML110C | (33.80/40) |
| M90 B9 | YML113W | datin an |
| | | oligo(dA).oligo(dT)- |
| | | binding protein |
| | | (27.49/30) |
| M90 G1 | YML114C | (56.13/60) |
| M92 B3 | YML115C | Vanadate resistance |
| | | protein (58.88/64) |
| M202 B6 | YML128C | (56.46/64) |
| M92 A7 | YMR002W | (17.37/28) |
| M91 A1 | YMR004W | Protein required for |
| | | sorting proteins to the |
| | | vacuole (56.32/48) |
| M233 A7 | YMR009W | (19.8/33) |
| M91 C3 | YMR014W | (57.2/57) |
| M310 A6 | YMR015C | cytochrome P450 |
| | | involved in C-22 |
| | | denaturation of the |
| | | ergosterol side-chain |
| | | (59.21/59) |
| M234 E7 | YMR017W | DBF2 Interacting |
| | | Protein\SNAP 25 |
| | | homolog (43.78/46) |
| M91 B1 | YMR020W | (55.99/50) |
| M93 C2 | YMR021C | metal-binding |
| | | transcriptional |
| | | activator (45.90/55) |
| M91 D3 | YMR022W | ubiquitin conjugating |
| | | enzyme (18.36/32) |
| M234 D6 | YMR024W | (43.01/43) |
| M91 B5 | YMR025W | (32.56/40) |
| M234 B4 | YMR030W | (41.47/50) |
| M93 C1 | YMR035W | Inner membrane |
| | | protease |
| | | (mitochondrial |
| | | protein) (19.58/32) |
| M234 H2 | YMR036C | (60.97/64) |
| M234 C4 | YMR037C | zinc finger protein |
| | | (77.47/100) |
| M233 C5 | YMR038C | (27.42/33) |
| M234 H8 | YMR041C | (36.88/48) |
| M233 | YMR042W | Regulator of arginine- |
| B10 | | responsive genes with |
| | | ARG81 and ARG82 |
| | | (19.58/34) |
| M93 D1 | YMR043W | putative |
| | | transcriptional |
| | | activator of alpha- |
| | | specific genes |
| | | (31.57/40) |
| M234 A3 | YMR044W | (52.46/98) |
| M93 G4 | YMR046C | (48.43/60) |
| M91 E5 | YMR048W | (34.98/35) |
| M91 E1 | YMR051C | (48.43/48) |
| M93 B6 | YMR055C | (33.69/40) |
| M91 F1 | YMR058W | multicopper oxidase |
| | | (70.07/60) |
| M234 A8 | YMR063W | (26.4/33) |
| M234 F10 | YMR065W | (55.55/64) |
| M234 H1 | YMR066W | (98.89/100) |
| M234 D3 | YMR067C | (45.79/45) |
| M234 F4 | YMR068W | (46.97/50) |
| M233 G5 | YMR069W | (31.46/35) |
| M234 A7 | YMR070W | unknown function\2 |
| | | Cys2-His2 zinc |
| | | fingers at c-terminus |
| | | glutamine and |
| | | asparagine rich. |
| | | (54.01/54) |
| M234 B8 | YMR071C | (18.40/18) |
| M93 C8 | YMR073C | (22.14/30) |
| M234 A2 | YMR074C | (15.98/25) |
| M234 G4 | YMR075W | (75.45/80) |
| M91 F4 | YMR077C | (24.34/32) |
| M303 E4 | YMR079W | 166-1071 |
| | | phosphatidylinositol |
| | | transfer protein |
| | | (33.55/35) |
| M255 D7 | YMR080C | putative helicase |
| | | (106.84/100) |
| M93 H1 | YMR081C | (37.21/50) |
| M234 F9 | YMR086W | (105.71/115) |
| M234 C2 | YMR088C | (61.85/64) |
| M93 C3 | YMR089C | mitochondrial |
| | | membrane ATPase of |
| | | the |
| | | CDC48\/PAS1\/SEC1 |
| | | 8 (AAA) family |
| | | (90.78/98) |
| M234 A5 | YMR090W | (25.08/33) |
| M234 B6 | YMR091C | (47.88/64) |
| M234 C7 | YMR092C | Protein localizes to |
| | | actin cortical patches. |
| | | Probable binding site |
| | | on actin lies on front |
| | | surface of subdomain |
| | | 3 and 4 (67.68/75) |
| M91 B6 | YMR093W | (56.54/56) |
| M242 A1 | YMR096W | Snooze: stationary |
| | | phase-induced gene |
| | | family (32.78/46) |
| M242 C2 | YMR097C | (40.40/44) |
| M242 A3 | YMR098C | (67.45/70) |
| M241 D4 | YMR099C | (32.70/45) |
| M112 D6 | YMR101C | (37.76/45) |
| M242 B7 | YMR102C | (91.77/98) |
| M242 B1 | YMR104C | protein kinase |
| | | (74.50/98) |
| M242 D2 | YMR105C | Phosphoglucomutase |
| | | (62.62/62) |
| M107 D3 | YMR106C | (69.22/90) |
| M242 D5 | YMR108W | acetolactate synthase |
| | | (75.68/85) |
| M107 A8 | YMR111C | (50.85/60) |
| M107 C1 | YMR112C | (14.44/20) |
| M242 E2 | YMR113W | (47.08/48) |
| M112 E3 | YMR114C | (40.51/55) |
| M112 F5 | YMR116C | 811-1233 |
| | | (35.2/38) |
| M242 E8 | YMR119W | (68.75/68) |
| M112 F3 | YMR121C | (22.47/38) |
| M107 G5 | YMR123W | (13.53/30) |
| M112 E6 | YMR124W | (103.84/110) |
| M242 E7 | YMR125W | transcriptional |
| | | activator of glycolytic |
| | | genes (94.49/94) |
| M242 E1 | YMR127C | Protein involved in |
| | | silencing HMR |
| | | homologous to |
| | | acetyltransferases |
| | | (37.21/45) |
| M107 F4 | YMR130W | (33.33/40) |
| M242 F5 | YMR131C | (56.24/75) |
| M107 E6 | YMR132C | (22.91/30) |
| M242 F7 | YMR133W | Meiosis-specific |
| | | recombination gene |
| | | (47.96/47) |
| M242 G8 | YMR134W | (26.28/34) |
| M112 F2 | YMR135W-A | (19.58/32) |
| M112 G3 | YMR136W | (61.71/60) |
| M242 G5 | YMR138W | GTP-binding protein |
| | | (21.12/31) |
| M107 F6 | YMR139W | protein kinase that |
| | | phosphorylates the |
| | | meiotic activator |
| | | IME1 (40.81/50) |
| M242 G7 | YMR140W | (53.9/59) |
| M112 G1 | YMR142C | 407-1002 |
| | | (22/38) |
| M107 H3 | YMR144W | (37./7350) |
| M242 G6 | YMR147W | (24.64/35) |
| M107 F7 | YMR148W | (16.49/30) |
| M242 G3 | YMR152W | (40.36/52) |
| M242 H1 | YMR157C | (28.08/32) |
| M242 H2 | YMR158W | (17.16/28) |
| M242 B5 | YMR159C | Protein homologous |
| | | to human Sin3 |
| | | complex component |
| | | SAP18 possible |
| | | coiled-coil protein |
| | | (16.53/26) |
| M242 H6 | YMR161W | Homologous to E coli |
| | | dnaJ protein |
| | | (24.75/34) |
| M242 A8 | YMR170C | aldehyde |
| | | dehydrogenase |
| | | (NAD(P)+) likely |
| | | cytosolic (55.69/62) |
| M242 A4 | YMR173W | flocculent specific |
| | | protein\contains >35 |
| | | repeats of the amino |
| | | acid sequence |
| | | NNNDSYGS |
| | | (47.41/55) |
| M242 D9 | YMR177W | (56.21/56) |
| M107 B2 | YMR178W | (30.25/36) |
| M242 B4 | YMR180C | (35.23/49) |
| M242 A7 | YMR183C | (32.48/46) |
| M242 B8 | YMR184W | (21.89/20) |
| M237 F4 | YMR197C | Vti1p |
| | | (23.90/34) |
| M104 C1 | YMR201C | 112-1200 |
| | | (40.92/50) |
| M237 H2 | YMR203W | Mitochondrial outer |
| | | membrane protein\ |
| | | forms the outer |
| | | membrane import |
| | | channel (42.68/48) |
| M237 G4 | YMR205C | phosphofructokinase |
| | | beta subunit |
| | | (105.52/125) |
| M237 C1 | YMR209C | (50.30/55) |
| M104 C3 | YMR211W | (52.46/55) |
| M104 F4 | YMR213W | (65.01/75) |
| M96 F6 | YMR214W | dnaJ homolog |
| | | (44.55/48) |
| M237 D1 | YMR217W | GMP synthase |
| | | (57.86/57) |
| M104 A4 | YMR220W | 48 kDa |
| | | Phosphomevalonate |
| | | kinase (49.72/62) |
| M104 E5 | YMR222C | (24.56/40) |
| M310 B6 | YMR224C | Protein required for |
| | | double-strand break |
| | | repair and meiotic |
| | | recombination |
| | | (76.25/80) |
| M104 E1 | YMR225C | 206-444 |
| | | Mitochondrial |
| | | ribosomal protein |
| | | MRPL44 (YmL44) |
| | | (10.89/14) |
| M104 D3 | YMR227C | TFIID subunit |
| | | (64.93/65) |
| M104 B4 | YMR228W | Mitochondrial RNA |
| | | polymerase |
| | | specificity factor |
| | | (37.62/40) |
| M104 F1 | YMR233W | (24.97/25) |
| M96 H3 | YMR235C | (44.80/55) |
| M237 B5 | YMR237W | (79.75/88) |
| M237 C7 | YMR239C | Ribonuclease III |
| | | (51.84/58) |
| M237 F1 | YMR241W | (34.65/35) |
| M237 A4 | YMR244C-A | (11.47/16) |
| M238 C5 | YMR244W | (39.26/43) |
| M202 D6 | YMR246W | long-chain fatty acid- |
| | | CoA ligase and |
| | | synthetase 4 |
| | | (76.45/98) |
| M202 C6 | YMR250W | (64.46/75) |
| M237 D2 | YMR251W | (40.47/48) |
| M237 B4 | YMR252C | (14.77/18) |
| M104 B7 | YMR255W | (20.79/36) |
| M237 C4 | YMR260C | Translation initiation |
| | | factor eIFeIF-1A |
| | | (16.86/21) |
| M237 E6 | YMR262W | (34.54/48) |
| M237 F7 | YMR263W | (22.22/30) |
| M104 D8 | YMR264W | (22.44/30) |
| M104 F3 | YMR267W | mitochondrial |
| | | inorganic |
| | | pyrophosphatase |
| | | (34.21/38) |
| M237 F5 | YMR269W | (15.73/25) |
| M104 B6 | YMR270C | Upstream activation |
| | | factor subunit |
| | | (40.28/55) |
| M104 D7 | YMR271C | Orotate |
| | | phosphoribosyl- |
| | | transferase 2 |
| | | (25.00/40) |
| M237 E2 | YMR274C | (34.68/38) |
| M237 E4 | YMR276W | ubiquitin-like protein |
| | | (41.14/42) |
| M237 G6 | YMR278W | (68.53/75) |
| M239 A1 | YMR281W | (33.55/45) |
| M240 C2 | YMR282C | basic hydrophilic 67.5 |
| | | kDa protein |
| | | (63.83/62) |
| M97 B3 | YMR283C | Initiator methionine |
| | | tRNA 2′ -O-ribosyl |
| | | phosphate transferase |
| | | (56.46/56) |
| M97 E5 | YMR285C | (56.68/55) |
| M274 D7 | YMR288W | (106.92/110) |
| M97 A1 | YMR289W | (41.35/40) |
| M97 H1 | YMR290C | (55.58/55) |
| M239 D3 | YMR290 | (12.76/12) |
| | W-A | |
| M102 C4 | YMR291W | (64.57/70) |
| M97 G6 | YMR293C | (51.07/55) |
| M102 A8 | YMR294W | Coiled-coil domain |
| | | protein required for |
| | | proper nuclear |
| | | migration during |
| | | mitosis (but not |
| | | during conjugation) |
| | | (41.14/55) |
| M239 C1 | YMR295C | (21.70/33) |
| M240 D2 | YMR296C | Probable component |
| | | of serine |
| | | palmitoyltransferase |
| | | which catalyzes the |
| | | first step in |
| | | biosynthesis of long- |
| | | chain sphingolipids |
| | | (61.41/64) |
| M97 D3 | YMR297W | carboxypeptidase Y |
| | | (58.63/58) |
| M239 D5 | YMR299C | (34.45/45) |
| M240 D6 | YMR300C | phosphoribosylpyrop |
| | | hosphate |
| | | amidotransferase |
| | | (56.13/53) |
| M239 D1 | YMR303C | alcohol |
| | | dehydrogenase II |
| | | (38.31/52) |
| M97 E4 | YMR305C | (42.82/60) |
| M97 D9 | YMR309C | ˜100 kDa |
| | | cytoplasmic protein |
| | | (89.45/90) |
| M239 E1 | YMR310C | (34.90/44) |
| M239 F2 | YMR311C | Regulates activity of |
| | | protein phosphatase 1 |
| | | Glc7p which is |
| | | involved in proper |
| | | chromosome |
| | | segregation |
| | | (25.32/36) |
| M97 E3 | YMR312W | (30.14/40) |
| M239 E5 | YMR314W | alpha-type of subunit |
| | | of 20S proteasome |
| | | (25.85/31) |
| M97 A7 | YMR315W | (38.5/40) |
| M240 H8 | YMR316C-B | (11.35/19) |
| M97 D1 | YMR316W | (37.07/37) |
| M239 H3 | YMR318C | (39.63/52) |
| M239 F5 | YMR320W | (11.22/14) |
| M240 F6 | YMR321C | (11.58/30) |
| M102 E8 | YMR322C | (26.10/36) |
| M97 E9 | YMR323W | (48.18/50) |
| M240 G2 | YMR325W | (13.75/19) |
| M102 G4 | YNL001W | an ORF of unknown |
| | | function located in a |
| | | centromeric region |
| | | duplicated between |
| | | chromosomes III and |
| | | XIV (DOM34 |
| | | homologue on |
| | | chromosome III is a |
| | | pseudogene) |
| | | (42.57/34) |
| M202 E6 | YNL002C | (35.45/52) |
| M240 H7 | YNL004W | hypothetical RNA- |
| | | binding protein |
| | | (47.3/48) |
| M239 B9 | YNL005C | Mitochondrial |
| | | ribosomal protein |
| | | MRP7 (YmL2) |
| | | (E. coli L27) (40.84/55) |
| M102 E1 | YNL006W | (33.44/40) |
| M240 H2 | YNL007C | sit4 suppressor dnaJ |
| | | homolog (38.75/48) |
| M97 A5 | YNL009W | peroxisomal NADP- |
| | | dependent isocitrate |
| | | dehydrogenase |
| | | (46.31/46) |
| M97 C6 | YNL010W | (26.62/30) |
| M239 A7 | YNL011C | (48.87/52) |
| M97 G8 | YNL012W | Transcription |
| | | regulator (59.07/60) |
| M240 A3 | YNL015W | Proteinase inhibitor |
| | | I2B (PBI2) that |
| | | inhibits protease |
| | | Prb1p (yscB) (8.46/8) |
| M97 G3 | YNL016W | poly(A)+RNA- |
| | | binding protein |
| | | (49.94/50) |
| M97 D6 | YNL018C | (67.45/67) |
| M239 B7 | YNL019C | (31.37/39) |
| M240 B8 | YNL020C | (70.21/71) |
| M240 H1 | YNL022C | (53.93/50) |
| M240 A4 | YNL024C | (27.09/36) |
| M97 B5 | YNL025C | C-type cyclin |
| | | (35.56/40) |
| M97 E6 | YNL026W | (53.45/55) |
| M240 B7 | YNL027W | (74.69/98) |
| M240 C8 | YNL028W | (11.66/16) |
| M240 E9 | YNL029C | Putative |
| | | mannosyltransferase |
| | | of the KRE2 family |
| | | (57.45/57) |
| M240 A2 | YNL030W | Histone H4 (HHF1 |
| | | and HHF2 code for |
| | | identical proteins) |
| | | (11.44/14) |
| M102 G2 | YNL031C | Histone H3 (HHT1 |
| | | and HHT2 code for |
| | | identical proteins) |
| | | (14.99/20) |
| M97 A4 | YNL032W | (31.02/55) |
| M240 A5 | YNL033W | (31.45/35) |
| M240 B6 | YNL034W | (67.43/71) |
| M239 C7 | YNL035C | (42.82/49) |
| M239 D8 | YNL036W | involved in secretion |
| | | of proteins that lack |
| | | classical secretory |
| | | signal sequences |
| | | (24.42/32) |
| M97 G9 | YNL037C | alpha-4-beta-4 |
| | | subunit of |
| | | mitochondrial |
| | | isocitrate |
| | | dehydrogenase 1 |
| | | (39.63/40) |
| M240 B2 | YNL038W | (23.43/20) |
| M97 A3 | YNL039W | 90 kd subunit of |
| | | TFIIIB also called |
| | | TFIIIB90 or B″ or |
| | | B″90 component |
| | | (65.45/65) |
| M97 B4 | YNL040W | (50.37/50) |
| M240 C6 | YNL042W | (43.67/50) |
| M97 H7 | YNL043C | (11.69/12) |
| M102 C9 | YNL044W | (17.49/20) |
| M97 H9 | YNL045W | (73.92/73) |
| M243 A1 | YNL046W | (19.03/28) |
| M243 B2 | YNL047C | (72.29/85) |
| M243 B8 | YNL052W | Cytochrome-c |
| | | oxidase chain Va |
| | | (16.94/18) |
| M105 B8 | YNL053W | (53.9/53) |
| M243 B1 | YNL054W | (128.3/6130) |
| M105 H1 | YNL055C | Outer mitochondrial |
| | | membrane porin |
| | | (voltage-dependent |
| | | anion channel or |
| | | VDAC) (31.26/31) |
| M105 G2 | YNL056W | (21.78/36) |
| M243 B6 | YNL058C | (34.79/48) |
| M243 C8 | YNL061W | 90-kDa protein |
| | | located in nucleolus |
| | | that is homologous to |
| | | a human |
| | | proliferation- |
| | | associated nucleolar |
| | | protein p120 |
| | | (68.09/68) |
| M105 C8 | YNL062C | RNA-binding (zeta) |
| | | subunit of translation |
| | | initiation factor 3 |
| | | (eIF-3) (52.61/52) |
| M243 C1 | YNL063W | (34.65/45) |
| M108 E2 | YNL065W | (64.57/64) |
| M105 A4 | YNL066W | Protein involved in |
| | | the aging process |
| | | (46.31/64) |
| M243 C6 | YNL067W | ribosomal protein |
| | | RPL9 (YL11) |
| | | (21.12/36) |
| M105 A7 | YNL069C | 478-1046 Ribosomal |
| | | protein (21.89/20) |
| M243 F9 | YNL070W | translocase of the |
| | | outer mito. membrane |
| | | (6.71/7) |
| M105 C1 | YNL071W | Dihydrolipoamide |
| | | acetyltransferase |
| | | component (E2) of |
| | | pyruvate |
| | | dehydrogenase |
| | | complex (53.13/50) |
| M247 A2 | YNL072W | RNase H(35) a 35 |
| | | kDa ribonuclease H |
| | | (33.88/48) |
| M243 G3 | YNL073W | mitochondrial lysine- |
| | | tRNA synthetase |
| | | (63.47/64) |
| M105 B4 | YNL074C | (49.75/50) |
| M243 D6 | YNL075W | (32.01/50) |
| M243 B7 | YNL076W | negative regulator of |
| | | Ras cAMP pathway |
| | | (64.45/70) |
| M243 E8 | YNL077W | (58.29/64) |
| M108 D8 | YNL078W | (44.88/52) |
| M105 D1 | YNL079C | tropomyosin I |
| | | (21.92/32) |
| M247 B2 | YNL080C | (40.39/47) |
| M243 E6 | YNL083W | (54.45/64) |
| M247 C6 | YNL084C | Protein necessary for |
| | | internalization of |
| | | alpha-factor receptor |
| | | when bound to ligand |
| | | (38.42/40) |
| M243 H9 | YNL086W | (11.33/18) |
| M247 C3 | YNL089C | (17.41/20) |
| M108 B4 | YNL090W | GTP-binding protein |
| | | of the rho subfamily |
| | | of ras-like proteins |
| | | (21.23/34) |
| M243 D7 | YNL092W | (44.11/48) |
| M243 F8 | YNL093W | rab5-like GTPase |
| | | involved in vacuolar |
| | | protein sorting and |
| | | endocytosis |
| | | (24.31/34) |
| M243 | YNL094W | (64.68/75) |
| A10 | | |
| M247 C2 | YNL096C | 490-918 |
| | | (21.01/31) |
| M105 D3 | YNL097C | (36.33/36) |
| M105 E4 | YNL098C | Ras proto-oncogene |
| | | homolog (35.45/48) |
| M105 D5 | YNL099C | (26.21/32) |
| M243 E7 | YNL100W | (25.85/34) |
| M243 H2 | YNL104C | alpha-isopropylmalate |
| | | synthase (2- |
| | | Isopropylmalate |
| | | Synthase) (68.12/68) |
| M247 E3 | YNL105W | (15.73/17) |
| M243 G6 | YNL107W | (24.97/36) |
| M108 E6 | YNL108C | (29.73/40) |
| M243 | YNL110C | (24.23/31) |
| C10 | | |
| M108 F1 | YNL111C | cytochrome b5 |
| | | (13.23/20) |
| M243 D4 | YNL113W | subunit common to |
| | | RNA polymerases I |
| | | (A) and III (C) |
| | | (15.73/16) |
| M247 F4 | YNL114C | (13.56/18) |
| M243 G7 | YNL116W | (57.53/64) |
| M243 A9 | YNL117W | carbon-catabolite |
| | | sensitive malate |
| | | synthase (61.05/64) |
| M105 H8 | YNL118C | (106.73/100) |
| M243 H1 | YNL119W | (54.34/68) |
| M243 A3 | YNL120C | (17.74/25) |
| M243 F5 | YNL122C | (12.68/20) |
| M247 H6 | YNL124W | (54.23/90) |
| M255 F7 | YNL128W | Similar to human |
| | | tumor suppressor |
| | | gene known as TEP1 |
| | | MMAC1 and PTEN1. |
| | | Contains sequence |
| | | motifs characteristic |
| | | of protein tyrosine |
| | | phosphatases. |
| | | (47.85/50) |
| M105 F3 | YNL129W | (26.51/30) |
| M243 A7 | YNL131W | Mitochondrial import |
| | | receptor complex |
| | | protein (16.83/30) |
| M243 C9 | YNL133C | (19.06/20) |
| M247 A9 | YNL134C | (41.49/50) |
| M243 A2 | YNL135C | peptidylprolyl cis- |
| | | trans isomerase |
| | | (12.57/18) |
| M243 C3 | YNL136W | (46.86/50) |
| M105 G4 | YNL138W | 70-kDa adenylyl |
| | | cyclase-associated |
| | | protein (57.97/64) |
| M247 A7 | YNL140C | (20.82/31) |
| M105 A8 | YNL141W | (38.38/52) |
| M243 | YNL142W | Ammonia transport |
| D10 | | protein (55/55) |
| M103 E4 | YNL148C | cofactor B (27./9748) |
| M103 C5 | YNL149C | (14.22/28) |
| M244 F2 | YNL152W | (45.1/57) |
| M244 H3 | YNL153C | Polypeptide 4 of a |
| | | Yeast Non-native |
| | | Actin Binding |
| | | Complex homolog of |
| | | a component of the |
| | | bovine NABC |
| | | complex (14.22/26) |
| M244 A5 | YNL154C | membrane-bound |
| | | casein kinase I |
| | | homolog (60.09/64) |
| M244 B6 | YNL155W | (30.25/32) |
| M103 F4 | YNL156C | (32.92/40) |
| M245 B8 | YNL157W | (18.59/34) |
| M245 D9 | YNL158W | (21.89/32) |
| M245 C1 | YNL159C | (31.82/32) |
| M274 E7 | YNL160W | Glycoprotein |
| | | synthesized in |
| | | response to nutrient |
| | | limitation (39.05/42) |
| M244 A4 | YNL161W | (83.37/95) |
| M110 E3 | YNL163C | (122.13/?) |
| M245 A7 | YNL164C | (38.64/49) |
| M103 D5 | YNL165W | (44.77/52) |
| M245 E9 | YNL166C | (49.31/49) |
| M245 G2 | YNL168C | (28.52/38) |
| M244 B4 | YNL169C | Phosphatidylserine |
| | | Decarboxylase 1 |
| | | (55.03/55) |
| M245 G5 | YNL171C | (13.45/48) |
| M244 H8 | YNL173C | (40.39/48) |
| M245 F9 | YNL174W | (21.01/37) |
| M103 D1 | YNL175C | (44.46/55) |
| M110 D2 | YNL176C | (69.99/?) |
| M244 B5 | YNL178W | Ribosomal protein |
| | | RPS3 (rp13) (YS3) |
| | | (Mammalian S3) |
| | | (26.51/35) |
| M245 H5 | YNL179C | (15.98/28) |
| M245 B7 | YNL180C | (36.22/36) |
| M110 A6 | YNL181W | (44.88/?) |
| M245 G9 | YNL182C | (61.08/98) |
| M244 F1 | YNL183C | protein kinase |
| | | homolog (86.93/88) |
| M244 C4 | YNL185C | (17.41/26) |
| M244 C5 | YNL186W | (87.23/115) |
| M103 G3 | YNL187W | (39.48/40) |
| M110 A5 | YNL188W | Protein involved in |
| | | spindle pole body |
| | | duplication and |
| | | karyogamy |
| | | (47.74/70) |
| M110 B6 | YNL189W | karyopherin alpha |
| | | homolog of 60 kDa |
| | | (59.73/59) |
| M245 H9 | YNL190W | (22.55/95) |
| M244 G1 | YNL191W | (39.48/44) |
| M245 C4 | YNL193W | (61.49/65) |
| M103 B3 | YNL194C | (33.14/42) |
| M103 H3 | YNL195C | (26.76/36) |
| M110 B5 | YNL196C | (32.81/40) |
| M24 F10 | YNL198C | (11.03/16) |
| M245 G1 | YNL199C | Activates |
| | | transcription of |
| | | glycolytic genes\ |
| | | homologous to |
| | | GCR1\may function |
| | | in complex with |
| | | Gcr2p (58.77/50) |
| M103 B2 | YNL200C | (27.09/38) |
| M103 C3 | YNL202W | sporulation-specific |
| | | protein (32.56/42) |
| M103 A4 | YNL203C | (22.46/32) |
| M110 C5 | YNL204C | sporulation-specific |
| | | protein (33.03/33) |
| M244 | YNL206C | (50.08/60) |
| G10 | | |
| M110 F1 | YNL207W | (46.86/46) |
| M244 D3 | YNL208W | (22.55/33) |
| M244 F4 | YNL209W | Heat shock protein of |
| | | HSP70 family |
| | | homolog of SSB1 |
| | | (67.54/68) |
| M244 F5 | YNL210W | mer2 splicing factor |
| | | (29.81/32) |
| M110 B4 | YNL211C | (9.496/9) |
| M110 D5 | YNL212W | (86.13/86) |
| M110 E6 | YNL213C | (23.57/30) |
| M110 G1 | YNL215W | (35.31/64) |
| M245 D3 | YNL216W | repressor activator |
| | | protein (91.08/87) |
| M244 G4 | YNL217W | (35.97/47) |
| M244 G5 | YNL218W | (64.68/74) |
| M110 E5 | YNL220W | adenylosuccinate |
| | | synthetase (47.74/47) |
| M244 | YNL222W | (22.77/28) |
| H10 | | |
| M245 B2 | YNL223W | (55.77/64) |
| M244 F3 | YNL224C | (84.40/94) |
| M110 D4 | YNL227C | (64.93/80) |
| M245 H7 | YNL228W | (28.49/33) |
| M244 G9 | YNL229C | transcriptional |
| | | regulator putative |
| | | glutathione |
| | | transferase (38.97/48) |
| M245 E10 | YNL230C | (41.72/41) |
| M110 A2 | YNL231C | (38.64/?) |
| M103 D2 | YNL232W | (32.23/40) |
| M110 D3 | YNL234W | (46.97/?) |
| M265 A8 | YNL236W | transcriptional |
| | | silencer general |
| | | repressor of diverse |
| | | set of genes |
| | | (107.35/107) |
| M103 F6 | YNL238W | Ca2+-dependent |
| | | serine protease |
| | | (89.65/100) |
| M254 A1 | YNL239W | Aminopeptidase of |
| | | cysteine protease |
| | | family (53.24/59) |
| M111 B2 | YNL240C | (54.04/64) |
| M111 E3 | YNL241C | Glucose-6-phosphate |
| | | dehydrogenase |
| | | (55.58/55) |
| M111 G5 | YNL243W | transmembrane |
| | | protein (106.59/106) |
| M254 E5 | YNL244C | translation factor |
| | | (11.91/25) |
| M254 H7 | YNL246W | 128-890 |
| | | (29.15/30) |
| M111 C2 | YNL248C | 49-kDa alpha subunit |
| | | of RNA polymerase |
| | | A (45.68/55) |
| M249 C3 | YNL249C | (59.65/58) |
| M111 D8 | YNL253W | (46.53/55) |
| M254 A8 | YNL254C | (44.14/53) |
| M249 B1 | YNL255C | (16.86/21) |
| M109 G1 | YNL256W | (95.25/95) |
| M254 E4 | YNL259C | Antioxidant protein |
| | | and metal |
| | | homeostasis factor |
| | | protects against |
| | | oxygen toxicity |
| | | (8.066/16) |
| M109 A6 | YNL260C | (21.81/21) |
| M254 G6 | YNL261W | Fifth largest subunit |
| | | of origin recognition |
| | | complex\contains |
| | | possible ATP-binding |
| | | site |
| | | (52.8/64) |
| M249 C1 | YNL263C | (34.57/38) |
| M109 H1 | YNL264C | (38.53/52) |
| M254 D2 | YNL272C | Protein with coiled- |
| | | coil domain essential |
| | | for vesicular transport |
| | | 83.52/97) |
| M254 F3 | YNL274C | (38.53/42) |
| M254 F4 | YNL275W | (63.47/63) |
| M254 H6 | YNL277W | homoserine O-trans- |
| | | acetylase (53.57/65) |
| M111 G9 | YNL278W | (116.71/116) |
| M254 D1 | YNL279W | (72.82/95) |
| M249 F3 | YNL281W | (16.94/30) |
| M109 F3 | YNL282W | (21.56/30) |
| M109 H4 | YNL283C | (55.46/80) |
| M109 D6 | YNL284C | (35.45/50) |
| M249 G7 | YNL285W | (13.64/27) |
| M111 H9 | YNL286W | Cold sensitive U2 |
| | | snRNA Supressor |
| | | (31.46/42) |
| M111 A5 | YNL290W | Subunit 3 of |
| | | Replication Factor C\ |
| | | homologous to human |
| | | RFC 36 kDa subunit |
| | | (37.51/45) |
| M109 A5 | YNL291C | plasma membrane |
| | | protein (60.31/98) |
| M111 | YNL294C | (58.66/64) |
| A10 | | |
| M249 E2 | YNL296W | (11.55/14) |
| M109 H3 | YNL298W | protein kinase |
| | | (92.73/98) |
| M111 A9 | YNL301C | 545-993 Ribosomal |
| | | protein rp28 (rat L18) |
| | | (20.57/34) |
| M111 A3 | YNL304W | (39.26/50) |
| M111 A4 | YNL305C | (32.70/32) |
| M109 A4 | YNL306W | (23.98/35) |
| M109 C5 | YNL307C | 43.1 kDa |
| | | Serine\/threonine\/tyr |
| | | osine protein kinase |
| | | (41.38/50) |
| M109 E6 | YNL308C | (65.04/65) |
| M254 D7 | YNL309W | sin3 binding protein |
| | | (50.6/57) |
| M111 | YNL310C | (22.58/34) |
| C10 | | |
| M111 B3 | YNL312W | 116-930 subunit 2 of |
| | | replication factor RF- |
| | | A\29\% identical to |
| | | the human p34 |
| | | subunit of RF-A |
| | | (30.14/34) |
| M249 E4 | YNL314W | positive regulator of |
| | | allophanate inducible |
| | | genes (28.26/36) |
| M109 D5 | YNL315C | (35.01/40) |
| M111 C9 | YNL317W | (51.36/55) |
| M254 H2 | YNL320W | (31.45/42) |
| M254 C4 | YNL322C | Cell wall beta-glucan |
| | | assembly (34.46/50) |
| M254 C5 | YNL323W | (45.65/50) |
| M111 E10 | YNL326C | (36.99/40) |
| M249 A3 | YNL328C | (16.09/20) |
| M109 F5 | YNL331C | (41.49/50) |
| M109 H6 | YNL332W | (37.51/35) |
| M249 D8 | YNL333W | Snooze: stationary |
| | | phase-induced gene |
| | | family (32.89/40) |
| M114 A1 | YNL335W | (24.86/34) |
| M113 F6 | YNR001C | citrate synthase. |
| | | Nuclear encoded |
| | | mitochondrial protein. |
| | | (52.72/67) |
| M257 G8 | YNR002C | Putative |
| | | transmembrane |
| | | protein (31.05/36) |
| M257 | YNR003C | 34-kDa subunit of |
| C10 | | RNA polymerase III |
| | | (C) (34.90/45) |
| M257 B1 | YNR004W | (16.27/26) |
| M257 E2 | YNR005C | (14.77/18) |
| M253 H3 | YNR006W | hydrophilic protein\ |
| | | has cysteine rich |
| | | putative zinc finger |
| | | essential for function |
| | | (68.53/100) |
| M114 C4 | YNR007C | (34.13/45) |
| M253 B6 | YNR008W | (72.82/90) |
| M253 D7 | YNR009W | (27.5/38) |
| M257 H8 | YNR010W | Protein required for |
| | | accurate mitotic |
| | | chromosome |
| | | segregation (16.5/25) |
| M257 C1 | YNR012W | Uridine kinase |
| | | (55.22/55) |
| M113 F3 | YNR014W | (23.43/45) |
| M253 C5 | YNR015W | Suppressor of |
| | | Mitochondrial |
| | | Mutation in the |
| | | tRNAasp gene |
| | | (42.45/47) |
| M257 G7 | YNR017W | 23 kDa mitochondrial |
| | | inner membrane |
| | | protein (24.53/32) |
| M114 E7 | YNR018W | (24.75/38) |
| M257 G2 | YNR021W | (44.55/47) |
| M114 D4 | YNR023W | 73 kDa subunit of the |
| | | SWI\/SNF |
| | | transcription |
| | | activation complex |
| | | (62.47/70) |
| M253 C6 | YNR024W | (20.57/35) |
| M114 F7 | YNR026C | integral membrane |
| | | protein involved in |
| | | protein transport to |
| | | the Golgi (51.84/64) |
| M253 | YNR027W | (34.98/48) |
| A10 | | |
| M114 D1 | YNR028W | (33.99/40) |
| M253 C4 | YNR030W | ExtraCellular Mutant |
| | | (60.72/70) |
| M114 D5 | YNR032W | (40.59/48) |
| M257 A8 | YNR033W | para-aminobenzoate |
| | | synthase PABA |
| | | synthase (86.68/98) |
| M114 G7 | YNR034W | (35.42/40) |
| M114 B9 | YNR035C | Arp Complex Subunit |
| | | (37.65/45) |
| M114 E1 | YNR036C | (16.86/30) |
| M113 H5 | YNR040W | (28.37/50) |
| M253 G7 | YNR041C | para |
| | | hydroxybenzoate: |
| | | polyprenyl transferase |
| | | (40.95/41) |
| M253 A9 | YNR042W | (15.73/30) |
| M257 | YNR043W | mevalonate |
| G10 | | pyrophosphate |
| | | decarboxylase |
| | | (43.67/49) |
| M253 E4 | YNR046W | (14.96/20) |
| M253 F6 | YNR048W | (43.44/60) |
| M113 B7 | YNR049C | Multicopy Suppressor |
| | | of sec1 (23.13/20) |
| M114 A8 | YNR050C | Saccharopine |
| | | dehydrogenase |
| | | (NADP+L-glutamate |
| | | forming) |
| | | (saccharopine |
| | | reductase) (EC |
| | | 1.5.1.10) (49.09/55) |
| M253 | YNR051C | (56.68/67) |
| D10 | | |
| M114 F1 | YNR052C | Putative transcription |
| | | factor (47.66/55) |
| M253 F4 | YNR054C | (34.79/50) |
| M113 C7 | YNR057C | (26.10/26) |
| M114 B8 | YNR058W | 7 8-diamino- |
| | | pelargonic acid |
| | | aminotransferase |
| | | (DAPA) |
| | | aminotransferase |
| | | (52.91/55) |
| M253 E10 | YNR059W | (63.91/64) |
| M253 C3 | YNR061C | (24.12/38) |
| M113 B6 | YNR064C | (31.93/64) |
| M257 G9 | YNR066C | (47.99/50) |
| M253 A2 | YNR068C | (29.95/32) |
| M257 C3 | YNR069C | (53.82/60) |
| M114 H4 | YNR071C | (37.65/45) |
| M113 E7 | YNR073C | (55.35/64) |
| M257 | YNR075W | Protein with strong |
| B11 | | similarity to |
| | | subtelomerically- |
| | | encoded proteins such |
| | | as Cos5p Ybr302p |
| | | Cos3p Cos1p Cos4p |
| | | Cos8p Cos6p Cos9p |
| | | (41.35/47) |
| M113 C4 | YOL001W | negative |
| | | transcriptional |
| | | regulator (32.34/50) |
| M257 C7 | YOL003C | (41.61/45) |
| M253 E9 | YOL005C | RNA polymerase II |
| | | subunit (13.23/17) |
| M257 E3 | YOL008W | (22.88/32) |
| M257 A5 | YOL009C | Component of |
| | | Mitochondrial |
| | | Inheritance located in |
| | | outer mitochondrial |
| | | membrane (29.84/40) |
| M113 F7 | YOL012C | Histone-related |
| | | protein that can |
| | | suppress histone H4 |
| | | point mutation |
| | | (14.77/25) |
| M257 | YOL013C | (60.64/64) |
| B10 | | |
| M116 B2 | YOL016C | calmodulin dependent |
| | | protein kinase |
| | | (49.20/55) |
| M258 B1 | YOL023W | mitochondrial |
| | | initiation factor 2 |
| | | (74.47/84) |
| M258 E3 | YOL02SW | Affects longevity |
| | | (72.71/80) |
| M258 G4 | YOL026C | (12.46/18) |
| M258 G6 | YOL028C | (26.98/38) |
| M117 A7 | YOL029C | (22.14/32) |
| M258 D2 | YOL032W | (27.17/34) |
| M258 F3 | YOL033W | Mitochondrial |
| | | glutamyl-tRNA |
| | | synthetase (59.07/69) |
| M258 A8 | YOL037C | (12.90/34) |
| M258 E2 | YOL040C | Ribosomal protein |
| | | RPS21 (rp52) (E. coli |
| | | S19) (rat S15) (RIG |
| | | protein) |
| | | (15.65/22) |
| M258 H4 | YOL042W | (40.04/48) |
| M258 G5 | YOL043C | Endonuclease III-like |
| | | glycosylase 2 |
| | | (41.83/47) |
| M258 H8 | YOL046C | (24.67/34) |
| M258 F2 | YOL048C | (11.69/16) |
| M116 H8 | YOL053W | (43.56/45) |
| M116 E1 | YOL054W | (44.77/80) |
| M258 G2 | YOL055C | (60.64/60) |
| M116 G3 | YOL056W | phosphoglycerate |
| | | mutase (33.44/38) |
| M258 B5 | YOL057W | (78.32/85) |
| M259 H5 | YOL058W | arginosuccinate |
| | | synthetase (46.31/55) |
| M258 B7 | YOL059W | Glycerol-3-phosphate |
| | | dehydrogenase |
| | | (NAD+) (48.51/54) |
| M116 F7 | YOL060C | (77.69/77) |
| M258 B9 | YOL061W | Phosphoribosylpyrop |
| | | hosphate synthetase |
| | | (ribose-phosphate |
| | | pyrophosphokinase) |
| | | (54.67/60) |
| M116 H3 | YOL064C | Putative phosphatase |
| | | gene involved in salt |
| | | tolerance and |
| | | methionine |
| | | biogenesis\ |
| | | halotolerance |
| | | (39.30/45) |
| M258 C5 | YOL065C | (42.37/48) |
| M117 C5 | YOL066C | DRAP deaminase |
| | | (65.04/70) |
| M116 A7 | YOL067C | Transcription factor |
| | | (bHLH) involved in |
| | | interorganelle |
| | | communication |
| | | between mitochondria |
| | | peroxisomes and |
| | | nucleus (19.50/28) |
| M303 D5 | YOL068C | (55.46/65) |
| M258 A3 | YOL080C | (31.82/44) |
| M117 C4 | YOL082W | (45.76/60) |
| M259 A6 | YOL083W | (45.43/53) |
| M116 D9 | YOL086C | Alcohol |
| | | dehydrogenase |
| | | (38.31/45) |
| M116 G2 | YOL088C | (30.50/40) |
| M258 E7 | YOL092W | (33.99/35) |
| M116 B8 | YOL093W | (32.34/40) |
| M116 E9 | YOL094C | Subunit 4 of |
| | | Replication Factor C\ |
| | | homologous to human |
| | | RFC 40 kDa subunit |
| | | (35.56/52) |
| M116 H2 | YOL096C | 3 4-dihydroxy-5- |
| | | hexaprenylbenzoate |
| | | methyltransferase |
| | | (34.79/40) |
| M117 F3 | YOL097C | (47.55/50) |
| M258 D6 | YOL099C | (17.96/25) |
| M116 C8 | YOL101C | (34.35/40) |
| M258 D3 | YOL104C | (38.75/45) |
| M258 E6 | YOL107W | (37.73/42) |
| M202 A7 | YOL108C | Transcription factor |
| | | involved in activation |
| | | of phospholipid |
| | | synthetic genes |
| | | (16.64/30) |
| M258 G8 | YOL109W | (12.54/24) |
| M118 A1 | YOL111C | (23.45/36) |
| M118 A4 | YOL114C | (22.35/33) |
| M202 E7 | YOL116W | 43 kDa protein |
| | | (42.13/64) |
| M282 G7 | YOL118C | (11.35/16) |
| M278 D2 | YOL120C | 560-1008 Ribosomal |
| | | protein rp28 (rat L18) |
| | | (RP28A and RP28B |
| | | code for identical |
| | | proteins) (20.57/30) |
| M115 F5 | YOL123W | Putative |
| | | polyadenylated-RNA- |
| | | binding protein |
| | | located in nucleus\ |
| | | similar to vertebrate |
| | | hnRNP A\/B protein |
| | | family (58.85/70) |
| M282 G5 | YOL124C | (47.66/50) |
| M115 H7 | YOL125W | (52.47/64) |
| M278 E8 | YOL126C | cytosolic malate |
| | | dehydrogenase |
| | | (46.56/60) |
| M278 E2 | YOL128C | (41.38/49) |
| M278 A5 | YOL131W | (11.99/16) |
| M115 A8 | YOL133W | (13.42/20) |
| M282 A8 | YOL134C | (14.22/14) |
| M282 B3 | YOL137W | (54.78/64) |
| M118 D5 | YOL139C | mRNA cap binding |
| | | protein eIF-4E |
| | | (23.46/27) |
| M282 B8 | YOL142W | (26.51/34) |
| M278 E1 | YOL143C | 6 7-dimethyl-8- |
| | | ribityllumazine |
| | | synthase (DMRL |
| | | synthase) (18.62/30) |
| M278 C4 | YOL146W | (26.07/34) |
| M118 E5 | YOL147C | Peroxisomal |
| | | biogenesis protein |
| | | (peroxin) involved in |
| | | peroxisome |
| | | inheritance and |
| | | peroxisomal |
| | | proliferation |
| | | (25.99/34) |
| M278 D6 | YOL148C | putative transcription |
| | | factor (66.47/75) |
| M115 F1 | YOL151W | (37.73/45) |
| M278 D4 | YOL154W | (27.5/37) |
| M278 D5 | YOL155C | (106.4/0116) |
| M278 E7 | YOL157C | (64.82/75) |
| M118 F1 | YOL159C | (18.84/29) |
| M282 D2 | YOL160W | (12.54/12) |
| M118 G3 | YOL161C | (13.23/20) |
| M282 A4 | YOL162W | (23.76/30) |
| M282 A5 | YOL163W | (18.7/27) |
| M282 A7 | YOL165C | (15.76/19) |
| M282 E8 | YOL166C | (12.45/12) |
| M118 G1 | YOR001W | Ribosomal RNA |
| | | Processing (80.74/80) |
| M118 H3 | YOR003W | subtilisin-like |
| | | protease III |
| | | (52.69/64) |
| M278 F4 | YOR004W | (28.05/39) |
| M282 B5 | YOR005C | ATP dependent DNA |
| | | ligase (103.87/105) |
| M278 F6 | YOR006C | (34.46/34) |
| M282 B7 | YOR007C | (38.09/38) |
| M115 G9 | YOR008C | (41.61/55) |
| M282 H1 | YOR009W | (53.68/52) |
| M282 F2 | YOR010C | Cold-shock induced |
| | | protein of the |
| | | Srp1p\/Tip1p family |
| | | of serine-alanine-rich |
| | | proteins (27.64/35) |
| M282 C5 | YOR013W | (17.37/27) |
| M278 G6 | YOR014W | high copy suppressor |
| | | of rox3 and a |
| | | multicopy suppressor |
| | | of hsp60-ts alleles |
| | | (83.48/90) |
| M118 G9 | YOR016C | (22.80/33) |
| M118 H5 | YOR021C | (23.46/36) |
| M282 D7 | YOR023C | (62.39/64) |
| M282 G8 | YOR024W | (11.88/14) |
| M115 B2 | YOR025W | Homolog of SIR2 |
| | | (49.38/55) |
| M282 G2 | YOR026W | (37.62/38) |
| M282 E3 | YOR027W | heat shock protein |
| | | (64.9/65) |
| M265 E8 | YOR028C | (32.48/31) |
| M278 A7 | YOR030W | ExtraCellular Mutant |
| | | (68.2/80) |
| M118 | YOR032C | (47.77/47) |
| A10 | | |
| M115 C2 | YOR033C | Protein that |
| | | complements a drug- |
| | | hypersensitive |
| | | mutation (77.35/75) |
| M278 H4 | YOR036W | integral membrane |
| | | protein\c-terminal |
| | | TMD\located in |
| | | endosome (31.79/40) |
| M278 B6 | YOR037W | cytochrome c |
| | | mitochondrial import |
| | | factor (44.55/49) |
| M282 F7 | YOR039W | Casein kinase II beta' |
| | | subunit (28.49/32) |
| M118 | YOR040W | Mitochondrial |
| B10 | | glyoxylase-II |
| | | (31.46/45) |
| M279 A1 | YOR041C | (15.76/12) |
| M119 H3 | YOR043W | Protein involved in |
| | | growth regulation |
| | | (53.57/60) |
| M279 G3 | YOR044W | (17.48/23) |
| M120 F7 | YOR046C | RNA helicase |
| | | (53.05/60) |
| M279 B1 | YOR049C | (38.97/43) |
| M279 A2 | YOR050C | (12.68/16) |
| M120 A4 | YOR051C | (45.45/45) |
| M119 D5 | YOR052C | (16.53/32) |
| M120 G7 | YOR054C | (74.27/?) |
| M279 D7 | YOR056C | (50.52/65) |
| M280 C2 | YOR058C | encodes component |
| | | of the spindle |
| | | midzone (97.48/98) |
| M279 A4 | YOR060C | (28.30/35) |
| M279 E5 | YOR062C | (29.51/53) |
| M279 E6 | YOR063W | ribosomal protein L3 |
| | | (42.68/50) |
| M279 E7 | YOR064C | (24.12/24) |
| M120 D1 | YOR065W | Cytochrome c1 |
| | | (34.1/?) |
| M119 E1 | YOR073W | (65.01/75) |
| M279 B2 | YOR074C | 236-915 Thymidylate |
| | | synthase (28.6/39) |
| M279 F5 | YOR078W | (23.65/36) |
| M119 A3 | YOR082C | (12.46/20) |
| M119 E4 | YOR083W | (32.56/52) |
| M119 H5 | YOR084W | (42.68/52) |
| M119 C7 | YOR085W | 34-kDa gamma |
| | | subunit of |
| | | oligosaccharyl |
| | | transferase |
| | | glycoprotein complex |
| | | (38.61/45) |
| M119 | YOR088W | (53.13/40) |
| B11 | | |
| M120 G1 | YOR089C | small GTP-binding |
| | | protein\ |
| | | geranylgeranylated\ |
| | | geranylgeranylation |
| | | required for |
| | | membrane |
| | | association\also |
| | | involved in |
| | | endocytosis post |
| | | vesicle internalization |
| | | (23.13/33) |
| M279 E1 | YOR089C | small GTP-binding |
| | | protein\ |
| | | geranylgeranylated\ |
| | | geranylgeranylation |
| | | required for |
| | | membrane |
| | | association\also |
| | | involved in |
| | | endocytosis post |
| | | vesicle internalization |
| | | (23.13/33) |
| M119 F4 | YOR091W | (44.22/55) |
| M119 A6 | YOR092W | ExtraCellular Mutant |
| | | (67.54/60) |
| M119 | YOR095C | (28.41/38) |
| B10 | | |
| M119 | YOR096W | 546-974 |
| C11 | | (21.01/32) |
| M280 D3 | YOR099W | type 2 membrane |
| | | protein\probable |
| | | secretory protein |
| | | (43.34/50) |
| M120 A6 | YOR100C | (36.00/47) |
| M279 G5 | YOR102W | 16-kDa epsilon |
| | | subunit of |
| | | oligosaccharyltransfer |
| | | ase complex\40\% |
| | | identical to vertebrate |
| | | DAD1 protein |
| | | (12.87/39) |
| M279 D2 | YOR106W | member of the |
| | | syntaxin family of |
| | | proteins\predicted C- |
| | | terminal TMD |
| | | (31.24/43) |
| M119 G4 | YOR107W | (34.1/40) |
| M120 B6 | YOR108W | (66.55/66) |
| M119 | YOR111W | (25.63/34) |
| D10 | | |
| M280 F2 | YOR114W | (32.45/36) |
| M280 E3 | YOR115C | (29.51/31) |
| M119 F7 | YOR117W | (47.85/55) |
| M120 | YOR119C | (53.27/70) |
| C10 | | |
| M202 G7 | YOR120W | Similar to mammalian |
| | | aldo\/keto reductases |
| | | (34.43/50) |
| M120 C2 | YOR121C | (11.14/11) |
| M119 A5 | YOR123C | (51.07/75) |
| M120 H8 | YOR126C | isoamyl acetate |
| | | hydrolytic enzyme |
| | | (26.21/50) |
| M280 H8 | YOR128C | phosphoribosylamino |
| | | -imidazole- |
| | | carboxylase |
| | | (62.84/62) |
| M279 F3 | YOR131C | (24.01/30) |
| M119 H7 | YOR133W | translation elongation |
| | | factor 2 (EF-2) |
| | | (92.73/98) |
| M119 C9 | YOR134W | GTPase activating |
| | | protein (GAP) |
| | | (45.1/50) |
| M120 F11 | YOR136W | NAD+-dependent |
| | | isocitrate |
| | | dehydrogenase |
| | | (40.7/50) |
| M122 C2 | YOR138C | (73.84/74) |
| M281 C7 | YOR142W | (36.3/44) |
| M281 E8 | YOR143C | Thiamin |
| | | pyrophosphokinase |
| | | (35.12/44) |
| M122 B1 | YOR145C | (30.17/38) |
| M122 | YOR152C | (28.29/36) |
| G10 | | |
| M283 F2 | YOR154W | (64.68/81) |
| M122 A4 | YOR155C | (49.53/55) |
| M274 C8 | YOR156C | Interacts with C- |
| | | terminus of CDC12 |
| | | (79.89/95) |
| M281 A6 | YOR157C | putative proteasome |
| | | subunit (28.74/36) |
| M122 E9 | YOR159C | (10.47/18) |
| M121 C4 | YOR163W | (20.79/20) |
| M281 A5 | YOR164C | (34.45/40) |
| M122 C8 | YOR166C | (50.41/34) |
| M122 | YOR168W | glutaminyl-tRNA |
| A11 | | synthetase (89.1/98) |
| M283 D1 | YOR169C | (16.97/25) |
| M122 D8 | YOR174W | (31.45/45) |
| M122 | YOR176W | ferrochelatase |
| B11 | | (protoheme |
| | | ferrolyase) (43.34/48) |
| M122 C4 | YOR179C | (20.71/30) |
| M281 C5 | YOR180C | (29.84/35) |
| M122 A7 | YOR181W | prolin rich protein |
| | | (69.74/80) |
| M122 | YOR184W | phosphoserine |
| C11 | | transaminase |
| | | (43.56/50) |
| M283 F1 | YOR185C | GTP-binding protein |
| | | (24.23/34) |
| M283 B3 | YOR186W | (15.95/26) |
| M122 D4 | YOR187W | (48.28/50) |
| M122 B7 | YOR189W | (12.87/20) |
| M122 F8 | YOR190W | Exo-1 3-beta- |
| | | glucanase (49.06/52) |
| M122 B3 | YOR194C | Transcription factor |
| | | IIA large chain |
| | | (31.49/42) |
| M121 E4 | YOR195W | (90.42/90) |
| M281 D5 | YOR196C | Involved in lipoic |
| | | acid metabolism |
| | | (45.57/55) |
| M281 F6 | YOR197W | (49.94/60) |
| M121 A2 | YOR201C | Ribose |
| | | methyltransferase for |
| | | mitochondrial 21S |
| | | rRNA (45.35/55) |
| M122 A6 | YOR204W | ATP-dependent RNA |
| | | helicase of DEAD |
| | | box family\ |
| | | suppressor of a pre- |
| | | mRNA splicing |
| | | mutation prp8-1 |
| | | (66.55/70) |
| M281 H6 | YOR213C | (27.31/36) |
| M281 C9 | YOR215C | (20.48/30) |
| M121 | YOR216C | (53.37/64) |
| G11 | | |
| M283 F3 | YOR218C | (15.32/20) |
| M122 C6 | YOR220W | (29.36/34) |
| M281 C8 | YOR222W | (33.88/42) |
| M121 | YOR224C | 16-kDa RNA |
| H11 | | polymerase subunit |
| | | (common to |
| | | polymerases I II and |
| | | III) |
| | | (16.09/20) |
| M281 D3 | YOR226C | (17.29/18) |
| M122 A5 | YOR227W | (137.27/138) |
| M121 G7 | YOR229W | Transcriptional |
| | | modulator (51.48/64) |
| M121 C9 | YOR230W | Transcriptional |
| | | modulator (48.28/65) |
| M121 | YOR232W | (25.29/33) |
| A12 | | |
| M123 A1 | YOR233W | protein kinase |
| | | (88.11/100) |
| M124 D4 | YOR236W | dihydrofolate |
| | | reductase (23.32/32) |
| M124 E8 | YOR239W | (30.8/36) |
| M284 B8 | YOR240W | (39.93/48) |
| M285 B1 | YOR241W | (60.49/60) |
| M285 H2 | YOR243C | (74.49/80) |
| M285 E4 | YOR245C | (46.01/48) |
| M123 F6 | YOR246C | (36.33/40) |
| M123 G7 | YOR247W | (23.21/34) |
| M124 H9 | YOR248W | (11.11/30) |
| M123 D2 | YOR250C | (48.98/55) |
| M124 G3 | YOR251C | (33.47/36) |
| M284 H3 | YOR252W | (15.62/25) |
| M124 B6 | YOR253W | (19.47/32) |
| M123 H7 | YOR255W | (30.69/40) |
| M124 D1 | YOR257W | Calcium-binding |
| | | protein of spindle |
| | | pole body (17.82/20) |
| M284 B2 | YOR258W | (23.98/32) |
| M123 F3 | YOR259C | ATPase\component |
| | | of the 26S |
| | | proteasome cap |
| | | subunit (48.10/55) |
| M124 G4 | YOR260W | negative regulator in |
| | | the general control of |
| | | amino acid |
| | | biosynthesis |
| | | (63.69/70) |
| M124 C6 | YOR261C | (37.21/38) |
| M124 F7 | YOR262W | (38.38/40) |
| M123 C9 | YOR264W | (47.41/55) |
| M284 E1 | YOR265W | Binds to beta-tubulin |
| | | and may participate in |
| | | microtubule |
| | | morphogenesis |
| | | (11.77/14) |
| M284 C2 | YOR266W | (46.64/50) |
| M285 G3 | YOR268C | (14.55/30) |
| M124 D6 | YOR269W | Required for viability |
| | | in the absence of the |
| | | kinesin-related Cin8p |
| | | mitotic motor. |
| | | (54.45/54) |
| M123 B8 | YOR271C | (36.00/45) |
| M123 D9 | YOR272W | microtubule- |
| | | associated protein |
| | | (50.71/60) |
| M284 D2 | YOR274W | transfer RNA |
| | | isopentenyl |
| | | transferase (47.29/52) |
| M284 C4 | YOR276W | mRNA cap-binding |
| | | protein (eIF-4F) 20K |
| | | subunit (17.82/30) |
| M124 G7 | YOR278W | uroporphyrinogen III |
| | | synthase (30.46/36) |
| M124 B9 | YOR279C | (34.13/36) |
| M124 | YOR280C | (29.39/36) |
| C10 | | |
| M124 G1 | YOR281C | (31.49/32) |
| M124 B5 | YOR284W | (26.84/36) |
| M124 F6 | YOR285W | (15.4/20) |
| M285 H5 | YOR286W | (16.5/16) |
| M123 D8 | YOR287C | (33.03/48) |
| M124 | YOR288C | Disulfide isomerase |
| D10 | | related protein |
| | | (35.01/40) |
| M123 F1 | YOR289W | (27.72/34) |
| M124 A8 | YOR294W | (22.44/36) |
| M123 E8 | YOR295W | (25.29/36) |
| M255 H8 | YOR298W | (52.8/52) |
| M123 C6 | YOR301W | (47.96/50) |
| M123 F8 | YOR303W | Carbamoyl phosphate |
| | | synthetase arginine |
| | | specific (45.32/48) |
| M285 G7 | YOR311C | (31.93/34) |
| M285 G1 | YOR312C | 415-932 60S |
| | | ribosomal protein |
| | | L18A (19.35/29) |
| M285 F2 | YOR313C | (37.21/47) |
| M123 D7 | YOR317W | long chain fatty |
| | | acyl:CoA synthetase |
| | | (77.11/80) |
| M124 F10 | YOR319W | homolog of |
| | | mammalian splicing |
| | | factor\/U2 snRNP |
| | | protein (23.54/34) |
| M124 G5 | YOR323C | gamma-glutamyl |
| | | phosphate reductase |
| | | (50.29/55) |
| M124 C7 | YOR324C | (66.35/70) |
| M285 C6 | YOR325W | (17.48/19) |
| M284 B9 | YOR327C | vesicle-associated |
| | | membrane protein |
| | | (synaptobrevin) |
| | | homolog (12.68/22) |
| M100 F1 | YOR329C | (95.95/98) |
| M293 H2 | YOR331C | 27-kDa subunit of the |
| | | vacuolar ATPase\E |
| | | subunit of V1 sector |
| | | (20.48/21) |
| M100 F5 | YOR335C | Cytoplasmic alanyl- |
| | | tRNA synthetase gene |
| | | (105.41/98) |
| M310 C7 | YOR339C | (17.29/28) |
| M310 D7 | YOR342C | (35.12/42) |
| M100 A1 | YOR344C | 33 kDa serine-rich |
| | | protein (32.04/45) |
| M100 G2 | YOR347C | (55.69/60) |
| M100 H5 | YOR351C | kinase involved in |
| | | meiotic chromosome |
| | | pairing and |
| | | recombination |
| | | (54.70/60) |
| M100 B1 | YOR352W | (37.84/45) |
| M100 H2 | YOR355W | (57.53/64) |
| M100 E3 | YOR356W | (69.52/69) |
| M100 D4 | YOR357C | (17.85/25) |
| M100 H4 | YOR358W | Component along |
| | | with Hap2p and |
| | | Hap3p of CCAAT- |
| | | binding transcription |
| | | factor (26.73/40) |
| M106 E6 | YOR359W | (57.64/57) |
| M293 H6 | YOR367W | (22.11/36) |
| M100 C1 | YOR368W | DNA Damage |
| | | checkpoint control |
| | | (44.22/64) |
| M100 D2 | YOR370C | Rab geranylgeranyl |
| | | transferase (66.46/70) |
| M100 G3 | YOR372C | (60.97/62) |
| M100 B5 | YOR374W | (57.2/60) |
| M288 E7 | YOR375C | NADP-specific |
| | | glutamate |
| | | dehydrogenase |
| | | (49.97/55) |
| M100 D1 | YOR376W | (13.53/20) |
| M100 A3 | YOR379C | (12.45/16) |
| M100 H3 | YOR380W | (60.17/60) |
| M100 H1 | YOR385W | (32.01/40) |
| M100 B3 | YOR387C | (22.69/30) |
| M100 C5 | YOR390W | (41.46/41) |
| M100 C6 | YOR391C | (26.10/32) |
| M293 E1 | YOR392W | (16.38/20) |
| M100 A2 | YOR393W | enolase homolog |
| | | (48.28/55) |
| M100 C3 | YPL001W | histone |
| | | acetyltransferase |
| | | (41.25/48) |
| M106 H5 | YPL004C | (37.54/52) |
| M106 G6 | YPL005W | (66.77/66) |
| M100 B2 | YPL007C | (64.71/50) |
| M293 B2 | YPL007C | (64.71/62) |
| M100 D3 | YPL009C | (114.21/48) |
| M100 B4 | YPL010W | Coatomer complex |
| | | zeta chain (zeta-COP) |
| | | of secretory pathway |
| | | vesicles (20.9/32) |
| M288 F5 | YPL011C | (38.86/52) |
| M100 E5 | YPL012W | (135.29/135) |
| M100 C2 | YPL015C | Homolog of SIR2 |
| | | (39.30/45) |
| M100 C4 | YPL026C | Serine\/threonine |
| | | protein kinase that |
| | | suppresses the growth |
| | | defect of snf3 mutants |
| | | on low glucose |
| | | (55.35/55) |
| M126 A1 | YPL030W | (62.48/64) |
| M126 C2 | YPL031C | 120-1020 negative |
| | | transcriptional |
| | | regulator protein |
| | | kinase homolog |
| | | (33.66/38) |
| M126 F3 | YPL032C | (90.78/90) |
| M286 D4 | YPL033C | (30.94/40) |
| M286 E5 | YPL034W | (18.36/31) |
| M127 G9 | YPL037C | (17.30/25) |
| M127 B1 | YPL038W | (19.58/32) |
| M126 G3 | YPL040C | nuclear encoded |
| | | mitochondrial |
| | | isoleucyl-tRNA |
| | | synthetase |
| | | (110.35/110) |
| M287 F4 | YPL042C | a cyclin (SSN8)- |
| | | dependent |
| | | serine\/threonine |
| | | protein kinase |
| | | (61.08/54) |
| M127 E7 | YPL043W | RNA recognition |
| | | motif-containing |
| | | protein (75.46/80) |
| M126 H3 | YPL048W | Calciuim and |
| | | phospholipid binding |
| | | protein homologous |
| | | to translation |
| | | elongation factor-1 |
| | | gamma (EF-1 |
| | | gamma) (45.76/45) |
| M126 D6 | YPL050C | Protein required for |
| | | complex |
| | | glycosylation |
| | | (43.48/45) |
| M286 G6 | YPL051W | (21.89/30) |
| M126 G8 | YPL052W | (22.22/30) |
| M126 | YPL053C | similar to KRE2 |
| A10 | | (49.09/50) |
| M126 C1 | YPL054W | (33.22/38) |
| M127 B2 | YPL055C | (36.55/42) |
| M287 B4 | YPL057C | Multicopy suppressor |
| | | of cls2-2\also |
| | | suppresses rvs161 |
| | | mutations (42.05/42) |
| M286 H6 | YPL059W | (16.61/16) |
| M287 D1 | YPL062W | (14.85/26) |
| M126 F2 | YPL063W | (52.47/55) |
| M126 B4 | YPL064C | (33.14/40) |
| M126 C5 | YPL065W | soluble hydrophilic |
| | | protein involved in |
| | | transport of |
| | | precursors for soluble |
| | | vauolar hydrolases |
| | | from the late |
| | | endosome to the |
| | | vacuole (26.73/32) |
| M126 A9 | YPL068C | (32.36/38) |
| M126 | YPL069C | geranylgeranyl |
| B10 | | diphosphate synthase |
| | | (36.88/38) |
| M126 E1 | YPL070W | (67.43/67) |
| M126 G2 | YPL071C | (17.29/20) |
| M286 G3 | YPL072W | (55/60) |
| M126 F6 | YPL074W | (83./0583) |
| M126 | YPL077C | (26.4/332) |
| C10 | | |
| M126 F1 | YPL078C | F(1)F(0)-ATPase |
| | | complex delta subunit |
| | | mitochondrial |
| | | (26.87/30) |
| M286 F2 | YPL079W | 433-904 |
| | | (17.71/27) |
| M287 G3 | YPL080C | (11.91/12) |
| M126 E5 | YPL081W | 509-1095 |
| | | (21.78/21) |
| M126 G1 | YPL086C | (61.30/62) |
| M126 D4 | YPL088W | (37.73/45) |
| M287 G5 | YPL091W | Glutathione |
| | | oxidoreductase |
| | | (53.24/53) |
| M127 E10 | YPL093W | (71.38/80) |
| M286 G1 | YPL094C | membrane component |
| | | of ER protein |
| | | translocation |
| | | apparatus (31.26/38) |
| M126 G5 | YPL097W | Tyrosyl-tRNA |
| | | synthetase (54.23/54) |
| M126 B8 | YPL099C | (20.05/20) |
| M286 E8 | YPL100W | (54.67/64) |
| M126 | YPL101W | (50.37/52) |
| D10 | | |
| M287 G1 | YPL102C | (11.03/11) |
| M286 A3 | YPL103C | (51.51/55) |
| M127 A7 | YPL106C | Member of the 70- |
| | | kDa heat-shock |
| | | protein family |
| | | (76.36/80) |
| M287 A6 | YPL107W | (27.49/36) |
| M126 D9 | YPL108W | (18.59/28) |
| M126 E10 | YPL109C | (59.76/59) |
| M126 D3 | YPL111W | arginase (36.74/40) |
| M126 H5 | YPL113C | (43.59/45) |
| M126 E9 | YPL116W | (76.78/76) |
| M287 H1 | YPL118W | (37.95/51) |
| M126 E3 | YPL119C | putative ATP- |
| | | dependent RNA |
| | | helicase\Dead box |
| | | protein (67.90/67) |
| M126 F4 | YPL120W | (61.48/64) |
| M126 D8 | YPL123C | (47.77/55) |
| M126 F9 | YPL124W | Nuclear import |
| | | protein (27.94/32) |
| M129 A2 | YPL127C | histone H1 (28.41/38) |
| M130 E3 | YPL128C | TTAGGG repeat |
| | | binding factor |
| | | (61.85/64) |
| M130 D4 | YPL129W | 115-840 |
| | | (26.95/30) |
| M129 G6 | YPL131W | Ribosomal protein |
| | | RPL1 (YL3) (rat L5) |
| | | (32.78/40) |
| M129 C8 | YPL132W | Putative heme A |
| | | biosynthetic enzyme |
| | | involved in forming |
| | | the formyl group at |
| | | position 8 of the |
| | | porphyrin ring |
| | | (33.11/40) |
| M291 C8 | YPL133C | (49.09/55) |
| M289 B1 | YPL134C | (34.13/40) |
| M130 E2 | YPL135W | (18.36/18) |
| M289 F5 | YPL138C | (38.86/48) |
| M291 G5 | YPL139C | Transcriptional |
| | | modulator (50.63/60) |
| M255 D9 | YPL141C | (95.28/98) |
| M129 C4 | YPL145C | Homologous to |
| | | human oxysterol- |
| | | binding protein\ |
| | | implicated in |
| | | ergosterol |
| | | biosynthesis and |
| | | regulation of Golgi- |
| | | derived transport |
| | | vesicle biogenesis |
| | | (47.77/55) |
| M130 F5 | YPL146C | (50.08/55) |
| M129 E8 | YPL148C | (19.50/30) |
| M129 | YPL149W | involved in autophagy |
| A10 | | (32.45/34) |
| M289 D1 | YPL150W | (99.22/105) |
| M129 D2 | YPL151C | (49.64/52) |
| M129 C3 | YPL152W | (39.49/50) |
| M289 E4 | YPL153C | (90.34/98) |
| M129 F8 | YPL156C | (31.37/36) |
| M129 | YPL157W | (34.76/40) |
| B10 | | |
| M130 E1 | YPL158C | (83.41/98) |
| M129 E2 | YPL159C | (27.86/34) |
| M129 H5 | YPL162C | (30.06/40) |
| M291 D7 | YPL164C | (78.68/79) |
| M130 E9 | YPL165C | (41.06/45) |
| M130 F1 | YPL166W | (23.54/33) |
| M289 F3 | YPL168W | (47.41/50) |
| M291 F3 | YPL169C | (65.92/100) |
| M129 D7 | YPL171C | NAD(P)H |
| | | dehydrogenase |
| | | (44.03/48) |
| M130 F9 | YPL173W | (32.78/36) |
| M130 G1 | YPL174C | Nuclear import |
| | | protein (95.51/98) |
| M291 G3 | YPL177C | Homeobox-domain |
| | | containing protein |
| | | (33.69/57) |
| M129 A6 | YPL178W | (22.99/34) |
| M291 C6 | YPL179W | protein phosphatase Q |
| | | (60.5/63) |
| M130 G9 | YPL181W | (55.77/50) |
| M291 B5 | YPL186C | (33.47/57) |
| M130 D8 | YPL188W | (45.65/52) |
| M289 H1 | YPL190C | (88.35/100) |
| M130 B3 | YPL191C | (39.63/49) |
| M129 A5 | YPL193W | (42.02/52) |
| M129 C6 | YPL194W | (67.43/70) |
| M130 E8 | YPL196W | (30.14/34) |
| M130 C3 | YPL199C | (26.43/38) |
| M129 B5 | YPL201C | (50.74/55) |
| M130 D7 | YPL203W | (41.91/50) |
| M130 F8 | YPL204W | casein kinase I |
| | | isoform (54.45/54) |
| M291 F1 | YPL206C | (35.34/37) |
| M130 B4 | YPL208W | (64.24/64) |
| M289 D5 | YPL209C | Protein kinase |
| | | (40.40/50) |
| M130 D6 | YPL210C | (71.09/71) |
| M130 E7 | YPL211W | (20.02/32) |
| M130 | YPL213W | (26.39/32) |
| A10 | | |
| M289 C2 | YPL214C | TMP |
| | | pyrophosphorylase |
| | | hydroxyethylthiazole |
| | | kinase (59.43/62) |
| M130 D3 | YPL215W | (36.96/38) |
| M130 F7 | YPL219W | (54.23/64) |
| M291 B8 | YPL220W | homologue of the E- |
| | | and Archebacterial L1 |
| | | ribisomal protein of |
| | | the 60S ribosomal |
| | | subunit (23.98/33) |
| M129 | YPL221W | (87.34/70) |
| H10 | | |
| M292 A1 | YPL222W | (75.79/76) |
| M132 G1 | YPL223C | (18.51/28) |
| M132 A3 | YPL225W | (16.27/20) |
| M132 G5 | YPL228W | (60.5/68) |
| M133 A1 | YPL230W | (43.12/55) |
| M132 E2 | YPL232W | (32.01/45) |
| M292 B3 | YPL233W | (23.87/33) |
| M292 H3 | YPL234C | 17-kDa subunit C of |
| | | vacuolar membrane |
| | | H(+)-ATPase |
| | | (18.07/32) |
| M132 H5 | YPL236C | (40.07/45) |
| M132 A7 | YPL237W | beta subunit of |
| | | translation initiation |
| | | factor eIF-2 |
| | | (31.46/34) |
| M133 B2 | YPL239W | (22.11/32) |
| M290 A3 | YPL240C | heat shock protein |
| | | (78.02/98) |
| M292 C3 | YPL241C | 106-887 involvement |
| | | in microtubule |
| | | function (29.59/33) |
| M290 D5 | YPL243W | Signal recognition |
| | | particle subunit |
| | | (66/70) |
| M292 F5 | YPL244C | (37./3237) |
| M133 C8 | YPL245W | (50.0/555) |
| M292 C1 | YPL246C | (28.85/29) |
| M133 C2 | YPL247C | (57.56/60) |
| M132 B6 | YPL252C | (18.95/30) |
| M133 D8 | YPL253C | (71.20/71) |
| M132 D1 | YPL254W | (53.79/55) |
| M292 D3 | YPL257W | (21.34/33) |
| M255 E9 | YPL258C | (60.64/60) |
| M290 F5 | YPL259C | medium subunit of |
| | | the clathrin-associated |
| | | protein complex |
| | | (52.38/64) |
| M133 B7 | YPL260W | (60.72/70) |
| M132 E1 | YPL262W | mitochondrial and |
| | | cytoplasmic fumarase |
| | | (fumarate hydralase) |
| | | (53.79/53) |
| M133 E2 | YPL263C | (71.64/75) |
| M132 C5 | YPL267W | (23.1/34) |
| M132 C7 | YPL269W | (70.95/70) |
| M290 C3 | YPL272C | (56.90/64) |
| M132 F3 | YPL273W | (35.86/36) |
| M290 H5 | YPL275W | (26.07/36) |
| M292 A6 | YPL276W | (16.06/31) |
| M133 F8 | YPL277C | (53.60/60) |
| M290 C1 | YPL278C | (11.03/20) |
| M290 D3 | YPL280W | (26.28/34) |
| M292 F3 | YPL281C | Enolase-related |
| | | subtelomeric |
| | | sequence (ERR1 and |
| | | ERR2 code for |
| | | identical proteins) |
| | | (48.10/41) |
| M133 B5 | YPL282C | (18.07/32) |
| M290 F6 | YPR001W | Citrate synthase |
| | | (53.57/54) |
| M132 G2 | YPR005C | polar 32k Da |
| | | cytoplasmic protein |
| | | (32.47/38) |
| M132 D4 | YPR007C | (74.83/80) |
| M133 C6 | YPR008W | (76.45/76) |
| M133 G1 | YPR011C | (35.89/50) |
| M290 E3 | YPR013C | (34.90/34) |
| M292 G3 | YPR014C | (12.02/56) |
| M132 E4 | YPR015C | (27.20/34) |
| M132 E5 | YPR016C | (26.98/32) |
| M290 H6 | YPR017C | GDP dissociation |
| | | factor for Sec4p |
| | | (15.76/25) |
| M132 B2 | YPR020W | (12.76/12) |
| M133 E5 | YPR023C | (44.14/55) |
| M132 F5 | YPR024W | Mitochondrial protein |
| | | of the |
| | | CDC48$$PAS1$$SEC1 |
| | | 8 family of ATPases |
| | | (82.38/82) |
| M133 G7 | YPR025C | cyclin |
| | | (43.36/50) |
| M290 E1 | YPR027C | (30.50/30) |
| M132 C2 | YPR028W | 185-676 |
| | | (19.91/20) |
| M133 H7 | YPR033C | cytoplasmic and |
| | | mitochondrial |
| | | histidine tRNA |
| | | synthetases |
| | | (60.09/60) |
| M294 A1 | YPR035W | glutamine synthetase |
| | | (40.81/47) |
| M134 E2 | YPR036W | 54-kDa vacuolar H(+) |
| | | ATPase subunit of V1 |
| | | sector (52.69/52) |
| M134 H3 | YPR037C | (21.59/25) |
| M295 A8 | YPR040W | (39.37/50) |
| M295 C9 | YPR041W | Translation initiation |
| | | factor eIF-5 |
| | | (44.66/52) |
| M131 A7 | YPR048W | (68.64/68) |
| M134 C1 | YPR051W | N-acetyltransferase |
| | | (19.47/30) |
| M294 A5 | YPR054W | Sporulation-specific |
| | | MAP kinase required |
| | | for completion of |
| | | sporulation (42.79/55) |
| M134 | YPR057W | Protein involved in |
| A10 | | snRNP biogenesis |
| | | (37.62/45) |
| M134 | YPR058W | putative |
| D11 | | mitochondrial carrier |
| | | protein (33.88/40) |
| M134 G2 | YPR060C | chorismate mutase |
| | | (28.29/34) |
| M294 G3 | YPR061C | (33.14/43) |
| M294 B5 | YPR062W | cytosine deaminase |
| | | (17.49/27) |
| M131 B6 | YPR063C | 128-509 |
| | | (15.51/30) |
| M295 E9 | YPR065W | site-specific DNA |
| | | binding protein |
| | | repressor (40.59/49) |
| M134 E11 | YPR066W | (33/36) |
| M131 C1 | YPR067W | (20./4630) |
| M294 D2 | YPR068C | Protein with |
| | | similarity to Hda1p |
| | | Rpd3p Hos2p and |
| | | Hos3p (51.73/60) |
| M131 D3 | YPR069C | putrescine |
| | | aminopropyltransfera |
| | | se (spermidine |
| | | synthase) (32.36/34) |
| M134 G5 | YPR070W | (62.47/64) |
| M134 C7 | YPR071W | (23.32/32) |
| M131 F8 | YPR073C | 18-kDa |
| | | phosphotyrosine |
| | | phosphatase of |
| | | unknown function |
| | | (17.74/25) |
| M294 | YPR074C | Transketolase 1 |
| D10 | | (74.83/85) |
| M134 F1 | YPR075C | (39.63/48) |
| M131 C6 | YPR079W | (42.02/42) |
| M134 H8 | YPR080W | translational |
| | | elongation factor EF- |
| | | 1 alpha (50.49/50) |
| M295 G9 | YPR081C | (68.01/68) |
| M134 | YPR082C | (15.76/25) |
| G11 | | |
| M294 F2 | YPR084W | (50.37/58) |
| M131 A5 | YPR086W | transcription factor |
| | | TFIIB homolog |
| | | (38.06/48) |
| M295 C7 | YPR087W | (11.77/14) |
| M134 H1 | YPR091C | (84.73/84) |
| M294 G2 | YPR092W | (11.22/16) |
| M134 G4 | YPR093C | (34.13/36) |
| M131 D1 | YPR099C | (13.01/13) |
| M294 D4 | YPR101W | (19.46/34) |
| M131 C5 | YPR102C | (19.27/20) |
| M134 C9 | YPR104C | Protein with a domain |
| | | similar to the fork |
| | | head DNA-binding |
| | | domain found in the |
| | | developmental fork |
| | | head protein of |
| | | Drosophila |
| | | melanogaster and in |
| | | the HNF-3 family of |
| | | hepatocyte |
| | | mammalian |
| | | transcription |
| | | (102.99/102) |
| M131 E1 | YPR107C | Yeast 30kDa |
| | | Homologue |
| | | (22.91/33) |
| M295 E3 | YPR108W | (47.3/54) |
| M131 D5 | YPR110C | RNA polymerase III |
| | | (C) subunit |
| | | (36.88/45) |
| M131 A8 | YPR112C | (97.60/98) |
| M134 C2 | YPR115W | (119.24/119) |
| M294 B3 | YPR116W | (30.58/42) |
| M131 E5 | YPR118W | (45.32/50) |
| M131 B8 | YPR120C | (47.88/47) |
| M301 C4 | YPR121W | (63.03/66) |
| M295 D2 | YPR123C | (15.87/25) |
| M295 G3 | YPR124W | High affinity copper |
| | | transporter into the |
| | | cell probable integral |
| | | membrane protein |
| | | (44.77/37) |
| M131 C4 | YPR125W | (50.05/55) |
| M131 G6 | YPR127W | (38.06/45) |
| M294 F8 | YPR128C | (36.11/40) |
| M134 | YPR129W | multicopy suppressor |
| B11 | | of clathrin deficiency |
| | | (38.5/50) |
| M263 A1 | YPR131C | (27.64/36) |
| M263 G2 | YPR133C | (45.13/55) |
| M263 E3 | YPR134W | Protein involved in |
| | | splicing intron |
| | | a15beta of COX1 |
| | | (29.59/34) |
| M263 C5 | YPR136C | (18.73/31) |
| M263 B6 | YPR137W | (63.14/80) |
| M264 F6 | YPR138C | Ammonia permease |
| | | of high capacity and |
| | | low affinity |
| | | (53.82/60) |
| M264 B1 | YPR139C | (33.03/36) |
| M255 H9 | YPR140W | (42.02/40) |
| M264 C4 | YPR143W | (27.61/47) |
| M264 B6 | YPR145W | asparagine synthetase |
| | | (63.03/67) |
| M264 G6 | YPR146C | (12.02/16) |
| M264 C1 | YPR147C | (33.47/40) |
| M264 B2 | YPR148C | (47.88/60) |
| M263 H2 | YPR149W | involved in secretion |
| | | of proteins that lack |
| | | classical secretory |
| | | signal sequences |
| | | (19.14/25) |
| M263 E4 | YPR151C | (22.69/32) |
| M263 E5 | YPR152C | (51.28/60) |
| M263 A3 | YPR157W | (51.48/64) |
| M263 H3 | YPR158W | (27.83/42) |
| M264 D1 | YPR163C | Translation initiation |
| | | factor eIF-4B |
| | | (47.99/50) |
| M263 B3 | YPR165W | GTP-binding protein |
| | | of the rho subfamily |
| | | of ras-like proteins |
| | | (23.1/30) |
| M263 A4 | YPR166C | 14 kDa mitochondrial |
| | | ribosomal protein\ |
| | | homologous to E. coli |
| | | S14 protein |
| | | (12.68/17) |
| M263 F4 | YPR167C | 3′ phosphoadenylylsul |
| | | fate reductase |
| | | (28.74/36) |
| M263 D6 | YPR169W | (56.65/62) |
| M263 C7 | YPR170C | (12.24/18) |
| M263 C2 | YPR172W | (22.11/33) |
| M263 C3 | YPR173C | defective in vacuolar |
| | | protein sorting |
| | | (48.10/56) |
| M264 A4 | YPR174C | (24.34/35) |
| M263 G4 | YPR175W | DNA polymerase |
| | | epsilon subunit B |
| | | (76.23/76) |
| M264 F5 | YPR176C | Geranylgeranyltransfe |
| | | rase Type II beta |
| | | subunit (35.78/39) |
| M264 D6 | YPR177C | (13.56/17) |
| M263 D7 | YPR178W | associated with the |
| | | U4$$U6 snRNP |
| | | (51.36/55) |
| M264 D2 | YPR180W | Along with Uba2p |
| | | forms a heterodimeric |
| | | activating enzyme for |
| | | Smt3p (38.38/40) |
| M264 B4 | YPR182W | Sm or Sm-like snRNP |
| | | protein (9.57/10) |
| M264 H4 | YPR183W | dolichol phosphate |
| | | mannose synthase |
| | | (29.48/36) |
| M263 E7 | YPR186C | Transcription factor |
| | | IIIA (47.22/51) |
| M264 E1 | YPR187W | 97-544 subunit |
| | | common to RNA |
| | | polymerases I II and |
| | | III (17.26/29) |
| M263 E2 | YPR188C | (17.96/30) |
| M263 A5 | YPR191W | 40 kDa ubiquinol |
| | | cytochrome-c |
| | | reductase core protein |
| | | 2 (40.59/46) |
| M263 H5 | YPR192W | (33.66/40) |
| M264 E6 | YPR193C | (17.29/28) |
| M264 B5 | YPR199C | (32.37/39) |
| M265 | YPR200C | (14.33/16) |
| A10 | | |
| M264 E7 | YPR202W | 168-865 |
| | | (26.39/26) |
| M264 G1 | YPR203W | (11.33/16) |

### Example 2

### High-Throughput Expression of Human Gene Sequences

The following example illustrates the construction of a library of expressible human gene sequences using the method of the invention. Primers were constructed based on sequences of human genes available from GenBank.

Fetal human heart tissue was obtained from the International Institute for the advancement of Medicine (IIAM). Poly A+mRNA was isolated using the FastTrack™ 2.0 Kit (Invitrogen, Carlsbad, Calif.) according to the manufacturer's instructions. The mRNA was converted to first-strand cDNA using a cDNA Cycle® Kit (Invitrogen) using the oligo dT primer provided and the protocols suggested. A single cDNA synthesis reaction was split into 12 separate wells of a 96-well PCR amplification plate, and PCR amplifications were performed using specific primer sets, essentially as described above, with the exception that the ratio of Taq to Pfu was 50:1 in the initial amplification (final conc. 2 U Taq:0.04 U Pfu/well). Sequence specific primers for each sequence being amplified were designed to start amplification at the start codon (ATG) of each sequence and end at the stop codon. In some cases, the primer design removed the stop codon from the DNA sequence, allowing for generation of a fusion protein when inserted into suitable expression vectors. Primers were synthesized using a Primerstation 960 (Intelligent Automation Systems, Inc.) used according to the manufacturer's instructions and were designed from sequences downloaded from Unigene and sent directly to the synthesizer Approximately 15 nMoles of each primer, having an average length of 25 basepairs (melting point between 60°-64° C., was synthesized in a 96-well format. After synthesis, the primers were cleaved from the supports, deprotected and dried in the same 96-well format (see manufacturer's instructions). zThe amplified gene sequences were purified and inserted into the pcDNA3.1/GS expression vector or pTYB2.2 expression vector (Invitrogen, Carlsbad, Calif.) essentially as described above. The expression vectors containing sequences verified to be in the correct orientation were transfected into CHO cells in 96-well deep-well blocks using the Pfx-6 PerFect Lipid system (Invitrogen, Cat #T930-16). Cell lysates were made 48 hours after transfection, and the lysates were separated by SDS-PAGE and analyzed by Western blot according to standard protocols using an anti-V5 epitope tag Mab/horseradish peroxidase conjugate. Table 2 below lists the human proteins successfully expressed using this methodology. The ORFs identified as HP—were expressed in pTYB2.2. All other ORFs were expressed in pDNA3.1.

Additional collections of human sequences were obtained from a variety of sources, including adult lung, heart and mammary tissue and fetal liver tissue. Sequences were inserted into several expression vectors having features suitable for different purposes, including pIND/GS, pRSET, pCRT7, pcDNA3.1/GS, and pBAD/Thio-V5-His (Invitrogen, Carlsbad, Calif.). Sequences obtained are listed in Table 2 below.

TABLE 2
Human ORFs

| | | | Predicted | Actual |
|---|---|---|---|---|
| Plate Number | Accession Number | Description | Size | Size |
| M235 C7 | H-A06977 | albumin | 67.1 | 67.0 kDa |
| E1 | H-AB002391 | Human mRNA for KIAA0393 | 68.09 | 68 |
| | | gene, complete cds | | |
| H3 | H-AB006969 | Homo sapiens hGAA1 mRNA, | 68.42 | 70 |
| | | complete cds | | |
| E2 | H-AB007875 | Homo sapiens KIAA0415 mRNA, | 51.48 | 51 |
| | | complete cds | | |
| D1 | H-AB007887 | Homo sapiens KIAA0427 mRNA, | 66.55 | 70 |
| | | complete cds | | |
| M421 D6 | H-AB010710 | Homo sapiens mRNA for lectin- | 30.14 | 45.0 kDa |
| | | like oxidized LDL receptor, | | |
| | | complete cds | | |
| G3 | H-AD001528 | Homo sapiens spermidine | 40.37 | 40 |
| | | aminopropyltransferase mRNA, | | |
| | | complete cds | | |
| B5 | H-AE000659 | Homo sapiens T-cell receptor | 12.39 | 16 |
| | | alpha delta locus from bases | | |
| | | 250472 to 501670 (section 2 of 5) | | |
| | | of the C | | |
| E2 | H-AF004022 | Homo sapiens protein kinase | 38.28 | 44 |
| | | mRNA, complete cds | | |
| M428 C1 | H-AF004231 | Homo sapiens | 65.78 | 70.0 kDa |
| | | monocyte/macrophage Ig-related | | |
| | | receptor MIR-10 (MIR cl-10) | | |
| | | mRNA, complete cds | | |
| A5 | H-AF004327 | Homo sapiens angiopoietin-2 | 54.67 | 60 |
| | | mRNA, complete cds | | |
| C1 | H-AF006501 | Homo sapiens chromosome 22 | 14.08 | 24 |
| | | cosmid clone c1155, RNA | | |
| | | polymerase II subunit 14.4 kDa | | |
| | | (POLRF) gene, complete cds | | |
| H4 | H-AF008936 | Homo sapiens syntaxin-16B | 35.75 | 47 |
| | | mRNA, complete cds | | |
| H5 | H-AF009243 | Homo sapiens proline-rich Gla | 22.33 | 36 |
| | | protein 2 (PRGP2) mRNA, | | |
| | | complete cds | | |
| M462 D6 | H-AF013249 | Homo sapiens leukocyte- | 31.68 | 40.0 kDa |
| | | associated Ig-like receptor-1 | | |
| | | (LAIR-1) mRNA, complete cds | | |
| A1 | H-AF013512 | untitled | 53.02 | 53 |
| A3 | H-AF013970 | Homo sapiens MTG8-like protein | 66.55 | 70 |
| | | (MTGR1) mRNA, complete cds | | |
| M467 A7 | H-AF014807 | Homo sapiens | 23.54 | 29.0 kDa |
| | | phosphatidylinositol synthase | | |
| | | (PIS) mRNA, complete cds | | |
| D2 | H-AF015257 | Homo sapiens flow-induced | 41.36 | 40 |
| | | endothelial G protein-coupled | | |
| | | receptor (FEG-1) mRNA, | | |
| | | complete cds | | |
| M422 B5 | H-AF017307 | Homo sapiens Ets-related | 40.92 | 49.0 kDa |
| | | transcription factor (ERT) mRNA, | | |
| | | complete cds | | |
| A6 | H-AF017656 | Homo sapiens G protein beta 5 | 38.94 | 48 |
| | | subunit mRNA, complete cds | | |
| E1 | H-AF017995 | Homo sapiens 3-phosphoinositide | 61.27 | 52 |
| | | dependent protein kinase-1 | | |
| | | (PDK1) mRNA, complete cds | | |
| G1 | H-AF019612 | Homo sapiens S2P mRNA, | 57.2 | 57 |
| | | complete cds | | |
| D3 | H-AF020591 | Homo sapiens zinc finger protein | 78.76 | 74 |
| | | mRNA, complete cds | | |
| A7 | H-AF022385 | Homo sapiens apoptosis-related | 23.43 | 33 |
| | | protein TFAR15 (TFAR15) | | |
| | | mRNA, complete cds | | |
| H6 | H-AF024714 | Homo sapiens interferon- | 37.84 | 48 |
| | | inducible protein (AIM2) mRNA, | | |
| | | complete cds | | |
| B1 | H-AF025527 | Homo sapiens leucocyte | 48.4 | 47 |
| | | immunoglobulin-like receptor-4 | | |
| | | (LIR-4) mRNA, complete cds | | |
| M424 B4 | H-AF025532 | Homo sapiens leucocyte | 49.39 | 59.0 kDa |
| | | immunoglobulin-like receptor-5 | | |
| | | (LIR-5) mRNA, complete cds | | |
| H5 | H-AF026071 | Homo sapiens soluble death | 30.58 | 50 |
| | | receptor 3 beta (DR3) mRNA, | | |
| | | complete cds | | |
| M428 A1 | H-AF026273 | Homo sapiens interleukin-1 | 65.01 | 68.0 kDa |
| | | receptor-associated kinase-2 | | |
| | | mRNA, complete cds | | |
| B6 | H-AF026293 | Homo sapiens chaperonin | 58.96 | 58 |
| | | containing t-complex polypeptide | | |
| | | 1, beta subunit (Cctb) mRNA, | | |
| | | complete cds | | |
| B5 | H-AF026548 | Homo sapiens branched chain | 45.43 | 50 |
| | | alpha-ketoacid dehydrogenase | | |
| | | kinase precursor, mRNA, nuclear | | |
| | | gene encoding mitochondrial | | |
| | | protein, complete cds | | |
| B2 | H-AF027204 | Homo sapiens putative tetraspan | 21.78 | 27 |
| | | transmembrane protein L6H | | |
| | | (TM4SF5) mRNA, complete cds | | |
| M426 D3 | H-AF028008 | Homo sapiens SP1-like zinc | 56.43 | 64.0 kDa |
| | | finger transcription factor SLP | | |
| | | mRNA, complete cds | | |
| B1 | H-AF029232 | Homo sapiens calpamodulin | 70.62 | 70 |
| | | (CalpM) mRNA, complete cds | | |
| M422 A7 | H-AF029761 | Homo sapiens decoy receptor 2 | 42.57 | 50.0 kDa |
| | | mRNA, complete cds | | |
| M477 F3 | H-AF029893 | Homo sapiens i-beta-1,3-N- | 45.76 | 50.0 kDa |
| | | acetylglucosaminyltransferase | | |
| | | mRNA, complete cds | | |
| C5 | H-AF032437 | Homo sapiens mitogen activated | 51.92 | 50 |
| | | protein kinase activated protein | | |
| | | kinase gene, complete cds | | |
| M416 F3 | H-AF035824 | Homo sapiens vesicle soluble | 25.63 | 36.0 kDa |
| | | NSF attachment protein receptor | | |
| | | (VTI1) mRNA, complete cds | | |
| F3 | H-AF037335 | Homo sapiens carbonic anhydrase | 39.05 | 39 |
| | | precursor (CA 12) mRNA, | | |
| | | complete cds | | |
| G1 | H-AF039019 | Homo sapiens zinc finger DNA | 87.45 | 87 |
| | | binding protein 89 kDa (ZBP-89) | | |
| | | mRNA, complete cds | | |
| G1 | H-AF039136 | Homo sapiens Fas binding protein | 81.51 | 98 |
| | | (hDaxx) mRNA, complete cds | | |
| A7 | H-AF040705 | Homo sapiens putative tumor | 31.57 | 41 |
| | | suppressor protein unspliced form | | |
| | | (Fus-2) mRNA, complete cds | | |
| M469 F1 | H-AF040958 | Homo sapiens lysosomal | 45.76 | 46.0 kDa |
| | | neuraminidase precursor, mRNA, | | |
| | | complete cds | | |
| G2 | H-AF043472 | Homo sapiens Shab-related | 54.12 | 64 |
| | | delayed-rectifier K+ channel | | |
| | | alpha subunit (Kv9.3) mRNA, | | |
| | | complete cds | | |
| E2 | H-AJ001340 | Homo sapiens mRNA for U3 | 52.36 | 60 |
| | | snoRNP associated 55 kDa | | |
| | | protein | | |
| G1 | H-D00096 | Transtyretin (prealbumin) | 16.28 | 20 |
| C4 | H-D00408 | Cytochrome P450 IIIA7 (P450- | 55.44 | 64 |
| | | HFLa) | | |
| M302 E7 | H-D00682 | cofilin | 18.37 | 30 |
| M383 G2 | H-D00726 | ferrochelatase | 46.64 | 50.0 kDa |
| M383 C3 | H-D00760 | proteasome, subunit HC3 | 25.85 | 34.0 kDa |
| M305 B4 | H-D00761 | proteasome, subunit HC5 | 26.62 | 33 |
| M266 F7 | H-D00763 | proteasome, subunit HC9 | 28.82 | 33 |
| E2 | H-D00860 | Phosphoribosyl pyrophosphate | 35.09 | 47 |
| | | synthetase subunit I | | |
| 215-13 | H-D10522 | human mRNA for 80 K-L protein | 35 | 36.59 |
| M423 F5 | H-D11086 | Interleukin 2 receptor gamma | 40.7 | 45.0 kDa |
| | | chain | | |
| M248 D2 | H-D11094 | positive modulator of HIV tat- | 47.74 | 40.0 kDa |
| | | mediated transactivation | | |
| G3 | H-D11428 | Peripheral myelin protein 22 | 17.71 | 17 |
| M424 D3 | H-D13168 | Human gene for endothelin-B | 48.73 | 48.0 kDa |
| | | receptor (hET-BR) | | |
| M271 B8 | H-D13315 | glyoxalase I, | 20.35 | 34.0 kDa |
| | | LACTOYLGLUTATHIONE | | |
| | | LYASE. CATALYZES THE | | |
| | | CONVERSION OF | | |
| | | HEMIMERCAPTAL, FORMED | | |
| | | FROM METHYLGLYOXAL | | |
| | | AND GLUTATHIONE, TO S- | | |
| | | LACTOYLGLUTATHIONE. | | |
| M306 F1 | H-D13627 | hypothetical protein | 60.39 | 90 |
| | | (GB: D13627) | | |
| M248 D1 | H-D13630 | hypothetical protein | 46.2 | 49 |
| | | (GB: D13630) , Human mRNA for | | |
| | | KIAA0005 gene, complete cds | | |
| M270 D5 | H-D13634 | hypothetical protein | 34.65 | 42.0 kDa |
| | | (GB: D13634) | | |
| M250 D2 | H-D13642 | hypothetical protein | 44 | 48.0 kDa |
| | | (GB: D13642), Human mRNA for | | |
| | | KIAA0017 gene, complete cds | | |
| M250 E6 | H-D13748 | translation initiation factor 4A | 44.77 | 49.0 kDa |
| M305 C3 | H-D13892 | carboxyl methyltransferase, | 25.19 | 34 |
| | | aspartate | | |
| D1 | H-D13900 | enoyl-Coenzyme A hydratase, | 32.01 | 58 |
| | | short chain, mitochondrial | | |
| E1 | H-D14446 | Human HFREP-1 mRNA for | 34.43 | 40 |
| | | unknown protein, complete cds | | |
| 167-14 | H-D14497 | H. sapiens (Ewing's sarcoma cell | 51.44 | 64 |
| | | line) mRNA encoding open | | |
| | | reading frame | | |
| M266 D2 | H-D14520 | basic transcription element- | 24.2 | 33.0 kDa |
| | | binding protein 2 | | |
| M318 D2 | H-D14658 | hypothetical protein | 13.64 | 17 |
| | | (GB: D14658) | | |
| D2 | H-D14661 | Human mRNA for KIAA0105 | 16.72 | 28 |
| | | gene, complete cds | | |
| M236 E2 | H-D14662 | HYPOTHETICAL 29.5 KD | 24.75 | 36.0 kDa |
| | | PROTEIN IN UBP13-KIP1 | | |
| | | INTERGENIC REGION | | |
| | | [Saccharomyces cerevisiae] | | |
| M271 G6 | H-D14695 | hypothetical protein | 43.12 | 50.0 kDa |
| | | (GB: D14695), Human mRNA for | | |
| | | KIAA0025 gene, complete cds. | | |
| M311 A3 | H-D14696 | hypothetical protein | 25.74 | 30.0 kDa |
| | | (GB: D14696) | | |
| H3 | H-D14697 | Farnesyl diphosphate synthase | 46.2 | 55 |
| | | (farnesyl pyrophosphate | | |
| | | synthetase, | | |
| | | dimethylallyltranstransferase, | | |
| | | geranyltranstransferase) | | |
| M271 E7 | H-D14705 | catenin, alpha 2(E). Catenin | 99.77 | 110 |
| | | (cadherin-associated protein), | | |
| | | alpha 1 (102 kD). ASSOCIATES | | |
| | | WITH THE CYTOPLASMIC | | |
| | | DOMAIN OF A VARIETY OF | | |
| | | CADHERINS. | | |
| M236 A6 | H-D14811 | hypothetical protein | 30.25 | 42 |
| | | (GB: D14811) | | |
| M250 A3 | H-D14812 | hypothetical protein | | |
| | | (GB: D14812), Human mRNA for | | |
| | | KIAA0026 gene, complete cds | | |
| A5 | H-D14874 | Human mRNA for | 20.46 | 33 |
| | | adrenomedullin, complete cds | | |
| F3 | H-D14887 | Human mRNA for TFIIA-42, | 41.47 | 50 |
| | | complete cds | | |
| M250 H6 | H-D16234 | phospholipase C, alpha, | 55.66 | 56.0 kDa |
| | | PROBABLE PROTEIN | | |
| | | DISULFIDE ISOMERASE ER- | | |
| | | 60 PRECURSOR [Homo sapiens] | | |
| M305 B1 | H-D16480 | enoyl-CoA hydratase/3- | 84.04 | 84 |
| | | hydroxyacyl-CoA dehydrogenase | | |
| | | trifunctional protein, alpha- | | |
| | | subunit, mitochobdrial | | |
| M271 G2 | H-D16481 | 3-ketoacyl-CoA thiolase, beta | | |
| | | subunit, mitochodrial, | | |
| | | Hydroxyacyl-Coenzyme A | | |
| | | dehydrogenase/3-ketoacyl- | | |
| | | Coenzyme Athiolase/enoyl- | | |
| | | Coenzyme A hydratase | | |
| | | (trifunctional protein), beta | | |
| | | subunit | | |
| H1 | H-D16626 | Histidine ammonia-lyase | 72.38 | 64 |
| A2 | H-D17532 | Human mRNA for RCK, | 52.03 | 53 |
| | | complete cds | | |
| M266 F4 | H-D17554 | DNA-binding protein TAX | 31.79 | 38 |
| M248 A3 | H-D21235 | xeroderma pigmentosum group C | 40.04 | 55 |
| | | repair complementing protein | | |
| | | HHR23A | | |
| M235 E1 | H-D21261 | SM22-ALPHA HOMOLOG, | 22 | 31 |
| | | hypothetical protein | | |
| | | (GB: D21261) | | |
| M311 E1 | H-D21262 | hypothetical protein | 77.950 | 63 |
| | | (GB: D21262) | | |
| M466 B4 | H-D21853 | Human mRNA for KIAA0111 | 45.32 | 49.0 kDa |
| | | gene, complete cds | | |
| M311 H3 | H-D23660 | ribosomal protein L4 | 47.08 | 47 |
| M419 E1 | H-D26309 | human mRNA for LIMK (LIM | 71.240 | 75.0 kDa |
| | | kinase) | | |
| M271 B9 | H-D26362 | hypothetical protein | 79.97 | 70 |
| | | (GB: D26362), Human mRNA for | | |
| | | KIAA0043 gene, complete cds | | |
| M361 H2 | H-D26598 | proteasome, subunit HsC10-II | 22.66 | 33.0 kDa |
| M302 G4 | H-D26599 | proteasome, subunit HsC7-I | 22.22 | 34 |
| G1 | H-D26600 | Human mRNA for proteasome | 29.15 | 36 |
| | | subunit HsN3, complete cds | | |
| G9 | H-D28540 | hypothetical protein, CDC10 | 44.77 | 60 |
| | | homolog | | |
| M266 A5 | H-D29011 | proteasome, subunit X | 22.99 | 23 |
| M236 F3 | H-D29012 | Proteasome (prosome, macropain) | 26.4 | 32.0 kDa |
| | | delta subunit, beta type, 6 | | |
| C1 | H-D30037 | Human mRNA for | 29.92 | 38 |
| | | phosphatidylinositol transfer | | |
| | | protein (PI-TPbeta), complete cds | | |
| M250 H4 | H-D30655 | translation initiation factor 4AII, | 44.88 | 45.0 kDa |
| | | and ribosomal binding protein | | |
| 167-26 | H-D30742 | human mRNA for calmodulin- | 52.10 | 55 |
| | | dependent protein kinase IV | | |
| M236 A4 | H-D31767 | hypothetical protein | 18.59 | 30 |
| | | (GB: D31767), Human mRNA for | | |
| | | KIAA0058 gene, complete cds | | |
| E1 | H-D31883 | Human mRNA for KIAA0059 | 50.93 | 64 |
| | | gene, complete cds | | |
| G2 | H-D32129 | MHC class I protein HLA-A | 40.26 | 50 |
| M422 A6 | H-D37965 | Human mRNA for PDGF receptor | 41.36 | 45.0 kDa |
| | | beta-like tumor suppressor | | |
| | | (PRLTS), complete cds | | |
| M305 H4 | H-D38047 | 26S proteasome regulatory | 28.340 | 34.0 kDa |
| | | subunit P31 | | |
| M423 B2 | H-D38081 | Thromboxane A2 receptor | 37.84 | 45.0 kDa |
| M317 D3 | H-D38305 | ErbB-2 transducer | 38.06 | 49 |
| M270 A8 | H-D38583 | calgizzarin, Human mRNA for | 11.66 | 12 |
| | | calgizzarin, complete cds | | |
| M270 A6 | H-D42038 | hypothetical protein | 15.29 | 27 |
| | | (GB: D42038), Human mRNA for | | |
| | | KIAA0087 gene, complete cds | | |
| M318 F3 | H-D42085 | hypothetical protein | 90.2 | 100 |
| | | (GB: D42085) | | |
| M311 C2 | H-D43642 | YL-1 protein homolog | 40.15 | 36 |
| E1 | H-D45213 | Human mRNA for zinc finger | 12.87 | 20 |
| | | protein, complete cds | | |
| M236 B2 | H-D45248 | proteasome activator hPA28, | 26.4 | 38 |
| | | subunit beta, may be cell | | |
| | | adhesion protein | | |
| H3 | H-D45887 | Human mRNA for calmodulin, | 16.5 | 20 |
| | | complete cds | | |
| 166-3 | H-D45906 | human mRNA for LIMK-2 | 70 | 70.25 |
| A7 | H-D49357 | Human mRNA for S- | 43.56 | 51 |
| | | adenosylmethionine synthetase, | | |
| | | complete cds | | |
| C5 | H-D49489 | Human mRNA for protein | 48.51 | 54 |
| | | disulfide isomerase-related | | |
| | | protein P5, complete cds | | |
| M482 E2 | H-D49958 | Human fetus brain mRNA for | 30.69 | 32.0 kDa |
| | | membrane glycoprotein M6, | | |
| | | complete cds | | |
| M305 G5 | H-D50063 | proteasome, subunit p40 | 35.75 | 39 |
| M250 B6 | H-D50310 | cyclin I, Human mRNA for cyclin | 41.58 | 47 |
| | | I, complete cds | | |
| E3 | H-D50419 | Homo sapiens mRNA for OTK18, | 78.32 | 64 |
| | | complete cds | | |
| M298 B1 | H-D50495 | transcription elongation factor h- | 33 | 33.0 kDa |
| | | SII-T1 (GB: D50495) | | |
| M302 A3 | H-D50840 | ceramide glucosyltransferase | 43.45 | 44 |
| 167-40 | H-D50863 | human mRNA for TESK1 | 68.93 | 70 |
| 166-28 | H-D50927 | human myeloblast mRNA for | 60.46 | 64 |
| | | KIAA0137 gene | | |
| D1 | H-D63521 | Homo sapiens mRNA for LECT2 | 16.72 | 16 |
| | | precursor, complete cds | | |
| M302 A5 | H-D78134 | glycine-rich binding protein CIRP | 19.03 | 30.0 kDa |
| M313 E5 | H-D78275 | proteasome subunit p42 | 42.9 | 48.0 kDa |
| B3 | H-D79205 | Human mRNA for ribosomal | 5.72 | 10 |
| | | protein L39, complete cds | | |
| A4 | H-D79206 | Human gene for ryudocan core | 21.89 | 33 |
| | | protein, exon1-5, complete cds | | |
| A1 | H-D80008 | Human mRNA for KIAA0186 | 21.67 | 32 |
| | | gene, complete cds | | |
| M298 H4 | H-D83004 | ubiquitin-conjugating enzyme E2 | 16.83 | 32.0 kDa |
| | | similar to Drosophila bendless | | |
| | | gene product | | |
| C3 | H-D83702 | Human brain mRNA for | 64.57 | 64 |
| | | photolyase homolog, complete | | |
| | | cds | | |
| M306 A1 | H-D83735 | neutral calponin | 34.1 | 34.0 kDa |
| H2 | H-D86322 | Homo sapiens mRNA for | 67.21 | 64 |
| | | calmegin, complete cds | | |
| B1 | H-D86979 | Human mRNA for KIAA0226 | 82.72 | 82 |
| | | gene, complete cds | | |
| 169-16 | H-D87116 | dual specificity mitogen-activated | 38.24 | 42 |
| | | protein kinase kinase 3 | | |
| 166-27 | H-D87119 | human cancellous bone osteoblast | 37.80 | 40 |
| | | mRNA for GS3955 | | |
| E2 | H-D88308 | Homo sapiens mRNA for very- | 68.31 | 64 |
| | | long-chain acyl-CoA synthetase, | | |
| | | complete cds | | |
| 166-26 | H-D89077 | human mRNA for Src-like | 30.43 | 38 |
| | | adapter protein | | |
| M440 H2 | H-D89479 | Homo sapiens mRNA for ST1B2, | 32.67 | 38.0 kDa |
| | | complete cds | | |
| H1 | H-D90086 | Human pyruvate dehydrogenase | 39.6 | 35 |
| | | (EC 1.2.4.1) beta subunit gene, | | |
| | | exons 1-10 | | |
| M362 F1 | H-D90209 | DNA-binding protein | 38.72 | 48.0 kDa |
| | | TAXREB67 | | |
| M316 B2 | H-J00068 | actin, alpha 1, skeletal muscle | 41.58 | 50 |
| M250 B2 | H-J00194 | major histocompatibility complex, | 28.05 | 36.0 kDa |
| | | MHC class II, DR alpha | | |
| G2 | H-J00212 | Interferon, alpha 21 | 20.9 | 30 |
| G1 | H-J00287 | Human pepsinogen gene | 42.79 | 48 |
| M298 C2 | H-J02611 | apolipoprotein D | 20.9 | 31.0 kDa |
| M266 C4 | H-J02683 | ADP/ATP carrier protein | 32.89 | 36 |
| M383 H2 | H-J02685 | plasminogen activator inhibitor, | 45.76 | 50.0 kDa |
| | | placenta | | |
| 167-3 | H-J02853 | “casein kinase II, alpha chain” | 43.08 | 50 |
| E3 | H-J02854 | Human 20-kDa myosin light | 19.03 | 31 |
| | | chain (MLC-2) mRNA, complete | | |
| | | cds | | |
| M248 F3 | H-J02874 | fatty-acid-binding protein 4, | 14.63 | 17 |
| | | adipocyte, LIPID TRANSPORT | | |
| | | PROTEIN IN ADIPOCYTES | | |
| M235 D5 | H-J02939 | antigen 4F2, heavy chain | 58.3 | 58 |
| C3 | H-J02943 | Corticosteroid binding globulin | 44.66 | 50 |
| M248 F2 | H-J02966 | adenine nucleotide translocator 1 | 32.78 | 33 |
| | | (skeletal muscle) [ANT1], | | |
| | | CATALYZES THE EXCHANGE | | |
| | | OF ADP AND ATP ACROSS | | |
| | | THE MITOCHONDRIAL | | |
| | | INNER MEMBRANE. | | |
| E1 | H-J02982 | Glycophorin B | 10.12 | 20 |
| 167-91 | H-J03075 | “protein kinase c substrate, 80 kD | 58.04 | 98 |
| | | protein heavy chain” | | |
| M266 A3 | H-J031891 | profilin 1 | 15.51 | 17.0 kDa |
| M248 H4 | H-J03231 | glucose-6-phosphate | 56.76 | 51 |
| | | dehydrogenase [G6PD] | | |
| M266 F2 | H-J03459 | LEUKOTRIENE A-4 | 67.32 | 64 |
| | | HYDROLASE [Homo sapiens] | | |
| A2 | H-J03460 | Prolactin-induced protein | 16.17 | 26 |
| M271 E5 | H-J03799 | laminin receptor 1, Laminin | 32.56 | |
| | | receptor (2H5 epitope). 40S | | |
| | | RIBOSOMAL PROTEIN SA | | |
| | | [Homo sapiens]. | | |
| M440 A4 | H-J03890 | Human pulmonary surfactant | 21.78 | 30.0 kDa |
| | | protein C (SP-C) and pulmonary | | |
| | | surfactant protein C1 (SP-C1) | | |
| | | genes, complete cds | | |
| M271 D8 | H-J03934 | NAD(P)H menadione | 30.25 | 38 |
| | | oxidoreductase 1, dioxin- | | |
| | | inducible. INVOLVED IN | | |
| | | DETOXICATION PATHWAYS. | | |
| M271 A8 | H-J04031 | trifunctional enzyme | 102.96 | 117.0 kDa |
| | | (GB: J04031). C-1- | | |
| | | TETRAHYDROFOLATE | | |
| | | SYNTHASE, CYTOPLASMIC | | |
| | | [Homo sapiens] | | |
| M305 F6 | H-J04046 | calmodulin 3 [CALM3] | 16.5 | 20 |
| M305 G7 | H-J04071 | cytotoxic T-lymphocyte- | 27.28 | 38 |
| | | associated serine esterase 1 | | |
| | | (cathepsin G-like 1, granzyme B) | | |
| | | [CTLA1] | | |
| M311 D2 | H-J04183 | lysosomal-associated membrane | 44.99 | 47 |
| | | protein 2 | | |
| M300 F4 | H-J04205 | Sjogren syndrome antigen B | 44.99 | 51.0 kDa |
| M416 G8 | H-J04430 | Acid phosphatase 5, tartrate | 35.64 | 45.0 kDa |
| | | resistant | | |
| B1 | H-J04501 | Glycogen synthase 1 (muscle) | 81.18 | 81 |
| M313 B5 | H-J04543 | synexin | 51.37 | 51 |
| B1 | H-J04605 | Peptidase D | 54.34 | 55 |
| M250 C6 | H-J04615 | small nuclear ribonucleoprotein | 26.51 | 34.0 kDa |
| | | SM-D, ROLE IN THE PRE- | | |
| | | mRNA SPLICING OR IN | | |
| | | SNRNPSTRUCTURE. | | |
| M248 E2 | H-J04964 | steroid sulfatase (microsomal) | 64.24 | 60.0 kDa |
| | | [STS] | | |
| M250 A7 | H-J05249 | replication protein A, 32 kDa | 29.81 | 36.0 kDa |
| | | subunit, REQUIRED FOR SV | | |
| | | 40 DNA REPLICATION IN | | |
| | | VITRO, RP-A IS SINGLE- | | |
| | | STRANDED DNA-BINDING | | |
| | | PROTEIN. | | |
| F1 | H-J05272 | IMP (inosine monophosphate) | 56.65 | 51 |
| | | dehydrogenase 1 | | |
| 169-15 | H-J05401 | “creatine kinase, sarcomeric | 50 | 46.16 |
| | | mitochondrial precursor” | | |
| M266 E4 | H-J05448 | RNA polymerase II, subunit B33 | 30.36 | 35.0 kDa |
| M305 C2 | H-K00558 | tubulin, alpha k1 [TUBA*] | 49.72 | 52.0 kDa |
| M416 H7 | H-K01571 | Human T-cell receptor active | 34.43 | 36.0 kDa |
| | | beta-chain, mRNA from cell line | | |
| | | MOLT-3, complete cds | | |
| M311 E4 | H-K01763 | haptoglobin | 38.28 | 47.0 kDa |
| G5 | H-K02100 | Human ornithine | 39.05 | 47 |
| | | transcarbamylase (OTC) mRNA, | | |
| | | complete coding sequence | | |
| M302 D5 | H-K02574 | purine nucleoside phosphorylase | 31.9 | 36.0 kDa |
| 169-39 | H-K02581 | “thymidine kinase, cytosolic” | 34 | 25.81 |
| M248 E4 | H-K03020 | phenylalanine hydroxylase [PAH] | 49.83 | 50 |
| M556 B3 | H-K03191 | Cytochrome P450, subfamily I | 56.43 | 53.02 kDa |
| | | (aromatic compound-inducible), | | |
| | | polypeptide 1 | | |
| H2 | H-L00190 | Antithrombin III | 51.15 | 55 |
| 169-62 | H-L01087 | “protein kinase c, theta type” | 80 | 77.7 |
| M318 C2 | H-L01124 | ribosomal protein S13 | 16.72 | 28 |
| M313 F1 | H-L02321 | glutathione S-transferase M5 | 24.09 | 28 |
| M305 E5 | H-L02426 | protease 26S, regulatory subunit 4 | 48.51 | 53 |
| M302 D4 | H-L02547 | cleavage stimulation factor, 50 | 47.52 | 50.0 kDa |
| | | kDa subunit | | |
| M266 H7 | H-L02648 | transcobalamin II | 47.08 | 48.0 kDa |
| E2 | H-L02932 | Human peroxisome proliferator | 51.59 | 59 |
| | | activated receptor mRNA, | | |
| | | complete cds | | |
| M270 A1 | H-L03380 | gonadotropin-releasing hormone | 36.19 | 36 |
| | | receptor [GRHR], THIS | | |
| | | RECEPTOR MEDIATES ITS | | |
| | | ACTION BY ASSOCIATION | | |
| | | WITH G PROTEINS | | |
| M270 H1 | H-L03411 | RD protein [RDBP], Radin blood | 41.91 | 59.0 kDa |
| | | group | | |
| D3 | H-L03426 | Human XE7 mRNA, complete | 42.46 | 45 |
| | | alternate coding regions | | |
| B1 | H-L03785 | Myosin, light polypeptide 5, | 19.14 | 32 |
| | | regulatory | | |
| A7 | H-L04483 | ribosomal protein S21 | 9.24 | 34 |
| M416 B2 | H-L05147 | Human dual specificity | 20.46 | 30.0 kDa |
| | | phosphatase tyrosine/serine | | |
| | | mRNA, complete cds | | |
| 215-38 | H-L05624 | dual specificity mitogen-activated | 50 | 43.30 |
| | | protein kinase kinase 1 | | |
| M271 D4 | H-L06132 | anion channel, voltage-gated, | 31.24 | 37 |
| | | isoform 1. FORMS A CHANNEL | | |
| | | THROUGH THE CELL | | |
| | | MEMBRANE, THAT ALLOWS | | |
| | | DIFFUSION FROM SMALL | | |
| | | HYDROPHYLIC MOLECULES. | | |
| 169-27 | H-L06139 | tyrosine-protein kinase receptor | 125 | 123.7 |
| | | TIE-2 precursor | | |
| H1 | H-L06147 | Human (clone SY11) golgin-95 | 68.31 | 68 |
| | | mRNA, complete cds | | |
| M250 A1 | H-L06419 | procollagen-lysine, 2-oxoglutarate | 80.08 | 80.0 kDa |
| | | 5-dioxygenase (lysine | | |
| | | hydroxylase) [PLOD] | | |
| M236 F6 | H-L06498 | ribosomal protein S20 | 13.2 | 23.0 kDa |
| M318 D1 | H-L06499 | ribosomal protein L37a | 10.23 | 27 |
| M270 D1 | H-L07414 | CD40 antigen ligand [CD40LG], | 28.82 | 36 |
| | | NVOLVED IN | | |
| | | IMMUNOGLOBULIN CLASS | | |
| | | SWITCHING. | | |
| M298 A6 | H-L07548 | aminoacylase 1 | 44.99 | 52.0 kDa |
| M424 C3 | H-L07592 | Human peroxisome proliferator | 48.62 | 48.0 kDa |
| | | activated receptor mRNA, | | |
| | | complete cds | | |
| M298 G6 | H-L07633 | proteasome (prosome, macropain) | 27.5 | 33.0 kDa |
| | | activator subunit 1 (PA28 alpha) | | |
| | | [PSME1] | | |
| M318 B1 | H-L08096 | CD70 antigen (CD27 ligand) | 21.34 | 28 |
| | | [CD70] | | |
| D2 | H-L08187 | cytokine receptor EB13 | 25.3 | 42 |
| M313 F4 | H-L08850 | amyloid, non-A beta component, | 15.51 | 31.0 kDa |
| | | Alzheimer's disease | | |
| M426 E1 | H-L08895 | MADS box transcription enhancer | 52.14 | 60.0 kDa |
| | | factor 2, polypeptide C (myocyte | | |
| | | enhancer factor 2C) | | |
| M266 A8 | H-L09235 | ATPase, vacuolar | 67.98 | 64.0 kDa |
| M266 D1 | H-L09604 | differentiation-dependent | 16.83 | 17.0 kDa |
| | | intestinal membrane A4 protein | | |
| | | (Homo sapiens) | | |
| M317 C1 | H-L10338 | sodium channel, voltage-gated, | 24.09 | 24 |
| | | type I, beta polypeptide [SCN1B] | | |
| M317 E1 | H-L10717 | tyrosine-protein kinase ITK/TSK | 68.270 | 68.0 kDa |
| M300 B5 | H-L10820 | formyl peptide receptor 1 [FPR1] | 38.61 | 37 |
| M312 A4 | H-L10838 | pre-mRNA splicing factor SRp20 | 18.15 | 31.0 kDa |
| M300 A5 | H-L10918 | chemokine (C-C) receptor 1 | 39.16 | 30 |
| | | [CMKBR1] | | |
| M311 F2 | H-L11245 | complement component 4-binding | 27.83 | 30 |
| | | protein, beta | | |
| M266 B7 | H-L11353 | neurofibromatosis 2 (bilateral | 65.56 | 63.0 kDa |
| | | acoustic neuroma) [NF2] | | |
| M311 B3 | H-L11667 | cyclophilin 40 | 40.81 | 50.0 kDa |
| 215-49 | H-L11695 | serine/threonine-protein kinase | 64 | 55.40 |
| | | receptor R4 precursor | | |
| M466 C2 | H-L11931 | Human cytosolic serine | 53.24 | 56.0 kDa |
| | | hydroxymethyltransferase | | |
| | | (SHMT) mRNA, complete cds | | |
| M271 B7 | H-L12168 | ADENYLYL CYCLASE- | 52.36 | 60.0 kDa |
| | | ASSOCIATED PROTEIN 1 | | |
| | | [Homo sapiens] | | |
| M416 D4 | H-L12964 | Interleukin-activated receptor, | 28.16 | 38.0 kDa |
| | | homolog of mouse Ly63 | | |
| B3 | H-L13203 | Human HNF-3/fork-head | 38.72 | 49 |
| | | homolog-3 HFH-3 mRNA, | | |
| | | complete cds | | |
| D2 | H-L13744 | Human AF-9 mRNA, complete | 62.59 | 63 |
| | | cds | | |
| 167-8 | H-L13943 | glycerol kinase | 60 | 57.71 |
| M311 G3 | H-L13974 | leucine zipper protein | 41.14 | 51 |
| | | (GB: L13974) | | |
| M271 H5 | H-L13977 | LYSOSOMAL PRO-X | 54.67 | 57 |
| | | CARBOXYPEPTIDASE | | |
| | | PRECURSOR [Homo sapiens]. | | |
| M270 G2 | H-L14283 | protein kinase C, zeta [PRKCZ], | 65.23 | 98 |
| | | SERINE- AND THREONINE- | | |
| | | SPECIFIC ENZYME. | | |
| M235 A3 | H-L14286 | antioxidant protein, thiol-specific | 21.89 | 32.0 kDa |
| M426 H3 | H-L14778 | Protein phosphatase 3 (formerly | 57.42 | 60.0 kDa |
| | | 2B), catalytic subunit, alpha | | |
| | | isoform (calcineurin A | | |
| | | alpha){alternative products} | | |
| B4 | H-L15702 | complement factor B | 84.15 | 100 |
| M426 A4 | H-L16794 | Human transcription factor | 57.42 | 60.0 kDa |
| | | (MEF2) mRNA, complete cds | | |
| 215-25 | H-L16862 | g protein-coupled receptor kinase | 70 | 63.4 |
| | | GRK6 | | |
| 167-74 | H-L16991 | thymidylate kinase | 36 | 23.39 |
| 169-3 | H-L18964 | “protein kinase c, iota type” | 80 | 64.64 |
| M305 E2 | H-L18972 | hypothetical protein (GB: L18972) | 75.24 | 78 |
| M426 D4 | H-L19067 | Human NF-kappa-B transcription | 59.18 | 63.0 kDa |
| | | factor p65 subunit mRNA, | | |
| | | complete cds | | |
| 215-26 | H-L19268 | Homo sapiens myotonic | 70 | 68.71 |
| | | dystrophy associated protein | | |
| | | kinase mRNA | | |
| M271 E1 | H-L19297 | carbonic anhydrase V [CA5], | 33.66 | 42 |
| | | Mitochondrial carbonic | | |
| | | anhydrase. REVERSIBLE | | |
| | | HYDRATATION OF CARBON | | |
| | | DIOXIDE. | | |
| M298 G4 | H-L19437 | transaldolase | 37.18 | 39.0 kDa |
| M423 C4 | H-L19593 | Interleukin 8 receptor, beta | 39.71 | 41.0 kDa |
| G1 | H-L19686 | Homo sapiens macrophage | 12.76 | 13 |
| | | migration inhibitory factor (MIF) | | |
| | | gene, complete cds | | |
| G2 | H-L19739 | metallopanstimulin 1 | 9.35 | 32 |
| M302 E3 | H-L19871 | activating transcription factor 3 | 20.02 | 36.0 kDa |
| 167-86 | H-L20422 | 14-3-3 protein eta | 34 | 27.13 |
| M440 B2 | H-L20492 | Human gamma-glutamyl | 24.86 | 35.0 kDa |
| | | transpeptidase mRNA, complete | | |
| | | cds | | |
| M315 B1 | H-L20688 | GDP-dissociation inhibitor | 22.22 | 32 |
| | | protein rhoA | | |
| M271 H3 | H-L20941 | ferritin, heavy polypeptide. | 20.24 | 32 |
| | | FERRITIN IS AN | | |
| | | INTRACELLULAR | | |
| | | MOLECULE THAT STORES | | |
| | | IRON IN A SOLUBLE, | | |
| | | NONTOXIC, READILY | | |
| | | AVAILABLE FORM. | | |
| M235 B7 | H-L21893 | Na+/taurocholate cotransporter, | | |
| | | STRICTLY DEPENDENT ON | | |
| | | THE | | |
| F1 | H-L21934 | Sterol O-acyltransferase (acyl- | 60.61 | 60 |
| | | Coenzyme A: cholesterol | | |
| | | acyltransferase) | | |
| C2 | H-L22075 | Human guanine nucleotide | 41.58 | 50 |
| | | regulatory protein (G13) mRNA, | | |
| | | complete cds | | |
| 169-18 | H-L22206 | vasopressin v2 receptor | 60 | 58.00 |
| M421 A10 | H-L22214 | Human adenosine A1 receptor | 35.97 | 38.0 kDa |
| | | (ADORA1) mRNA exons 1-6, | | |
| | | complete cds | | |
| M424 F1 | H-L23959 | Homo sapiens E2F-related | 45.21 | 53.0 kDa |
| | | transcription factor (DP-1) | | |
| | | mRNA, complete cds | | |
| C2 | H-L24498 | Human gadd45 gene, complete | 18.26 | 28 |
| | | cds | | |
| M302 E2 | H-L25080 | proto-oncogene rhoA, multidrug | 21.34 | 31 |
| | | resistance protein | | |
| M270 B8 | H-L25081 | guanine nucleotide-binding and | 21.34 | 30 |
| | | transforming protein rhoC, | | |
| | | Aplysia ras-related homolog 9 | | |
| M236 E3 | H-L25085 | Sec61 complex, beta subunit, | 10.67 | 19 |
| | | PROTEIN TRANSLOCATION | | |
| | | IN THE ENDOPLASMIC | | |
| | | RETICULUM | | |
| 167-85 | H-L25610 | cyclin-dependent kinase inhibitor 1 | 32 | 18.11 |
| B2 | H-L25610 | cyclin-dependent kinase inhibitor 1 | 18.110 | 40 |
| M297 H2 | H-L26232 | cathepsin A/phospholipid transfer | 54.34 | 64.0 kDa |
| | | protein | | |
| 167-4 | H-L26318 | stress-activated protein kinase | 52 | 42.31 |
| | | JNK1 | | |
| M428 F1 | H-L27586 | Human TR4 orphan receptor | 67.76 | 67.0 kDa |
| | | mRNA, complete cds | | |
| M302 E5 | H-L27711 | protein phosphatase KAP1 | 23.43 | 28 |
| M250 A6 | H-L28010 | Homo sapiens HnRNP F protein | | |
| | | mRNA, complete cds, | | |
| F1 | H-L28821 | Alpha mannosidase II isozyme | 87.67 | 87 |
| 167-89 | H-L28824 | tyrosine-protein kinase SYK | 70 | 69.92 |
| M298 E6 | H-L28997 | ADP-ribosylation factor-like gene 1 | 20.02 | 33.0 kDa |
| D4 | H-L29219 | Homo sapiens clk1 mRNA, | 53.35 | 60 |
| | | complete cds | | |
| 169-63 | H-L29222 | Homo sapiens clk1 mRNA | 25 | 15.03 |
| M429 B3 | H-L29277 | Signal transducer and activator of | 84.81 | 88.0 kDa |
| | | transcription 3 (acute-phase | | |
| | | response factor) | | |
| C1 | H-L29433 | Human factor X (blood | 53.79 | 64 |
| | | coagulation factor) gene | | |
| G3 | H-L31860 | Glycophorin A | 16.61 | 26 |
| D1 | H-L31881 | Nuclear factor I/X (CCAAT- | 48.62 | 48 |
| | | binding transcription factor) | | |
| 169-13 | H-L31951 | human protein kinase (JNK2) | 55 | 46.71 |
| | | mRNA | | |
| A1 | H-L32179 | Arylacetamide deacetylase | 44 | 50 |
| | | (esterase) | | |
| B2 | H-L33404 | Human stratum corneum | 27.94 | 36 |
| | | chymotryptic enzyme mRNA, | | |
| | | complete cds | | |
| M312 D3 | H-L33799 | procollagen C-proteinase | 49.5 | 51.0 kDa |
| | | enhancer | | |
| 169-77 | H-L33801 | human protein kinase mRNA | 55 | 46.27 |
| | | GSK-3 | | |
| M305 D6 | H-L34041 | L-glycerol-3-phosphate: NAD+ | 38.5 | 42.0 kDa |
| | | oxidoreductase | | |
| B4 | H-L34355 | Homo sapiens (clone p4) 50 kD | 42.68 | 47 |
| | | dystrophin-associated | | |
| | | glycoprotein mRNA, complete | | |
| | | cds | | |
| M297 B3 | H-L35013 | spliceosomal protein SAP 49 | 46.75 | 52.0 kDa |
| 167-32 | H-L35253 | human CSaids binding protein | 52 | 39.67 |
| | | (CSBP1) mRNA | | |
| M266 D6 | H-L35545 | C/activated protein C receptor, | 26.29 | 38.0 kDa |
| | | endothelial | | |
| M300 F1 | H-L35594 | autotaxin | 100.76 | 91.0 kDa |
| M318 E2 | H-L36720 | bystin | 33.77 | 29 |
| M305 H2 | H-L37127 | RNA polymerase II | 12.98 | 16 |
| M300 D1 | H-L38490 | ADP-ribosylation factor | 22.22 | 32 |
| | | (GB: L38490) | | |
| M318 E1 | H-L38941 | ribosomal protein L34 | 12.98 | 18 |
| C2 | H-L38969 | Homo sapiens thrombospondin 3 | 105.27 | 110 |
| | | (THBS3) gene, complete cds | | |
| M476 F4 | H-L39060 | Homo sapiens transcription factor | 49.61 | 53.0 kDa |
| | | SL1 mRNA, complete cds | | |
| M300 E4 | H-L40399 | hypothetical protein (GB: L40399) | 29.26 | 36 |
| E3 | H-L40802 | Homo sapiens 17-beta- | 42.68 | 60 |
| | | hydroxysteroid dehydrogenase | | |
| | | (17-HSD) gene | | |
| M478 F1 | H-L40904 | H. sapiens peroxisome | 52.69 | 60.0 kDa |
| | | proliferator activated receptor | | |
| | | gamma, complete cds | | |
| M306 C2 | H-L41268 | natural killer associated transcript | 37.62 | 40 |
| | | 2 [NKAT2*] | | |
| M306 E2 | H-L41270 | natural killer associated transcript | 50.16 | 65.0 kDa |
| | | 4 [NKAT4*] | | |
| M306 F2 | H-L41347 | natural killer associated transcript | 33.55 | 40 |
| | | 5 [NKAT5*] | | |
| M468 C3 | H-L41351 | Homo sapiens prostasin mRNA, | 37.84 | 45.0 kDa |
| | | complete cds | | |
| 169-53 | H-L41816 | Homo sapiens cam kinase I | 48 | 40.77 |
| | | mRNA | | |
| 167-25 | H-L41939 | tyrosine-protein kinase receptor | 108 | 108.6 |
| | | EPH-3 precursor | | |
| C3 | H-L42374 | Homo sapiens protein | 54.78 | 64 |
| | | phosphatase 2A B56-beta (PP2A) | | |
| | | mRNA, complete cds | | |
| M306 B1 | H-L42531 | glutathione synthetase | 52.25 | 54.0 kDa |
| M302 F6 | H-L42856 | RNA polymerase II transcription | 13.09 | 20.0 kDa |
| | | factor SIII, p18 subunit | | |
| M313 C7 | H-L76200 | guanylate kinase (GUK1) | 21.78 | 32.0 kDa |
| M428 E1 | H-L76702 | Homo sapiens protein | 66.33 | 68.0 kDa |
| | | phosphatase 2A B56-delta (PP2A) | | |
| | | mRNA, complete cds | | |
| M478 A1 | H-L76703 | Homo sapiens protein | 51.48 | 60.0 kDa |
| | | phosphatase 2A B56-epsilon | | |
| | | (PP2A) mRNA, complete cds | | |
| 166-52 | H-L77213 | H. sapiens phosphomevalonate | 34 | 21.19 |
| | | kinase mRNA | | |
| 169-64 | H-L77964 | H. sapiens ERK3 mRNA | 100 | 79.38 |
| M360 C3 | H-M10050 | fatty-acid-binding protein 2, | 14.08 | 20.0 kDa |
| | | intestinal | | |
| D5 | H-M10050 | fatty-acid-binding protein 2, | 14.08 | 36 |
| | | intestinal | | |
| M421 E7 | H-M10058 | Asialoglycoprotein receptor 1 | 32.12 | 48.0 kDa |
| M429 D3 | H-M10901 | Glucocorticoid receptor | 85.58 | 85.0 kDa |
| M312 G1 | H-M11025 | asialoglycoprotein receptor 2 | 34.32 | 34.0 kDa |
| 167-44 | H-M11026 | interferon alpha-4 precursor | 33 | 20.86 |
| F2 | H-M11321 | Human group-specific component | 52.25 | 56 |
| | | vitamin D-binding protein | | |
| | | mRNA, complete cds | | |
| M236 B5 | H-M11354 | histone H3.2, CENTRAL ROLE | 15.07 | 24 |
| | | IN NUCLEOSOME | | |
| | | FORMATION. | | |
| M236 G2 | H-M11433 | retinol-binding protein 1, cellular | 14.96 | 28 |
| | | transport protein | | |
| M270 G7 | H-M11560 | aldolase A, FRUCTOSE- | 40.15 | 40 |
| | | BISPHOSPHATE ALDOLASE A | | |
| | | [Homo sapiens] | | |
| H3 | H-M11717 | Human heat shock protein (hsp | 70.51 | 60 |
| | | 70) gene, complete cds | | |
| E1 | H-M12523 | Human serum albumin (ALB) | 67.1 | 70 |
| | | gene, complete cds | | |
| B5 | H-M12963 | Alcohol dehydrogenase 1 (class | 41.36 | 48 |
| | | I), alpha polypeptide | | |
| D6 | H-M13228 | | 51.15 | 50 |
| D4 | H-M13981 | Inhibin, alpha | 40.37 | 50 |
| M236 G4 | H-M13982 | interleukin 4 [IL4] precursor, B- | 16.94 | 30 |
| | | cell activator | | |
| M271 B6 | H-M14043 | lipocortin II, Annexin II | 37.4 | 45.0 kDa |
| | | (lipocortin II). CALCIUM- | | |
| | | REGULATED MEMBRANE- | | |
| | | BINDING PROTEIN | | |
| M271 F4 | H-M14218 | argininosuccinate lyase | 51.04 | 56 |
| M297 A3 | H-M14221 | cathepsin B | 37.4 | 32.0 kDa |
| M305 B2 | H-M14328 | enolase, alpha | 47.85 | 50 |
| 167-54 | H-M14333 | human c-syn protooncogene | 60 | 59.14 |
| 167-51 | H-M14505 | H. sapiens mRNA (open reading | 36 | 33.40 |
| | | frame; patient SK29(AV)) | | |
| 215-74 | H-M14676 | human src-like kinase (slk) | 60 | 59.14 |
| | | mRNA | | |
| 167-55 | H-M14780 | “creatine kinase, m chain” | 52 | 41.98 |
| M416 F8 | H-M15059 | Fc fragment of IgE, low affinity | 35.42 | 45.0 kDa |
| | | II, receptor for (CD23A) | | |
| M271 F1 | H-M15182 | glucuronidase, beta [GUSB], | 71.72 | 72 |
| | | PLAYS AN IMPORTANT ROLE | | |
| | | IN THE DEGRADATION OF | | |
| | | DERMATAN AND KERATAN | | |
| | | SULFATES. | | |
| 215-37 | H-M15465 | human pyruvate kinase type L | 64 | 59.80 |
| | | mRNA | | |
| M298 A4 | H-M15796 | cyclin | 28.82 | 43.0 kDa |
| C3 | H-M15800 | Mal, T-cell differentiation protein | 16.94 | 17 |
| M440 E1 | H-M15841 | Human U2 small nuclear RNA- | 24.86 | 34.0 kDa |
| | | associated B” antigen mRNA, | | |
| | | complete cds | | |
| M248 C3 | H-M15887 | endozepine | 9.68 | 15.0 kDa |
| M463 A2 | H-M15990 | human c-yes-1 mRNA | 59.800 | 65.0 kDa |
| M418 E2 | H-M16038 | tyrosine-protein kinase LYN | 56.390 | 64.0 kDa |
| M266 D3 | H-M16342 | HETEROGENEOUS NUCLEAR | 32.01 | 49 |
| | | RIBONUCLEOPROTEINS | | |
| | | C1/C2 [Homo sapiens]; small | | |
| | | nuclear ribonucleoprotein, | | |
| | | polypeptide C | | |
| 167-20 | H-M16591 | tyrosine-protein kinase HCK | 60 | 55.62 |
| C7 | H-M16591 | tyrosine-protein kinase HCK | 55.620 | 70 |
| M305 E7 | H-M16660 | heat shock 90 kD protein 1, beta | 79.75 | 80 |
| | | [HSPCB] | | |
| 167-65 | H-M16750 | PIM-1 proto-oncogene | 38 | 34.50 |
| | | serine/threonine-protein kinase | | |
| M311 A1 | H-M16827 | acyl-Coenzyme A dehydrogenase, | 46.42 | 50.0 kDa |
| | | C-4 to C-12 straight-chain | | |
| D3 | H-M16961 | Alpha-2-HS-glycoprotein alpha | 40.48 | 50 |
| | | and beta chain | | |
| D3 | H-M16974 | Complement component 8, alpha | 64.35 | 55 |
| | | polypeptide | | |
| M248 C2 | H-M17017 | INTERLEUKIN-8 PRECURSOR | 11 | 11 |
| | | [Homo sapiens] | | |
| M305 E4 | H-M17885 | ribosomal phosphoprotein P0, | 34.98 | 37.0 kDa |
| | | acidic | | |
| M339 E2 | H-M17887 | ribosomal phosphoprotein P2 | 12.76 | 19.0 kDa |
| M248 D5 | H-M18731 | galactose-1-phosphate | 41.91 | 42 |
| | | uridylyltransferase [GALT] | | |
| F2 | H-M19309 | Troponin T1, skeletal, slow | 30.69 | 40 |
| M385 E2 | H-M19713 | tropomyosin, alpha, muscle | 31.35 | 41.0 kDa |
| 167-79 | H-M19722 | proto-oncogene tyrosine-protein | 64 | 58.26 |
| | | kinase FGR | | |
| M248 H1 | H-M20560 | Annexin III (lipocortin III), | 35.64 | 37 |
| | | INHIBITOR OF | | |
| | | PHOSPHOLIPASE A2 | | |
| M235 H1 | H-M20681 | GLUCOSE TRANSPORTER | 54.67 | 50 |
| | | TYPE 3, BRAIN | | |
| 167-29 | H-M21616 | beta platelet-derived growth | 121 | 121.7 |
| | | factor receptor precursor | | |
| M305 A3 | H-M21812 | myosin light chain 2 | 18.81 | 30 |
| 167-30 | H-M22146 | “40S ribosomal protein S4, x | 34 | 26.91 |
| | | isoform” | | |
| M302 D6 | H-M22430 | phospholipase A2 RASF-A | 15.95 | 31.0 kDa |
| E2 | H-M22491 | Bone morphogenetic protein 3 | 52.03 | 55 |
| | | (osteogenic) | | |
| M340 A2 | H-M22538 | NADH-ubiquinone reductase, 24 | 27.5 | 33 |
| | | kDa subunit, mitochondrial | | |
| B2 | H-M22632 | Glutamic-oxaloacetic | 47.41 | 47 |
| | | transaminase 2, mitochondrial | | |
| | | (aspartate aminotransferase 2) | | |
| B4 | H-M22960 | Protective protein for beta- | 52.91 | 60 |
| | | galactosidase (galactosialidosis) | | |
| M250 C4 | H-M22995 | ras-related protein RAP1A, | | |
| | | member of RAS oncogene family | | |
| B3 | H-M23254 | Calpain, large polypeptide L2 | 77.11 | 77 |
| M266 B4 | H-M23613 | Nucleophosmin (nucleolar | 32.45 | 42 |
| | | phosphoprotein B23, numatrin), | | |
| | | BELIEVED TO BIND SINGLE- | | |
| | | STRANDED NUCLEIC ACIDS | | |
| M469 D2 | H-M23668 | Homo sapiens adrenodoxin gene | 20.35 | 25.0 kDa |
| M478 H3 | H-M24439 | Human liver/bone/kidney-type | 57.75 | 64.0 kDa |
| | | alkaline phosphatase (ALPL) | | |
| | | gene | | |
| F5 | H-M24470 | Glucose-6-phosphate | 38.06 | 44 |
| | | dehydrogenase | | |
| M270 E5 | H-M24898 | thyroid hormone triiodothyronine | 67.65 | 85 |
| | | receptor c-erbA, ear-1, Thyroid | | |
| | | hormone receptor, alpha (avian | | |
| | | erythroblastic leukemia viral (v- | | |
| | | erb-a) oncogene homolog) | | |
| D3 | H-M24902 | Acid phosphatase, prostate | 42.57 | 54 |
| D6 | H-M25809 | ATPase, H+ transporting, | 56.32 | 57 |
| | | lysosomal (vacuolar proton | | |
| | | pump), beta polypeptide, | | |
| | | 56/58 kD, isoform 1 | | |
| 167-77 | H-M26252 | “pyruvate kinase, M2 isozyme” | 60 | 58.48 |
| M271 F8 | H-M26326 | keratin 18 | 47.41 | 50.0 kDa |
| B1 | H-M26901 | Human renin gene | 44.44 | 50 |
| M271 G4 | H-M27396 | asparagine synthetase | 61.82 | 62 |
| M338 B3 | H-M27542 | globulin, sex hormone-binding | 39.200 | 40 |
| M512 B6 | H-M27602 | Protease, serine, 2 (trypsin 2) | 27.28 | 36.0 kDa |
| M270 B6 | H-M27691 | DNA-binding protein CREB, | 36.08 | 50 |
| | | cAMP-responsive | | |
| C1 | H-M27878 | Zinc finger protein 84 (HPF2) | 81.29 | 81 |
| M270 F6 | H-M28209 | guanine nucleotide-binding | 22.66 | 30.0 kDa |
| | | protein rab1 | | |
| M512 H5 | H-M28210 | RAB3A, member RAS oncogene | 24.31 | 36.0 kDa |
| | | family | | |
| B3 | H-M28214 | Homo sapiens GTP-binding | 24.2 | 34 |
| | | protein (RAB3B) mRNA, | | |
| | | complete cds | | |
| M300 C5 | H-M28249 | integrin, alpha 2 (CD49B, alpha 2 | 130.02 | 130.0 kDa |
| | | subunit of VLA-2 receptor) | | |
| | | [ITGA2] | | |
| M248 B6 | H-M28372 | zinc finger protein 9 (a cellular | 19.58 | 28.0 kDa |
| | | retroviral nucleic acid binding | | |
| | | protein) [ZNF9] | | |
| M248 C5 | H-M28983 | interleukin 1, alpha [IL1A] | 29.92 | 42 |
| M298 C1 | H-M29536 | translation initiation factor 2, beta | 36.74 | 50.0 kDa |
| | | subunit | | |
| M425 A5 | H-M29696 | Interleukin 7 receptor | 50.6 | 63.0 kDa |
| E1 | H-M29960 | Human steroid receptor (TR2-11) | 66.44 | 65 |
| | | mRNA, complete cds | | |
| M361 D3 | H-M29971 | 6-O-methylguanine-DNA | 22.88 | 33.0 kDa |
| | | methyltransferase [MGMT] | | |
| 167-67 | H-M30448 | “casein kinase II, beta chain” | 34 | 23.72 |
| M250 E2 | H-M31211 | MYOSIN LIGHT CHAIN 1, | 22.99 | 30.0 kDa |
| | | SLOW-TWITCH MUSCLE A | | |
| | | ISOFORM [Homo sapiens] | | |
| M311 C4 | H-M31452 | proline-rich protein | 65.78 | 68 |
| M312 H3 | H-M31469 | ras-like protein TC4 | 23.87 | 32.0 kDa |
| 167-41 | H-M31606 | “phosphorylase B kinase gamma | 50 | 44.7 |
| | | catalytic chain, testis isoform” | | |
| B4 | H-M31642 | Hypoxanthine | 24.09 | 36 |
| | | phosphoribosyltransferase 1 | | |
| | | (Lesch-Nyhan syndrome) | | |
| M416 D8 | H-M31932 | Fc fragment of IgG, low affinity | 34.98 | 45.0 kDa |
| | | IIa, receptor for (CD32) | | |
| M305 A8 | H-M32011 | neutrophil cytosolic factor 2 | 57.97 | 58 |
| | | (65 kD, chronic granulomatous | | |
| | | disease, autosomal 2) [NCF2] | | |
| B2 | H-M32315 | Human tumor necrosis factor | 50.82 | 60 |
| | | receptor mRNA, complete cds | | |
| M266 C2 | H-M33374 | cell adhesion protein SQM1 | 14.96 | 18.0 kDa |
| M431 F1 | H-M33375 | dihydrodiol dehydrogenase 4 | 33.99 | 40.0 kDa |
| G6 | H-M33680 | Human 26-kDa cell surface | 26.07 | 24 |
| | | protein TAPA-1 mRNA, complete | | |
| | | cds | | |
| F1 | H-M33772 | Human fast skeletal muscle | 17.71 | 29 |
| | | troponin C gene | | |
| 167-15 | H-M34065 | m-phase inducer phosphatase 3 | 55 | 52.10 |
| F4 | H-M34079 | Human immunodeficiency virus | 44.55 | 52 |
| | | tat transactivator binding protein- | | |
| | | 1 (tbp-1) mRNA, complete cds | | |
| 169-86 | H-M34181 | “cAMP-dependent protein kinase, | 50 | 38.68 |
| | | beta-catalytic subunit” | | |
| D1 | H-M34379 | Elastatse 2, neutrophil | 29.48 | 35 |
| M314 E1 | H-M34671 | CD59 glycoprotein precursor | 14.150 | 20 |
| M266 C3 | H-M35252 | CO-029 (GB: M35252) | 26.18 | 30 |
| M315 A4 | H-M36035 | benzodiazapine receptor | 18.7 | 19 |
| | | (peripheral) [BZRP] | | |
| M300 C1 | H-M36340 | ADP-ribosylation factor 1 | 20.02 | 30 |
| M312 C3 | H-M36341 | ADP-ribosylation factor 2 | 19.91 | 29 |
| D6 | H-M36634 | Vasoactive intestinal peptide | 18.81 | 28 |
| 169-26 | H-M36881 | proto-oncogene tyrosine-protein | 60 | 56.06 |
| | | kinase LCK | | |
| 167-76 | H-M36981 | nucleoside diphosphate kinase B | 26 | 16.79 |
| M298 D6 | H-M37400 | aspartate aminotransferase, | 45.54 | 50.0 kDa |
| | | cytosolic | | |
| 167-88 | H-M37712 | galactosyltransferase associated | 55 | 48.36 |
| | | protein kinase P58/GTA | | |
| M424 F4 | H-M38258 | Retinoic acid receptor, gamma 1 | 50.05 | 58.0 kDa |
| M266 H3 | H-M38690 | CD9 antigen, INVOLVED IN | 25.19 | 26.0 kDa |
| | | PLATELET ACTIVATION AND | | |
| | | AGGREGATION. | | |
| M270 A5 | H-M55265 | casein kinase II, alpha catalytic | 43.12 | 50 |
| | | subunit | | |
| 169-74 | H-M55284 | human protein kinase C-L | 80 | 75.09 |
| | | (PRKCL) mRNA | | |
| M512 B3 | H-M55514 | Potassium voltage-gated channel, | 71.94 | 100.0 kDa |
| | | shaker-related subfamily, member 4 | | |
| M271 F5 | H-M57567 | ADP-ribosylation factor 5 [AR5]. | 19.91 | 32.0 kDa |
| | | INVOLVED IN PROTEIN | | |
| | | TRAFFICKING AND ACTS AS | | |
| | | AN ALLOSTERIC ACTIVATOR | | |
| | | OF CHOLERA TOXIN. | | |
| M250 D1 | H-M57627 | interleukin 10 [IL10], | 19.69 | 27 |
| | | SUPPRESSOR FACTOR FOR | | |
| | | TH1 IMMUNE RESPONSES | | |
| | | (BY SIMILARITY). | | |
| M302 D3 | H-M57730 | EPH-related receptor tyrosine | 22.620 | 36.0 kDa |
| | | kinase ligand 1 precursor | | |
| M248 B5 | H-M58458 | ribosomal protein S4, X-linked | 29.04 | 36.0 kDa |
| | | [RPS4X] | | |
| M248 A5 | H-M58459 | ribosomal protein S4, Y-linked | 29.04 | 36 |
| | | [RPS4Y] | | |
| M248 G5 | H-M58525 | CATECHOL O- | 29.92 | 36 |
| | | METHYLTRANSFERASE, | | |
| | | MEMBRANE-BOUND FORM | | |
| | | [Homo sapiens], COMT | | |
| M482 B2 | H-M59916 | Sphingomyelin phosphodiesterase | 69.3 | 69.0 kDa |
| | | 1, acid lysosomal (acid | | |
| | | sphingomyelinase) | | |
| M390 C1 | H-M60091 | galactose-1-phosphate | 41.8 | 50.0 kDa |
| | | uridylyltransferase | | |
| M316 B1 | H-M60314 | bone morphogenetic protein 5 | 50.05 | 55 |
| | | [BMP5] | | |
| B4 | H-M60459 | Erythropoietin receptor | 55.99 | 60 |
| C7 | H-M60483 | Human protein phosphatase 2A | 34.1 | 56 |
| | | catalytic subunit-alpha gene, | | |
| | | complete cds | | |
| M462 D7 | H-M60484 | Human protein phosphatase 2A | 34.1 | 44.0 kDa |
| | | catalytic subunit-beta gene, | | |
| | | complete cds | | |
| A12 | H-M60527 | deoxycytidine kinase | 28.670 | 50 |
| 167-5 | H-M60724 | human p70 ribosomal S6 kinase | 66 | 57.82 |
| | | alpha-I mRNA | | |
| 167-17 | H-M60725 | human p70 ribosomal S6 kinase | 62 | 55.29 |
| | | alpha-II mRNA | | |
| M271 A4 | H-M61199 | cleavage signal 1, ESTs, Highly | 27.5 | 36.0 kDa |
| | | similar to CLEAVAGE SIGNAL- | | |
| | | 1 PROTEIN [Homo sapiens] | | |
| B1 | H-M61733 | Homo sapiens erythroid | 70.62 | 71 |
| | | membrane protein 4.1 mRNA, | | |
| | | complete cds | | |
| M298 A1 | H-M61764 | tubulin, gamma | 49.72 | 55.0 kDa |
| M422 E2 | H-M62505 | Complement component 5 | 38.61 | 38.0 kDa |
| | | receptor 1 (C5a ligand) | | |
| M313 G5 | H-M62810 | transcription factor 1, | 27.17 | 35.0 kDa |
| | | mitochondrial | | |
| C9 | H-M62839 | apolipoprotein H | 38.06 | 60 |
| G5 | H-M63154 | Gastric intrinsic factor (vitamin B | 45.98 | 52 |
| | | synthesis) | | |
| 167-6 | H-M63167 | RAC-alpha serine/threonine | 64 | 52.87 |
| | | kinase | | |
| B1 | H-M63573 | Peptidylprolyl isomerase B | 23.87 | 33 |
| | | (cyclophilin B) | | |
| M302 H2 | H-M63603 | phospholamban | 5.83 | 6 |
| M306 D1 | H-M63838 | interferon, gamma-inducible | 80.3 | 108 |
| | | protein 16 | | |
| M423 H3 | H-M63959 | Low density lipoprotein-related | 39.38 | 48.0 kDa |
| | | protein-associated protein 1 | | |
| | | (alpha-2-macroglobulin receptor- | | |
| | | associated protein 1 | | |
| G3 | H-M64099 | Human gamma-glutmyl | 64.57 | 52 |
| | | transpeptidase-related protein | | |
| | | (GGT-Rel) mRNA, complete cds | | |
| M475 B8 | H-M64673 | Human heat shock factor 1 | 58.3 | 65.0 kDa |
| | | (TCF5) mRNA, complete cds | | |
| M266 D5 | H-M64716 | ribosomal protein S25 | 13.86 | 17.0 kDa |
| M248 C6 | H-M64752 | glutamate receptor, ionotropic, | 99.88 | 100 |
| | | AMPA 1 [GRIA1] | | |
| M312 G3 | H-M64925 | palmitoylated membrane protein, | 51.37 | 51.0 kDa |
| | | erythrocyte, 55 kDa | | |
| M302 C7 | H-M65292 | complement factor H-related | 36.41 | 50 |
| | | protein (GB: M65292) | | |
| D3 | H-M68516 | Human protein C inhibitor gene, | 44.77 | 54 |
| | | complete cds | | |
| 167-27 | H-M68520 | cell division protein kinase 2 | 38 | 32.85 |
| M236 D5 | H-M68867 | Cellular retinoic acid-binding | 15.29 | 19.0 kDa |
| | | protein 2, MAY REGULATE | | |
| | | THE ACCESS OF RETINOIC | | |
| | | ACID TO THE NUCLEAR | | |
| | | RETINOIC ACID RECEPTORS. | | |
| M441 E1 | H-M69226 | monoamine oxidase A [MAOA] | 58.08 | 64.0 kDa |
| M298 D5 | H-M72393 | calcium-dependent phospholipid- | 82.5 | 117.0 kDa |
| | | binding protein [PLA2*] | | |
| M422 D5 | H-M73238 | Ciliary neurotrophic factor | 41.03 | 51.0 kDa |
| | | receptor | | |
| C1 | H-M73255 | Human vascular cell adhesion | 81.4 | 81 |
| | | molecule-1 (VCAM1) gene, | | |
| | | complete CDS | | |
| M422 G6 | H-M73481 | Human gastrin releasing peptide | 42.35 | 45.0 kDa |
| | | receptor (GRPR) mRNA, | | |
| | | complete cds | | |
| M235 G6 | H-M73499 | carboxylesterase, INVOLVED IN | 62.48 | 90.0 kDa |
| | | THE DETOXIFICATION OF | | |
| | | XENOBIOTICS AND THE | | |
| | | ACTIVATION OF ESTER AND | | |
| | | AMIDE PRODRUGS. | | |
| M302 D1 | H-M73547 | polyposis locus DP1 | 20.46 | 28 |
| M300 H4 | H-M73969 | interleukin 8 receptor, beta | 39.71 | 36 |
| | | [IL8RB] | | |
| G1 | H-M74491 | ADP-ribosylation factor 3 | 20.02 | 31 |
| B4 | H-M74816 | | 49.5 | 50 |
| B2 | H-M75110 | H,K-ATPase, beta subunit | 32.12 | 37 |
| M416 B8 | H-M76766 | General transcription factor IIB | 34.87 | 44.0 kDa |
| 167-18 | H-M77198 | RAC-beta serine/threonine kinase | 64 | 57.27 |
| 167-87 | H-M77348 | PMEL 17 protein precursor | 74 | 73.55 |
| C4 | H-M77698 | YY1 transcription factor | 45.65 | 48 |
| M248 G6 | H-M80261 | apurinic/apyrimidinic (abasic) | 35.09 | 37.0 kDa |
| | | endonuclease [APE], REPAIRS | | |
| | | OXIDATIVE DNA DAMAGES | | |
| | | IN VITRO | | |
| 169-50 | H-M80359 | putative serine/threonine-protein | 80 | 78.50 |
| | | kinase P78 | | |
| M330 H1 | H-M80461 | immunoglobulin-associated beta | 25.370 | 27.0 kDa |
| | | (B29) [IGB] | | |
| 169-1 | H-M80613 | ring3 protein | 100 | 83.01 |
| M298 A2 | H-M80783 | B12 protein | 34.87 | 43.0 kDa |
| 217-1 | H-M81457 | calpactin I light chain | 10 | 10.74 |
| M422 C6 | H-M81589 | Homo sapiens serotonin 1D | 41.58 | 41.0 kDa |
| | | receptor (5-HT1D‘) mRNA, | | |
| | | complete cds | | |
| M424 A1 | H-M81590 | Homo sapiens serotonin 1D | 43.01 | 48.0 kDa |
| | | receptor (5-HT1D˜) mRNA, | | |
| | | complete cds | | |
| M250 H1 | H-M81592 | gamma-glutamyl carboxylase | 83.49 | 85 |
| | | [GGCX], CONVERTS | | |
| | | GLUTAMATE RESIDUES TO | | |
| | | GAMMA- | | |
| | | CARBOXYGLUTAMATE | | |
| M250 F2 | H-M81601 | TRANSCRIPTION | 33.22 | 36.0 kDa |
| | | ELONGATION FACTOR S-II | | |
| | | [Homo sapiens] | | |
| C2 | H-M81650 | Human semenogelin I (SEMGI) | 50.93 | 52 |
| | | gene, complete cds | | |
| M266 A4 | H-M81757 | ribosomal protein S19 | 16.06 | 18 |
| 169-61 | H-M81933 | m-phase inducer phosphatase 1 | 57 | 57.60 |
| M302 H1 | H-M82809 | annexin IV | 35.42 | 38.0 kDa |
| M300 C4 | H-M83653 | cytoplasmic phosphotyrosyl | 17.49 | 28.0 kDa |
| | | protein phosphatase, type 1 | | |
| 169-14 | H-M83941 | tyrosne-protein kinase receptor | 108 | 108.2 |
| | | ETK1 precursor | | |
| F1 | H-M84443 | Galactokinase 2 | 50.49 | 52 |
| M305 H6 | H-M84747 | interleukin 9 receptor [IL9R] | 57.53 | 58 |
| 167-53 | H-M86400 | 14-3-3 protein zeta/delta | 33 | 27.02 |
| M271 C8 | H-M86521 | transketolase | 68.64 | 68.0 kDa |
| 169-51 | H-M86699 | human kinase (TTK) mRNA | 92 | 92.58 |
| M316 F2 | H-M86752 | transformation-sensitive protein | 59.84 | 60.0 kDa |
| M270 C8 | H-M86921 | membrane glycoprotein mb-1, | 24.97 | 34 |
| | | Immunoglobulin-associated | | |
| | | alpha, ASSOCIATED TO | | |
| | | SURFACE IGM-RECEPTOR; | | |
| | | MAY BE INVOLVED IN | | |
| | | SIGNAL TRANSDUCTION | | |
| A5 | H-M87507 | Homo sapien interleukin-1 beta | 44.55 | 50 |
| | | convertase (IL1BCE) mRNA, | | |
| | | complete cds | | |
| M305 B7 | H-M88011 | glucokinase [GCK] | 51.26 | 60 |
| M305 H1 | H-M88279 | immunophilin FKBP52 | 50.6 | 64.0 kDa |
| M420 F1 | H-M88468 | mevalonate kinase | 43.600 | 47.0 kDa |
| M305 A7 | H-M89913 | dUTP pyrophosphatase | 15.62 | 19 |
| | | (dUTPase) [DUT*] | | |
| M316 E2 | H-M90657 | tumor-associated antigen L6 | 22.33 | 28 |
| 167-31 | H-M90813 | human D-type cyclin (CCND2) | 36 | 31.86 |
| | | mRNA | | |
| A1 | H-M91036 | H. sapiens G-gamma globin and | 16.28 | 18 |
| | | A-gamma globin genes, complete | | |
| | | cds's | | |
| G2 | H-M91463 | Human glucose transporter | 55.66 | 52 |
| | | (GLUT4) gene, complete cds | | |
| A1 | H-M91670 | Human ubiquitin carrier protein | 24.86 | 36 |
| | | (E2-EPF) mRNA, complete cds | | |
| E4 | H-M92444 | Homo sapiens | 35.09 | 45 |
| | | apurinic/apyrimidinic | | |
| | | endonuclease (HAP1) gene, | | |
| | | complete cds | | |
| M305 C4 | H-M94556 | single-stranded DNA-binding | 16.39 | 20 |
| | | protein, mitochondrial | | |
| G12 | H-M94856 | fatty-acid-binding protein | 14.96 | 36 |
| | | homolog | | |
| M453 C3 | H-M95623 | Homo sapiens | 39.82 | 50.0 kDa |
| | | hydroxymethylbilane synthase | | |
| | | gene, complete cds | | |
| M302 F2 | H-M95787 | smooth muscle protein SM22 | 22.22 | 33.0 kDa |
| A1 | H-M95809 | Human basic transcription factor | 60.39 | 64 |
| | | 62 kD subunit (BTF2), complete | | |
| | | cds | | |
| M271 E8 | H-M96982 | small nuclear ribonucleoprotein | 26.51 | 39.0 kDa |
| | | U2 auxiliary factor, 35 kDa, | | |
| | | SPLICING FACTOR U2AF 35 | | |
| | | KD SUBUNIT. NECESSARY | | |
| | | FOR THE SPLICING OF PRE- | | |
| | | mRNA. | | |
| M416 B3 | H-M96995 | Growth factor receptor-bound | 23.98 | 32.0 kDa |
| | | protein 2 | | |
| G2 | H-M96995 | Growth factor receptor-bound | 23.98 | 49 |
| | | protein 2 | | |
| H4 | H-M97016 | Bone morphogenetic protein 8 | 44.33 | 61 |
| | | (osteogenic protein 2) | | |
| M271 D1 | H-M97190 | Sp2 transcription factor [SP2], | 54.56 | 60 |
| | | BINDS TO GC BOX | | |
| | | PROMOTERS ELEMENTS AND | | |
| | | SELECTIVELY ACTIVATES | | |
| | | mRNA SYNTHESIS FROM | | |
| | | GENES THAT CONTAIN | | |
| | | FUNCTIONAL RECOGNITION | | |
| | | SITES. | | |
| M271 C1 | H-M97191 | Sp3 transcription factor [SP3], | 71.94 | 72 |
| | | BINDS TO GT AND GC BOXES | | |
| | | PROMOTERS ELEMENTS. | | |
| | | PROBABLE | | |
| | | TRANSCTRIPTIONAL | | |
| | | ACTIVATOR. | | |
| M305 C7 | H-M97388 | transcription repressor (interacting | 19.47 | 30 |
| | | with the TATA-binding protein) | | |
| | | [DR1*] | | |
| 217-13 | H-M97675 | human transmembrane receptor | 100 | 103.1 |
| | | (ror1) mRNA | | |
| B3 | H-M97856 | Nuclear autoantigenic sperm | 86.68 | 87 |
| | | protein (histone-binding) | | |
| M429 G2 | H-M97935 | Homo sapiens transcription factor | 82.61 | 89.0 kDa |
| | | ISGF-3 mRNA, complete cds | | |
| D1 | H-M99487 | Human prostate-specific | 82.61 | 92 |
| | | membrane antigen (PSM) mRNA, | | |
| | | complete cds | | |
| M363 A1 | H-P0002 | riboflavin synthase beta chain | 17.27 | |
| | | (ribE) | | |
| M363 B1 | H-P0004 | carbonic anhydrase (icfA) | 24.42 | |
| M363 C1 | H-P0005 | orotidine 5′-phosphate | 25.08 | |
| | | decarboxylase (pyrF) | | |
| M363 D1 | H-P0006 | pantoate-beta-alanine ligase | 30.47 | |
| | | (panC) | | |
| M379 A1 | H-P0010-2 | chaperone and heat shock protein | 60.17 | |
| | | (groEL) | | |
| M363 E1 | H-P0011 | co-chaperone (groES) | 13.09 | |
| M363 F1 | H-P0012 | DNA primase (dnaG) | 61.6 | |
| M363 G1 | H-P0013 | hypothetical protein | 38.61 | |
| M363 H1 | H-P0014 | hypothetical protein | 30.36 | |
| M363 A2 | H-P0015 | hypothetical protein | 10.34 | |
| M363 B2 | H-P0016 | hypothetical protein | 9.68 | |
| M363 C2 | H-P0017 | virB4 homolog (virB4) | 86.68 | |
| M363 D2 | H-P0018 | hypothetical protein | 51.7 | |
| M363 E2 | H-P0021 | hypothetical protein | 21.01 | |
| M363 F2 | H-P0022 | conserved hypothetical integral | 57.42 | |
| | | membrane protein | | |
| M363 G2 | H-P0026 | citrate synthase (gltA) | 46.97 | |
| M363 H2 | H-P0027 | isocitrate dehydrogenase (icd) | 46.86 | |
| M363 A3 | H-P0028 | conserved hypothetical secreted | 19.58 | |
| | | protein | | |
| M363 B3 | H-P0030 | hypothetical protein | 65.34 | |
| M363 C3 | H-P0031 | hypothetical protein | 15.18 | |
| M363 D3 | H-P0034 | aspartate 1-decarboxylase (panD) | 12.98 | |
| M363 E3 | H-P0035 | conserved hypothetical protein | 10.78 | |
| M363 F3 | H-P0037 | NADH-ubiquinone | 38.72 | |
| | | oxidoreductase subunit | | |
| M363 G3 | H-P0044 | GDP-D-mannose dehydratase | 42.02 | |
| | | (rfbD) | | |
| M363 H3 | H-P0047 | hydrogenase expression/formation | 36.63 | |
| | | protein (hypE) | | |
| M363 A4 | H-P0048 | transcriptional regulator (hypF) | 84.7 | |
| M363 B4 | H-P0052 | hypothetical protein | 36.41 | |
| M363 C4 | H-P0055 | proline permease (putP) | 54.67 | |
| M363 D4 | H-P0056 | delta-1-pyrroline-5-carboxylate | 130.46 | |
| | | dehydrogenase | | |
| M363 E4 | H-P0057 | hypothetical protein | 7.7 | |
| M363 F4 | H-P0063 | hypothetical protein | 54.67 | |
| M363 G4 | H-P0064 | hypothetical protein | 15.4 | |
| M363 H4 | H-P0066 | conserved hypothetical ATP- | 91.52 | |
| | | binding protein | | |
| M363 A5 | H-P0067 | urease accessory protein (ureH) | 29.26 | |
| M363 B5 | H-P0068 | urease accessory protein (ureG) | 22 | |
| M363 C5 | H-P0075 | urease protein (ureC) | 49.06 | |
| M363 D5 | H-P0077 | peptide chain release factor RF-1 | 38.83 | |
| | | (prfA) | | |
| M363 E5 | H-P0082 | methyl-accepting chemotaxis | 74.14 | |
| | | transducer (tlpC) | | |
| M363 F5 | H-P0086 | conserved hypothetical protein | 49.61 | |
| M363 G5 | H-P0087 | hypothetical protein | 50.38 | |
| M363 H5 | H-P0088 | RNA polymerase sigma-70 factor | 73.92 | |
| | | (rpoD) | | |
| M363 A6 | H-P0089 | pfs protein (pfs) | 25.52 | |
| M363 B6 | H-P0090 | malonyl coenzyme A-acyl carrier | 34.1 | |
| | | protein transacylase (fabD) | | |
| M363 C6 | H-P0093 | hypothetical protein | 12.21 | |
| M363 D6 | H-P0096 | phosphoglycerate dehydrogenase | 34.65 | |
| M304 A1 | H-P0099 | methyl-accepting chemotaxis | 74.36 | |
| | | protein (tlpA) | | |
| M304 B1 | H-P0100 | conserved hypothetical protein | 40.59 | |
| M304 C1 | H-P0101 | hypothetical protein | 27.94 | |
| M304 D1 | H-P0104 | 2′,3′-cyclic-nucleotide 2′- | 64.02 | |
| | | phosphodiesterase (cpdB) | | |
| M304 E1 | H-P0105 | conserved hypothetical protein | 17.16 | |
| M304 F1 | H-P0106 | cystathionine gamma-synthase | 41.91 | |
| | | (metB) | | |
| M304 G1 | H-P0107 | cysteine synthetase (cysK) | 33.77 | |
| M304 H1 | H-P0108 | hypothetical protein | 20.57 | |
| M304 A2 | H-P0109 | chaperone and heat shock protein | 68.31 | |
| | | 70 (dnaK) | | |
| M304 B2 | H-P0110 | co-chaperone and heat shock | 20.9 | |
| | | protein (grpE) | | |
| M304 C2 | H-P0111 | hypothetical protein | 30.47 | |
| M304 D2 | H-P0113 | hypothetical protein | 10.89 | |
| M304 E2 | H-P0114 | hypothetical protein | 69.19 | |
| M304 F2 | H-P0115 | flagellin B (flaB) | 56.65 | |
| M304 G2 | H-P0116 | DNA topoisomerase I (topA) | 81.07 | |
| M304 H2 | H-P0117 | conserved hypothetical protein | 33.99 | |
| M304 A3 | H-P0118 | hypothetical protein | 43.56 | |
| M304 B3 | H-P0119 | hypothetical protein | 50.82 | |
| M304 C3 | H-P0120 | hypothetical protein | 43.89 | |
| M304 D3 | H-P0121 | phosphoenolpyruvate synthase | 89.43 | |
| | | (ppsA) | | |
| M304 E3 | H-P0122 | hypothetical protein | 4.84 | |
| M304 F3 | H-P0123 | threonyl-tRNA synthetase (thrS) | 67.43 | |
| M304 G3 | H-P0124 | translation initiation factor IF-3 | 22.44 | |
| | | (infC) | | |
| M304 H3 | H-P0125 | ribosomal protein L35 (rpl35) | 7.15 | |
| M304 A4 | H-P0126 | ribosomal protein L20 (rpl20) | 12.87 | |
| M304 B4 | H-P0127 | outer membrane protein (omp4) | 31.57 | |
| M304 C4 | H-P0128 | hypothetical protein | 4.62 | |
| M304 D4 | H-P0129 | hypothetical protein | 15.62 | |
| M304 E4 | H-P0130 | hypothetical protein | 31.57 | |
| M304 F4 | H-P0131 | hypothetical protein | 3.74 | |
| M304 G4 | H-P0132 | L-serine deaminase (sdaA) | 50.16 | |
| M304 H4 | H-P0133 | serine transporter (sdaC) | 45.54 | |
| M304 A5 | H-P0134 | 3-deoxy-D-arabino-heptulosonate | 49.5 | |
| | | 7-phosphate synthase (dhs1) | | |
| M304 B5 | H-P0135 | hypothetical protein | 4.95 | |
| M304 C5 | H-P0136 | bacterioferritin comigratory | 16.83 | |
| | | protein (bcp) | | |
| M304 D5 | H-P0137 | hypothetical protein | 23.32 | |
| M304 E5 | H-P0138 | conserved hypothetical iron-sulfur | 53.02 | |
| | | protein | | |
| M304 F5 | H-P0139 | conserved hypothetical secreted | 26.73 | |
| | | protein | | |
| M304 G5 | H-P0140 | L-lactate permease (lctP) | 60.5 | |
| M304 H5 | H-P0141 | L-lactate permease (lctP) | 60.72 | |
| M304 A6 | H-P0142 | A/G-specific adenine glycosylase | 36.19 | |
| | | (mutY) | | |
| M304 B6 | H-P0144 | cytochrome c oxidase, heme b | 53.79 | |
| | | and copper-binding subunit, | | |
| | | membrane-bound (fixN) | | |
| M304 C6 | H-P0145 | cytochrome c oxidase, monoheme | 25.63 | |
| | | subunit, membrane-bound (fixO) | | |
| M304 D6 | H-P0146 | cbb3-type cytochrome c oxidase | 8.14 | |
| | | subunit Q (CcoQ) | | |
| M304 E6 | H-P0147 | cytochrome c oxidase, diheme | 31.57 | |
| | | subunit, membrane-bound (fixP) | | |
| M304 F6 | H-P0148 | hypothetical protein | 7.59 | |
| M304 G6 | H-P0150 | hypothetical protein | 21.67 | |
| M304 H6 | H-P0152 | hypothetical protein | 31.68 | |
| M304 A7 | H-P0153 | recombinase (recA) | 38.28 | |
| M304 B7 | H-P0154 | enolase (eno) | 46.97 | |
| M304 C7 | H-P0155 | hypothetical protein | 10.12 | |
| M304 D7 | H-P0157 | shikimic acid kinase I (aroK) | 17.93 | |
| M304 E7 | H-P0158 | hypothetical protein | 35.09 | |
| M304 F7 | H-P0159 | lipopolysaccharide 1,2- | 41.03 | |
| | | glucosyltransferase (rfaJ) | | |
| M304 G7 | H-P0161 | hypothetical protein | 4.07 | |
| M304 H7 | H-P0162 | conserved hypothetical protein | 26.51 | |
| M304 A8 | H-P0163 | delta-aminolevulinic acid | 35.64 | |
| | | dehydratase (hemB) | | |
| M304 B8 | H-P0164 | signal-transducing protein, | 28.05 | |
| | | histidine kinase | | |
| M304 C8 | H-P0165 | hypothetical protein | 19.14 | |
| M304 D8 | H-P0166 | response regulator (ompR) | 24.86 | |
| M304 E8 | H-P0167 | hypothetical protein | 17.38 | |
| M304 F8 | H-P0168 | hypothetical protein | 9.68 | |
| M304 G8 | H-P0170 | hypothetical protein | 27.94 | |
| M304 H8 | H-P0171 | peptide chain release factor RF-2 | 40.04 | |
| | | (prfB) | | |
| M304 A9 | H-P0172 | molybdopterin biosynthesis | 43.12 | |
| | | protein (moeA) | | |
| M304 B9 | H-P0173 | flagellar biosynthetic protein | 28.16 | |
| | | (fliR) | | |
| M304 C9 | H-P0174 | hypothetical protein | 28.49 | |
| M304 D9 | H-P0175 | cell binding factor 2 | 33 | |
| M304 E9 | H-P0176 | fructose-bisphosphate aldolase | 33.88 | |
| | | (tsr) | | |
| M304 F9 | H-P0177 | translation elongation factor EF-P | 20.68 | |
| | | (efp) | | |
| M304 G9 | H-P0178 | spore coat polysaccharide | 37.51 | |
| | | biosynthesis protein E | | |
| M304 H9 | H-P0179 | ABC transporter, ATP-binding | 23.54 | |
| | | protein | | |
| M304 A10 | H-P0180 | apolipoprotein N-acyltransferase | 46.86 | |
| | | (cute) | | |
| M304 B10 | H-P0182 | lysyl-tRNA synthetase (lysS) | 55.22 | |
| M304 C10 | H-P0183 | serine hydroxymethyltransferase | 45.87 | |
| | | (glyA) | | |
| M304 D10 | H-P0184 | hypothetical protein | 19.91 | |
| M304 E10 | H-P0185 | hypothetical protein | 29.48 | |
| M304 F10 | H-P0186 | hypothetical protein | 44.55 | |
| M304 G10 | H-P0187 | hypothetical protein | 10.56 | |
| M304 H10 | H-P0188 | hypothetical protein | 3.74 | |
| M304 A11 | H-P0189 | conserved hypothetical integral | 19.58 | |
| | | membrane protein | | |
| M304 B11 | H-P0190 | conserved hypothetical secreted | 55.33 | |
| | | protein | | |
| M304 C11 | H-P0191 | fumarate reductase, iron-sulfur | 27.06 | |
| | | subunit (frdB) | | |
| M304 D11 | H-P0192 | fumarate reductase, flavoprotein | 78.65 | |
| | | subunit (frdA) | | |
| M304 E11 | H-P0193 | fumarate reductase, cytochrome b | 28.16 | |
| | | subunit (frdC) | | |
| M304 F11 | H-P0194 | triosephosphate isomerase (tpi) | 25.85 | |
| M304 G11 | H-P0195 | enoyl-(acyl-carrier-protein) | 30.36 | |
| | | reductase (NADH) (fabI) | | |
| M365 A1 | H-P0197 | S-adenosylmethionine synthetase | 42.46 | |
| | | 2 (metX) | | |
| M365 B1 | H-P0203 | hypothetical protein | 10.12 | |
| M365 C1 | H-P0209 | hypothetical protein | 49.61 | |
| M365 D1 | H-P0213 | glucose inhibited division protein | 68.42 | |
| | | (gidA) | | |
| M381 E1 | H-P0218 | hypothetical protein | 20.24 | |
| M365 E1 | H-P0221 | nifU-like protein | 35.97 | |
| M365 F1 | H-P0227 | outer membrane protein (omp5) | 76.12 | |
| M365 G1 | H-P0228 | conserved hypothetical integral | 43.01 | |
| | | membrane protein | | |
| M365 H1 | H-P0230 | CTP: CMP-3-deoxy-D-manno- | 26.84 | |
| | | octulosonate-cytidylyl-transferase | | |
| | | (kdsB) | | |
| M365 A2 | H-P0233 | conserved hypothetical protein | 43.01 | |
| M365 B2 | H-P0235 | conserved hypothetical secreted | 39.16 | |
| | | protein | | |
| M365 C2 | H-P0236 | hypothetical protein | 13.64 | |
| M365 D2 | H-P0238 | prolyl-tRNA synthetase (proS) | 63.58 | |
| M381 E2 | H-P0243 | neutrophil activating protein | 15.95 | |
| | | (napA) (bacterioferritin) | | |
| M365 E2 | H-P0244 | signal-transducing protein, | 42.02 | |
| | | histidine kinase (atoS) | | |
| M365 F2 | H-P0246 | flagellar basal-body P-ring protein | 37.73 | |
| | | (flgI) | | |
| M365 G2 | H-P0247 | ATP-dependent RNA helicase, | 54.23 | |
| | | DEAD-box family (deaD) | | |
| M365 H2 | H-P0248 | conserved hypothetical protein | 39.93 | |
| M379 B1 | H-P0249-2 | hypothetical protein | 19.8 | |
| M379 C1 | H-P0250-2 | oligopeptide ABC transporter, | 56.87 | |
| | | ATP-binding protein (oppD) | | |
| M381 A3 | H-P0251 | oligopeptide ABC transporter, | 37.29 | |
| | | permease protein (oppC) | | |
| M379 E1 | H-P0252-2 | outer membrane protein (omp7) | 53.68 | |
| M365 A3 | H-P0254 | outer membrane protein (omp8) | 47.52 | |
| M365 B3 | H-P0255 | adenylosuccinate synthetase | 45.32 | |
| | | (purA) | | |
| M365 C3 | H-P0257 | conserved hypothetical secreted | 24.2 | |
| | | protein | | |
| M365 D3 | H-P0259 | exonuclease VII, large subunit | 46.31 | |
| | | (xseA) | | |
| M381 D3 | H-P0260 | adenine specific DNA | 42.35 | |
| | | methyltransferase (mod) | | |
| M365 E3 | H-P0263 | adenine specific DNA | 27.83 | |
| | | methyltransferase (hpaim) | | |
| M365 F3 | H-P0264 | ATP-dependent protease binding | 94.27 | |
| | | subunit (clpB) | | |
| M365 G3 | H-P0266 | dihydroorotase (pyrC) | 41.69 | |
| M365 H3 | H-P0267 | chlorohydrolase | 45.1 | |
| M365 A4 | H-P0271 | hypothetical protein | 36.08 | |
| M365 B4 | H-P0275 | ATP-dependent nuclease (addB) | 47.41 | |
| M381 G3 | H-P0276 | hypothetical protein | 20.46 | |
| M365 C4 | H-P0278 | guanosine pentaphosphate | 53.35 | |
| | | phosphohydrolase (gppA) | | |
| M365 D4 | H-P0279 | lipopolysaccharide | 37.51 | |
| | | heptosyltransferase-1 (rfaC) | | |
| M365 E4 | H-P0280 | heat shock protein B (ibpB) | 36.19 | |
| M365 F4 | H-P0282 | hypothetical protein | 52.91 | |
| M365 G4 | H-P0283 | 3-dehydroquinate synthase (aroB) | 37.84 | |
| M365 H4 | H-P0284 | conserved hypothetical integral | 57.64 | |
| | | membrane protein | | |
| M365 A5 | H-P0285 | conserved hypothetical protein | 46.09 | |
| M381 A4 | H-P0287 | hypothetical protein | 19.03 | |
| M381 C4 | H-P0288 | hypothetical protein | 17.38 | |
| M366 A1 | H-P0389 | superoxide dismutase (sodB) | 23.54 | |
| M366 B1 | H-P0390 | adhesin-thiol peroxidase (tagD) | 18.37 | |
| M366 C1 | H-P0391 | purine-binding chemotaxis | 18.26 | |
| | | protein (cheW) | | |
| M366 D1 | H-P0392 | histidine kinase (cheA) | 88.44 | |
| M366 E1 | H-P0393 | chemotaxis protein (cheV) | 34.32 | |
| M366 F1 | H-P0394 | hypothetical protein | 27.83 | |
| M366 G1 | H-P0395 | conserved hypothetical protein | 24.53 | |
| M366 H1 | H-P0396 | conserved hypothetical protein | 67.87 | |
| M366 A2 | H-P0397 | phosphoglycerate dehydrogenase | 57.75 | |
| | | (serA) | | |
| M366 B2 | H-P0398 | hypothetical protein | 20.13 | |
| M366 C2 | H-P0399 | ribosomal protein S1 (rps1) | 61.27 | |
| M366 D2 | H-P0403 | phenylalanyl-tRNA synthetase, | 36.19 | |
| | | alpha subunit (pheS) | | |
| M366 E2 | H-P0404 | protein kinase C inhibitor | 11.55 | |
| | | (SP: P16436) | | |
| M366 F2 | H-P0405 | nifS-like protein | 48.51 | |
| M366 G2 | H-P0406 | hypothetical protein | 21.67 | |
| M366 H2 | H-P0407 | biotin sulfoxide reductase (bisC) | 87.67 | |
| M381 D1 | H-P0409 | GMP synthase (guaA) | 55.99 | |
| M381 F1 | H-P0410 | putative neuraminyllactose- | 27.5 | |
| | | binding hemagglutinin homolog | | |
| | | (hpaA) | | |
| M366 A3 | H-P0411 | hypothetical protein | 11.66 | |
| M366 B3 | H-P0412 | hypothetical protein | 3.63 | |
| M366 C3 | H-P0413 | transposase-like protein, PS3IS | 29.59 | |
| M366 D3 | H-P0414 | IS200 insertion sequence from | 15.29 | |
| | | SARA17 | | |
| M366 E3 | H-P0415 | conserved hypothetical integral | 68.64 | |
| | | membrane protein | | |
| M366 F3 | H-P0416 | cyclopropane fatty acid synthase | 42.9 | |
| | | (cfa) | | |
| M366 G3 | H-P0417 | methionyl-tRNA synthetase | 71.61 | |
| | | (metS) | | |
| M366 H3 | H-P0418 | hypothetical protein | 36.96 | |
| M366 A4 | H-P0419 | conserved hypothetical protein | 28.82 | |
| M366 B4 | H-P0420 | hypothetical protein | 15.73 | |
| M366 C4 | H-P0421 | type 1 capsular polysaccharide | 42.9 | |
| | | biosynthesis protein J (capJ) | | |
| M366 D4 | H-P0422 | arginine decarboxylase (speA) | 67.76 | |
| M366 E4 | H-P0424 | hypothetical protein | 68.2 | |
| M366 F4 | H-P0425 | hypothetical protein | 45.98 | |
| M366 G4 | H-P0427 | hypothetical protein | 12.32 | |
| M366 H4 | H-P0433 | hypothetical protein | 16.28 | |
| M366 A5 | H-P0436 | hypothetical protein | 13.42 | |
| M366 B5 | H-P0437 | IS605 transposase (tnpA) | 15.73 | |
| M366 C5 | H-P0438 | IS605 transposase (tnpB) | 47.08 | |
| M366 D5 | H-P0442 | hypothetical protein | 9.79 | |
| M366 E5 | H-P0445 | hypothetical protein | 6.82 | |
| M366 F5 | H-P0452 | hypothetical protein | 57.09 | |
| M366 G5 | H-P0455 | hypothetical protein | 11.44 | |
| M366 H5 | H-P0457 | hypothetical protein | 9.68 | |
| M366 A6 | H-P0463 | type I restriction enzyme M | 53.68 | |
| | | protein (hsdM) | | |
| M366 B6 | H-P0464 | type I restriction enzyme R | 116.16 | |
| | | protein (hsdR) | | |
| M366 C6 | H-P0465 | conserved hypothetical protein | 69.52 | |
| M366 D6 | H-P0466 | conserved hypothetical protein | 28.16 | |
| M366 E6 | H-P0467 | conserved hypothetical integral | 12.76 | |
| | | membrane protein | | |
| M366 F6 | H-P0468 | conserved hypothetical protein | 54.56 | |
| M366 G6 | H-P0469 | conserved hypothetical protein | 17.93 | |
| M366 H6 | H-P0471 | glutathione-regulated potassium- | 45.87 | |
| | | efflux system protein (kefB) | | |
| M366 A7 | H-P0472 | outer membrane protein (omp11) | 20.57 | |
| M366 B7 | H-P0473 | molybdenum ABC transporter, | 27.17 | |
| | | periplasmic molybdate-binding | | |
| | | protein (modA) | | |
| M366 C7 | H-P0474 | molybdenum ABC transporter, | 24.75 | |
| | | permease protein (modB) | | |
| M366 D7 | H-P0475 | molybdenum ABC transporter, | 29.26 | |
| | | ATP-binding protein (modD) | | |
| M366 E7 | H-P0476 | glutamyl-tRNA synthetase (gltX) | 51.04 | |
| M366 F7 | H-P0477 | outer membrane protein (omp12) | 40.48 | |
| M366 G7 | H-P0478 | adenine specific DNA | 60.06 | |
| | | methyltransferase (VSPIM) | | |
| M366 H7 | H-P0479 | hypothetical protein | 31.13 | |
| M366 A8 | H-P0481 | adenine specific DNA | 23.32 | |
| | | methyltransferase (MFOKI) | | |
| M366 B8 | H-P0482 | hypothetical protein | 18.81 | |
| M366 C8 | H-P0483 | cytosine specific DNA | 36.3 | |
| | | methyltransferase (H-PHIMC) | | |
| M367 A1 | H-P0486 | hypothetical protein | 58.19 | |
| M367 B1 | H-P0487 | hypothetical protein | 52.91 | |
| M367 C1 | H-P0489 | hypothetical protein | 32.56 | |
| M367 D1 | H-P0490 | putative potassium channel | 41.69 | |
| | | protein, putative | | |
| M367 E1 | H-P0491 | ribosomal protein L28 (rpL28) | 6.93 | |
| M367 F1 | H-P0492 | hypothetical protein | 30.69 | |
| M367 G1 | H-P0494 | UDP-N-acetylmuramoylalanine- | 46.53 | |
| | | D-glutamate ligase (murD) | | |
| M367 H1 | H-P0495 | hypothetical protein | 9.57 | |
| M367 A2 | H-P0496 | conserved hypothetical protein | 14.74 | |
| M367 B2 | H-P0498 | sodium- and chloride-dependent | 48.73 | |
| | | transporter | | |
| M367 C2 | H-P0499 | phospholipase A1 precursor (DR- | 39.16 | |
| | | phospholipase A) | | |
| M367 D2 | H-P0500 | DNA polymerase III beta-subunit | 41.25 | |
| | | (dnaN) | | |
| M367 E2 | H-P0501 | DNA gyrase, sub B (gyrB) | 85.14 | |
| M367 F2 | H-P0503 | hypothetical protein | 27.17 | |
| M367 G2 | H-P0504 | hypothetical protein | 5.5 | |
| M367 H2 | H-P0505 | hypothetical protein | 17.05 | |
| M367 A3 | H-P0507 | conserved hypothetical protein | 23.43 | |
| M367 B3 | H-P0509 | glycolate oxidase subunit (glcD) | 50.6 | |
| M367 C3 | H-P0510 | dihydrodipicolinate reductase | 28.05 | |
| | | (dapB) | | |
| M367 D3 | H-P0512 | glutamine synthetase (glnA) | 53.02 | |
| M367 E3 | H-P0514 | ribosomal protein L9 (rpl9) | 16.61 | |
| M367 F3 | H-P0515 | heat shock protein (hslV) | 19.91 | |
| M367 G3 | H-P0516 | heat shock protein (hslU) ORF1 | 48.84 | |
| M367 H3 | H-P0517 | GTP-binding protein (era) | 33.33 | |
| M367 A4 | H-P0519 | conserved hypothetical protein | 30.47 | |
| M367 B4 | H-P0520 | cag pathogenicity island protein | 12.76 | |
| | | (cag1) | | |
| M367 C4 | H-P0522 | cag pathogenicity island protein | 53.02 | |
| | | (cag3) | | |
| M367 D4 | H-P0523 | cag pathogenicity island protein | 18.7 | |
| | | (cag4) | | |
| M367 E4 | H-P0525 | virB11 homolog | 36.41 | |
| M367 F4 | H-P0526 | cag pathogenicity island protein | 22 | |
| | | (cag6) | | |
| M367 G4 | H-P0528 | cag pathogenicity island protein | 57.53 | |
| | | (cag8) | | |
| M379 H1 | H-P0531-2 | cag pathogenicity island protein | 24.09 | |
| | | (cag11) | | |
| M367 H4 | H-P0532 | cag pathogenicity island protein | 30.91 | |
| | | (cag12) | | |
| M367 A5 | H-P0534 | cag pathogenicity island protein | 21.67 | |
| | | (cag13) | | |
| M367 B5 | H-P0541 | cag pathogenicity island protein | 40.81 | |
| | | (cag20) | | |
| M367 C5 | H-P0542 | cag pathogenicity island protein | 15.73 | |
| | | (cag21) | | |
| M367 D5 | H-P0545 | cag pathogenicity island protein | 22.88 | |
| | | (cag24) | | |
| M367 E5 | H-P0549 | glutamate racemase (glr) | 28.16 | |
| M367 F5 | H-P0550 | transcription termination factor | 48.29 | |
| | | Rho (rho) | | |
| M367 G5 | H-P0551 | ribosomal protein L31 (rpl31) | 7.48 | |
| M367 H5 | H-P0552 | conserved hypothetical protein | 31.68 | |
| M367 A6 | H-P0553 | conserved hypothetical protein | 25.08 | |
| M367 B6 | H-P0554 | hypothetical protein | 35.42 | |
| M367 C6 | H-P0555 | hypothetical protein | 30.14 | |
| M367 D6 | H-P0556 | hypothetical protein | 16.06 | |
| M367 E6 | H-P0557 | acetyl-coenzyme A carboxylase | 34.43 | |
| | | (accA) | | |
| M367 F6 | H-P0558 | beta ketoacyl-acyl carrier protein | 45.43 | |
| | | synthase II (fabF) | | |
| M367 G6 | H-P0561 | 3-ketoacyl-acyl carrier protein | 27.28 | |
| | | reductase (fabG) | | |
| M367 H6 | H-P0562 | ribosomal protein S21 (rps21) | 7.81 | |
| M367 A7 | H-P0563 | hypothetical protein | 45.87 | |
| M367 B7 | H-P0566 | diaminopimelate epimerase | 30.14 | |
| | | (dapF) | | |
| M367 C7 | H-P0568 | hypothetical protein | 28.16 | |
| M367 D7 | H-P0570 | aminopeptidase a/i (pepA) | 54.67 | |
| M367 E7 | H-P0571 | conserved hypothetical integral | 21.23 | |
| | | membrane protein | | |
| M379 A2 | H-P0572-2 | adenine | 19.8 | |
| | | phosphoribosyltransferase (apt) | | |
| M379 B2 | H-P0573-2 | hypothetical protein | 12.21 | |
| M379 C2 | H-P0574-2 | galactosidase acetyltransferase | 16.72 | |
| | | (lacA) | | |
| M379 D2 | H-P0575-2 | conserved hypothetical membrane | 25.63 | |
| | | protein | | |
| M379 E2 | H-P0576-2 | signal peptidase I (lepB) | 32.01 | |
| M367 F7 | H-P0577 | methylene-tetrahydrofolate | 32.23 | |
| | | dehydrogenase (folD) | | |
| M367 G7 | H-P0579 | hypothetical protein | 20.35 | |
| M367 H7 | H-P0580 | hypothetical protein | 41.03 | |
| M367 A8 | H-P0581 | dihydroorotase (pyrC) | 37.4 | |
| M367 B8 | H-P0582 | hypothetical protein | 35.75 | |
| M367 C8 | H-P0583 | hypothetical protein | 32.34 | |
| M368 A1 | H-P0584 | flagellar switch protein (fliN) | 13.64 | |
| M368 B1 | H-P0585 | endonuclease III (nth) | 24.09 | |
| M368 C1 | H-P0587 | aminodeoxychorismate lyase | 36.3 | |
| | | (pabC) | | |
| M368 D1 | H-P0591 | ferredoxin oxidoreductase, | 20.57 | |
| | | gamma subunit | | |
| M368 E1 | H-P0593 | adenine specific DNA | 65.89 | |
| | | methyltransferase (mod) | | |
| M368 F1 | H-P0594 | hypothetical protein | 6.05 | |
| M368 G1 | H-P0596 | hypothetical protein | 21.23 | |
| M368 H1 | H-P0597 | penicillin-binding protein 1A | 72.6 | |
| | | (PBP-1A) | | |
| M368 A2 | H-P0599 | hemolysin secretion protein | 47.74 | |
| | | precursor (hylB) | | |
| M368 B2 | H-P0601 | flagellin A (flaA) | 56.21 | |
| M368 C2 | H-P0602 | endonuclease III | 24.09 | |
| M368 D2 | H-P0603 | hypothetical protein | 20.9 | |
| M379 F2 | H-P0608-2 | hypothetical protein | 17.71 | |
| M368 E2 | H-P0614 | hypothetical protein | 12.32 | |
| M368 F2 | H-P0616 | chemotaxis protein (cheV) | 34.54 | |
| M368 G2 | H-P0617 | aspartyl-tRNA synthetase (aspS) | 63.58 | |
| M368 H2 | H-P0621 | DNA mismatch repair protein | 83.93 | |
| | | (MutS) | | |
| M368 A3 | H-P0622 | hypothetical protein | 13.31 | |
| M368 B3 | H-P0623 | UDP-N-acetylmuramate-alanine | 49.5 | |
| | | ligase (murC) | | |
| M368 C3 | H-P0625 | protein E (gcpE) | 39.6 | |
| M368 D3 | H-P0626 | tetrahydrodipicolinate N- | 44.22 | |
| | | succinyltransferase (dapD) | | |
| M368 E3 | H-P0627 | hypothetical protein | 12.21 | |
| M368 F3 | H-P0629 | hypothetical protein | 75.02 | |
| M368 G3 | H-P0630 | modulator of drug activity | 21.45 | |
| | | (mda66) | | |
| M368 H3 | H-P0631 | quinone-reactive Ni/Fe | 42.35 | |
| | | hydrogenase, small subunit | | |
| | | (hydA) | | |
| M368 A4 | H-P0632 | quinone-reactive Ni/Fe | 63.69 | |
| | | hydrogenase, large subunit | | |
| | | (hydB) | | |
| M368 B4 | H-P0633 | quinone-reactive Ni/Fe | 24.75 | |
| | | hydrogenase, cytochrome b | | |
| | | subunit (hydC) | | |
| M368 C4 | H-P0634 | quinone-reactive Ni/Fe | 19.69 | |
| | | hydrogenase (hydD) | | |
| M368 D4 | H-P0635 | hypothetical protein | 56.43 | |
| M368 E4 | H-P0636 | hypothetical protein | 10.23 | |
| M368 F4 | H-P0637 | hypothetical protein | 16.61 | |
| M368 G4 | H-P0638 | outer membrane protein (omp13) | 33.66 | |
| M368 H4 | H-P0643 | glutamyl-tRNA synthetase (gltX) | 48.4 | |
| M368 A5 | H-P0644 | conserved hypothetical integral | 10.78 | |
| | | membrane protein | | |
| M368 B5 | H-P0645 | soluble lytic murein | 61.71 | |
| | | transglycosylase (slt) | | |
| M368 C5 | H-P0646 | UDP-glucose pyrophosphorylase | 30.14 | |
| | | (galU) | | |
| M368 D5 | H-P0647 | hypothetical protein | 14.96 | |
| M368 E5 | H-P0648 | UDP-N-acetylglucosamine | 46.53 | |
| | | enolpyruvyl transferase (murZ) | | |
| M368 F5 | H-P0649 | aspartate ammonia-lyase (aspA) | 51.59 | |
| M368 G5 | H-P0650 | hypothetical protein | 21.67 | |
| M379 A3 | H-P0651-2 | fucosyltransferase | 52.47 | |
| M381 E3 | H-P0652 | phosphoserine phosphatase (serB) | 22.88 | |
| M368 H5 | H-P0653 | nonheme iron-containing ferritin | 18.48 | |
| | | (pfr) | | |
| M379 G2 | H-P0654-2 | conserved hypothetical protein | 39.71 | |
| M379 H2 | H-P0655-2 | protective surface antigen D15 | 100.87 | |
| M368 A6 | H-P0656 | conserved hypothetical protein | 42.24 | |
| M368 B6 | H-P0657 | processing protease (ymxG) | 47.63 | |
| M368 C6 | H-P0658 | PET112-like protein | 52.36 | |
| M368 D6 | H-P0659 | hypothetical protein | 45.65 | |
| M368 E6 | H-P0660 | hypothetical protein | 37.29 | |
| M368 F6 | H-P0661 | ribonuclease H (rnhA) | 15.84 | |
| M368 G6 | H-P0662 | ribonuclease III (rnc) | 26.51 | |
| M368 H6 | H-P0663 | chorismate synthase (aroC) | 40.26 | |
| M368 A7 | H-P0665 | oxygen-independent | 50.38 | |
| | | coproporphyrinogen III oxidase | | |
| | | (hemN) | | |
| M368 B7 | H-P0667 | hypothetical protein | 9.46 | |
| M368 C7 | H-P0668 | hypothetical protein | 66.88 | |
| M368 D7 | H-P0671 | outer membrane protein (omp14) | 29.81 | |
| M368 E7 | H-P0672 | solute-binding signature and | 43.01 | |
| | | mitochondrial signature protein | | |
| | | (aspB) | | |
| M379 B3 | H-P0673-2 | hypothetical protein | 46.97 | |
| M381 H3 | H-P0674 | hypothetical protein | 25.19 | |
| M368 F7 | H-P0676 | methylated-DNA-protein- | 18.59 | |
| | | cysteine methyltransferase (dat1) | | |
| M368 G7 | H-P0677 | conserved hypothetical integral | 28.16 | |
| | | membrane protein | | |
| M368 H7 | H-P0679 | lipopolysaccharide biosynthesis | 31.9 | |
| | | protein (wbpB) | | |
| M369 A1 | H-P0681 | hypothetical protein | 18.59 | |
| M369 B1 | H-P0682 | hypothetical protein | 13.97 | |
| M369 C1 | H-P0683 | UDP-N-acetylglucosamine | 47.74 | |
| | | pyrophosphorylase (glmU) | | |
| M369 D1 | H-P0685 | flagellar biosynthetic protein | 19.03 | |
| | | (fliP) | | |
| M369 E1 | H-P0687 | iron(II) transport protein (feoB) | 70.73 | |
| M369 F1 | H-P0688 | hypothetical protein | 18.37 | |
| M369 G1 | H-P0690 | acetyl coenzyme A | 43.12 | |
| | | acetyltransferase (thiolase) (fadA) | | |
| M381 A1 | H-P0691 | 3-oxoadipate coA-transferase | 25.63 | |
| | | subunit A (yxjD) | | |
| M381 B1 | H-P0692 | 3-oxoadipate coA-transferase | 22.88 | |
| | | subunit B (yxjE) | | |
| M369 H1 | H-P0694 | hypothetical protein | 28.38 | |
| M369 A2 | H-P0695 | hydantoin utilization protein A | 78.54 | |
| | | (hyuA) | | |
| M369 B2 | H-P0697 | hypothetical protein | 18.59 | |
| M369 C2 | H-P0699 | hypothetical protein | 37.73 | |
| M369 D2 | H-P0700 | diacylglycerol kinase (dgkA) | 14.19 | |
| M369 E2 | H-P0701 | DNA gyrase, sub A (gyrA) | 91.08 | |
| M369 F2 | H-P0703 | response regulator | 42.02 | |
| M369 G2 | H-P0707 | conserved hypothetical protein | 33.99 | |
| M369 H2 | H-P0711 | hypothetical protein | 44.77 | |
| M369 A3 | H-P0715 | ABC transporter, ATP-binding | 26.51 | |
| | | protein | | |
| M369 B3 | H-P0716 | conserved hypothetical protein | 14.74 | |
| M369 C3 | H-P0718 | conserved hypothetical integral | 23.21 | |
| | | membrane protein | | |
| M369 D3 | H-P0719 | hypothetical protein | 12.1 | |
| M369 E3 | H-P0723 | L-asparaginase II (ansB) | 36.41 | |
| M369 F3 | H-P0724 | anaerobic C4-dicarboxylate | 48.84 | |
| | | transport protein (dcuA) | | |
| M369 G3 | H-P0727 | transcriptional regulator, putative | 36.19 | |
| M369 H3 | H-P0728 | conserved hypothetical protein | 37.07 | |
| M369 A4 | H-P0730 | hypothetical protein | 11.22 | |
| M369 B4 | H-P0732 | hypothetical protein | 13.09 | |
| M369 C4 | H-P0734 | conserved hypothetical protein | 48.4 | |
| M369 D4 | H-P0735 | xanthine guanine phosphoribosyl | 16.94 | |
| | | transferase (gpt) | | |
| M369 E4 | H-P0737 | conserved hypothetical integral | 17.49 | |
| | | membrane protein | | |
| M381 H2 | H-P0738 | D-alanine: D-alanine ligase A | 38.28 | |
| | | (ddlA) | | |
| M369 F4 | H-P0739 | 2-hydroxy-6-oxohepta-2,4- | 26.62 | |
| | | dienoate hydrolase | | |
| M369 G4 | H-P0741 | conserved hypothetical protein | 17.82 | |
| M369 H4 | H-P0745 | conserved hypothetical protein | 36.08 | |
| M369 A5 | H-P0747 | conserved hypothetical protein | 43.34 | |
| M369 B5 | H-P0748 | cell division protein (ftsE) | 24.64 | |
| M369 C5 | H-P0749 | cell division membrane protein | 29.59 | |
| | | (ftsX) | | |
| M369 D5 | H-P0750 | hypothetical protein | 44.11 | |
| M369 E5 | H-P0752 | flagellar hook-associated protein | 74.25 | |
| | | 2 (fliD) | | |
| M381 F3 | H-P0755 | molybdopterin biosynthesis | 23.21 | |
| | | protein (moeB) | | |
| M379 C3 | H-P0757-2 | beta-alanine synthetase homolog | 32.23 | |
| M369 F5 | H-P0758 | conserved hypothetical integral | 48.18 | |
| | | membrane protein | | |
| M369 G5 | H-P0759 | conserved hypothetical integral | 45.98 | |
| | | membrane protein | | |
| M369 H5 | H-P0761 | hypothetical protein | 22.11 | |
| M369 A6 | H-P0762 | hypothetical protein | 20.46 | |
| M369 B6 | H-P0767 | hypothetical protein | 2.75 | |
| M369 C6 | H-P0768 | molybdenum cofactor | 35.42 | |
| | | biosynthesis protein A (moaA) | | |
| M369 D6 | H-P0769 | molybdopterin-guanine | 22.22 | |
| | | dinucleotide biosynthesis protein | | |
| | | A (mobA) | | |
| M369 E6 | H-P0771 | hypothetical protein | 27.06 | |
| M369 F6 | H-P0772 | N-acetylmuramoyl-L-alanine | 48.51 | |
| | | amidase (amiA) | | |
| M369 G6 | H-P0773 | hypothetical protein | 40.04 | |
| M369 H6 | H-P0777 | uridine 5′-monophosphate (UMP) | 26.51 | |
| | | kinase (pyrH) | | |
| M370 A1 | H-P0782 | hypothetical protein | 50.16 | |
| M370 B1 | H-P0783 | hypothetical protein | 18.26 | |
| M370 C1 | H-P0792 | sigma-54 interacting protein | 55.77 | |
| M370 D1 | H-P0793 | polypeptide deformylase (def) | 19.25 | |
| M370 E1 | H-P0794 | ATP-dependent clp protease | 21.67 | |
| | | proteolytic component (clpP) | | |
| M370 F1 | H-P0796 | outer membrane protein (omp18) | 30.69 | |
| M379 G3 | H-P0797-2 | flagellar sheath adhesin hpaA | 28.71 | |
| M379 H3 | H-P0798-2 | molybdenum cofactor | 17.49 | |
| | | biosynthesis protein C (moaC) | | |
| M370 G1 | H-P0799 | molybdopterin biosynthesis | 19.47 | |
| | | protein (mog) | | |
| M370 H1 | H-P0800 | molybdopterin converting factor, | 16.06 | |
| | | subunit 2 (moaE) | | |
| M379 A4 | H-P0801-2 | molybdopterin converting factor, | 8.25 | |
| | | subunit 1 (moaD) | | |
| M379 B4 | H-P0802-2 | GTP cyclohydrolase II (ribA) | 21.23 | |
| M379 D3 | H-P0803-2 | hypothetical protein | 30.8 | |
| M379 E3 | H-P0804-2 | GTP cyclohydrolase II/3,4- | 37.95 | |
| | | dihydroxy-2-butanone 4- | | |
| | | phosphate synthase (ribA, ribB) | | |
| M379 F3 | H-P0805-2 | lipooligosaccharide 5G8 epitope | 31.35 | |
| | | biosynthesis-associated protein | | |
| | | (lex2B) | | |
| M370 A2 | H-P0806 | hypothetical protein | 22.77 | |
| M379 C4 | H-P0807-2 | iron(III) dicitrate transport protein | 86.68 | |
| | | (fecA) | | |
| M370 B2 | H-P0808 | holo-acp synthase (acpS) | 13.2 | |
| M370 C2 | H-P0809 | hypothetical protein | 20.24 | |
| M370 D2 | H-P0810 | conserved hypothetical protein | 22.11 | |
| M370 E2 | H-P0811 | hypothetical protein | 11.99 | |
| M370 F2 | H-P0812 | hypothetical protein | 37.07 | |
| M370 G2 | H-P0813 | conserved hypothetical protein | 22.66 | |
| M370 H2 | H-P0814 | thiamin biosynthesis protein | 28.16 | |
| | | (thiF) | | |
| M370 A3 | H-P0815 | flagellar motor rotation protein | 28.38 | |
| | | (motA) | | |
| M370 B3 | H-P0831 | conserved hypothetical ATP | 21.67 | |
| | | binding protein | | |
| M379 D4 | H-P0832-2 | spermidine synthase (speE) | 28.93 | |
| M379 E4 | H-P0833-2 | hypothetical protein | 32.23 | |
| M370 C3 | H-P0834 | GTP-binding protein homologue | 50.49 | |
| | | (yphC) | | |
| M370 D3 | H-P0835 | histone-like DNA-binding protein | 10.45 | |
| | | HU (hup) | | |
| M370 E3 | H-P0836 | hypothetical protein | 13.2 | |
| M370 F3 | H-P0837 | hypothetical protein | 11.33 | |
| M370 G3 | H-P0838 | hypothetical protein | 22.66 | |
| M370 H3 | H-P0839 | outer membrane protein P1 | 64.68 | |
| | | (ompP1) | | |
| M370 A4 | H-P0840 | flaA1 protein | 36.74 | |
| M370 B4 | H-P0841 | pantothenate metabolism | 46.86 | |
| | | flavoprotein (dfp) | | |
| M370 C4 | H-P0843 | thiamin phosphate | 24.2 | |
| | | pyrophosphorylase/hyroxyethylthi | | |
| | | azole kinase (thiB) | | |
| M370 D4 | H-P0845 | thiamin phosphate | 30.14 | |
| | | pyrophosphorylase/hyroxyethylthi | | |
| | | azole kinase (thiM) | | |
| M370 E4 | H-P0850 | type I restriction enzyme M | 58.08 | |
| | | protein (hsdM) | | |
| M370 F4 | H-P0851 | conserved hypothetical integral | 25.08 | |
| | | membrane protein | | |
| M370 G4 | H-P0854 | GMP reductase (guaC) | 36.08 | |
| M370 H4 | H-P0858 | ADP-heptose synthase (rfaE) | 50.82 | |
| M370 A5 | H-P0859 | ADP-L-glycero-D-mannoheptose- | 36.41 | |
| | | 6-epimerase (rfaD) | | |
| M370 B5 | H-P0861 | hypothetical protein | 27.17 | |
| M370 C5 | H-P0862 | hypothetical protein | 24.64 | |
| M379 F4 | H-P0863-2 | hypothetical protein | 59.73 | |
| M370 D5 | H-P0865 | deoxyuridine 5′-triphosphate | 16.06 | |
| | | nucleotidohydrolase (dut) | | |
| M370 E5 | H-P0866 | transcription elongation factor | 18.15 | |
| | | GreA (greA) | | |
| M379 G4 | H-P0867-2 | lipid A disaccharide synthetase | 39.71 | |
| | | (lpxB) | | |
| M379 H4 | H-P0870-2 | flagellar hook (flgE) | 79.09 | |
| M370 F5 | H-P0871 | CDP-diglyceride hydrolase (cdh) | 26.95 | |
| M370 G5 | H-P0872 | alkylphosphonate uptake protein | 12.1 | |
| | | (phnA) | | |
| M370 H5 | H-P0873 | hypothetical protein | 7.92 | |
| M371 A1 | H-P0879 | hypothetical protein | 22.33 | |
| M371 B1 | H-P0883 | Holliday junction DNA helicase | 20.24 | |
| | | (ruvA) | | |
| M371 C1 | H-P0885 | virulence factor mviN protein | 50.82 | |
| | | (mviN) | | |
| M371 D1 | H-P0886 | cysteinyl-tRNA synthetase (cysS) | 51.26 | |
| M371 E1 | H-P0889 | iron(III) dicitrate ABC | 35.97 | |
| | | transporter, permease protein | | |
| | | (fecD) | | |
| M371 F1 | H-P0890 | conserved hypothetical protein | 28.27 | |
| M371 G1 | H-P0891 | conserved hypothetical protein | 19.25 | |
| M371 H1 | H-P0892 | conserved hypothetical protein | 10.01 | |
| M371 A2 | H-P0894 | conserved hypothetical protein | 9.79 | |
| M371 B2 | H-P0895 | hypothetical protein | 13.86 | |
| M371 C2 | H-P0896 | outer membrane protein (omp19) | 77.99 | |
| M371 D2 | H-P0897 | hypothetical protein | 22.99 | |
| M371 E2 | H-P0898 | hydrogenase expression/formation | 40.81 | |
| | | protein (hypD) | | |
| M371 F2 | H-P0899 | hydrogenase expression/formation | 8.58 | |
| | | protein (hypC) | | |
| M371 G2 | H-P0900 | hydrogenase expression/formation | 26.73 | |
| | | protein (hypB) | | |
| M371 H2 | H-P0905 | phosphotransacetylase (pta) | 24.64 | |
| M371 A3 | H-P0906 | hypothetical protein | 58.08 | |
| M371 B3 | H-P0907 | hook assembly protein, flagella | 33.22 | |
| | | (flgD) | | |
| M371 C3 | H-P0909 | hypothetical protein | 22.22 | |
| M371 D3 | H-P0912 | outer membrane protein (omp20) | 56.76 | |
| M371 E3 | H-P0913 | outer membrane protein (omp21) | 58.3 | |
| M371 F3 | H-P0914 | hypothetical protein | 56.65 | |
| M371 G3 | H-P0915 | iron-regulated outer membrane | 61.93 | |
| | | protein (frpB) | | |
| M371 H3 | H-P0916 | iron-regulated outer membrane | 27.5 | |
| | | protein (frpB) | | |
| M380 A1 | H-P0917-2 | hypothetical protein | 2.64 | |
| M371 A4 | H-P0918 | hypothetical protein | 15.84 | |
| M371 B4 | H-P0920 | conserved hypothetical integral | 25.41 | |
| | | membrane protein | | |
| M371 C4 | H-P0921 | glyceraldehyde-3-phosphate | 36.63 | |
| | | dehydrogenase (gap) | | |
| M371 D4 | H-P0923 | outer membrane protein (omp22) | 40.7 | |
| M371 E4 | H-P0925 | recombinational DNA repair | 21.34 | |
| | | protein (recR) | | |
| M371 F4 | H-P0927 | heat shock protein (htpX) | 35.97 | |
| M371 G4 | H-P0928 | GTP cyclohydrolase I (folE) | 19.91 | |
| M371 H4 | H-P0929 | geranyltranstransferase (ispA) | 33.44 | |
| M371 A5 | H-P0930 | stationary-phase survival protein | 29.48 | |
| | | (surE) | | |
| M371 B5 | H-P0931 | hypothetical protein | 16.17 | |
| M371 C5 | H-P0932 | hypothetical protein | 11.11 | |
| M371 D5 | H-P0933 | hypothetical protein | 22.11 | |
| M371 E5 | H-P0934 | conserved hypothetical protein | 27.72 | |
| M371 F5 | H-P0935 | hypothetical protein | 17.82 | |
| M371 G5 | H-P0936 | proline/betaine transporter (proP) | 42.9 | |
| M371 H5 | H-P0938 | hypothetical protein | 12.76 | |
| M371 A6 | H-P0939 | amino acid ABC transporter, | 26.18 | |
| | | permease protein (yckJ) | | |
| M371 B6 | H-P0940 | amino acid ABC transporter, | 28.27 | |
| | | periplasmic binding protein | | |
| | | (yckK) | | |
| M371 C6 | H-P0941 | alanine racemase, biosynthetic | 41.58 | |
| | | (alr) | | |
| M371 D6 | H-P0942 | D-alanine glycine permease | 49.61 | |
| | | (dagA) | | |
| M371 E6 | H-P0943 | D-amino acid dehydrogenase | 45.21 | |
| | | (dadA) | | |
| M371 F6 | H-P0944 | translation initiation inhibitor, | 13.86 | |
| | | putative | | |
| M371 G6 | H-P0946 | conserved hypothetical integral | 54.67 | |
| | | membrane protein | | |
| M371 H6 | H-P0947 | hypothetical protein | 13.31 | |
| M371 A7 | H-P0949 | conserved hypothetical secreted | 16.61 | |
| | | protein | | |
| M371 B7 | H-P0950 | acetyl-CoA carboxylase beta | 31.9 | |
| | | subunit (accD) | | |
| M371 C7 | H-P0951 | hypothetical protein | 22.66 | |
| M371 D7 | H-P0952 | conserved hypothetical integral | 24.09 | |
| | | membrane protein | | |
| M371 E7 | H-P0953 | hypothetical protein | 20.79 | |
| M371 F7 | H-P0955 | prolipoprotein diacylglyceryl | 31.35 | |
| | | transferase (lgt) | | |
| M371 G7 | H-P0956 | conserved hypothetical protein | 26.73 | |
| M371 H7 | H-P0957 | 3-deoxy-d-manno-octulosonic- | 43.34 | |
| | | acid transferase (kdtA) | | |
| M371 A8 | H-P0958 | hypothetical protein | 28.05 | |
| M371 B8 | H-P0960 | glycyl-tRNA synthetase, alpha | 33.44 | |
| | | subunit (glyQ) | | |
| M371 C8 | H-P0961 | glycerol-3-phosphate | 34.43 | |
| | | dehydrogenase (NAD(P)+) | | |
| M380 B1 | H-P0965-2 | hypothetical protein | 48.84 | |
| M371 D8 | H-P0966 | conserved hypothetical protein | 60.5 | |
| M380 F1 | H-P0968-2 | hypothetical protein | 2.42 | |
| M371 E8 | H-P0969 | cation efflux system protein | 112.31 | |
| | | (czcA) | | |
| M371 F8 | H-P0970 | nickel-cobalt-cadmium resistance | 39.6 | |
| | | protein (nccB) | | |
| M371 G8 | H-P0971 | hypothetical protein | 45.54 | |
| M371 H8 | H-P0972 | glycyl-tRNA synthetase, beta | 77.22 | |
| | | subunit (glyS) | | |
| M371 A9 | H-P0973 | hypothetical protein | 38.94 | |
| M380 C1 | H-P0974-2 | phosphoglycerate mutase (pgm) | 54.12 | |
| M380 D1 | H-P0975-2 | conserved hypothetical protein | 10.34 | |
| M380 E1 | H-P0976-2 | adenosylmethionine-8-amino-7- | 48.07 | |
| | | oxononanoate aminotransferase | | |
| | | (bioA) | | |
| M380 H1 | H-P0994-2 | hypothetical protein | 29.48 | |
| M380 G1 | H-P1000-2 | PARA protein | 24.09 | |
| M380 A2 | H-P1001-2 | hypothetical protein | 10.45 | |
| M380 B2 | H-P1002-2 | hypothetical protein | 43.45 | |
| M380 C2 | H-P1003-2 | hypothetical protein | 40.81 | |
| M380 D2 | H-P1004-2 | hypothetical protein | 30.14 | |
| M380 E2 | H-P1005-2 | hypothetical protein | 11.55 | |
| M380 F2 | H-P1006-2 | conjugal transfer protein (traG) | 19.58 | |
| M380 G2 | H-P1017-2 | amino acid permease (rocE) | 57.2 | |
| M380 H2 | H-P1042-2 | hypothetical protein | 38.39 | |
| M380 A3 | H-P1056-2 | hypothetical protein | 31.35 | |
| M380 B3 | H-P1075-2 | conserved hypothetical secreted | 48.29 | |
| | | protein | | |
| M373 A1 | H-P1076 | hypothetical protein | 18.92 | |
| M373 B1 | H-P1077 | nickel transport protein (nixA) | 36.52 | |
| M373 C1 | H-P1080 | conserved hypothetical integral | 20.9 | |
| | | membrane protein | | |
| M373 D1 | H-P1081 | hypothetical protein | 22.88 | |
| M373 E1 | H-P1082 | multidrug resistance protein | 60.72 | |
| | | (msbA) | | |
| M373 F1 | H-P1083 | hypothetical protein | 52.8 | |
| M373 G1 | H-P1084 | aspartate transcarbamoylase | 33.88 | |
| | | (pyrB) | | |
| M373 H1 | H-P1085 | hypothetical protein | 18.92 | |
| M373 A2 | H-P1086 | hemolysin (tly) | 25.96 | |
| M373 B2 | H-P1087 | riboflavin biosynthesis regulatory | 30.91 | |
| | | protein (ribC) | | |
| M373 C2 | H-P1088 | transketolase A (tktA) | 70.62 | |
| M373 D2 | H-P1091 | alpha-ketoglutarate permease | 46.97 | |
| | | (kgtP) | | |
| M373 E2 | H-P1092 | flagellar basal-body rod protein | 29.7 | |
| | | (flgG) | | |
| M373 F2 | H-P1096 | IS605 transposase (tnpA) | 15.73 | |
| M373 G2 | H-P1098 | conserved hypothetical secreted | 32.01 | |
| | | protein | | |
| M373 H2 | H-P1101 | glucose-6-phosphate | 46.86 | |
| | | dehydrogenase (g6pD) | | |
| M373 A3 | H-P1102 | glucose-6-phosphate 1- | 25.08 | |
| | | dehydrogenase (devB) | | |
| M373 B3 | H-P1103 | glucokinase (glk) | 37.07 | |
| M373 C3 | H-P1108 | pyruvate ferredoxin | 20.57 | |
| | | oxidoreductase, gamma subunit | | |
| M373 D3 | H-P1109 | pyruvate ferredoxin | 14.41 | |
| | | oxidoreductase, delta subunit | | |
| M373 E3 | H-P1110 | pyruvate ferredoxin | 44.88 | |
| | | oxidoreductase, alpha subunit | | |
| M373 F3 | H-P1111 | pyruvate ferredoxin | 34.65 | |
| | | oxidoreductase, beta subunit | | |
| M373 G3 | H-P1112 | adenylosuccinate lyase (purB) | 48.51 | |
| M380 C3 | H-P1113-2 | outer membrane protein (omp24) | 30.58 | |
| M373 H3 | H-P1117 | conserved hypothetical secreted | 28.27 | |
| | | protein | | |
| M373 A4 | H-P1120 | hypothetical protein | 15.95 | |
| M373 B4 | H-P1121 | cytosine specific DNA | 34.43 | |
| | | methyltransferase (BSP6IM) | | |
| M380 D3 | H-P1122-2 | hypothetical protein | 8.47 | |
| M373 C4 | H-P1123 | peptidyl-prolyl cis-trans | 20.46 | |
| | | isomerase, FKBP-type rotamase | | |
| | | (slyD) | | |
| M373 D4 | H-P1124 | hypothetical protein | 36.52 | |
| M373 E4 | H-P1125 | peptidoglycan associated | 19.8 | |
| | | lipoprotein precursor (omp18) | | |
| M373 F4 | H-P1126 | colicin tolerance-like protein | 45.98 | |
| | | (tolB) | | |
| M373 G4 | H-P1128 | hypothetical protein | 9.35 | |
| M373 H4 | H-P1129 | biopolymer transport protein | 14.74 | |
| | | (exbD) | | |
| M373 A5 | H-P1131 | ATP synthase F1, subunit epsilon | 13.75 | |
| | | (atpC) | | |
| M373 B5 | H-P1134 | ATP synthase F1, subunit alpha | 55.44 | |
| | | (atpA) | | |
| M373 C5 | H-P1135 | ATP synthase F1, subunit delta | 19.91 | |
| | | (atpH) | | |
| M373 D5 | H-P1137 | ATP synthase F0, subunit b' | 15.95 | |
| | | (atpF') | | |
| M373 E5 | H-P1138 | plasmid replication-partition | 32.01 | |
| | | related protein | | |
| M373 F5 | H-P1139 | SpoOJ regulator (soj) | 29.15 | |
| M373 G5 | H-P1140 | biotin operon repressor/biotin | 23.43 | |
| | | acetyl coenzyme A carboxylase | | |
| | | synthetase (birA) | | |
| M373 H5 | H-P1141 | methionyl-tRNA | 33.44 | |
| | | formyltransferase (fmt) | | |
| M373 A6 | H-P1144 | hypothetical protein | 9.46 | |
| M373 B6 | H-P1145 | hypothetical protein | 11.44 | |
| M373 C6 | H-P1147 | ribosomal protein L19 (rpl19) | 13.09 | |
| M373 D6 | H-P1148 | tRNA (guanine-N1)- | 25.3 | |
| | | methyltransferase (trmD) | | |
| M373 E6 | H-P1149 | conserved hypothetical protein | 20.35 | |
| M380 F3 | H-P1150-2 | hypothetical protein | 12.76 | |
| M373 F6 | H-P1152 | signal recognition particle protein | 49.39 | |
| | | (ffh) | | |
| M380 G3 | H-P1153-2 | valyl-tRNA synthetase (valS) | 96.25 | |
| M380 E3 | H-P1157-2 | outer membrane protein (omp26) | 135.41 | |
| M373 G6 | H-P1158 | pyrroline-5-carboxylate reductase | 28.38 | |
| | | (proC) | | |
| M373 H6 | H-P1159 | cell filamentation protein (fic) | 19.58 | |
| M373 A7 | H-P1160 | conserved hypothetical protein | 15.51 | |
| M380 A4 | H-P1163-2 | hypothetical protein | 7.04 | |
| M373 B7 | H-P1165 | tetracycline resistance protein | 42.57 | |
| | | tetA(P), putative | | |
| M373 C7 | H-P1168 | carbon starvation protein (cstA) | 75.68 | |
| M373 D7 | H-P1169 | glutamine ABC transporter, | 23.98 | |
| | | permease protein (glnP) | | |
| M380 H3 | H-P1169-2 | glutamine ABC transporter, | 23.98 | |
| | | permease protein (glnP) | | |
| M374 A1 | H-P1170 | glutamine ABC transporter, | 24.64 | |
| | | permease protein (glnP) | | |
| M374 B1 | H-P1171 | glutamine ABC transporter, ATP- | 27.39 | |
| | | binding protein (glnQ) | | |
| M374 C1 | H-P1172 | glutamine ABC transporter, | 30.58 | |
| | | periplasmic glutamine-binding | | |
| | | protein (glnH) | | |
| M374 D1 | H-P1173 | hypothetical protein | 20.24 | |
| M374 E1 | H-P1174 | glucose/galactose transporter | 44.88 | |
| | | (gluP) | | |
| M374 F1 | H-P1175 | conserved hypothetical integral | 47.96 | |
| | | membrane protein | | |
| M374 G1 | H-P1177 | outer membrane protein (omp27) | 70.62 | |
| M374 H1 | H-P1178 | purine-nucleoside phosphorylase | 25.74 | |
| | | (deoD) | | |
| M374 A2 | H-P1179 | phosphopentomutase (deoB) | 45.54 | |
| M374 B2 | H-P1180 | pyrimidine nucleoside transport | 46.09 | |
| | | protein (nupC) | | |
| M374 C2 | H-P1183 | NA+/H+ antiporter (napA) | 42.24 | |
| M374 D2 | H-P1184 | conserved hypothetical integral | 50.6 | |
| | | membrane protein | | |
| M374 E2 | H-P1185 | conserved hypothetical integral | 43.12 | |
| | | membrane protein | | |
| M374 F2 | H-P1186 | carbonic anhydrase | 22.33 | |
| M374 G2 | H-P1187 | hypothetical protein | 42.46 | |
| M374 H2 | H-P1188 | hypothetical protein | 29.7 | |
| M374 A3 | H-P1189 | aspartate-semialdehyde | 38.17 | |
| | | dehydrogenase (asd) | | |
| M374 B3 | H-P1191 | ADP-heptose-lps | 38.5 | |
| | | heptosyltransferase II (rfaF) | | |
| M374 C3 | H-P1196 | ribosomal protein S7 (rps7) | 17.16 | |
| M374 D3 | H-P1200 | ribosomal protein L10 (rpl10) | 18.15 | |
| M374 E3 | H-P1201 | ribosomal protein L1 (rpl1) | 25.85 | |
| M374 F3 | H-P1202 | ribosomal protein L11 (rpl11) | 15.62 | |
| M374 G3 | H-P1203 | transcription termination factor | 19.47 | |
| | | NusG (nusG) | | |
| M380 B4 | H-P1205-2 | translation elongation factor EF- | 44 | |
| | | Tu (tufB) | | |
| M374 H3 | H-P1206 | multidrug resistance protein | 63.69 | |
| | | (hetA) | | |
| M374 A4 | H-P1207 | hypothetical protein | 24.53 | |
| M374 B4 | H-P1210 | serine acetyltransferase (cysE) | 18.92 | |
| M380 F4 | H-P1213-2 | polynucleotide phosphorylase | 75.79 | |
| | | (pnp) | | |
| M380 G4 | H-P1214-2 | conserved hypothetical protein | 26.51 | |
| M380 C4 | H-P1215-2 | hypothetical protein | 8.91 | |
| M380 D4 | H-P1216-2 | conserved hypothetical secreted | 72.71 | |
| | | protein | | |
| M380 E4 | H-P1217-2 | hypothetical protein | 17.6 | |
| M374 C4 | H-P1220 | ABC transporter, ATP-binding | 25.19 | |
| | | protein (yhcG) | | |
| M374 D4 | H-P1221 | conserved hypothetical protein | 25.85 | |
| M374 E4 | H-P1222 | D-lactate dehydrogenase (dld) | 104.39 | |
| M374 F4 | H-P1224 | uroporphyrinogen III cosynthase | 24.97 | |
| | | (hemD) | | |
| M374 G4 | H-P1225 | conserved hypothetical integral | 14.41 | |
| | | membrane protein | | |
| M374 H4 | H-P1226 | oxygen-independent | 38.83 | |
| | | coproporphyrinogen III oxidase | | |
| | | (hemN) | | |
| M380 H4 | H-P1227-2 | cytochrome c553 | 10.67 | |
| M380 A5 | H-P1228-2 | invasion protein (invA) | 17.16 | |
| M380 B5 | H-P1229-2 | aspartokinase (lysC) | 44.66 | |
| M374 A5 | H-P1230 | hypothetical protein | 19.91 | |
| M374 B5 | H-P1231 | DNA polymerase III delta prime | 24.09 | |
| | | subunit (holB) | | |
| M374 C5 | H-P1232 | dihydropteroate synthase (folP) | 41.91 | |
| M380 D5 | H-P1233-2 | hypothetical protein | 16.94 | |
| M374 D5 | H-P1234 | conserved hypothetical integral | 32.89 | |
| | | membrane protein | | |
| M374 E5 | H-P1235 | conserved hypothetical integral | 45.76 | |
| | | membrane protein | | |
| M374 F5 | H-P1236 | hypothetical protein | 20.24 | |
| M374 G5 | H-P1237 | carbamoyl-phosphate synthetase | 41.36 | |
| | | (pyrAa) | | |
| M374 H5 | H-P1240 | conserved hypothetical protein | 21.01 | |
| M380 C5 | H-P1241-2 | alanyl-tRNA synthetase (alaS) | 93.28 | |
| M374 A6 | H-P1242 | conserved hypothetical protein | 8.47 | |
| M380 H5 | H-P1243-2 | outer membrane protein (omp28) | 80.74 | |
| M374 B6 | H-P1244 | ribosomal protein S18 (rps18) | 9.46 | |
| M374 C6 | H-P1245 | single-strand DNA-binding | 19.8 | |
| | | protein (ssb) | | |
| M374 D6 | H-P1246 | ribosomal protein S6 (rps6) | 15.73 | |
| M380 A6 | H-P1247-2 | hypothetical protein | 37.51 | |
| M374 E6 | H-P1248 | virulence associated protein | 70.95 | |
| | | homolog (vacB) | | |
| M380 B6 | H-P1249-2 | shikimate 5-dehydrogenase (aroE) | 29.04 | |
| M380 E5 | H-P1251-2 | oligopeptide ABC transporter, | 38.39 | |
| | | permease protein (oppB) | | |
| M380 F5 | H-P1252-2 | oligopeptide ABC transporter, | 65.45 | |
| | | periplasmic oligopeptide-binding | | |
| | | protein (oppA) | | |
| M380 G5 | H-P1253-2 | tryptophanyl-tRNA synthetase | 37.4 | |
| | | (trpS) | | |
| M374 F6 | H-P1254 | biotin synthesis protein (bioC) | 26.51 | |
| M374 G6 | H-P1255 | protein translocation protein, low | 22.22 | |
| | | temperature (secG) | | |
| M374 H6 | H-P1256 | ribosome releasing factor (frr) | 20.46 | |
| M374 A7 | H-P1257 | orotate phosphoribosyltransferase | 22.22 | |
| | | (pyrE) | | |
| M374 B7 | H-P1258 | conserved hypothetical | 17.05 | |
| | | mitochondrial protein 4 | | |
| M374 C7 | H-P1260 | NADH-ubiquinone | 14.74 | |
| | | oxidoreductase, NQO7 subunit | | |
| | | (NQO7) | | |
| M374 D7 | H-P1262 | NADH-ubiquinone | 29.37 | |
| | | oxidoreductase, NQO5 subunit | | |
| | | (NQO5) | | |
| M374 E7 | H-P1263 | NADH-ubiquinone | 45.1 | |
| | | oxidoreductase, NQO4 subunit | | |
| | | (NQO4) | | |
| M380 C6 | H-P1264-2 | hypothetical protein | 8.47 | |
| M374 F7 | H-P1265 | hypothetical protein | 36.19 | |
| M375 A1 | H-P1268 | NADH-ubiquinone | 24.31 | |
| | | oxidoreductase, NQO9 subunit | | |
| | | (NQO9) | | |
| M375 B1 | H-P1275 | phosphomannomutase (algC) | 50.6 | |
| M375 C1 | H-P1277 | tryptophan synthase, alpha | 28.93 | |
| | | subunit (trpA) | | |
| M375 D1 | H-P1278 | tryptophan synthase, beta subunit | 43.34 | |
| | | (trpB) | | |
| M375 E1 | H-P1279 | anthranilate isomerase (trpC) | 49.83 | |
| M375 F1 | H-P1282 | anthranilate synthase component I | 55.11 | |
| | | (trpE) | | |
| M375 G1 | H-P1285 | conserved hypothetical secreted | 25.41 | |
| | | protein | | |
| M375 H1 | H-P1286 | conserved hypothetical secreted | 20.13 | |
| | | protein | | |
| M375 A2 | H-P1287 | transcriptional regulator (tenA) | 23.98 | |
| M375 B2 | H-P1288 | hypothetical protein | 14.63 | |
| M375 C2 | H-P1289 | hypothetical protein | 17.82 | |
| M375 D2 | H-P1290 | nicotinamide mononucleotide | 24.31 | |
| | | transporter (pnuC) | | |
| M375 E2 | H-P1291 | conserved hypothetical protein | 22.55 | |
| M375 F2 | H-P1292 | ribosomal protein L17 (rpl17) | 12.87 | |
| M375 G2 | H-P1293 | DNA-directed RNA polymerase, | 37.95 | |
| | | alpha subunit (rpoA) | | |
| M375 H2 | H-P1294 | ribosomal protein S4 (rps4) | 22.99 | |
| M375 A3 | H-P1295 | ribosomal protein S11 (rps11) | 14.52 | |
| M375 B3 | H-P1296 | ribosomal protein S13 (rps13) | 13.31 | |
| M380 D6 | H-P1298-2 | translation initiation factor EF-1 | 8.03 | |
| | | (infA) | | |
| M375 C3 | H-P1299 | methionine amino peptidase | 27.94 | |
| | | (map) | | |
| M375 D3 | H-P1302 | ribosomal protein S5 (rps5) | 16.94 | |
| M375 E3 | H-P1303 | ribosomal protein L18 (rpl18) | 13.2 | |
| M375 F3 | H-P1305 | ribosomal protein S8 (rps8) | 14.52 | |
| M375 G3 | H-P1307 | ribosomal protein L5 (rpl5) | 20.02 | |
| M375 H3 | H-P1308 | ribosomal protein L24 (rpl24) | 8.14 | |
| M375 A4 | H-P1309 | ribosomal protein L14 (rpl14) | 13.53 | |
| M375 B4 | H-P1310 | ribosomal protein S17 (rps17) | 9.57 | |
| M375 C4 | H-P1312 | ribosomal protein L16 (rpl16) | 15.62 | |
| M375 D4 | H-P1314 | ribosomal protein L22 (rpl22) | 13.53 | |
| M375 E4 | H-P1315 | ribosomal protein S19 (rps19) | 10.34 | |
| M375 F4 | H-P1318 | ribosomal protein L4 (rpl4) | 23.76 | |
| M375 G4 | H-P1319 | ribosomal protein L3 (rpl3) | 21.12 | |
| M375 H4 | H-P1320 | ribosomal protein S10 (rps10) | 11.55 | |
| M375 A5 | H-P1321 | conserved hypothetical ATP- | 41.58 | |
| | | binding protein | | |
| M375 B5 | H-P1322 | hypothetical protein | 22.22 | |
| M375 C5 | H-P1323 | ribonuclease HII (rnhB) | 23.1 | |
| M375 D5 | H-P1324 | hypothetical protein | 9.24 | |
| M375 E5 | H-P1326 | hypothetical protein | 13.86 | |
| M375 F5 | H-P1327 | hypothetical protein | 45.43 | |
| M375 G5 | H-P1328 | cation efflux system protein | 37.29 | |
| | | (czcA) | | |
| M375 H5 | H-P1330 | conserved hypothetical integral | 12.76 | |
| | | membrane protein | | |
| M375 A6 | H-P1331 | conserved hypothetical integral | 25.19 | |
| | | membrane protein | | |
| M375 B6 | H-P1332 | co-chaperone and heat shock | 40.7 | |
| | | protein (dnaJ) | | |
| M375 C6 | H-P1333 | hypothetical protein | 42.13 | |
| M375 D6 | H-P1335 | conserved hypothetical protein | 39.71 | |
| M375 E6 | H-P1336 | hypothetical protein | 27.94 | |
| M375 F6 | H-P1337 | conserved hypothetical protein | 19.25 | |
| M375 G6 | H-P1338 | conserved hypothetical protein | 16.39 | |
| M375 H6 | H-P1340 | biopolymer transport protein | 14.3 | |
| | | (exbD) | | |
| M375 A7 | H-P1341 | siderophore-mediated iron | 31.46 | |
| | | transport protein (tonB) | | |
| M375 B7 | H-P1342 | outer membrane protein (omp29) | 76.12 | |
| M375 C7 | H-P1343 | conserved hypothetical integral | 26.73 | |
| | | membrane protein | | |
| M375 D7 | H-P1344 | magnesium and cobalt transport | 35.09 | |
| | | protein (corA) | | |
| M375 E7 | H-P1345 | phosphoglycerate kinase | 44.33 | |
| M375 F7 | H-P1346 | glyceraldehyde-3-phosphate | 36.41 | |
| | | dehydrogenase (gap) | | |
| M375 G7 | H-P1347 | uracil-DNA glycosylase (ung) | 25.74 | |
| M375 H7 | H-P1349 | hypothetical protein | 42.68 | |
| M375 A8 | H-P1350 | protease | 50.6 | |
| M375 B8 | H-P1355 | nicotinate-nucleotide | 30.14 | |
| | | pyrophosphorylase (nadC) | | |
| M375 C8 | H-P1356 | quinolinate synthetase A (nadA) | 37.07 | |
| M375 D8 | H-P1357 | phosphatidylserine decarboxylase | 29.48 | |
| | | proenzyme (psd) | | |
| M375 E8 | H-P1358 | hypothetical protein | 18.59 | |
| M375 F8 | H-P1360 | 4-hydroxybenzoate | 32.45 | |
| | | octaprenyltransferase (ubiA) | | |
| M375 G8 | H-P1361 | competence locus E (comE3) | 45.98 | |
| M375 H8 | H-P1362 | replicative DNA helicase (dnaB) | 53.79 | |
| M375 A9 | H-P1363 | conserved hypothetical integral | 51.37 | |
| | | membrane protein | | |
| M376 A1 | H-P1364 | signal-transducing protein, | 43.78 | |
| | | histidine kinase | | |
| M376 B1 | H-P1365 | response regulator | 23.54 | |
| M376 C1 | H-P1371 | type III restriction enzyme R | 106.59 | |
| | | protein | | |
| M376 D1 | H-P1372 | rod shape-determining protein | 27.39 | |
| | | (mreC) | | |
| M376 E1 | H-P1373 | rod shape-determining protein | 38.28 | |
| | | (mreB) | | |
| M376 F1 | H-P1374 | ATP-dependent protease ATPase | 49.17 | |
| | | subunit (clpX) | | |
| M376 G1 | H-P1375 | UDP-N-acetylglucosamine | 29.81 | |
| | | acyltransferase (lpxA) | | |
| M376 H1 | H-P1376 | (3R)-hydroxymyristoyl-(acyl | 17.6 | |
| | | carrier protein) dehydratase | | |
| | | (fabZ) | | |
| M376 A2 | H-P1377 | hypothetical protein | 16.17 | |
| M376 B2 | H-P1378 | competence lipoprotein (comL) | 24.31 | |
| M376 C2 | H-P1379 | ATP-dependent protease (lon) | 91.96 | |
| M376 D2 | H-P1380 | prephenate dehydrogenase (tyrA) | 29.26 | |
| M381 C1 | H-P1381 | hypothetical protein | 8.58 | |
| M376 E2 | H-P1382 | hypothetical protein | 14.41 | |
| M376 F2 | H-P1383 | restriction modification system S | 17.71 | |
| | | subunit | | |
| M376 G2 | H-P1384 | hypothetical protein | 7.59 | |
| M376 H2 | H-P1385 | fructose-1,6-bisphosphatase | 32.01 | |
| M376 A3 | H-P1386 | D-ribulose-5-phosphate 3 | 23.98 | |
| | | epimerase (rpe) | | |
| M376 B3 | H-P1388 | hypothetical protein | 16.5 | |
| M376 C3 | H-P1389 | hypothetical protein | 6.71 | |
| M376 D3 | H-P1390 | hypothetical protein | 18.37 | |
| M376 E3 | H-P1391 | hypothetical protein | 10.89 | |
| M376 F3 | H-P1392 | fibronectin/fibrinogen-binding | 47.96 | |
| | | protein | | |
| M376 G3 | H-P1393 | DNA repair protein (recN) | 57.75 | |
| M376 H3 | H-P1394 | conserved hypothetical protein | 31.35 | |
| M376 A4 | H-P1395 | outer membrane protein (omp30) | 26.73 | |
| M376 B4 | H-P1396 | hypothetical protein | 31.79 | |
| M376 C4 | H-P1398 | alanine dehydrogenase (ald) | 41.91 | |
| M376 D4 | H-P1399 | arginase (rocF) | 35.53 | |
| M376 E4 | H-P1400 | iron(III) dicitrate transport protein | 92.73 | |
| | | (fecA) | | |
| M376 F4 | H-P1401 | conserved hypothetical protein | 25.96 | |
| M381 A2 | H-P1402 | type I restriction enzyme R | 109.34 | |
| | | protein (hsdR) | | |
| M381 B2 | H-P1403 | type I restriction enzyme M | 89.98 | |
| | | protein (hsdM) | | |
| M376 G4 | H-P1405 | hypothetical protein | 3.85 | |
| M376 H4 | H-P1406 | biotin synthetase (bioB) | 31.13 | |
| M376 A5 | H-P1407 | conserved hypothetical integral | 32.23 | |
| | | membrane protein | | |
| M381 C2 | H-P1408 | hypothetical protein | 12.32 | |
| M381 D2 | H-P1409 | hypothetical protein | 63.69 | |
| M376 B5 | H-P1410 | hypothetical protein | 43.45 | |
| M376 C5 | H-P1411 | hypothetical protein | 68.2 | |
| M376 D5 | H-P1412 | hypothetical protein | 33.99 | |
| M376 E5 | H-P1413 | conserved hypothetical protein | 16.39 | |
| M376 F5 | H-P1414 | conserved hypothetical protein | 12.54 | |
| M376 G5 | H-P1415 | tRNA delta(2)- | 29.37 | |
| | | isopentenylpyrophosphate | | |
| | | transferase (miaA) | | |
| M376 H5 | H-P1418 | UDP-N- | 28.6 | |
| | | acetylenolpyruvoylglucosamine | | |
| | | reductase (murB) | | |
| M376 A6 | H-P1419 | flagellar biosynthetic protein | 9.79 | |
| | | (fliQ) | | |
| M376 B6 | H-P1420 | flagellar export protein ATP | 47.85 | |
| | | synthase (fliI) | | |
| M376 C6 | H-P1421 | conjugative transfer regulon | 33.55 | |
| | | protein (trbB) | | |
| M376 D6 | H-P1423 | conserved hypothetical protein | 9.35 | |
| M376 E6 | H-P1424 | hypothetical protein | 22.77 | |
| M376 F6 | H-P1425 | hypothetical protein | 8.36 | |
| M376 G6 | H-P1427 | histidine-rich, metal binding | 6.71 | |
| | | polypeptide (hpn) | | |
| M376 H6 | H-P1428 | conserved hypothetical protein | 39.38 | |
| M376 A7 | H-P1429 | polysialic acid capsule expression | 36.3 | |
| | | protein (kpsF) | | |
| M376 B7 | H-P1430 | conserved hypothetical ATP- | 75.9 | |
| | | binding protein | | |
| M376 C7 | H-P1431 | 16S rRNA (adenosine-N6,N6-)- | 29.92 | |
| | | dimethyltransferase (ksgA) | | |
| M376 D7 | H-P1432 | histidine and glutamine-rich | 8.03 | |
| | | protein | | |
| M376 E7 | H-P1433 | hypothetical protein | 94.27 | |
| M376 F7 | H-P1434 | formyltetrahydrofolate hydrolase | 32.34 | |
| | | (purU) | | |
| M376 G7 | H-P1435 | protease IV (PspA) | 32.23 | |
| M376 H7 | H-P1436 | hypothetical protein | 9.13 | |
| M376 A8 | H-P1438 | conserved hypothetical | 37.29 | |
| | | lipoprotein | | |
| M376 B8 | H-P1439 | hypothetical protein | 9.02 | |
| M376 C8 | H-P1440 | hypothetical protein | 28.6 | |
| M376 D8 | H-P1441 | peptidyl-prolyl cis-trans | 18.04 | |
| | | isomerase B, cyclosporin-type | | |
| | | rotamase (ppi) | | |
| M376 E8 | H-P1442 | carbon storage regulator (csrA) | 8.47 | |
| M376 F8 | H-P1443 | conserved hypothetical protein | 29.59 | |
| M376 G8 | H-P1444 | small protein (smpB) | 16.83 | |
| M376 H8 | H-P1445 | biopolymer transport protein | 16.61 | |
| | | (exbB) | | |
| M376 A9 | H-P1446 | biopolymer transport protein | 14.74 | |
| | | (exbD) | | |
| M376 B9 | H-P1447 | ribosomal protein L34 (rpl34) | 4.95 | |
| M376 C9 | H-P1448 | ribonuclease P, protein | 17.82 | |
| | | component (rnpA) | | |
| M376 D9 | H-P1449 | conserved hypothetical protein | 12.98 | |
| M376 E9 | H-P1450 | 60 kDa inner-membrane protein | 60.28 | |
| M376 F9 | H-P1451 | hypothetical protein | 29.15 | |
| M376 G9 | H-P1452 | thiophene and furan oxidizer | 50.82 | |
| | | (tdhF) | | |
| M376 H9 | H-P1453 | conserved hypothetical protein | 82.17 | |
| M376 A10 | H-P1454 | hypothetical protein | 33.44 | |
| M376 B10 | H-P1455 | hypothetical protein | 14.41 | |
| M376 C10 | H-P1456 | membrane-associated lipoprotein | 19.36 | |
| | | (lpp20) | | |
| M376 D10 | H-P1457 | hypothetical protein | 23.21 | |
| M376 E10 | H-P1458 | thioredoxin | 11.55 | |
| M376 F10 | H-P1461 | cytochrome c551 peroxidase | 38.61 | |
| M377 A1 | H-P1462 | secreted protein involved in | 19.03 | |
| | | flagellar motility | | |
| M377 B1 | H-P1463 | hypothetical protein | 24.86 | |
| M377 C1 | H-P1464 | conserved hypothetical secreted | 29.92 | |
| | | protein | | |
| M377 D1 | H-P1465 | ABC transporter, ATP-binding | 28.82 | |
| | | protein (HI1087) | | |
| M377 E1 | H-P1466 | conserved hypothetical integral | 41.58 | |
| | | membrane protein | | |
| M377 F1 | H-P1467 | hypothetical protein | 25.52 | |
| M377 G1 | H-P1468 | branched-chain-amino-acid | 37.51 | |
| | | aminotransferase (ilvE) | | |
| M377 H1 | H-P1469 | outer membrane protein (omp31) | 27.39 | |
| M377 A2 | H-P1473 | hypothetical protein | 21.12 | |
| M377 B2 | H-P1474 | thymidylate kinase (tmk) | 21.12 | |
| M377 C2 | H-P1475 | lipopolysaccharide core | 17.38 | |
| | | biosynthesis protein (kdtB) | | |
| M377 D2 | H-P1476 | phenylacrylic acid decarboxylase | 20.68 | |
| M377 E2 | H-P1479 | hypothetical protein | 92.95 | |
| M377 F2 | H-P1480 | seryl-tRNA synthetase (serS) | 45.76 | |
| M377 G2 | H-P1481 | hypothetical protein | 29.26 | |
| M377 H2 | H-P1482 | hypothetical protein | 9.57 | |
| M377 A3 | H-P1483 | gerC2 protein (gerC2) | 27.17 | |
| M377 B3 | H-P1484 | conserved hypothetical integral | 16.39 | |
| | | membrane protein | | |
| M377 C3 | H-P1485 | proline dipeptidase (pepQ) | 21.01 | |
| M377 D3 | H-P1486 | conserved hypothetical integral | 41.47 | |
| | | membrane protein | | |
| M377 E3 | H-P1487 | conserved hypothetical integral | 40.26 | |
| | | membrane protein | | |
| M377 F3 | H-P1488 | conserved hypothetical secreted | 36.3 | |
| | | protein | | |
| M377 G3 | H-P1489 | lipase-like protein | 56.21 | |
| M381 G1 | H-P1490 | hemolysin | 49.5 | |
| M377 H3 | H-P1491 | phosphate permease | 58.74 | |
| M377 A4 | H-P1492 | conserved hypothetical nifU-like | 9.9 | |
| | | protein | | |
| M377 B4 | H-P1493 | hypothetical protein | 22.44 | |
| M377 C4 | H-P1494 | UDP-MurNac-tripeptide | 49.28 | |
| | | synthetase (murE) | | |
| M377 D4 | H-P1495 | transaldolase (tal) | 34.87 | |
| M377 E4 | H-P1496 | general stress protein (ctc) | 19.69 | |
| M377 F4 | H-P1497 | peptidyl-tRNA hydrolase (pth) | 20.57 | |
| M377 G4 | H-P1499 | hypothetical protein | 30.03 | |
| M377 H4 | H-P1501 | outer membrane protein (omp32) | 42.79 | |
| M377 A5 | H-P1502 | hypothetical protein | 16.06 | |
| M377 B5 | H-P1503 | cation-transporting ATPase, P- | 86.79 | |
| | | type (copA) | | |
| M377 C5 | H-P1504 | conserved hypothetical protein | 26.29 | |
| M377 D5 | H-P1505 | riboflavin biosynthesis protein | 37.95 | |
| | | (ribG) | | |
| M377 E5 | H-P1506 | glutamate permease (gltS) | 44.99 | |
| M377 F5 | H-P1507 | conserved hypothetical ATP- | 42.46 | |
| | | binding protein | | |
| M381 F2 | H-P1508 | ferrodoxin-like protein | 50.49 | |
| M377 G5 | H-P1509 | conserved hypothetical integral | 28.93 | |
| | | membrane protein | | |
| M377 H5 | H-P1510 | conserved hypothetical protein | 12.98 | |
| M377 A6 | H-P1511 | hypothetical protein | 11.99 | |
| M377 B6 | H-P1512 | iron-regulated outer membrane | 96.58 | |
| | | protein (frpB) | | |
| M377 C6 | H-P1513 | selenocystein synthase (selA) | 42.57 | |
| M377 D6 | H-P1514 | transcription termination factor | 43.56 | |
| | | NusA (nusA) | | |
| M377 E6 | H-P1518 | hypothetical protein | 10.56 | |
| M381 B3 | H-P1521 | type III restriction enzyme R | 106.48 | |
| | | protein (res) | | |
| M381 C3 | H-P1523 | DNA recombinase (recG) | 68.64 | |
| M377 F6 | H-P1524 | hypothetical protein | 12.76 | |
| M377 G6 | H-P1525 | hypothetical protein | 23.32 | |
| M377 H6 | H-P1526 | exodeoxyribonuclease (lexA) | 27.61 | |
| M377 A7 | H-P1527 | hypothetical protein | 52.8 | |
| M377 B7 | H-P1530 | purine nucleoside phosphorylase | 19.91 | |
| | | (punB) | | |
| M377 C7 | H-P1531 | hypothetical protein | 8.8 | |
| M377 D7 | H-P1532 | glucosamine fructose-6-phosphate | 65.78 | |
| | | aminotransferase (isomerizing) | | |
| | | (glmS) | | |
| M377 E7 | H-P1533 | conserved hypothetical protein | 25.52 | |
| M377 F7 | H-P1534 | IS605 transposase (tnpB) | 47.08 | |
| M377 G7 | H-P1535 | IS605 transposase (tnpA) | 15.73 | |
| M377 H7 | H-P1541 | transcription-repair coupling | 110 | |
| | | factor (trcF) | | |
| M377 A8 | H-P1548 | conserved hypothetical integral | 12.43 | |
| | | membrane protein | | |
| M377 B8 | H-P1551 | conserved hypothetical secreted | 14.08 | |
| | | protein | | |
| M377 C8 | H-P1552 | Na+/H+ antiporter (nhaA) | 48.29 | |
| M381 B4 | H-P1554 | ribosomal protein S2 (rps2) | 29.15 | |
| M381 D4 | H-P1555 | translation elongation factor EF- | 39.16 | |
| | | Ts (tsf) | | |
| M377 D8 | H-P1556 | cell division protein (ftsI) | 67.76 | |
| M381 E4 | H-P1557 | flagellar basal-body protein (fliE) | 12.1 | |
| M381 F4 | H-P1558 | flagellar basal-body rod protein | 17.82 | |
| | | (flgC) (proximal rod protein) | | |
| M381 G4 | H-P1559 | flagellar basal-body rod protein | 15.51 | |
| | | (flgB) (proximal rod protein) | | |
| M378 A1 | H-P1560 | cell division protein (ftsW) | 42.79 | |
| M378 B1 | H-P1561 | iron(III) ABC transporter, | 36.96 | |
| | | periplasmic iron-binding protein | | |
| | | (ceuE) | | |
| M378 C1 | H-P1562 | iron(III) ABC transporter, | 36.74 | |
| | | periplasmic iron-binding protein | | |
| | | (ceuE) | | |
| M378 D1 | H-P1563 | alkyl hydroperoxide reductase | 21.89 | |
| | | (tsaA) | | |
| M378 E1 | H-P1564 | outer membrane protein | 29.92 | |
| M378 F1 | H-P1565 | penicillin-binding protein 2 | 64.79 | |
| | | (pbp2) | | |
| M378 G1 | H-P1566 | hypothetical protein | 16.28 | |
| M378 H1 | H-P1567 | conserved hypothetical ATP- | 22.99 | |
| | | binding protein | | |
| M378 A2 | H-P1568 | hypothetical protein | 20.24 | |
| M378 B2 | H-P1569 | hypothetical protein | 21.78 | |
| M378 C2 | H-P1570 | conserved hypothetical protein | 18.15 | |
| M378 D2 | H-P1571 | rare lipoprotein A (rlpA) | 34.76 | |
| M378 E2 | H-P1572 | regulatory protein DniR | 41.03 | |
| M378 F2 | H-P1573 | conserved hypothetical protein | 28.05 | |
| M378 G2 | H-P1576 | ABC transporter, ATP-binding | 36.08 | |
| | | protein (abc) | | |
| M378 H2 | H-P1577 | ABC transporter, permease | 23.76 | |
| | | protein (yaeE) | | |
| M378 A3 | H-P1580 | hypothetical protein | 24.31 | |
| M378 B3 | H-P1581 | methicillin resistance protein | 37.07 | |
| | | (llm) | | |
| M378 C3 | H-P1582 | pyridoxal phosphate biosynthetic | 28.93 | |
| | | protein J (pdxJ) | | |
| M378 D3 | H-P1583 | pyridoxal phosphate biosynthetic | 33.88 | |
| | | protein A (pdxA) | | |
| M378 E3 | H-P1584 | sialoglycoprotease (gcp) | 37.51 | |
| M378 F3 | H-P1585 | flagellar basal-body rod protein | 28.93 | |
| | | (flgG) | | |
| M378 G3 | H-P1587 | conserved hypothetical protein | 17.16 | |
| M378 H3 | H-P1588 | conserved hypothetical protein | 27.94 | |
| M381 H1 | H-P1590 | hypothetical protein | 4.4 | |
| M318 G2 | H-S38729 | autoimmune antigen Ku, p70 | 67.1 | 67 |
| | | subunit | | |
| H1 | H-S39329 | Kallikrein 1 | 24.64 | 30 |
| | | (renal/pancreas/salivary) | | |
| | | {alternative products} | | |
| M270 G4 | H-S43855 | Recoverin, photoreceptor protein | 22.11 | 32.0 kDa |
| M300 C2 | H-S56151 | milk fat globule protein HMFG | 24.09 | 30 |
| M318 C1 | H-S57153 | retinoblastoma-binding protein 1, | 101.31 | 101 |
| | | isoform I [RBBP1] | | |
| M271 B2 | H-S57162 | retinoblastoma-binding protein 1, | 93.72 | 110 |
| | | isoform III [RBBP1], | | |
| | | INTERACTS WITH THE VIRAL | | |
| | | PROTEIN-BINDING DOMAIN | | |
| | | OF THE RETINOBLASTOMA | | |
| | | PROTEIN. | | |
| M317 H3 | H-S62027 | transducin, gamma subunit | 8.25 | 11 |
| M270 G6 | H-S66793 | arrestin, X-arrestin = S-antigen | 42.79 | 50.0 kDa |
| | | homolog [human, retina, mRNA, | | |
| | | 1314 nt], MAY PLAY A ROLE | | |
| | | IN AN AS YET UNDEFINED | | |
| | | RETINA-SPECIFIC SIGNAL | | |
| | | TRANSDUCTION. | | |
| M419 C2 | H-S67859 | “transcription initiation factor IIe, | 48.360 | 64.0 kDa |
| | | alpha subunit” | | |
| M302 D7 | H-S69022 | myosin, light polypeptide 2, | 18.26 | 31 |
| | | ventricular | | |
| H5 | H-S69272 | cytoplasmic antiproteinase = 38 | 41.47 | 50 |
| | | kda intracellular serine proteinase | | |
| | | inhibitor [human, placenta, | | |
| | | mRNA, 1465 nt] | | |
| D1 | H-S72043 | GIF = growth inhibitory factor | 7.59 | 19 |
| | | [human, brain, Genomic, 2015 nt] | | |
| M266 B3 | H-S74221 | cytokine IK factor | 17.93 | 36.0 kDa |
| D1 | H-S74445 | cellular retinoic acid-binding | 15.18 | 23 |
| | | protein [human, skin, mRNA, 735 | | |
| | | nt] | | |
| E3 | H-S74728 | antiquitin = 26 g turgor protein | 56.32 | 53 |
| | | homolog [human, kidney, mRNA, | | |
| | | 1809 nt] | | |
| D4 | H-S75174 | E2F transcription factor 4, | 45.87 | 58 |
| | | p107/p130-binding | | |
| 166-61 | H-S76474 | “trkB {alternately spliced} | 55 | 52.54 |
| | | [human, brain, mRNA]” | | |
| 169-40 | H-S76617 | “Blk = protein tyrosine kinase | 60 | 55.62 |
| | | [human, B lymphocytes, mRNA, | | |
| | | 2608 nt]” | | |
| M250 D3 | H-S79522 | ubiquitin carboxyl-terminal | 17.27 | 17.0 kDa |
| | | extension protein, Ubiquitin A-52 | | |
| | | residue ribosomal protein fusion | | |
| | | product 1 | | |
| M236 B4 | H-S80562 | calponin, acidic | 36.3 | 49 |
| G1 | H-S82470 | BB1 = malignant cell expression- | 37.73 | 34 |
| | | enhanced gene/tumor | | |
| | | progression-enhanced gene | | |
| | | [human, UM-UC-9 bladder | | |
| | | carcinoma cell line, mRNA, 1897 | | |
| | | nt] | | |
| M313 E1 | H-S85655 | prohibitin [PHB] | 30.03 | 40.0 kDa |
| M465 A6 | H-S87759 | protein phosphatase 2C alpha | 42.13 | 52.0 kDa |
| | | [human, teratocarcinoma, mRNA, | | |
| | | 2346 nt] | | |
| M472 B1 | H-U00803 | tyrosine-protein kinase FRK | 55.620 | 64.0 kDa |
| B2 | H-U02390 | Human adenylyl cyclase- | 52.58 | 55 |
| | | associated protein homolog CAP2 | | |
| | | (CAP2) mRNA, complete cds | | |
| 167-2 | H-U02680 | human protein tyrosine kinase | 36 | 38.57 |
| | | mRNA | | |
| G2 | H-U03056 | Human tumor suppressor (LUCA- | 47.96 | 47 |
| | | 1) mRNA, complete cds | | |
| M512 E3 | H-U03100 | Human alpha2(E)-catenin mRNA, | 102.52 | 102.0 kDa |
| | | complete cds | | |
| M306 G3 | H-U03187 | | 72.93 | 95.0 kDa |
| H3 | H-U03398 | Human receptor 4-1BB ligand | 28.05 | 51 |
| | | mRNA, complete cds | | |
| D3 | H-U03486 | Human connexin40 gene, | 39.49 | 40 |
| | | complete cds | | |
| M300 C3 | H-U03643 | leukophysin | 25.96 | 34 |
| F5 | H-U03749 | Human chromogranin A (CHGA) | 50.38 | 50 |
| | | gene, promoter and | | |
| M314 C3 | H-U03886 | GS2 (GB: U03886) | 27.94 | 32.0 kDa |
| M306 E3 | H-U04343 | CD86 antigen (CD28 antigen | 35.64 | 47 |
| | | ligand 2, B7-2 antigen) [CD86] | | |
| 167-61 | H-U05012 | TrkC | 92 | 90.82 |
| M302 G5 | H-U05340 | cell division cycle protein p55 | 55 | 55 |
| A4 | H-U05659 | Hydroxysteroid (17-beta) | 34.21 | 36 |
| | | dehydrogenase 3 | | |
| F1 | H-U05861 | Human hepatic dihydrodiol | 35.64 | 40 |
| | | dehydrogenase gene | | |
| M302 B2 | H-U06452 | antigen MART-1, melanoma | 13.09 | 20.0 kDa |
| 169-52 | H-U06454 | human AMP-activated protein | 70 | 60.79 |
| | | kinase (hAMPK) mRNA | | |
| M315 A3 | H-U06643 | lectin, epidermal | 15.07 | 18 |
| H1 | H-U06715 | Cytochrome B561 | 27.06 | 25 |
| M476 E5 | H-U07132 | Human steroid hormone receptor | 50.82 | 55.0 kDa |
| | | Ner-I mRNA, complete cds | | |
| M236 D3 | H-U07151 | guanine nucleotide-binding | 20.13 | 34 |
| | | protein ADP-ribosylation factor | | |
| | | like gene 3 | | |
| M317 G3 | H-U07559 | homeotic protein Islet-1 | 38.17 | 38 |
| M266 H1 | H-U07681 | Human NAD(H)-specific | 40.37 | 40 |
| | | isocitrate dehydrogenase alpha | | |
| | | subunit precursor mRNA, | | |
| | | complete cds | | |
| E3 | H-U07919 | Aldehyde dehydrogenase 6 | 56.43 | 53 |
| M298 A3 | H-U08021 | nicotinamide N-methyltransferase | 29.15 | 36.0 kDa |
| M297 B1 | H-U08024 | alcohol/hydroxysteroid | 31.46 | 50.0 kDa |
| | | sulfotransferase | | |
| A2 | H-U08336 | Human basic helix-loop-helix | 21.89 | 42 |
| | | transcription factor mRNA, | | |
| | | complete cds | | |
| E2 | H-U09303 | Human T cell leukemia LERK-2 | 38.17 | 40 |
| | | (EPLG2) mRNA, complete cds | | |
| M250 H5 | H-U09559 | RCH1, RAG (recombination | 58.3 | 58.0 kDa |
| | | activating gene) cohort 1 | | |
| 167-50 | H-U09564 | human serine kinase mRNA | 72 | 72.12 |
| 166-74 | H-U09578 | human MAPKAP kinase (3pK) | 50 | 42.09 |
| | | mRNA | | |
| M302 C4 | H-U09813 | ATP synthase, subunit 9, | 15.73 | 30 |
| | | mitochondrial | | |
| A1 | H-U09850 | Zinc finger protein 143 (clone | 68.97 | 68 |
| | | pHZ-1) | | |
| M423 E1 | H-U09937 | Human urokinase-type | 36.96 | 49.0 kDa |
| | | plasminogen receptor | | |
| M450 H4 | H-U10117 | Human endothelial-monocyte | 34.43 | 38.0 kDa |
| | | activating polypeptide II mRNA, | | |
| | | complete cds | | |
| M314 G1 | H-U10248 | ribosomal protein L29 | 17.6 | 27 |
| M298 H1 | H-U10323 | nuclear factor 45 | 44.77 | 45 |
| E1 | H-U10492 | Human Mox1 protein (MOX1) | 28.05 | 37 |
| | | mRNA, complete cds | | |
| F3 | H-U10686 | Human MAGE-11 antigen | 35.2 | 35 |
| | | (MAGE11) gene, complete cds | | |
| 167-38 | H-U11050 | human NIMA-like protein kinase | 55 | 49.02 |
| | | 1 (NLK1) mRNA | | |
| M266 B2 | H-U11292 | Human Ki nuclear autoantigen | 29.48 | 32 |
| | | mRNA, complete cds, may play a | | |
| | | rol in cell adhesion | | |
| 167-62 | H-U11791 | human cyclin H mRNA | 40 | 35.60 |
| M423 D5 | H-U12255 | immunoglobulin gamma heavy | 40.26 | 48.0 kDa |
| | | chain Fc receptor RI, high affinity | | |
| M302 F7 | H-U12404 | Csa-19 | 23.98 | 32 |
| M236 A2 | H-U12465 | ribosomal protein L35 | 13.64 | 24 |
| 169-4 | H-U12535 | human epidermal growth factor | 100 | 90.49 |
| | | receptor kinase substrate (Eps8) | | |
| | | mRNA | | |
| F3 | H-U12597 | Human tumor necrosis factor type | 55.22 | 64 |
| | | 2 receptor associated protein | | |
| | | (TRAP3) mRNA, complete cds | | |
| M314 D1 | H-U12979 | transcriptional coactivator PC4 | 14.08 | 23 |
| M476 G4 | H-U13044 | GA-binding protein transcription | 50.05 | 53.0 kDa |
| | | factor, alpha subunit (60 kD) | | |
| M302 F3 | H-U13665 | cathepsin O (GB: U13665) | 36.3 | 50.0 kDa |
| M311 G4 | H-U13831 | cellular retinol binding protein II | 14.85 | 20.0 kDa |
| A2 | H-U13991 | Human TATA-binding protein | 24.09 | 34 |
| | | associated factor 30 kDa subunit | | |
| | | (tafII30) mRNA, complete cds | | |
| M416 A4 | H-U14187 | Human receptor tyrosine kinase | 26.29 | 29.0 kDa |
| | | ligand LERK-3 (EPLG3) mRNA, | | |
| | | complete cds | | |
| M250 A2 | H-U14188 | eph-related receptor tyrosine | 22.22 | 27 |
| | | kinase ligand 4 [EPLG4] | | |
| M302 D2 | H-U14193 | human TFIIA gamma subunit | 12.060 | 28.0 kDa |
| | | mRNA | | |
| M416 G1 | H-U14603 | Human protein-tyrosine | 18.48 | 30.0 kDa |
| | | phosphatase (HU-PP-1) mRNA, | | |
| | | partial sequence | | |
| E2 | H-U14747 | Visinin-like 1 | 21.12 | 25 |
| M266 D4 | H-U14966 | ribosomal protein L5 | 32.78 | 38 |
| M314 E2 | H-U14967 | ribosomal protein L21 | 17.71 | 29 |
| M266 F5 | H-U14968 | ribosomal protein L27a | 16.39 | 19.0 kDa |
| M248 E3 | H-U14969 | ribosomal protein L28 | 15.18 | 27 |
| M266 E1 | H-U14971 | ribosomal protein S9 | 21.45 | 30 |
| M250 C2 | H-U15009 | small nuclear ribonucleoprotein, | 13.97 | 17.0 kDa |
| | | Sm D3 | | |
| M311 D4 | H-U16660 | enoyl-Coenzyme A hydratase-like | 36.19 | 38 |
| | | protein, peroxisomal | | |
| M302 H4 | H-U17074 | cyclin-dependent kinase 6 | 18.59 | 29 |
| | | inhibitor p18 | | |
| M306 A2 | H-U17195 | A-kinase anchor protein 100 | 72.05 | 100 |
| | | [AKAP100*] | | |
| D1 | H-U17280 | Steroidogenic acute regulatory | 31.46 | 35 |
| | | protein | | |
| M316 F1 | H-U18291 | cell division cycle protein 16 | 68.2 | 71.0 kDa |
| C5 | H-U18420 | Human ras-related small GTP | 23.87 | 33 |
| | | binding protein Rab5 (rab5) | | |
| | | mRNA, complete cds | | |
| M311 A2 | H-U18423 | spinal muscular atrophy gene | 32.45 | 41 |
| M248 D4 | H-U18914 | hypothetical protein, (Human | 20.35 | 32 |
| | | 19.8 kDa protein mRNA, | | |
| | | complete cds) | | |
| M302 B5 | H-U19718 | microfibril-associated | 20.24 | 34.0 kDa |
| | | glycoprotein 2 | | |
| M305 E3 | H-U20240 | CCAAT/enhancer-binding protein | 16.61 | 29 |
| | | gamma | | |
| M302 A8 | H-U20352 | malate dehydrogenase | 36.85 | 40 |
| M416 F4 | H-U20391 | Human folate receptor (FOLR1) | 28.38 | 34.0 kDa |
| | | gene, complete cds | | |
| M311 D1 | H-U20536 | apoptotic cysteine protease Mch2 | 32.34 | 38.0 kDa |
| M431 G2 | H-U20659 | RNA polymerase II, subunit B7 | 19.03 | 31.0 kDa |
| M499 C1 | H-U20938 | Human lymphocyte | 112.86 | 100.0 kDa |
| | | dihydropyrimidine dehydrogenase | | |
| | | mRNA, complete cds | | |
| M305 F2 | H-U20972 | 14-3-3 protein, epsilon | 28.16 | 36 |
| M271 D3 | H-U21049 | hypothetical protein | 12.65 | 16 |
| | | (GB: U21049), ESTs, Highly | | |
| | | similar to DD96 [H. sapiens]. | | |
| M421 G5 | H-U21858 | Human transcriptional activation | 29.15 | 38.0 kDa |
| | | factor TAFII32 mRNA, complete | | |
| | | cds | | |
| M424 H3 | H-U22662 | Human nuclear orphan receptor | 49.28 | 49.0 kDa |
| | | LXR-alpha mRNA, complete cds | | |
| M271 D2 | H-U24074 | killer cell inhibitory receptor | 37.62 | 43 |
| | | [KIR], Homo sapiens natural | | |
| | | killer-associated transcript 3 | | |
| | | (NKAT3), complete cds. | | |
| | | RECEPTOR ON NATURAL | | |
| | | KILLER (NK) CELLS FOR | | |
| | | HLA-C ALLELES. | | |
| 169-29 | H-U24153 | human p21-activated protein | 60 | 57.82 |
| | | kinase (Pak2) gene | | |
| M385 H2 | H-U24166 | EB1 | 29.59 | 36.0 kDa |
| G1 | H-U24169 | Human JTV-1 (JTV-1) mRNA, | 34.43 | 40 |
| | | complete cds | | |
| E1 | H-U24576 | Human breast tumor autoantigen | 18.26 | 27 |
| | | mRNA, complete sequence | | |
| G4 | H-U24577 | Human LDL-phospholipase A2 | 48.62 | 52 |
| | | mRNA, complete cds | | |
| H1 | H-U25789 | Human ribosomal protein L21 | 17.71 | 32 |
| | | mRNA, complete cds | | |
| M416 D1 | H-U25849 | Human red cell-type low | 17.49 | 28.0 kDa |
| | | molecular weight acid | | |
| | | phosphatase (ACP1) gene, 5′ | | |
| | | flanking region and | | |
| M300 A3 | H-U26312 | heterochromatin protein H-P1Hs- | 19.14 | 30 |
| | | gamma | | |
| M416 D3 | H-U26403 | Human receptor tyrosine kinase | 25.19 | 30.0 kDa |
| | | ligand LERK-7 precursor | | |
| | | (EPLG7) mRNA, complete cds | | |
| M317 E2 | H-U27143 | human protein kinase C inhibitor- | 13.900 | 17.0 kDa |
| | | I cDNA | | |
| E5 | H-U28249 | Human 11 kd protein mRNA, | 12.32 | 12 |
| | | complete cds | | |
| F4 | H-U28386 | Human nuclear localization | 58.3 | 54 |
| | | sequence receptor hSRP1 alpha | | |
| | | mRNA, complete cds | | |
| M423 E3 | H-U28694 | Chemokine (C-C) receptor 3 | 39.16 | 39.0 kDa |
| M266 G6 | H-U28963 | Gps2 | 36.08 | 36 |
| M306 D3 | H-U30610 | CD94 antigen (NK/T-cell C-type | 19.8 | 27 |
| | | lectin receptor) [CD94] | | |
| B1 | H-U31116 | Human beta-sarcoglycan A3b | 35.09 | 33 |
| | | mRNA, complete cds | | |
| M297 C2 | H-U31278 | mitotic feedback control protein | 22.66 | 31.0 kDa |
| | | Madp2 homolog | | |
| M302 G2 | H-U31384 | guanine nucleotide-binding | 8.14 | 10 |
| | | protein, gamma 11 subunit | | |
| F4 | H-U31986 | Human cartilage-specific | 35.97 | 47 |
| | | homeodomain protein Cart-1 | | |
| | | mRNA, complete cds | | |
| M390 F3 | H-U32114 | caveolin 2 | 17.93 | 18.0 kDa |
| E4 | H-U32324 | Human interleukin-11 receptor | 46.53 | 54 |
| | | alpha chain mRNA, complete cds | | |
| F1 | H-U32576 | Apolipoprotein C-IV | 14.08 | 16 |
| M298 C4 | H-U32907 | p37NB protein | 34.54 | 39 |
| M300 D3 | H-U32944 | dynein, light chain 1, cytoplasmic | 9.9 | 15 |
| M297 D1 | H-U32989 | tryptophan 2,3-dioxygenase | 44.77 | 50.0 kDa |
| 166-51 | H-U33052 | “protein kinase PRK2 [human, | 110 | 108.3 |
| | | DX3 B-cell myeloma cell line, | | |
| | | mRNA]” | | |
| 166-64 | H-U33054 | “human G protein-coupled | 52 | 63.65 |
| | | receptor kinase GRK4 mRNA, | | |
| | | alpha splice variant” | | |
| 166-88 | H-U33055 | “human G protein-coupled | 60 | 60.1 |
| | | receptor kinase GRK4 mRNA, | | |
| | | beta splice variant” | | |
| 166-76 | H-U33056 | “human G protein-coupled | 58 | 58.59 |
| | | receptor kinase GRK4 mRNA, | | |
| | | gamma splice variant” | | |
| A2 | H-U34584 | | 17.71 | 31 |
| 169-87 | H-U34820 | human MAP kinase mRNA | 55 | 46.49 |
| 215-2 | H-U34822 | human JNK1 alpha2 protein | 55 | 47.04 |
| | | kinase (JNK1A2) mRNA | | |
| 169-37 | H-U35002 | human JNK2 betal protein kinase | 50 | 42.09 |
| | | (JNK2B1) mRNA | | |
| 169-25 | H-U35003 | human JNK2 beta2 protein kinase | 55 | 46.71 |
| | | (JNK2B2) mRNA | | |
| 167-16 | H-U35004 | human JNK1 betal protein kinase | 52 | 42.31 |
| | | (JNK1B1) mRNA | | |
| M300 B2 | H-U35048 | TSC-22 protein | 15.95 | 27 |
| M423 E5 | H-U35398 | Human G protein-coupled | 40.26 | 48.0 kDa |
| | | receptor mRNA, complete cds | | |
| A3 | H-U35735 | Human RACH1 (RACH1) | 42.9 | 78 |
| | | mRNA, complete cds | | |
| M250 E5 | H-U36764 | Eukaryotic translation initiation | 35.86 | 36.0 kDa |
| | | factor 3 (eIF-3) p36 subunit, | | |
| | | transforming growth factor-beta | | |
| | | receptor II interacting protein 1 | | |
| M270 E4 | H-U37283 | microfibril-associated | 19.14 | 32 |
| | | glycoprotein-2 (GB: U37283) | | |
| M426 F3 | H-U37352 | Protein phosphatase 2A, | 56.65 | 55.0 kDa |
| | | regulatory subunit B′alpha-1 | | |
| E1 | H-U37529 | Human substance P beta-PPT-A | 14.3 | 22 |
| | | mRNA, complete cds | | |
| M305 H5 | H-U37547 | apoptosis inhibitor | 68.09 | 64 |
| M424 D5 | H-U38480 | Human retinoid X receptor- | 51.04 | 61.0 kDa |
| | | gamma mRNA, complete cds | | |
| M270 F4 | H-U38810 | Human mab-21 cell fate- | | |
| | | determining protein homolog | | |
| | | (CAGR1) mRNA, | | |
| M467 F6 | H-U38904 | Human zinc finger protein C2H2- | 40.48 | 47.0 kDa |
| | | 25 mRNA, complete cds | | |
| E2 | H-U39318 | Human E2 ubiquitin conjugating | 16.28 | 22 |
| | | enzyme UbcH5C (UBCH5C) | | |
| | | mRNA, complete cds | | |
| 166-75 | H-U39657 | human MAP kinase kinase 6 | 40 | 36.81 |
| | | (MKK6) mRNA | | |
| M298 E4 | H-U39945 | human adenylate kinase 2 (adk2) | 26.3633 | 38.0 kDa |
| | | mRNA | | |
| 166-38 | H-U40282 | human integrin-linked kinase | 55 | 49.68 |
| | | (ILK) mRNA | | |
| 169-65 | H-U40343 | human CDK inhibitor p19INK4d | 18 | 18.33 |
| | | mRNA | | |
| E2 | H-U40705 | Homo sapiens telomeric repeat | 48.4 | 52 |
| | | binding factor (TRF1) mRNA, | | |
| | | complete cds | | |
| 166-50 | H-U40989 | human tat interactive protein | 60 | 53.09 |
| | | mRNA | | |
| M266 H6 | H-U41767 | metargidin precursor | 89.65 | 90 |
| M270 F3 | H-U41804 | Human putative T1/ST2 receptor | 25.08 | 35.0 kDa |
| | | binding protein precursor mRNA, | | |
| | | complete cds | | |
| D5 | H-U42360 | Human N33 gene | 38.28 | 38 |
| A1 | H-U43368 | Vascular endothelial growth | 22.88 | 33 |
| | | factor B | | |
| M421 G7 | H-U43901 | Human 37 kD laminin receptor | 32.56 | 58.0 kDa |
| | | precursor/p40 ribosome | | |
| | | associated protein gene, complete | | |
| | | cds | | |
| M392 C2 | H-U43923 | transcription factor SUPTH4 | 12.98 | 16.0 kDa |
| E2 | H-U46024 | Myotubular myopathy 1 | 66.44 | 58 |
| M330 A1 | H-U46838 | p105MCM | 90.42 | 97 |
| M476 E2 | H-U47677 | Human transcription factor E2F1 | 48.18 | 53.0 kDa |
| | | (E2F1) gene, promoter and | | |
| M421 H1 | H-U48707 | Human protein phosphatase-1 | 18.92 | 36.0 kDa |
| | | inhibitor mRNA, complete cds | | |
| M302 B7 | H-U49070 | peptidyl-prolyl isomerase PIN1 | 18.04 | 28.0 kDa |
| C1 | H-U49188 | Human placenta (Diff33) mRNA, | 54.45 | 70 |
| | | complete cds | | |
| M485 H2 | H-U49837 | Human LIM protein MLP mRNA, | 21.45 | 34.0 kDa |
| | | complete cds | | |
| D2 | H-U49897 | Homo sapiens phenylalanine | 49.83 | 64 |
| | | hydroxylase (PAH) mRNA, | | |
| | | complete cds | | |
| B2 | H-U49957 | Human LIM protein (LPP) | 67.43 | 67 |
| | | mRNA, partial cds | | |
| 166-16 | H-U50196 | human adenosine kinase mRNA | 50 | 38.02 |
| A4 | H-U50939 | Human amyloid precursor | 58.85 | 60 |
| | | protein-binding protein 1 mRNA, | | |
| | | complete cds | | |
| G3 | H-U51224 | Human U2AFBPL gene, complete | 52.8 | 55 |
| | | cds | | |
| M486 E3 | H-U51333 | Hexokinase 3 (white cell) | 101.64 | 100.0 kDa |
| M305 D1 | H-U51478 | ATPase, Na+/K+ transporting, | 30.8 | 36 |
| | | beta 3 subunit | | |
| M416 H3 | H-U52112 | Homo sapiens Xq28 genomic | 25.96 | 36.0 kDa |
| | | DNA in the region of the L1CAM | | |
| | | locus containing the genes for | | |
| | | neural cell adhesion molecule L1 | | |
| | | (L1CAM), arginine-vasopressin | | |
| | | receptor (AVPR2), C1 p115 (C1), | | |
| | | ARD1 N-acetyltransferase related | | |
| | | protein (TE2), renin-binding | | |
| | | protein (RbP), host cell factor 1 | | |
| | | (HCF1), and interleukin-1 | | |
| | | receptor-associated kinase | | |
| | | (IRAK) genes, complete cds, and | | |
| | | Xq28lu2 gene | | |
| M463 E1 | H-U53442 | human p38Beta MAP kinase | 40.99 | 49.0 kDa |
| | | mRNA | | |
| G3 | H-U53446 | Human mitogen-responsive | 84.81 | 98 |
| | | phosphoprotein DOC-2 mRNA, | | |
| | | complete cds | | |
| M463 C1 | H-U54617 | human pyruvate dehydrogenase | 45.28 | 52.0 kDa |
| | | kinase isoform 4 mRNA | | |
| 169-38 | H-U54645 | methylmalonyl-coA mutase | 38 | 25.59 |
| | | precursor | | |
| M300 H3 | H-U56255 | t-complex sterility protein | 12.54 | 16 |
| | | homolog CW-1 | | |
| C4 | H-U56417 | Human lysophosphatidic acid | 31.24 | 46 |
| | | acyltransferase-alpha mRNA, | | |
| | | complete cds | | |
| M305 A2 | H-U56637 | actin-capping protein alpha | 31.57 | 31 |
| | | subunit isoform 1 | | |
| M235 E6 | H-U56814 | Human DNase1-Like III protein | 33.66 | 40.0 kDa |
| | | (DNAS1L3) mRNA, complete | | |
| | | cds, involved in apoptosis Binds | | |
| | | specifically to G-ACTIN AND | | |
| | | BLOCKS ACTIN | | |
| | | POLYMERIZATION. | | |
| D5 | H-U57059 | | 31.02 | 36 |
| B3 | H-U57093 | Human small GTP-binding | 24.09 | 34 |
| | | protein rab27b mRNA, complete | | |
| | | cds | | |
| D3 | H-U57099 | Human APEG-1 mRNA, | 12.54 | 20 |
| | | complete cds | | |
| F1 | H-U58331 | Sarcoglycan, delta (35 kD | 28.27 | 24 |
| | | dystrophin-associated | | |
| | | glycoprotein) | | |
| M512 F4 | H-U58334 | Human Bcl2, p53 binding protein | 110.66 | 108.0 kDa |
| | | Bbp/53BP2 (BBP/53BP2) mRNA, | | |
| | | complete cds | | |
| B3 | H-U58516 | Human breast epithelial antigen | 42.68 | 50 |
| | | BA46 mRNA, complete cds | | |
| M250 E4 | H-U58522 | Human huntingtin interacting | 22.11 | 30 |
| | | protein (HIP2) mRNA, complete | | |
| | | cds | | |
| M419 G2 | H-U60207 | human stress responsive | 53.640 | 63.0 kDa |
| | | serine/threonine protein kinase | | |
| | | Krs-2 mRNA | | |
| M298 B2 | H-U60276 | arsA homolog (hASNA-I) | 36.63 | 47.0 kDa |
| B2 | H-U60521 | Human protease proMch6 (Mch6) | 45.87 | 52 |
| | | mRNA, complete cds | | |
| F3 | H-U61166 | Human SH3 domain-containing | 57.31 | 57 |
| | | protein SH3P17 mRNA, complete | | |
| | | cds | | |
| M250 B5 | H-U61232 | cofactor E (tubulin-folding | | |
| | | protein), REQUIRED FOR | | |
| | | VIABILITY IN THE ABSENCE | | |
| | | OF THE KINESIN-RELATED | | |
| | | CIN8 | | |
| A5 | H-U62392 | Homo sapiens zinc finger protein | 43.45 | 52 |
| | | mRNA, complete cds | | |
| G1 | H-U62801 | Human protease M mRNA, | 26.95 | 33 |
| | | complete cds | | |
| M266 B1 | H-U62962 | Int-6 , Human Int-6 mRNA, | 49.06 | 52.0 kDa |
| | | complete cds | | |
| M300 G1 | H-U63295 | seven in absentia homolog | 31.13 | 36 |
| M306 H3 | H-U64198 | | 94.93 | 98 |
| H3 | H-U64863 | Human hPD-1 (hPD-1) mRNA, | 31.79 | 37 |
| | | complete cds | | |
| B3 | H-U65581 | Human ribosomal protein L3-like | 44.88 | 52 |
| | | mRNA, complete cds | | |
| M341 D1 | H-U65918 | DAZ homologue [DAZLA] | 32.56 | 36.0 kDa |
| M302 E1 | H-U65928 | Jun activation domain binding | 36.85 | 48.0 kDa |
| | | protein | | |
| M512 D3 | H-U66347 | Homo sapiens cAMP | 46.97 | 60.0 kDa |
| | | phosphodiesterase (PDE4C) | | |
| | | mRNA, 4C-426 isoform, | | |
| | | complete cds | | |
| M306 F3 | H-U66867 | ubiquitin-conjugating enzyme E2I | 17.49 | 28 |
| | | [UBE2I] | | |
| M416 E2 | H-U68111 | Human protein phosphatase | 22.66 | 37.0 kDa |
| | | inhibitor 2 (PPP1R2) gene | | |
| F2 | H-U68382 | Mannosidase, alpha B, lysosomal | 35.64 | 36 |
| G2 | H-U69141 | Glutaryl-Coenzyme A | 48.29 | 56 |
| | | dehydrogenase | | |
| B2 | H-U70660 | Human copper transport protein | 7.59 | 16 |
| | | HAH1 (HAH1) mRNA, complete | | |
| | | cds | | |
| M297 B2 | H-U71374 | peroxisomal membrane protein | 40.15 | 40.0 kDa |
| | | (Pex13p) | | |
| M306 A3 | H-U75272 | progastricsin [PGC] | 42.79 | 49.0 kDa |
| A2 | H-U75285 | Homo sapiens apoptosis inhibitor | 15.73 | 25 |
| | | survivin gene, complete cds | | |
| B2 | H-U77456 | Human nucleosome assembly | 41.36 | 50 |
| | | protein 2 mRNA, complete cds | | |
| C2 | H-U78294 | Homo sapiens 15S-lipoxygenase | 74.47 | 74 |
| | | mRNA, complete cds | | |
| F6 | H-U78302 | Human 2,4-dienoyl-CoA | 36.96 | 40 |
| | | reductase gene | | |
| M478 G3 | H-U78798 | Human TNF receptor associated | 57.53 | 65.0 kDa |
| | | factor 6 (TRAF6) mRNA, | | |
| | | complete cds | | |
| G3 | H-U80982 | Human myeloid-specific C/EBP- | 27.5 | 51 |
| | | epsilon transcription factor | | |
| | | (CEBPE) gene, complete cds | | |
| M468 B7 | H-U82256 | Homo sapiens arginase type II | 39.05 | 45.0 kDa |
| | | mRNA, complete cds | | |
| M465 B2 | H-U82812 | Human scavenger receptor | 38.28 | 48.0 kDa |
| | | cysteine rich Sp alpha mRNA, | | |
| | | complete cds | | |
| M484 D7 | H-U83410 | Human CUL-2 (cul-2) mRNA, | 82.06 | 85.0 kDa |
| | | complete cds | | |
| M467 E6 | H-U83460 | Human high-affinity copper | 21.01 | 32.0 kDa |
| | | uptake protein (hCTR1) mRNA, | | |
| | | complete cds | | |
| D2 | H-U84763 | Homo sapiens UCP3 mRNA, | 34.43 | 42 |
| | | complete cds | | |
| B2 | H-U86070 | Homo sapiens | 28.93 | 36 |
| | | phosphomannomutase mRNA, | | |
| | | complete cds | | |
| C2 | H-U90441 | Human prolyl 4-hydroxylase | 58.96 | 64 |
| | | alpha (II) subunit mRNA, | | |
| | | complete cds | | |
| B2 | H-U90543 | Human butyrophilin (BTF1) | 58.08 | 54 |
| | | mRNA, complete cds | | |
| H2 | H-U90545 | Human sodium phosphate | 44.22 | 36 |
| | | transporter (NPT4) mRNA, | | |
| | | complete cds | | |
| G2 | H-U90552 | Human butyrophilin (BTF5) | 56.54 | 48 |
| | | mRNA, complete cds | | |
| C3 | H-U91521 | Peroxisomal biogenesis factor 12 | 39.6 | 48 |
| H1 | H-U91641 | Human alpha2,8-sialyltransferase | 41.47 | 45 |
| | | mRNA, complete cds | | |
| C1 | H-U93869 | Human RNA polymerase III | 34.98 | 36 |
| | | subunit (RPC39) mRNA, | | |
| | | complete cds | | |
| F2 | H-U94346 | Human calpain-like protease | 70.4 | 65 |
| | | (htra-3) mRNA, complete cds | | |
| C2 | H-U94855 | Human translation initiation | 39.38 | 36 |
| | | factor 3.47 kDa subunit mRNA, | | |
| | | complete cds | | |
| M271 F7 | H-U95089 | Epidermal growth factor receptor. | 44.66 | 47 |
| M424 A5 | H-U95847 | Human GDNF receptor alpha | 50.71 | 52.0 kDa |
| | | mRNA, complete cds | | |
| D2 | H-U96094 | Human sarcolipin (SLN) mRNA, | 3.52 | 10 |
| | | complete cds | | |
| B3 | H-U96769 | Homo sapiens chondroadherin | 39.6 | 43 |
| | | gene, 5'flanking region and | | |
| M298 G2 | H-V00566 | prolactin | 25.08 | 35 |
| M298 H2 | H-V00571 | corticotropin-releasing factor | 21.67 | 49 |
| 217-61 | H-V00572 | phosphoglycerate kinase 1 | 50 | 45.94 |
| M314 B3 | H-V00597 | parathyroid hormone | 12.76 | 14 |
| M305 B8 | H-X00129 | retinol-binding protein 4, | 22 | 51 |
| | | interstitial [RBP4] | | |
| F2 | H-X00351 | Human mRNA for beta-actin | 41.36 | 41 |
| A4 | H-X00570 | apolipoprotein C-I | 9.24 | 35 |
| M362 E1 | H-X01057 | interleukin 2 receptor, alpha | 30.03 | 40.0 kDa |
| | | [IL2RA] | | |
| A4 | H-X01677P | Human liver mRNA for | 10.45 | 10 |
| | | glyceraldehyde-3-phosphate | | |
| | | dehydrogenase (G3PD, EC | | |
| | | 1.2.1.12) | | |
| M271 D6 | H-X02152 | lactate dehydrogenase A [LDHA], | 36.63 | 45.0 kDa |
| | | L-LACTATE | | |
| | | DEHYDROGENASE M CHAIN | | |
| A1 | H-X02158 | Human gene for erythropoietin | 21.34 | 32 |
| H4 | H-X02415 | Human gene for fibrinogen | 48.18 | 50 |
| | | gamma chain | | |
| A5 | H-X02750 | Protein C (inactivator of | 50.82 | 53 |
| | | coagulation factors Va and VIIIa) | | |
| M302 B3 | H-X02751 | proto-oncogene N-ras | 20.9 | 25.0 kDa |
| D3 | H-X02812 | Human mRNA for transforming | 43.12 | 50 |
| | | growth factor-beta (TGF-beta) | | |
| M302 C1 | H-X03124 | tissue inhibitor of | 22.88 | 36.0 kDa |
| | | metalloproteinase 1 | | |
| M362 B1 | H-X03342 | ribosomal protein L32 | 14.96 | 24.0 kDa |
| M235 A2 | H-X03484 | human mRNA for raf oncogene | 71.350 | 73.0 kDa |
| M318 A3 | H-X03557 | interferon-induced protein 56 | 52.69 | 50.0 kDa |
| A3 | H-X03747 | ATPase, Na+/K+ transporting, | 33.44 | 45 |
| | | beta 1 polypeptide | | |
| M305 D2 | H-X04297 | ATPase, Na+/K+ transporting, | 112.64 | 99 |
| | | alpha subunit | | |
| M305 A5 | H-X04327 | 2,3-bisphosphoglycerate mutase | 28.6 | 36 |
| M271 G5 | H-X04588 | tropomyosin TM30 nm, | 26.29 | 40.0 kDa |
| | | cytoskeletal | | |
| M305 C8 | H-X04741 | ubiquitin related protein | 23.43 | 28.0 kDa |
| M236 A5 | H-X05231 | matrix metalloproteinase 1 | 51.7 | 53.0 kDa |
| | | (interstitial collagenase) [MMP1], | | |
| | | CLEAVES COLLAGENS | | |
| 166-53 | H-X05246 | “phosphoglycerate kinase, testis | 50 | 45.94 |
| | | specific” | | |
| M236 A1 | H-X05908 | annexin I, REGULATES | 38.17 | 40 |
| | | PHOSPHOLIPASE A2 | | |
| | | ACTIVITY, Binds CALCIUM | | |
| | | IONS | | |
| M250 A4 | H-X06234 | S100 calcium-binding protein A8 | 10.34 | 10.0 kDa |
| | | (calgranulin A) | | |
| M266 B6 | H-X06323 | ribosomal protein L3, isoform 1 | 38.39 | 39 |
| M313 A7 | H-X06617 | ribosomal protein S11 | 17.49 | 27 |
| M416 E4 | H-X06948 | High affinity IgE receptor alpha- | 28.38 | 36.0 kDa |
| | | subunit (FcERI) | | |
| M421 H7 | H-X07203 | Human mRNA for CD20 receptor | 32.78 | 40.0 kDa |
| | | (S7) | | |
| 217-2 | H-X07743 | pleckstrin | 38 | 38.57 |
| 217-73 | H-X07767 | “cAMP-dependent protein kinase, | 45 | 38.68 |
| | | alpha-catalytic subunit” | | |
| M305 B3 | H-X07898 | troponin C, skeletal, fast | 17.71 | 25 |
| M306 E1 | H-X07979 | integrin,beta 1 | 87.89 | 110 |
| A11 | H-X08004 | ras-related protein rap 1 B | 20.24 | 38 |
| M235 A7 | H-X12387 | Cytochrome P450 IIIA3 | 55.44 | 60.0 kDa |
| | | (nifedipine oxidase chain 3) | | |
| M315 F1 | H-X12496 | glycophorin C | 14.19 | 24 |
| M316 D3 | H-X12517 | small nuclear ribonucleoprotein | 17.6 | 30.0 kDa |
| | | U1, C | | |
| M236 E5 | H-X12534 | guanine nucleotide-binding | 20.24 | 34.0 kDa |
| | | protein rap2, ras-oncogene related | | |
| M266 E3 | H-X12597 | High-mobility group (nonhistone | 23.76 | 37 |
| | | chromosomal) protein 1, placenta | | |
| 217-14 | H-X12656 | human mRNA for protein | 40 | 34.06 |
| | | phosphatase 2A (beta type) | | |
| H4 | H-X12662 | H. sapiens arginase gene exon 1 | 35.53 | 50 |
| | | and flanking regions (EC 3.5.3.1) | | |
| | | (and joined CDS) | | |
| C1 | H-X12953 | RAB2, member RAS oncogene | 23.43 | 29 |
| | | family | | |
| F5 | H-X13956 | Human 12S RNA induced by | 9.13 | 19 |
| | | poly(rI), poly(rC) and Newcastle | | |
| | | disease virus | | |
| M297 A1 | H-X15005 | laminin receptor 1 | 33.11 | 48.0 kDa |
| M315 E3 | H-X15088 | guanine nucleotide binding | 38.61 | 45 |
| | | protein (G protein), alpha | | |
| | | transducing (transducin) activity | | |
| | | polypeptide 1 [GNAT1] | | |
| G2 | H-X15183 | Human mRNA for 90-kDa heat- | 80.63 | 80 |
| | | shock protein | | |
| M385 C1 | H-X15422 | mannose-binding lectin, soluble | 27.39 | 27.0 kDa |
| | | (opsonic defect) [MBL] | | |
| M271 D7 | H-X15606 | INTERCELLULAR ADHESION | 30.36 | 37.0 kDa |
| | | MOLECULE-2 PRECURSOR | | |
| | | [Homo sapiens]. | | |
| M298 C5 | H-X15653 | uracil-DNA glycosylase | 33.55 | 37 |
| M302 B4 | H-X15822 | cytochrome-c oxidase, VIIa | 9.24 | 20 |
| | | subunit, liver | | |
| M305 A6 | H-X15940 | ribosomal protein L31 | 13.86 | 18 |
| M236 G5 | H-X15949 | interferon regulatory factor 2, | 38.5 | 54.0 kDa |
| | | BINDS AND REPRESSES | | |
| | | REGULATORY REGION OF | | |
| | | TYPE I IFN AND IFN- | | |
| | | INDUCIBLE MHC CLASS I | | |
| | | GENES. | | |
| M236 C2 | H-X16064 | translationally-controlled tumor | 19.03 | 35 |
| | | protein | | |
| M512 B5 | H-X16323 | Hepatocyte growth factor | 80.19 | 100.0 kDa |
| | | (hepapoietin A) | | |
| M315 C3 | H-X16461 | cell division cycle 2, G1 to S and | 32.78 | 40 |
| | | G2 to M [CDC2] | | |
| M297 G2 | H-X16832 | cathepsin H | 36.96 | 45.0 kDa |
| M271 B1 | H-X16983 | integrin, alpha 4 (CD49D, alpha 4 | 114.29 | 114 |
| | | subunit of VLA-4 receptor) | | |
| | | [ITGA4], IMPORTANT FOR | | |
| | | CELL-CELL ADHESION | | |
| | | FUNCTION. | | |
| M270 A7 | H-X17025 | plasminogen activator-inducible | 25.19 | 34 |
| | | c54, Human homolog of yeast IPP | | |
| | | isomerase | | |
| M302 C3 | H-X17042 | proteoglycan 1, secretory granule | 17.49 | 26 |
| B1 | H-X17206 | ribosomal protein S2 | 24.42 | 45 |
| B4 | H-X17254 | Transcription factor Eryf1 | 45.54 | 53 |
| M311 H2 | H-X17610 | beta-1-glycoprotein, pregnancy- | 46.97 | 48.0 kDa |
| | | specific (GB: X17610) | | |
| M315 D1 | H-X17644 | G1 to S phase transition protein | 55 | 55 |
| | | (GST1) | | |
| M340 G1 | H-X51415 | lipase, hormone-sensitive [LIPE] | 84.59 | 98.0 kDa |
| M464 A7 | H-X51688 | Cyclin A | 47.63 | 47.0 kDa |
| M313 G1 | H-X51745 | major histocompatibility complex, | 40.26 | 50 |
| | | class I, A | | |
| M297 A2 | H-X51804 | putative receptor protein PMI | 21.23 | 30 |
| D4 | H-X51952 | Human UCP gene for uncoupling | 33.88 | 37 |
| | | protein exons 1 and 2 | | |
| M300 B1 | H-X52011 | muscle determining factor | 26.73 | 39 |
| M419 G1 | H-X52479 | “protein kinase c, alpha type” | 82.28 | 85.0 kDa |
| A2 | H-X52486 | Uracil-DNA glycosylase | 35.97 | 36 |
| E3 | H-X52520 | Tyrosine aminotransferase | 50.05 | 58 |
| B1 | H-X52638 | 6-phosphofructo-2- | 51.92 | 47 |
| | | kinase/fructose-2,6- | | |
| | | bisphosphatase | | |
| M509 C4 | H-X52730 | Human gene for | 31.13 | 35.0 kDa |
| | | phenylethanolamine N-methylase | | |
| | | (PNMT) (EC 2.1.1.28) | | |
| M235 C5 | H-X52839 | ribosomal protein L17 | 15.51 | 18 |
| M426 C2 | H-X52943 | Human mRNA for ATF-a | 53.24 | 64.0 kDa |
| | | transcription factor | | |
| M266 G5 | H-X53777 | ribosomal protein L23 | 20.35 | 31 |
| B4 | H-X53961 | Lactotransferrin | 78.32 | 78 |
| M462 C6 | H-X54150 | Fc fragment of IgA, receptor for | 31.68 | 37.0 kDa |
| M302 A6 | H-X54304 | myosin, light polypeptide 2, | 18.92 | 32.0 kDa |
| | | regulatory | | |
| M311 G2 | H-X54802 | cytochrome-c oxidase, IV subunit | 18.7 | 23.0 kDa |
| M270 H3 | H-X54871 | guanine nucleotide-binding | 23.76 | 33.0 kDa |
| | | protein Rab5B, ras-oncogene | | |
| | | related [RAB5B], PROTEIN | | |
| | | TRANSPORT. PROBABLY | | |
| | | INVOLVED IN VESICULAR | | |
| | | TRAFFIC (BYSIMILARITY): | | |
| M313 B6 | H-X54936 | placenta growth factor [PLGF*] | 16.5 | 22.0 kDa |
| M496 B2 | H-X55079 | Human lysosomal alpha- | 104.83 | 98.0 kDa |
| | | glucosidase gene exon 1 | | |
| D1 | H-X55330 | Aspartylglucosaminidase | 38.17 | 36 |
| E1 | H-X55448 | H. sapiens G6PD gene for | 25.41 | 30 |
| | | glucose-6-phosphate | | |
| | | dehydrogenase | | |
| M421 G6 | H-X56253 | Human MPR46 gene for 46 kd | 30.58 | 52.0 kDa |
| | | mannose 6-phosphate receptor | | |
| 169-89 | H-X56468 | 14-3-3 protein tau | 34 | 27.02 |
| M300 B4 | H-X56549 | fatty-acid-binding protein, muscle | 14.74 | 17 |
| M298 D2 | H-X56740 | guanine nucleotide-binding | 23.87 | 31.0 kDa |
| | | protein rab11 [RAB11*] | | |
| M266 E5 | H-X56932 | highly basic protein, 23 kDa | 22.44 | 30.0 kDa |
| M318 G1 | H-X57025 | insulin-like growth factor I | 16.94 | 18 |
| M305 F5 | H-X57348 | protein kinase C inhibitor | 27.39 | 35.0 kDa |
| M236 D6 | H-X57351 | interferon-induced protein 1-8D | 14.63 | 24 |
| H3 | H-X57352 | interferon-induced protein 1-8U | 14.74 | 38 |
| M305 B6 | H-X58079 | S-100 protein, alpha chain | 10.45 | 11 |
| E6 | H-X59131 | H. sapiens D13S106 mRNA for a | 34.76 | 50 |
| | | highly charged amino acid | | |
| | | sequene | | |
| M248 H5 | H-X59268 | transcription factor IIB [TCF2B*] | 34.87 | 49 |
| E2 | H-X59357 | Epstein-Barr virus small RNA- | 14.19 | 36 |
| | | associated protein | | |
| M236 D4 | H-X59417 | macropain, iota subunit, THE | 27.17 | 36 |
| | | INTERACTION OF CALPONIN | | |
| | | WITH ACTIN INHIBITS | | |
| | | ACTOMYOSIN MG-ATPASE | | |
| | | ACTIVITY | | |
| M271 H4 | H-X59618 | ribonucleotide reductase, small | 42.9 | 46 |
| | | subunit | | |
| M250 G3 | H-X59710 | CAAT-box DNA-binding protein, | 22.66 | 34 |
| | | subunit B, CCAAT-BINDING | | |
| | | TRANSCRIPTION FACTOR | | |
| | | SUBUNIT A [Homo sapiens] | | |
| M423 E2 | H-X59711 | Nuclear transcription factor Y, | 38.28 | 48.0 kDa |
| | | alpha | | |
| M271 C7 | H-X59798 | Cyclin D1 (PRAD1; parathyroid | 32.56 | 40.0 kDa |
| | | adenomatosis 1). ESSENTIAL | | |
| | | FOR THE CONTROL OF THE | | |
| | | CELL CYCLE AT THE G1/S | | |
| | | (START) TRANSITION. | | |
| M270 H5 | H-X59834 | calmodulin | 41.14 | 53.0 kDa |
| M416 D5 | H-X59871 | Transcription factor 7 (T-cell | 29.59 | 36.0 kDa |
| | | specific) | | |
| M485 D6 | H-X60036 | Phosphate carrier, mitochondrial | 39.82 | 37.0 kDa |
| M250 D4 | H-X60489 | translation elongation factor 1, | 24.86 | 33.0 kDa |
| | | beta | | |
| F5 | H-X60592 | Human CDw40 mRNA for nerve | 30.58 | 46 |
| | | growth factor receptor-related B- | | |
| | | lymphocyte activation molecule | | |
| M312 F3 | H-X61587 | ras-related rhoG | 21.12 | 21.0 kDa |
| F9 | H-X61622 | cyclin-dependent kinase 2 | 32.89 | 56 |
| | | [CDK2] | | |
| M313 E3 | H-X61970 | macropain, zeta subunit | 26.62 | 35.0 kDa |
| M428 D1 | H-X62055 | tyrosine phosphatase, non- | 65.78 | 66.0 kDa |
| | | receptor type 6 | | |
| M248 C4 | H-X62534 | high mobility group protein 2, | 23.1 | 37 |
| | | BINDS PREFERENTIALLY | | |
| | | SINGLE-STRANDED DNA | | |
| | | AND UNWINDS DOUBLE | | |
| | | STRANDED DNA. | | |
| M305 F3 | H-X62753 | folate-binding protein | 28.38 | 36 |
| M476 G2 | H-X63468 | H. sapiens mRNA for transcription | 48.4 | 53.0 kDa |
| | | factor TFIIE alpha | | |
| G6 | H-X63469 | General transcription factor TFIIE | 32.12 | 56 |
| | | beta subunit, 34 kD | | |
| G4 | H-X63522 | H. sapiens mRNA DAUD16 for | 58.74 | 54 |
| | | retinoic acid X receptor b | | |
| M316 G2 | H-X63526 | translation elongation factor 1, | 48.18 | 52.0 kDa |
| | | gamma | | |
| M305 C5 | H-X63527 | ribosomal protein L19 | 21.67 | 33 |
| E2 | H-X63629 | Cadherin 3 (P-cadherin) | 91.3 | 110 |
| D4 | H-X64037-2 | General transcription factor IIF, | 56.98 | 64 |
| | | polypeptide 1 (74 kD subunit) | | |
| M302 C6 | H-X64559 | tetranectin | 22.33 | 32.0 kDa |
| M271 H1 | H-X64728 | choroideremia-like [CHML], | 72.27 | 98 |
| | | H. sapiens CHML mRNA | | |
| M270 E1 | H-X64810 | proprotein convertase | 82.94 | 90 |
| | | subtilisin/kexin type 1 [PCSK1], | | |
| | | INVOLVED IN PROCESSING | | |
| | | OF HORMONE AND OTHER | | |
| | | PROTEIN PRECURSORS | | |
| M311 F4 | H-X64877 | complement factor H-related | 29.81 | 36.0 kDa |
| | | protein | | |
| M388 D1 | H-X65293 | protein kinase C, epsilon | 81.18 | 96.0 kDa |
| | | [PRKCE] | | |
| B5 | H-X65873 | kinesin, heavy polypeptide | 106.04 | 34 |
| F4 | H-X66079 | Spi-B transcription factor (Spi- | 28.93 | 54 |
| | | 1/PU.1 related) | | |
| F3 | H-X66114 | 2-oxoglutarate carrier protein | 0 | 37 |
| | | [OGMT*] | | |
| M305 C6 | H-X66141 | myosin, light polypeptide 2, | 18.37 | 31 |
| | | regulatory, ventricular | | |
| M419 H1 | H-X66357 | cell division protein kinase 3 | 33.620 | 44.0 kDa |
| 166-13 | H-X66358 | serine/threonine-protein kinase | 45 | 39.45 |
| | | KKIALRE | | |
| 166-25 | H-X66360 | serine/threonine-protein kinase | 60 | 57.60 |
| | | PCTAIRE-2 | | |
| M419 A2 | H-X66363 | serine/threonine-protein kinase | 54.600 | 64.0 kDa |
| | | PCTAIRE-1 | | |
| 166-37 | H-X66364 | H. sapiens mRNA PSSALRE for | 38 | 32.19 |
| | | serine/threonine protein kinase | | |
| M419 B2 | H-X66365 | cell division protein kinase 6 | 35.900 | 46.0 kDa |
| H3 | H-X66839 | H. sapiens MaTu MN mRNA for | 50.6 | 54 |
| | | p54/58N protein | | |
| M266 G3 | H-X67325 | interferon, alpha-inducible gene | 13.53 | 13 |
| | | p27 | | |
| M462 H7 | H-X67594 | Melanocortin 1 receptor (alpha | 34.98 | 44.0 kDa |
| | | melanocyte stimulating hormone | | |
| | | receptor) | | |
| M236 C5 | H-X67951 | Proliferation-associated gene A | 22 | 34 |
| | | (natural killer-enhancing factor | | |
| | | A), PAGA | | |
| H3 | H-X68486 | Adenosine receptor A2 | 45.43 | 45 |
| M429 E3 | H-X68561 | Sp4 transcription factor | 86.35 | 86.0 kDa |
| M430 F2 | H-X69151 | ATP synthase, H+ transporting, | 42.13 | 58.0 kDa |
| | | subunit C, vacuolar | | |
| M236 C3 | H-X69392 | ribosomal protein L26 | 16.06 | 29 |
| B3 | H-X69532 | H. sapiens gene for inter-alpha- | 100.32 | 98 |
| | | trypsin inhibitor heavy chain H1, | | |
| | | exons 1-3 | | |
| M236 F5 | H-X69654 | ribosomal protein S26 | 12.76 | 18 |
| M421 C8 | H-X70218 | Protein phosphatase 4 (formerly | 33.88 | |
| | | X), catalytic subunit | | |
| M266 H5 | H-X70848 | protein phosphatase 1, alpha | 36.41 | 37 |
| | | catalytic subunit | | |
| E1 | H-X70940 | Eukaryotic translation elongation | 51.04 | 60 |
| | | factor 1 alpha 2 | | |
| M270 F1 | H-X72215 | [PIT1], POU domain, class 1, | 32.12 | 40.0 kDa |
| | | transcription factor 1 (Pit1, | | |
| | | growth hormone factor 1) | | |
| M271 A7 | H-X72760 | Laminin, beta 2 (laminin S), S- | 67.87 | 75.0 kDa |
| | | LAMININ IS A LAMININ-LIKE | | |
| | | ADHESIVE PROTEIN | | |
| | | CONCENTRATED IN THE | | |
| | | SYNAPTIC CLEFT OF THE | | |
| | | NEUROMUSCULAR | | |
| | | JUNCTION. | | |
| M235 B1 | H-X72841 | Human retinoblastoma-binding | 46.86 | 52.0 kDa |
| | | protein (RbAp46) mRNA, | | |
| | | complete cds, IEF 7442 | | |
| | | (GB: X72841) | | |
| 217-25 | H-X73428 | DNA-binding protein inhibitor | 20 | 17.08 |
| | | ID-3 | | |
| M305 B5 | H-X73459 | signal recognition particle, | 15.07 | 20 |
| | | subunit 14 | | |
| M250 D6 | H-X73460 | ribosomal protein L3, isoform 2, | 44.44 | 50.0 kDa |
| | | COMPONENT OF THE LARGE | | |
| | | SUBUNIT OF CYTOPLASMIC | | |
| | | RIBOSOMES | | |
| M462 D8 | H-X74008 | Protein phosphatase 1, catalytic | 35.64 | 46.0 kDa |
| | | subunit, gamma isoform | | |
| M266 G2 | H-X74104 | Signal sequence receptor, beta; | 20.24 | 27 |
| | | translocon-associated protein, | | |
| | | beta subunit | | |
| M266 E7 | H-X74262 | retinoblastoma binding protein | 46.86 | 50.0 kDa |
| | | RbAp48 | | |
| H1 | H-X74330 | DNA primase polypeptide 1 | 46.31 | 51 |
| | | (49 kD) | | |
| M313 F3 | H-X74570 | gal beta (1-3/1-4) GlcNAc alpha- | 36.3 | 46.0 kDa |
| | | 2,3 sialyltransferase (GB: X74570) | | |
| M429 H3 | H-X74764 | H. sapiens mRNA for receptor | 94.120 | 98.0 kDa |
| | | protein tyrosine kinase | | |
| M271 E6 | H-X75042 | V-rel avian reticuloendotheliosis | 68.2 | 88 |
| | | viral oncogene homolog | | |
| M305 G2 | H-X75252 | phosphatidylethanolamine- | 20.68 | 30 |
| | | binding protein | | |
| M302 G1 | H-X75593 | guanine nucleotide-binding | 22.44 | 32.0 kDa |
| | | protein rab13 | | |
| 166-49 | H-X75958 | H. sapiens trkB mRNA for | 55 | 52.54 |
| | | protein-tyrosine kinase | | |
| C4 | H-X76013 | H. sapiens QRSHs mRNA for | 85.36 | 85 |
| | | glutaminyl-tRNA synthetase | | |
| A2 | H-X76029 | H. sapiens mRNA for neuromedin U | 19.25 | 20 |
| M305 D5 | H-X76228 | ATP synthase, H+transporting, | 24.97 | 36 |
| | | subunit E, vacuolar | | |
| M298 F6 | H-X76648 | glutaredoxin | 11.77 | 11.0 kDa |
| M311 A4 | H-X76717 | metallothionein 11 | 6.82 | 14 |
| C4 | H-X77533 | H. sapiens mRNA for activin type | 56.43 | 61 |
| | | II receptor | | |
| H2 | H-X77548 | H. sapiens cDNA for RFG | 67.65 | 67 |
| 169-41 | H-X77743 | H. sapiens CDK activating kinase | 45 | 38.13 |
| | | mRNA | | |
| A4 | H-X77909 | H. sapiens IKBL mRNA | 42.02 | 52 |
| M305 C1 | H-X78136 | heterogeneous nuclear | 40.26 | 40.0 kDa |
| | | ribonucleoprotein E2 | | |
| M306 G2 | H-X78416 | casein, alpha [CSN1] | 20.46 | 33 |
| M271 C2 | H-X78678 | ketohexokinase (fructokinase) | 32.89 | 39 |
| | | [KHK], H. sapiens KHK mRNA | | |
| | | for ketohexokinase, clone | | |
| | | pHKHK3a | | |
| M305 D4 | H-X79193 | cyclin-dependent kinase 7 | 38.17 | 35 |
| | | (homolog of Xenopus MO15 cdk- | | |
| | | activating kinase) [CDK7] | | |
| M431 F2 | H-X79389 | glutathione S-transferase T1 | 26.51 | 34.0 kDa |
| M298 C6 | H-X79537 | glycogenin | 30.8 | 34.0 kDa |
| M440 C1 | H-X79865 | H. sapiens Mrp17 mRNA | 21.89 | 31.0 kDa |
| M298 F5 | H-X80229 | protein kinase PKN | 52.8 | 64.0 kDa |
| 167-39 | H-X80230 | H. sapiens mRNA (clone C-2k) | 42 | 40.99 |
| | | mRNA for serine/threonine | | |
| | | protein kinase | | |
| 217-49 | H-X80343 | H. sapiens p35 mRNA for | 40 | 33.84 |
| | | regulatory subunit of cdk5 kinase | | |
| M270 D7 | H-X80695 | cytochrome oxidase-assembly | 47.96 | 50 |
| | | protein, OXA1, H. sapiens | | |
| | | OXA1Hs mRNA | | |
| M266 B5 | H-X80909 | nascent polypeptide-associate | 23.76 | 37.0 kDa |
| | | complex, alpha | | |
| M416 D9 | H-X80910 | Protein phosphatase 1, catalytic | 36.08 | 45.0 kDa |
| | | subunit, beta isoform | | |
| E2 | H-X81198 | Archain | 52.03 | 63 |
| 169-6 | H-X81817 | H. sapiens BAP31 mRNA | 32 | 27.13 |
| E4 | H-X82018 | H. sapiens mRNA for ZID protein | 46.75 | 57 |
| M313 D7 | H-X82456 | MLN50 | 28.82 | 33 |
| A2 | H-X82629 | H. sapiens mRNA for Mox-2 | 33.44 | 42 |
| M236 D1 | H-X83006 | lipocalin, neutrophil gelatinase | 21.89 | 34.0 kDa |
| | | associated | | |
| 166-40 | H-X83107 | H. sapiens Bmx mRNA for | 75 | 74.32 |
| | | cytoplasmic tyrosine kinase | | |
| E3 | H-X83425 | H. sapiens LU gene for Lutheran | 69.19 | 59 |
| | | blood group glycoprotein | | |
| C6 | H-X83703 | H. sapiens mRNA for cytokine | 35.2 | 54 |
| | | inducible nuclear protein | | |
| M416 H2 | H-X83928 | H. sapiens mRNA for transcription | 23.32 | 33.0 kDa |
| | | factor TFIID subunit TAFII28 | | |
| 166-17 | H-X85106 | H. sapiens mRNA for ribosomal | 90 | 80.70 |
| | | S6 kinase | | |
| 166-39 | H-X85337 | H. sapiens mRNA for myosin light | 110 | 109.0 |
| | | chain kinase | | |
| D2 | H-X85750 | H. sapiens mRNA for transcript | 26.29 | 30 |
| | | associated with monocyte to | | |
| | | macrophage differentiation | | |
| M266 E6 | H-X87176 | 17-beta-hydroxysteroid | 81.07 | 65 |
| | | dehydrogenase, type 4 | | |
| M297 F2 | H-X87689 | CLCP | 23.21 | 33.0 kDa |
| M300 A2 | H-X87843 | cyclin H assembly factor | 34.1 | 47 |
| M271 E3 | H-X89750 | homeotic protein, TGIF, | 30.03 | 32.0 kDa |
| | | H. sapiens mRNA for TGIF | | |
| | | protein | | |
| M235 G1 | H-X90529 | guanine nucleotide-binding | 34.54 | 40 |
| | | protein ragA [RAGA] | | |
| M302 E6 | H-X90583 | translocon-associated protein, | 19.14 | 28.0 kDa |
| | | delta | | |
| M306 G1 | H-X90872 | gp2512 | 23.65 | 33 |
| M416 D2 | H-X91504 | Transcription factor COUP 2 | 22.22 | 32.0 kDa |
| | | (a.k.a. ARP1) | | |
| M250 B3 | H-X92098 | transmembrane protein rnp24 | 22.22 | 30 |
| M271 G7 | H-X92106 | bleomycin hydrolase. | 50.16 | 55.0 kDa |
| | | PROTECTING NORMAL AND | | |
| | | MALIGNANT CELLS FROM | | |
| | | BLM TOXICITY. | | |
| F3 | H-X92715 | Zinc finger protein 74 (Cos52) | 63.03 | 47 |
| M270 H6 | H-X92720 | H. sapiens mRNA for | 70.51 | 71 |
| | | phosphoenolpyruvate | | |
| | | carboxykinase | | |
| H5 | H-X92762 | H. sapiens mRNA for tafazzins | 32.23 | 37 |
| | | protein | | |
| M298 D3 | H-X93036 | MAT-8 | 9.68 | 16.0 kDa |
| M476 A5 | H-X93595 | H. sapiens mRNA for NK receptor | 50.16 | 56.0 kDa |
| | | (clone 17.1C) | | |
| M417 D2 | H-X93920 | protein tyrosine phosphatase | 41.980 | 48.0 kDa |
| | | foreskin | | |
| A5 | H-X95592 | H. sapiens mRNA for C1D protein | 15.62 | 28 |
| M298 B4 | H-X95648 | translation initiation factor 2B, | 33.66 | 34.0 kDa |
| | | alpha subunit | | |
| F3 | H-X95735 | H. sapiens mRNA for zyxin 2 | 63.03 | 72 |
| M386 B1 | H-X96752 | L-3-hydroxyacyl-CoA | 34.65 | 45.0 kDa |
| | | dehydrogenase, SCHAD gene | | |
| M422 B6 | H-X97229 | H. sapiens mRNA for NK | 41.58 | 48.0 kDa |
| | | receptor, clone library 15.212 | | |
| B3 | H-X98173 | H. sapiens mRNA for MACH- | 51.15 | 51 |
| | | alpha-2 protein | | |
| 166-14 | H-X99325 | H. sapiens mRNA for Ste20-like | 55 | 46.93 |
| | | kinase | | |
| C4 | H-X99459 | H. sapiens mRNA for sigma 3B | 21.34 | 30 |
| | | protein | | |
| M424 C4 | H-Y00291 | Human hap mRNA encoding a | 49.39 | 59.0 kDa |
| | | DNA-binding hormone receptor | | |
| M386 H1 | H-Y00345 | polyadenylate-binding protein | 69.74 | 70.0 kDa |
| M469 A2 | H-Y00630 | Plasminogen activator inhibitor, | 45.76 | 46.0 kDa |
| | | type II (arginine-serpin) | | |
| M305 E1 | H-Y00711 | lactate dehydrogenase B | 36.85 | 38.0 kDa |
| H2 | H-Y00764 | ubiquinol/cytochrome c reductase | 10.12 | 33 |
| | | hinge protein | | |
| F5 | H-Y07848 | H. sapiens EWS, gar22, rrp22 and | 36.3 | 50 |
| | | bam22 genes | | |
| M305 G6 | H-Z11559 | iron-responsive element binding | 97.9 | 98 |
| | | protein 1 [IREB1] | | |
| M250 F3 | H-Z11566 | Pr22 protein , STATHMIN | 16.5 | 22.0 kDa |
| | | [Homo sapiens], SERVES AS | | |
| | | RELAY (VIA | | |
| | | PHOSPHORYLATION) FOR | | |
| | | DIVERSE SECOND | | |
| | | MESSENGER PATHWAYS | | |
| 169-73 | H-Z11695 | H. sapiens 40 kDa protein kinase | 50 | 38.35 |
| | | related to rat ERK2 | | |
| M475 C8 | H-Z11737 | Flavin-containing | 61.49 | 70.0 kDa |
| | | monooxygenase 4 | | |
| C1 | H-Z11898 | Octamer binding protein 3 | 39.71 | 50 |
| M266 H4 | H-Z12830 | SSR, alpha subunit | 31.57 | 42.0 kDa |
| A3 | H-Z14000 | Ring finger protein 1 | 41.58 | 50 |
| M300 E1 | H-Z14978 | actin-related protein | 41.47 | 49 |
| G1 | H-Z19002 | H. sapiens of PLZF gene encoding | 74.14 | 84 |
| | | kruppel-like zinc finger protein | | |
| H1 | H-Z21966 | POU homeobox protein | 33.22 | 43 |
| M248 G3 | H-Z23139 | CLASS II | 29.04 | 34 |
| | | HISTOCOMPATIBILITY | | |
| | | ANTIGEN, M BETA CHAIN | | |
| | | PRECURSOR [Homo sapiens] | | |
| D3 | H-Z26876 | ribosomal protein L38 | 7.81 | 35 |
| F2 | H-Z28339 | H. sapiens mRNA for delta 4-3- | 35.97 | 43 |
| | | oxosteroid 5 beta-reductase | | |
| M298 B3 | H-Z28407 | ribosomal protein L8 | 28.38 | 39.0 kDa |
| M313 C3 | H-Z29330 | ubiquitin-conjugating enzyme | 20.24 | 34 |
| | | UbcH2, 23 kDa | | |
| M271 F3 | H-Z29677 | guanine nucleotide-binding | 20.35 | 28.0 kDa |
| | | protein, ras-related | | |
| M465 C2 | H-Z30425 | H. sapiens mRNA for orphan | 38.39 | 34.0 kDa |
| | | nuclear hormone receptor | | |
| M302 F5 | H-Z31357 | cysteine dioxygenase | 22.11 | 31.0 kDa |
| M340 C1 | H-Z31695 | inositol polyphosphate 5- | 40.04 | 49.0 kDa |
| | | phosphatase, 43 kDa | | |
| E3 | H-Z32564-2 | H. sapiens FRGAMMA mRNA | 26.84 | 36 |
| | | (819 bp) for folate receptor | | |
| M236 H1 | H-Z35227 | small G protein, TTF, RAS- | 21.12 | 30.0 kDa |
| | | RELATED PROTEIN RAC1 | | |
| A10 | H-Z35491 | H. sapiens mRNA for novel | 30.25 | 60 |
| | | glucocorticoid receptor-associated | | |
| | | protein | | |
| M440 G5 | H-Z37986 | H. sapiens mRNA for | 25.41 | 28.0 kDa |
| | | phenylalkylamine binding protein | | |
| M297 E2 | H-Z47087 | cyclin A/cyclin-dependent kinase | 18.04 | 30.0 kDa |
| | | 2-associated p19 | | |
| F1 | H-Z48051 | H. sapiens gene for myelin | 27.28 | 31 |
| | | oligodendrocyte glycoprotein | | |
| | | (MOG) | | |
| A2 | H-Z48475 | Glucokinase regulator | 68.86 | 70 |
| M302 E4 | H-Z48570 | sperm zona pellucida-binding | 16.72 | 24 |
| | | protein | | |
| M266 A2 | H-Z68907 | Human clone ID 193225 NAD | 43.34 | 45.0 kDa |
| | | (H)-specific isocitrate | | |
| | | dehydrogenase gamma subunit | | |
| | | mRNA, alternatively spliced, | | |
| | | partial cds | | |
| G1 | H-Z83850 | Human DNA sequence from PAC | 45.76 | 60 |
| | | 82J11 and cosmid U134E6 on | | |
| | | chromosome Xq22. Contains NIK | | |
| | | like and Thyroxin-binding | | |
| | | globulin precursor (T4-binding | | |
| | | globulin, TBG) genes, ESTs and | | |
| | | STSs | | |
| H4 | H-Z97171 | Homo sapiens GLC1A (trabecular | 55.55 | 55 |
| | | meshwork induced glucocortcoid | | |
| | | response) gene, exon I, joined | | |
| | | CDS | | |
| M421 D5 | H-Z97632 | Human DNA sequence from PAC | 28.49 | 38.0 kDa |
| | | 196E23 on chromosome Xq26.1- | | |
| | | 27.2. Contains the TAT-SF1 | | |
| | | (HIV-1 transcriptional elongation | | |
| | | factor TAT cofactor TAT-SF1) | | |
| | | gene, the BRS3 (Bombesin | | |
| | | Receptor subtype-3 (Uterine | | |
| | | Bombesin Receptor, BRS-3) | | |
| | | gene, an unknown gene coding | | |
| | | for two isoforms, a predicted CpG | | |
| | | island, ESTs and STSs | | |

### Example 3

### Construction of Expression Plasmids

The following example illustrates the construction of the expression vectors used in the Examples above. Similar modifications can be made in other vectors for use in creating libraries of expressible gene sequences.

The vector pcDNA3.1/V5-His was obtained from Invitrogen (cat #V810-20) and modified slightly so that it carried an gene sequence for Zeocin™ resistance and lacked the multiple cloning site. A 100 μg aliquot was suspended in 200 μl medical irrigation (MI) water. A 5 μi aliquot was saved for gel analysis. The remainder was transfered to a 1.7 ml eppendorf tube. The vector was digested with HindIII (400 U) using Promega Buffer E (final volume=400 pi). The reaction ran 3 hours at 37° C. An aliquot was checked for completeness of digestion by running on an 0.8% agarose gele in 1×TAE, and visualizing with ethidium bromide.

The digested vector was treated with 200 μl phenolchloroform (pH 7.5) according to standard procedures, and the DNA precipitated from the aqueous phase using 1/10 volume 3M NaOAc and 2 volumes 100% EtOH at room temperature, followed by washing with 80% EtOH. The pellet was resuspended in 100 μl MI water.

Two oligonucleotides were added to the resuspended DNA (Topo-H (40 pg) 5′-(P)AGCTCGCCCTTATTCCGATAGTG (SEQ. ID. NO.: 3), Topo-4 (12 μg) 5′-(P)AGGGCG (SEQ. ID. NO.: 4)), plus 17 μl 10×Promega T4 Ligase buffer. The tube was placed on ice and the volume increased to 170 μl with MI water. The oligos were ligated to the vector using 20U Promega T4 DNA ligase, incubated at 12° C. overnight.

The vector was treated with 100 μl phenol/chloroform and the aqueous phase precipitated as described above. The pelleted DNA was resuspended in 150 μl of steril water the redigested with HindIII (17 μl Promega Buffer E, 200 U HindIII −37° C., 1 hour). The redigested DNA was re-extracted with phenol/chloroform and precipitated with 1/10 volume 3M NaOAc and 7/10 volume isopropanol, then washed with 80% EtOH.

The pelleted DNA was resuspended in 82 μl TE buffer (10 mM Tris, pH 8.0, 1 mM EDTA, pH 8.0). A 2 μl aliquot was used to check the foregoing procedure using agorose gel electrophoresis as described above. The remaining 80 μl was transfered to a Falcon tube and mixed with 16 μg Topo-5 oligonucleotide (5′-(P)CAACACTATCGGAATA (SEQ ID NO: 5). To this mixture was added 190 μl NEB Restriction Buffer #1 (room temperature). The total reaction mixture was adjusted to 1.9 mls with MI water. Vaccinia Topoisomerase I enzyme was added (80 μg) and the reaction tube placed in a 37° C. water bath for 15 minutes.

After 15 minutes, 200 μl of room temperature Topo-10×stop buffer was added (100 mM Tris 7.4, 110 mM EDTA, bromophenol blue). The entire volume was loaded onto an agarose gell (1.2 gr agarose/130 mls 1×TAE) and run at 70 volts until the bromophenol blue dye had run down about ½ in (volume in the loading well was kept constant by the addition of 1×TE). The voltage was reversed for 90 seconds. The contents of the loading well were transfered to a 15 ml Falcon tube and placed on ice. 2 mls of cold Topo-2×Wash Buffer (60 mM Tris 7.4, 1 mM EDTA, 4 mM dithiothreitol (DTT), 200 μg/ml bovine serum albumin (BSA)) was added and the volume then adjusted to 4 mls with cold Topo-1×Enzyme Dilution Buffer (50% glycerol, 50 mM Tris 7.4, 1 mM EDTA, 2 mM DTT, 0.1% Triton X-100, 100 μg.ml BSA) plus 4 mls Topo-Glycerol mix (90% glycerol, 10% 50 mM TE pH 7.4, 0.1% Triton X-100) and stored until needed.

A similar procedure was used to make Topo-adapted pYES2 (Invitrogen cat #V825-20).

While the foregoing has been presented with reference to particular embodiments of the invention, it will be appreciated by those skilled in the art that changes in these embodiments may be made without departing from the principles and spirit of the invention, the scope of which is defined by the appended claims.

That which is claimed is:

1. A nucleic acid construct comprising 1) a gene sequence encoding a protein listed in Table 1 or an ORF listed in Table 2 and 2) an expression vector.

2. A nucleic acid construct according to claim 1 wherein the expression vector comprises one or more elements selected from: a promoter-enhancer sequence, a selection marker sequence, an origin of replication, an epitope-tag encoding sequence or an affinity purification-tag encoding sequence.

3. A nucleic acid construct according to claim 2 wherein the promoter-enhancer sequence is the T7 promoter, gall promoter, metallothionein promoter, AraC promoter, or CMV promoter-enhancer.

4. A nucleic acid construct according to claim 2 wherein the selection marker sequence encodes an antibiotic resistance gene.

5. A nucleic acid construct according to claim 2 wherein the epitope-tag sequence encodes V5, the peptide Phe-His-His-Thr-Thr, hemaglutinin, or glutathione-S-transferase.

6. A nucleic acid construct according to claim 2 wherein the affinity purification-tag sequence encodes a polyamino acid sequence or a polypeptide.

7. A nucleic acid construct according to claim 6 wherein said polyamino acid sequence is polyhistidine.

8. A nucleic acid construct according to claim 6 wherein said polypeptide is chitin binding domain or glutathione-S-transferase.

9. A nucleic acid construct according to claim 6 wherein said polypeptide encoding sequence includes an intein encoding sequence.

10. A nucleic acid construct according to claim 1 wherein the expression vector is a eukaryotic expression vector or a prokaryotic expression vector.

11. A nucleic acid construct according to claim 10 wherein the eukaryotic expression vector is pYES2/GS, pMT, pIND, or pcDNA3.1/GS.

12. A nucleic acid construct according to claim 1 wherein the protein is selected from the group of proteins listed as number 1 through number 20 in Table 1.

13. A nucleic acid construct according to claim 1 wherein the protein is selected from the group of proteins listed as number 21 through number 40 in Table 1.

14. A nucleic acid construct according to claim 1 wherein the protein is selected from the group of proteins listed as number 41 through number 60 in Table 1.

15. A nucleic acid construct according to claim 1 wherein the protein selected from the group of proteins listed as number 61 through number 80 in Table 1.

16. A nucleic acid construct according to claim 1 wherein the protein is selected from the group of proteins listed as number 81 through number 100 in Table 1.

17. A nucleic acid construct according to claim 1 wherein the protein is selected from the group of proteins listed as number 101 through number 118 in Table 1.

18. A nucleic acid construct according to claim 1 wherein the construct comprises an ORF listed in Table 2.

19. A recombinant cell comprising a nucleic acid construct of claim 1.

20. A recombinant cell of claim 19 wherein the cell is a non-adherent cell.

21. A recombinant cell of claim 20 wherein the non-adherent cell is a bacterial cell, a yeast cell, plant cell, an insect cell or a mammalian cell.

22. A recombinant cell of claim 21 wherein the mammalian cell is CHO or 32D.

23. A method of producing recombinant protein, said method comprising:
(a) growing recombinant cells comprising a nucleic acid construct of claim 1 under suitable growth conditions; and
(b) isolating the recombinant protein expressed thereby.

24. A method according to claim 23 wherein the nucleic acid construct comprises an epitope-tag encoding sequence and the isolation step utilizes an antibody specific for said epitope-tag.

25. A method according to claim 24 wherein the nucleic acid construct comprises a polyamino acid encoding sequence and the isolation step utilizes a resin comprising a polyamino acid binding substance.

26. A method according to claim 23 wherein the nucleic acid construct comprises a polypeptide encoding sequence and the isolation step utilizes a resin comprising a polypeptide binding substance.

27. A method according to claim 25 wherein the polyamino acid is polyhistidine and the polyamino binding resin is nickel-charged agarose resin.

28. A method according to claim 26 wherein the polypeptide is chitin binding domain and the resin comprises chitin-Sepharose.

29. A kit comprising a plurality of expression constructs, wherein each expression construct comprises a gene sequence encoding a protein listed in Table 1 and an expression vector.

30. A kit according to claim 29 wherein the expression vector is pYES2/GS or pcDNA3.1/GS.

31. A kit comprising a plurality of recombinant cells, wherein each cell comprises a gene sequence encoding a protein listed in Table 1 and an expression vector.

32. A kit according to claim 31 wherein the expression vector is pYES2/GS or pcDNA3.1/GS and the recombinant cell is a yeast cell or a mammalian cell.

33. A kit according to claim 32 wherein the mammalian cell is a CHO cell.

34. A kit comprising a plurality of expression constructs, wherein each expression construct comprises an ORF listed in Table 2 and an expression vector.

35. A kit according to claim 34 wherein the expression vector is pYES2/GS or pcDNA3.1/GS.

36. A kit comprising a plurality of recombinant cells, wherein each cell comprises an ORF listed in Table 2 and an expression vector.

37. A kit according to claim 36 wherein the expression vector is pYES2/GS or pcDNA3.1/GS and the recombinant cell is a yeast cell or a mammalian cell.

38. A kit comprising one or more of: expression construct(s) comprising a gene sequence encoding a protein listed in Table 1 and an expression vector; recombinant cells comprising an expression construct comprising a gene sequence encoding a protein listed in Table 1 and an expression vector; and an isolated protein listed in Table 1 or an antibody specific for said isolated protein.

39. A binding partner of an expressed gene product of a gene sequence listed in Table 1.

40. A binding partner of an expressed gene product of a gene sequence listed in Table, 2.


SEQUENCE LISTING (6 sequences)

SEQ ID NO: 1 - DNA, 27 bp, Artificial sequence
  Common 5′ primer
gcagtcctggaattccagctgaccacc

SEQ ID NO: 2 - DNA, 28 bp, Artificial sequence
  H6stopprevu primer
aaactcaatggtgatggtgatgatgacc

SEQ ID NO: 3 - DNA, 23 bp, Artificial sequence
  Topo-H oligonucleotide
agctcgcccttattccgatagtg

SEQ ID NO: 4 - DNA, 6 bp, Artificial sequence
  Topo-4 oligonucleotide
agggcg

SEQ ID NO: 5 - DNA, 16 bp, Artificial sequence
  Topo-5 oligonucleotide
caacactatcggaata

SEQ ID NO: 6 - protein, 5 aa, Artificial sequence
  Epitope tag
FHHTT